(19) United States
(12) Patent Application Publication
(10) Pub. No.: US 2007/0105105 A1
(43) Pub. Date: May 10, 2007

(54) Surrogate cell gene expression signatures for evaluating the physical state of a subject

(75) Inventors: Catherine Clelland, New York, NY (US); F. Carter Bancroft, Huntington, NY (US); James Clelland, New York, NY (US)

Correspondence Address: DARBY & DARBY P.C., P. O. BOX 5257, NEW YORK, NY, 10150-5257, US

(73) Assignees: Mount Sinai School of Medicine of New York University, New York, NY (US); Research Foundation for Mental Hygiene, Menands, NY (US)

(21) Appl. No.: 10/558,277

(22) PCT Filed: May 24, 2004
(86) PCT No.: PCT/US04/16365

§ 371 Date: Dec. 15, 2006

Related U.S. Application Data
(60) Provisional application No. 60/473,089, filed on May 23, 2003.

(51) Int. Cl. C12Q 1/68; C12P 19/34

(52) U.S. Cl. 435/6; 435/91.2

(57) ABSTRACT

The present invention relates to non-invasive and minimally invasive techniques for evaluating the physical state of a subject, including diagnosing a disease, disorder, or physical state of the subject, determining the prognosis of the subject, determining a subject's susceptibility for a disease, disorder, or physical state and determining, developing and monitoring treatment for the same. The invention also relates to identifying genetic alterations contributing to, or susceptibility for, development of a disease, disorder, or physical state, and for diagnosis, prognosis and treatment of the disease, disorder, or physical state.

This application claims priority from U.S. Provisional Patent Application No. 60/473,089, filed May 23, 2003, which is herein incorporated by reference in its entirety.

The research leading to this invention was supported, in part, by Grant No. 1RO3 MH62428-01 awarded by the National Institutes of Mental Health. Accordingly, the United States government may have certain rights to this invention.

### FIELD OF THE INVENTION

The present invention relates to non-invasive and minimally invasive techniques for evaluating the physical state of a subject, including diagnosing a disease, disorder, or physical state of the subject, determining the prognosis of the subject, determining a subject's susceptibility for a disease, disorder, or physical state and determining, developing and monitoring treatment for the same. The invention also relates to identifying genetic alterations contributing to, or susceptibility for, development of a disease, disorder, or physical state, and for diagnosis, prognosis and treatment of the disease, disorder, or physical state.

### BACKGROUND OF THE INVENTION

Although cancer mortality rates have decreased over the past decade, through pre-symptomatic screening programs and major improvements in cancer treatment, survival rates are still low in patients presenting with a more advanced stage cancer at the time of diagnosis. Thus, effective management of any cancer relies heavily on an early diagnosis, coupled with a need to obtain accurate information on the classification and stage of the cancer itself, and thus limitations of traditional diagnostic and prognostic techniques may currently hinder the management of cancer.

Current techniques for the screening and risk assessment of chronic disease states in general, and cancer in particular, are frequently based upon the measurement of either individual serum biomarkers, or expression of individual genes in circulating cells, such as disseminated tumor cells. In disease states for which such non-invasive tests are available they usually comprise a prerequisite to more invasive, surgical biopsy procedures.

Examples of serum biomarkers used in the clinical diagnosis of cancer include CA 125 (ovarian cancer), CA 15-3 and CA 27-29 (breast cancer), carcinoembryonic antigen, CEA (ovarian, lung, breast, pancreas, and gastrointestinal tract cancers), prostate specific antigen, PSA (prostate cancer), alpha fetoprotein, AFP (primary liver cancer or germ cell cancer), human chorionic gonadotropin, HCG (choriocarcinoma, cancers of the testis, ovary, liver, stomach, pancreas, and lung) CA 19-9 (colorectal cancer pancreatic, stomach, and bile duct cancer) neuron-specific enolase, NSE (neuroblastoma; small cell lung cancer; Wilms' tumor; melanoma; and cancers of the thyroid, kidney, testicle, and pancreas (Source: National Cancer Institute, on the Worldwide Web at nci.nih.gov)

Diagnosis of psychiatric and neurological diseases for which the molecular etiology is largely unknown, such as schizophrenia or not too well understood such as in Alzheimer's disease, still depend mainly on behavioral evaluation of patients, and no clinically proven, blood-based, tests are available to date. Individual circulating biomarkers, however, are beginning to be discovered. In Alzheimer's disease, for instance, a serum elevation of the iron transporter p97 (Kim D K, et al. Neuropsychopharmacology 2001; 25(1):84-90) or an increase in antibody-mediated brain to plasma amyloid-beta efflux (DeMattos R B, et al., Science 2002, 295:2264-2267) have been described. Furthermore, Ilani et al. have shown an increased level of D3 dopamine receptor mRNA in circulating blood lymphocytes in individuals with schizophrenia (Ilani et al. Proc Natl Acad Sci USA 2001; 98(2):625-8).

For cancer, diagnostic tests based on single circulating biomarkers possess a number of limitations, including lack of specificity and sensitivity in the diagnosis and, also a lack of prognostic information. This ultimately yields high numbers of false positive diagnoses, and consequently unnecessarily large numbers of surgical biopsies. Alternatively, in a significant number of patients malignancies evade detection due to the inherent rate of false negative test results.

There is growing evidence that individuals with a malignant disease such as breast cancer or prostate cancer, exhibit immune responses that can be detected at the level of altered gene expression in leukocytes circulating in peripheral blood. Quantitation of the mRNA transcripts in leukocytes of a number of individual genes has demonstrated associations between gene expression levels and the presence of a tumor in patients with breast and prostate cancer.

The recent development of microarray technology has permitted simultaneous measurement of the expression levels of thousands of genes, and also allowed a comparison of multiple data sets between multiple experiments. Investigators have begun to employ this technology, based upon sample cDNA probe hybridization to DNA-based microarrays, to identify and isolate genes differentially expressed among many tissues and cell lines. Microarray technology will become a global gene expression diagnostic tool (Cole et al., Nat. Genet. 1999: 21(1 Suppl):38-1; Howell S B, Mol Urol. 1999; 3(3):295-300). Already, breakthrough experiments have shown that molecular profiles, or gene expression signatures, can be deduced from microarray expression analysis of tumor samples. Researchers have used statistical algorithms to compare individual expression signatures, and then employed these comparisons to distinguish between forms of myeloid leukemia (Golub et al., Science 1999; 286(5439):531-7), and B-cell lymphoma (Alizadeh et al, Nature 2000; 403(6769):503-11). Furthermore, analysis of tumor tissue from individual patients has permitted identification of both stages and individual classes of breast cancer (Perou et al., Nature 2000; 406(6797):747-52), malignant melanoma (Bittner et al., Nature 2000; 406(6795):536-40), and prostate cancer (Dhanasekaran et al., Nature 2001; 412(6849):822-6; Luo et al., Mol Carcinog 2002; 33(1):25-35). Additionally, utilizing microarray technology van't Veer et al., have shown that the clinical status and clinical outcome of breast cancer can be predicted by gene expression analysis of tumor tissue (Nature 2002; 415(6871):530-6).

Even at this early stage in the clinical development of this technology, it is becoming clear that microarray analysis will be able to provide important diagnostic and prognostic information for many tumor types. However, although these investigations of solid tumors provide detailed information on the pathology and malignant process of the tumor, invasive surgery or biopsy is always necessary to obtain the tumor tissue studied, and although investigations are underway to determine the feasibility of minimally traumatic biopsy sampling procedures for obtaining tissue for microarray analysis, a current report documents problems such as very low yields of extracted RNA (Assersohn et al., Clin Cancer Res. 2002; 8(3):794-801).

A link between cancer and altered gene expression in the immune system has been previously documented. Tumor-induced immunosuppression allows malignant cells to evade the immune system, and some tumors are commonly found in individuals with compromised immune function. For example, Karposi's sarcoma has become a very common and highly aggressive neoplastic complication of AIDS (Ensoli & Sirianni., Crit Rev Oncog 1998; 9(2):107-24), and it has been proposed that chronic inflammation, resulting from infective and/or non-infective agents, may provide the ideal environment for the cellular development of cancer (O'Byrne & Dagleish, Br J Cancer 2001; 85(4):473-83).

Many mechanisms are thought to be involved in the altered immune response of cancer patients. These include decreased natural killer (NK) cell cytotoxicity (Kono et al., Clin Cancer Res. 1996; 2(11): 1825-8), the production in the tumor of cytokines and growth factors that have known suppressive effects on leukocyte function (e.g. interleukin 6 (IL-6), IL4 and TGF-beta1), (Oliver and Nouri., Cancer Surv. 1992; 173-204), and defective cytokine release from T-cells, such as a decrease in IL-2 (Lopez et al., Cell Immunol. 1998; 190(2):141-55).

Research to further elucidate the immune responses observed in cancer patients has mainly focused on measuring the level of genes or protein products from cells within the microenvironment of the tumor, such as IL-2 mRNA transcript levels in tumor infiltrating lymphocytes within primary human breast carcinomas (Lopez et al., 1998) and adenocarcinomas of the prostate (Elsasser-Beile et al., J. Cancer Res Clin Oncol. 1993; 119(7):430-3). However, a number of groups have also reported altered levels of the mRNA and/or protein products of individual genes in leukocyte cells within the circulating peripheral blood of cancer patients.

Veltri and colleagues have reported that IL-8 mRNA expression is up-regulated in patients with metastatic prostate cancer relative to control subjects (Veltri et al., Urology 1999; 53(1):139-47). Specifically, these investigators carried out an analysis of IL-8 mRNA levels in peripheral blood from metastatic patients and normal control subjects (pooled into one sample), employing IL-8 gene specific primers for RT-PCR experiments, and 25 cycles of amplification in the PCR. The results documented the presence of IL-8 products in all metastatic prostate cancer patient samples, while in the pool of control samples no amplification of IL-8 was observed (Veltri et al., 1999). As discussed below, the present inventors have performed similar experiments to seek to confirm these results.

Recently, a study was initiated to investigate expression levels of the mismatch repair genes, MSH2 and MLH1 in prostate cancer (Strom et al., Prostate 2001; 1; 47(4):269-75). Strom et al., performed RT-PCR analysis of leukocyte samples from 70 prostate cancer subjects (metastatic patients were excluded from this study), and 97 matched controls. Their results implied that although considerable variation occurred among patients, the mean expression levels for both genes were significantly lower in the patients than controls. Although the authors conclude that decreased expression of MLH1 is a risk factor for prostate cancer, they also state that the decreased expression of both genes may be caused by disease status, a conclusion that is consistent with this hypothesis. The present invention provides preliminary data that reproduces these results. Veltri et al., further reported that increasing concentrations of serum IL-8 protein could be positively correlated with increasing tumor burden, and that serum IL-8 levels correctly distinguished among patients classified to one of four known stages of prostate cancer (Veltri et al., 1999). Additionally, the authors reported unpublished data showing that quantitative RT-PCR analysis of IL-6 and IL-10 mRNA levels also yielded a marked difference between prostate cancer patients and control subjects, and these results have been published by Ralph et al., (U.S. Pat. No. 6,190,857). Earlier studies that have measured serum levels of IL-6 and IL-10 proteins in cancer patients support these observations. Specifically, IL-6 serum levels have been shown to provide prognostic information on prostate tumors (Nakashima et al., Cancer Res. 2000; 6(7):2702-6), and serum IL-10 levels have been correlated with the presence of a prostate tumor (Filella et al., Prostate 2000; 44(4):2714). A decrease in IL-10 serum levels has also been reported to be a prognostic indicator for multiple advanced solid tumors (De Vita et al., Oncol Rep. 2000; 7(2):357-61). In addition to these cytokine investigations, studies by Elsasser-Beile et al., 1993, supra have indicated that decreasing levels of serum IFN gamma protein correlate with increasing prostate tumor mass (Elsasser-Beile et al., 1993), and the present inventors have detected mRNA levels of IFN-gamma in peripheral blood leukocytes that are consistent with this study ( ). The diseases described above, such as schizophrenia and cancer, can be considered complex disorders, in as much as multiple gene abnormalities contribute to the etiology. Genome scans are widely used in the search for linkage regions, as a prerequisite for identification and mutation screening of candidate susceptibility genes. Linkage studies possess a number of limitations, often including some lack of reproducible, strong linkage findings, and the large breadth of chromosomal areas identified, which can contain potentially hundreds of genes. It is also considered that multiple genes of small or moderate effect may contribute to for example schizophrenia susceptibility, and therefore each need to be identified. However, linkage studies have highlighted a number of chromosomal regions that may harbor genes that contribute to schizophrenia and cancer. The difficult task is to identify susceptibility alleles among the large numbers of genes within or near these regions. Sequence analysis and association testing for all the genes within regions of linkage would be an overwhelming task.

An alternatively investigation of candidate genes that does not rely on genetic linkage data, allows a potential direct route to discovery of a gene mutation that may be involved in the etiology and pathogenesis of a disease, for example schizophrenia (O'Donovan et al., Am J Hum Genet 1999; 65, 587-592). To date however, although plausible candidates, particularly neurotransmitter metabolism and transport pathway members, and genes implicated in neurodevelopment, have been investigated, no candidate gene to date has produced strong association with schizophrenia.

A few recent studies have explored the possibility of finding candidate genes for complex disorders and/or traits, by involving a paradigm of candidate gene discovery or identification within regions of linkage, following or in parallel with measurement of gene expression of the tissue of interest. The paradigm of utilizing gene expression for the identification of candidate disease genes was also explored by Blackshaw et al. in a study of gene expression in murine rod cells, to identify gene candidates for retinal diseases (Blackshaw et al., Cell 2001; 107:579-89). The study, performed by serial analysis of gene expression (SAGE) found 264 previously uncharacterized genes that were expressed in the rod cells. Of those, 87 mapped to 37 different retinal disease loci, and are therefore considered candidate disease genes. In another study, Oestreicher et al. performed microarray gene expression using biopsied skin from patients with psoriasis and healthy controls and found 159 genes that were differentially expressed in the psoriasis biopsy samples. 27 of the differentially expressed genes mapped to psoriasis susceptibility loci, and were then considered as candidate genes for psoriasis (Oestreicher et al., Pharmacogenomics J. 2001; 1(4):272-87). Additional problems with this approach however, are that for the analysis of, for example brain tissue for research in psychiatric disorders such as schizophrenia, research problems are encountered by investigators collecting post mortem brain samples, where the subject's physiological state at death can be poorly defined, and the delay between time of death and brain sample collection can be lengthy (Li et al., Hum Mol Genet. 2004 13(6):609-16). This can lead to variability between samples, as also recently reported by Loring et al., following a study of gene expression in brain tissue from Alzheimer's disease patients (Loring et al., DNA Cell Biol. 2001; 20:683-95). In addition, biopsy would also be required for the analysis of tumor tissue for cancer research, and thus many subjects would have to undergo additional invasive surgery to obtain tissues for study.

### SUMMARY OF THE INVENTION

In one embodiment, the invention provides a method for evaluating a physical state of a subject (e.g., a “test subject”). This method comprises comparing an expression profile of surrogate cells from the subject, with a normal expression profile of surrogate cells from a normal subject not having the physical state, wherein a difference between the expression profiles is indicative of the physical state of the test subject.

In an alternative embodiment, evaluating a physical state of a subject (e.g., a “test subject”), which method involves comparing an expression profile of surrogate cells from the test subject with an expression profile of surrogate cells from a known subject or subjects determined to have the physical state. In this case, similarity in the expression profiles indicates that the test subject has the physical state of the known subject or subjects

In yet another embodiment, the invention provides a method for evaluating a treatment or therapy, such as a therapeutic compound, in a test subject. This method comprises comparing an expression profile of surrogate cells from the subject after exposing the subject to the compound, with an expression profile of surrogate cells from the subject prior to exposure to the compound, wherein a difference in the expression profiles indicates an effect of the compound on the test subject. In a further aspect, this method compares the expression profile of the test subject after exposing the subject to the compound, with a normal expression profile of surrogate cells from a normal subject. Similarity of the expression profiles indicates a therapeutic benefit of the compound.

In yet another aspect, this method compares the expression profile of the test subject after exposing the subject to the treatment or therapy, with an expression profile of surrogate cells from other subjects with the same physical state following exposure to different therapies and improvement of physical state, wherein a similarity of the expression profiles is indicative of the treatment or therapy efficacy on the test subject. In another alternative method, the expression profile of the test subject after exposing the subject to the treatment or therapy, is compared with an expression profile of surrogate cells from other subjects with the same physical state following exposure to different therapies, and lack of improvement or worsening of the physical state. Similarity of the expression profiles indicates a lack of therapeutic benefit of the compound.

In yet another embodiment, the invention provides a method for predicting a response to treatment or therapy, which comprises comparing an expression profile from the test subject prior to exposing the subject to a treatment or therapy, with an expression profile from surrogate cells from other subjects with the same physical state also profiled prior to exposure to different therapies, wherein a similarity in the expression profiles predicts an effect of the treatment or therapy on the test subject based on the effect of that therapy on another subject or subjects having a similar pre-treatment expression profile. In a further aspect, this method would be employed for choice of treatments.

In yet another embodiment the present invention provides for a method of treating a disease, disorder or physical state or to prevent onset of a disease, disorder or physical state, comprising administering a nucleic acid found to have altered expression in surrogate tissues, between a test subjects with the physical state, and a normal subject or subjects, including, but not limited to gene therapy with nucleic acid transcripts, antisense mRNA, or other inhibitory RNAs.

In an additional embodiment, this invention provides a method for identifying nucleic acids containing sequence alterations that may have a role in the etiology of a disease or disorder or physical state, in the pathogenesis of, or in the susceptibility for developing a disease or disorder or physical state. This method comprises identifying a nucleic acid that has altered gene expression in surrogate cells from a test subject when compared to surrogate cells from a normal subject or subjects, and then comparing the genomic sequence of the nucleic acid, to identify the sequence change. In a further aspect, this nucleic acid may be found to map within the human genome within or close to or adjacent to a region that has been previously identified in a linkage study or genome scan, or associated with the disease, disorder or physical state. In yet another embodiments the present invention provides for a method of treating a disease, disorder or physical state, comprising administering a normal counterpart of a nucleic acid found to have a sequence change using methods described in this invention, including but not limited to gene therapy with nucleic acid transcripts, antisense mRNA, or other inhibitory RNAs.

According to the invention, the physical state can be a disease or disorder such as the presence of cancer, a neurological disorder, or a psychiatric or mood disorder, or other diseases, disorders or physical states. In specific embodiments exemplified infra, the physical state is prostate cancer, breast cancer, schizophrenia, bipolar disorder, or Alzheimer's disease. Naturally, the subject can be any multi-celled organism that can offer surrogate cells (as hereinafter defined); the examples demonstrate these methods in humans.

The surrogate cells can be, but are not limited to, peripheral blood leukocytes, such as monocytes, macrophages, lymphocytes, granulocytes, eosinophils neutrophils, and basophils, or other white blood cell types or subtypes. They can also be mucosal epithelia, skin, hair follicle, or CSF cells (which are predominantly leukocytes).

Various types of physical state evaluations can be made in accordance with the invention. For example, evaluating a physical state can involve diagnosing the presence of a disease or disorder, determining the prognosis of the subject, determining susceptibility of a subject for a disease or disorder, monitoring a therapy for a disease or disorder, developing or selecting a therapy for a disease or disorder, or classifying a disease or disorder.

Although the robust methods of the invention do not require it, the methods envision further testing for a biochemical marker of the physical state in the blood or some other tissue sample, or evaluating a biopsy tissue sample for the presence of the physical state.

The expression profiling can be accomplished using any technology to measure nucleic acid transcript levels. For example, the method could employ a nucleic acid microarray, such as an oligonucleotide microarray or a cDNA microarray. Alternatively, one could simply employ reverse transcriptase-polymerase chain reaction (RT-PCR) or Northern blot hybridization. Additional methods that could be employed include, but are not limited to, Serial Analysis of Gene Expression (SAGE), high performance liquid chromatography (HPLC), mass spectrometry, differential display, quantitative measures of allelic specific expression, Taqman assays, Molecular Beacon assays, and phage display.

### DESCRIPTION OF THE DRAWINGS

FIG. 1. TreeView Representation of Cluster patterns of gene expression among men with prostate cancer and age-matched control subjects. 1A. Data are represented in matrix format. Each row represents a single gene (for space gene names have been omitted). Each column represents an experimental leukocyte patient or control sample. For each sample the ratio of the abundance of transcripts of each gene, to the median abundance of the genes's transcript among the individuals leukocytes, is represented by a rectangle in the corresponding matrix. The rectangles each represent the magnitude of the ratio relative to the median for the total set of samples. The dendrogram along the horizontal axis indicates the clusters of most similar subjects, based on gene expression levels of 1535 genes. The dendrogram along the vertical axis represents sample nodes of the total Cluster results, where genes appear together on the branches of the tree if they have similar patterns of gene expression. Example of Cluster nodes are taken from the total TreeView data, showing genes that are generally expressed at lower levels in the prostate cancer samples (A1 to A13), than control subject samples (B1 to B7). 1B. A scaled representation of the horizontal dendrogram showing patient and control cluster results is shown.

FIG. 2A-B. TreeView representation of Cluster patterns of actual and randomized expression levels of 1535 genes. Relationships among samples are represented by a dendrogram “tree”, where branch lengths reflect the degree of similarity, such that short branch lengths between nodes indicate similarity between samples. The arrows indicate the direction of subject divergence along the branches from each node.

FIG. 3. Partial TreeView Representation of Cluster patterns of gene expression among SZ men and control subjects. 3A Scaled representation of the horizontal dendrogram showing patient and control cluster results, based on the expression levels of 948 genes. Control Samples (C-401, 492, 536, 634 and 641) cluster into one node, SZ samples (P-493, 494, 495, 535, 588, 630, 631 and 964 (non-medicated subject)) cluster into a separate node. The sub-clusters within the SZ group do not seem to represent drug profiles, and the non-medicated subject (P-964) clusters within the SZ cluster node. The rectangles beneath each subject number represent the average signal intensity of a sample node of genes down regulated in SZ subjects.

FIG. 4. TreeView Representations of Cluster patterns of gene expression among SZ and BPD subjects. Data are represented in matrix format. Each row represents a single gene (for space gene names have been omitted). Each column represents an experimental leukocyte sample. For each sample the ratio of the abundance of transcripts of each gene, to the median abundance of the genes's transcript among the individuals leukocytes, is represented a rectangle in the corresponding matrix. The rectangles each represent the magnitude of the ratio relative to the median for the total set of samples. The dendrogram along the horizontal axis indicates the clusters of most similar subjects, based on gene expression levels of 1002 genes. The dendrogram along the vertical axis represents nodes, where genes appear together on the branches of the tree if they have similar patterns of gene expression. 4A. Example of Cluster nodes taken from the total TreeView data, showing genes that are expressed at lower levels (green) or absent (grey) in the SZ patients (SZ-493, 494, 495, 535, 588, 630, 631, and 964 (non-medicated), than the leukocyte samples taken from men with BPD (BPD-767, 846). 4B. A scaled representation of the horizontal dendrogram showing subject cluster results.

FIG. 5. TreeView representation of Cluster patterns of actual and randomized expression levels of 1002 genes. Relationships among samples are represented by a dendrogram “tree”, where branch lengths reflect the degree of similarity, such that short branch lengths between nodes indicate similarity between samples. The arrows indicate the direction of subject divergence along the branches from each node. 5A. A scaled representation of the horizontal dendrogram described in FIG. 4, where BPD subjects (BPD-747, and 846) cluster in one sub-node. 5B. A scaled representation of the TreeView readout generated when the gene expression levels of 1002 genes were randomized for each subject. Short branch length between nodes (in comparison to those observed in 5A) suggests only minor differences between samples.

FIG. 6.—The proportion of top ranked genes/ESTs that map to regions of schizophrenia linkage, filtered by increasing expression level cutoffs. Genes/ESTs were sorted by t-test p value (lowest to highest). The dataset was then subjected to a filtering step using increasing stringency in the form of signal intensity cutoffs (20 intensity unit steps). For each intensity cutoff, genes/ESTs that did not have 2 or more subjects with expression levels 2 the cutoff value were removed, and the number of genes/ESTs that map to regions of schizophrenia linkage within the top 10 of all genes/ESTs that passed the filters, were then plotted on the Y axis for each intensity cutoff level (X-axis). Filled grey circles indicate the sum total of linked genes/ESTs for each intensity cutoff. Thirty sets of randomized linkage data were also analyzed at each intensity cutoff point, and are shown by the filled black circles.

### DETAILED DESCRIPTION

The present invention provides novel “gene signatures” that are indicative of a physical state, e.g., a disease or disorder of a subject. These gene signatures, or expression profiles, are obtained from surrogate cells, such as blood cells, mucosal epithelial cells, and the like, that are available through non-invasive or minimally invasive procedures. Using the power of informative multiple gene expression profiling, or alternatively the coupling of multiple single gene expression measurements, the expression profile as described in the present invention permits the accurate classification, diagnosis, staging, and prognosis of diseases, determination of a biological, psychiatric, neurological or physical state including aging. The present invention also permits the prediction and evaluation of efficacy of therapeutic and treatment regimens and monitoring of subjects, and evaluation of candidates compounds for development and/or use as therapeutics. This invention also allows for the identification of candidate nucleic acids involved in the etiology and or susceptibility for a physical state.

This invention has significant advantages over current diagnostic and prognostic technologies. It does not require highly invasive techniques, such as tumor biopsy, that are required for confirming diagnosis of a cancer or other tissue conditions. Furthermore, it provides a biological measurement that permits a more conclusive diagnosis of diseases and conditions that are presently only conditionally diagnosed with conflation available only upon post-mortem examination, such as Alzheimer's disease, or for which no specific biological markers may be available, such as schizophrenia. In addition, this approach for discovery and validation of candidate genes for a physical state, utilizes a surrogate tissue, and therefore expands diagnostic choice and does not depend on the ability to access postmortem brain tissue, biopsied tumor tissue, or other involved tissues through invasive procedures. Indeed, invasive mechanisms of collection can greatly effect downstream gene expression, leading to great variability and inconsistencies between samples. The present invention is based, in part, on experiments which gave a complete classification of peripheral leukocyte expression clusters of prostate cancer patients (irrespective of race) when compared to age-matched normal controls, and a classification into expression clusters for schizophrenia and bipolar disorder patients compared to age- and race-matched controls (in this case with no significant effect of drug treatment for the schizophrenia on the expression profiles). Furthermore, the expression clusters of the schizophrenia subjects were distinct from those of the bipolar subjects.

In particular, for both prostate cancer and the psychiatric conditions, specific patterns, or signatures, of leukocyte gene expression that can both distinguish between control subjects and patients, and also differentiate between different psychiatric illnesses, have been identified.

Experiments showing the accurate classification of prostate cancer patients and healthy control subjects into their respective groups, based on the expression levels of over 1500 genes, support breast cancer diagnosis though leukocyte expression signatures. Specifically, while the genes employed above for classification of prostate cancer will not necessarily be the exact genes employed for classification of breast cancer, common similarities between breast and prostate cancer, including incidence and mortality rates, risk factors, initiation of transformation, and roles of androgens and estrogens (reviewed in Lopez-Otin & Diamandis 1998; Coffey S. 2001; Cavalieri & Rogan. 2002; Liao et al., 2002; Grover & Martin 2002) indicate that growth and development of a breast cancer will exert an effecton the immune system, similar to that predicted for prostate cancer, that can be detected at the level of altered gene expression in peripheral blood leukocytes.

It seems clear that the use of multiple nucleic acid transcripts for the determination of expression signatures provides considerably more detailed information on disease stage and prognosis than can be provided by the quantitation of individual serum protein levels, as described in the Background to the Invention. It should also be noted that although surrogate cell gene expression levels will be measured, if, e.g., malignant breast cells were also present in the blood of patients, then gene expression of these cells will also be quantified. It seems likely that the detection of gene expression in affected cells within blood might actually increase the specificity of the analysis, as mRNA levels arising from circulating involved cells would differ from mRNA levels in prostate cancer patients with no such cells in their blood stream, and to a even greater degree than normal control subjects.

These results form the basis of a diagnostic screen. A clinical assay would initially involve extraction of a surrogate tissue, such as a blood sample, from the subject at risk for the condition to be tested. A labeled probe synthesized from RNA extracted from the surrogate cells can be hybridized to a microarray containing a number of genes (determined according to this invention) that are differentially expressed between patients and control individuals to identify whether the test subject has the particular condition. The resultant expression pattern can then be compared to a set of known multigene signatures that more specifically characterize the condition, e.g., expression profiles that are specific for individual stages of tumor progression. The invention represents a non-invasive diagnostic assay that can yield both diagnostic and staging information for each individual at risk.

Since this assay will measure gene expression within surrogate cells such as leukocytes, instead of cells directly involved in the physical state, and does not rely on the measurement of biomolecules secreted from involved cells, the resultant assay is sensitive and accurate, and capable of detecting conditions that are still at an early stage. Such an assay serves as an important pre-screen that can, with a minimum of patient discomfort, identify subjects who have the particular condition.

### Definitions

### Specialized

As used herein, the term “physical state” refers to the physiological, psychological, and health status of a subject. Various physical states include diseases and disorders, such as: proliferative disorders including cancer; pulmonary disorders; dermatological diseases; developmental disorders; muscular disorders; respiratory diseases; sexual, fertility and gynecological disorders; allergic disorders; inflammatory disorders (e.g. ulcerative colitis etc.); infectious diseases; parasitic infestations; growth abnormalities, a hyperactive or hypoactive endocrine syndrome (e.g., hyperthyroidism, hypothyroidism, growth hormone deficiency or dwarfism, type I diabetes, type II diabetes, etc.); neurological diseases (e.g., Alzheimer's, Parkinson's, Huntington's, ALS, etc.); psychiatric and mood disorders (e.g., schizophrenia, bipolar disorder, depression, obsessive-compulsive disorder, etc.); obesity; sleep disorders; other pathological conditions; and normal and abnormal aging. Physical states also include altered metabolic states, which may be due to ingestion of exposure to, pharmaceuticals, chemicals, alcohol, environmental toxins, food toxins, and the like; metabolic or nutritional conditions or deficiencies, such as but not limited to hyperlipidemia, hypercholesterolemia, malnutrition, and vitamin deficiencies. The data show a possible hierarchy of effects: a disease like schizophrenia seems to have greater impact on expression profiles of blood cells than the neuroleptic drugs that the schizophrenic patients are taking for the condition. A normal physiological state is a special kind of physical state, which can be determined from the methods of the invention.

The term “expression profile” refers to expression of two or more, preferably three or more, for example 5, 10, 20, 50, 100, 500, or 1000 or more, genes/EST or other transcribed nucleic acids. Genes/ESTs or nucleic acids within a subject's expression profile can be expressed at different levels (either to a greater or lesser extent, e.g., by about 2-fold of more, or less than 2-fold, and preferably within the error limits of the detection) to the gene expression profile levels of a subject or subjects with a physical state, and also for example, between subjects treated with therapeutic compounds, or between treated and untreated subjects. The differences between subjects expression profiles can then for example, be employed for diagnosing the presence of a physical state, determining the prognosis of the subject, determining susceptibility of a subject for a physical state, monitoring a therapy for a physical state, developing or selecting a therapy for a physical state, or classifying a physical state. In certain embodiments, genes in an expression profile may not include known markers of the involved cells, e.g., PSA in prostate cancer (given the highly sensitive detection technologies available, efforts are made to detect cancer cell genes in the low population of circulating metastatic cells), but in early stage non-disseminated disease such markers may well be expressed in the surrogate cells and be informative. The expression profile is indicative of a particular physical state. As used herein, the expression profile of a gene is preferably the level of mRNA, e.g., measured using microarrays or RT-PCR as described herein. In particular embodiments, nucleic acids (e.g., mRNA) expressed by a cell are reverse transcribed into either cDNA or cRNA, and the abundances of the cDNA and/or cRNA molecules are measured. Expression profiles can be presented in various forms, as discussed below, including through dendograms, TreeView readouts, color matrixes, charts, graphs, or by computer analysis without visualization. Determination of expression profiles involves analyzing expression of genes in subjects diagnosed, for example using statistical analyses, or hierarchical clustering or classification algorithms (with as much accuracy and precision as possible, including through post-mortem confirmation if necessary) with the particular physical state.

As used herein, the term “surrogate cells” refers to cells from a tissue source that is not the primary involved tissue of the physical state of the subject (except of course to the extent that “normal” is a special type of physical state, then the surrogate cells exhibit “normal” expression patterns). The term includes but need not be limited to blood cells, mucosal epithelial cells, skin cells, cells of hair follicles, cells from cerebrospinal fluid (CSF), and cells from lymphatic fluid. One of the advantages of the invention lies in the power to analyze expression patterns from complex mixtures of cells that might be present in any given tissue source, as discussed in the Examples. Thus, blood cells include leukocytes (monocytes, macrophages, lymphocytes, granulocytes, eosinophils, etc.), as well as platelets and megakaryocytes. Skin cells include Langerhans cells, keratinocytes, and dermal cells. Furthermore, the surrogate cells can be purified populations or subpopulations of these cells, e.g., T or B lymphocytes separated from the blood cells. However, this is not necessary for practicing the invention.

Surrogate cells are predominantly not the cells affected by the physical state (except, of course, for a normal physical state or normal aging) but the term does not exclude the possibility that disease cells are present in the surrogate cells. Thus, if the disease is cancer and the surrogate cells are blood cells, there may be some metastatic cells in the blood cells. However, tumor cells from a biopsy would clearly not be surrogate cells for purposes of this invention. Furthermore, purification of involved cells is not necessary, and falls outside the definition of surrogate cells.

The term “subject” can mean patient, test subject, animal including laboratory animals, or any entity capable of testing for physical state by obtaining an expression profile or signature of surrogate cells, including plants, for example, a genetically modified plant species. Preferably a patient is a human, but can also be a domestic animal or pet (e.g., a dog, cat, etc.), a farm animal (e.g., horse, cow, sheep, pig, goat, etc.), or a wild animal, such as in a zoo. A test subject can be a human or animal involved in a clinical trial of a drug or in a trial, as exemplified herein, for determining new, expanded, or refined expression profiles. Clearly the groups of “patients” and “test subjects” can overlap. Laboratory animals include mice, rats, rabbits, hamsters, cats, dogs, etc.

The term “genetically linked” refers to the proximity of two or more genes and/or traits within the genome of an organism that causes those genes or traits to be inherited, transferred, or moved together with a frequency greater than for genes or traits not linked. The linkage is a continuous variable and is inversely related to the distance between genes/traits on the genome. For investigating linkage of diseases, disorders or physical states, such as schizophrenia, genetic linkage is measured by the heritability within a family (and families) of genes or markers of interest, whereby genes or markers within a particular chromosome location are linked to a disease, disorder or physical state if allelic variation of the gene or marker segregates within the family with the disease, disorder or physical state. Those genomic regions are considered likely to contain genes which, when mutated or altered or deleted, contribute to susceptibility, or the cause or pathogenesis or etiology of a disease, disorder or physical state. For example, for schizophrenia linkage has been suggested for multiple genomic regions including chromosomes 1q23.3-q31.1, 2 p12-q22.1, 3p25.3-p22.1, 5q23.2-q34, 11q22.3-24.1, 6pter-p22.3, 2q22.1-q23.3, 1p13.3-q23.3, 8p22-p21.1, 6q15-q23.2, 6p22.3-p21.1, 10pter-p14, 14pter-q13.1, 15q21.3-q26.1, 16 p13-q12.2, 17q21.33-q24.3, 18q22.1-qter, 20 p12.3-p11, 22pter-q12.3 (Lewis et al., Am J Hum Genet. 2003; 73(1):34-48) According to one embodiment of the presently claimed methods, nucleic acids representing genes or ESTs that have a different expression profiles in surrogate cells from a subject having or suspected of having a physical state compared with cells from normal individuals not having a physical state, which can also be linked to that disease, disorder or physical state, will be chosen for genetic mutation analysis, i.e., by sequencing. As used herein, the term genetically linked also includes nucleic acid sequences representing genes or ESTs on chromosomal regions that are proximal or distal to the linked site.

In a specific embodiment, exemplified below, one can identify relevant genes whose expression is up- or down-regulated in disease conditions such as prostate cancer or disorders such as schizophrenia. For further diagnosis and testing, one can prepare arrays with all or a subset of all of the genes. For example, such an array employs a probe for at least one such gene, preferably at least 5, more preferably at least 10, more preferably at least 50, and more preferably at least 100, or 500, or 1000 or more such genes. Furthermore, genes are selected for inclusion in an array on the basis of the significance level of the differential expression. A significance level of less p<0.1 (e.g., using the Student's two-tailed test) indicates a trend towards significance; a significance level of p<0.05 provides greater certainty; a significance level of p<0.01 even greater certainty. It should be understood that the value of p may change with greater sample size.

Thus, in one embodiment, one can diagnose the presence of prostate cancer using expression data for one or more, preferably 5 or more, more preferably 10, 50, or 100 or more genes from Table 1, below. Preferably, the genes are selected as having a trend level of p<0.1, or more preferably a significance level of p<0.05, and more preferably p<0.01. In one embodiment, the gene probe on the expression array detects one or more of proteasome (prosome, macropain) subunit, alpha type, 5; S-phase kinase-associated protein 1A (p19A); KIAA0542 gene product; endothelial differentiation, G-protein-coupled receptor 6; tubulin, alpha 1 (testis specific); chromosome 10 open reading frame 6; G-rich RNA sequence binding factor 1; Rab acceptor 1 (prenylated); solute carrier family 17 (sodium-dependent inorganic phosphate cotransporter), member 7; cAMP responsive element modulator; Wiskott-Aldrich syndrome (eczema-thrombocytopenia); glutamate receptor, metabotropic 4; dynamin 2; glycosyltransferase AD-017; dimethylarginine dimethylaminohydrolase 2; similar to transcription factor TBX10; Tubulin, Alpha 1, Isoform 44; pyruvate kinase, muscle; splicing factor, arginine/serine-rich 1 (splicing factor 2, alternate splicing factor); ubiquitin-activating enzyme E1 (A1S9T and BN75 temperature sensitivity complementing); huntingtin-associated protein 1 (neuroan 1); ubiquitin ligase E3 alpha-II; ubiquitin-conjugating enzyme E2N (UBC13 homolog, yeast); potassium voltage-gated channel, shaker-related subfamily, beta member 2; farnesyltransferase, CAAX box, alpha; ATPase, H+ transporting, lysosomal 16 kDa, V0 subunit c; eukaryotic translation initiation factor 2B, subunit 4 delta, 67 kDa; and likely ortholog of mouse variant polyadenylation protein CSTF-64. In another embodiment, an expression array of the invention can include any genes with a significance of e.g. p<0.0005, or alternatively with a significance of p<0.001, or a trend level of significance of p<0.07, from Table 1.

Thus, in one embodiment, one can diagnose the presence of schizophrenia using expression data for one or more, preferably 5 or more, more preferably 10, 50, or 100 or more genes from Table 2, below. Preferably, the genes are selected as having a trend level of p<0.1, or more preferably a significance of p<0.05, and more preferably p<0.01. In one embodiment, the gene probe on the expression array detects one or more of par-6 partitioning defective 6 homolog alpha (C. elegans) (also called homo sapiens tax interaction protein 40), transmembrane 4 superfamily member tetraspan NET-5, neural cell adhesion molecule 1, cadherin 16, KSP-cadherin WD repeat domain 1, growth hormone releasing hormone B-cell translocation gene 1, anti-proliferative solute carrier family 10 (sodium/bile acid cotransporter family), and member 1 HRIHFB2206 protein. In another embodiment, an expression array of the invention can include any genes with a significance of e.g. p<0.0005, or alternatively with a significance of p<0.001, or a trend level of significance of p<0.07, from Table 2.

### Generalized

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes isolated DNA, a PCR product, isolated RNA (mRNA, cRNA, tRNA, rRNA), a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, mass spectrometry and other methods known in the art.

Methods for purification are well known in the art. For example, nucleic acids can be purified by precipitation, chromatography (including preparative solid phase chromatography, oligonucleotide hybridization, and triple helix chromatography), ultracentrifugation, and other means. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art.

A “sample” as used herein refers to a biological material which can be tested, e.g., a tissue, for example a surrogate tissue, comprising cells, that are tested or analyzed for the presence or absence of certain particular nucleic acid sequences, corresponding to certain genes that may be expressed by the cell or present in the cell.

A “gene” is a sequence of nucleotides which code for a functional “gene product”. Generally, a gene product is a functional protein. However, a gene product can also be another type of molecule in a cell, such as an RNA. For the purposes of the present invention, a gene product also refers to an mRNA sequence which may be found in a cell. For example, measuring gene expression levels according to the invention may correspond to measuring mRNA levels.

The term “express” and “expression” means allowing or causing the information in a gene or DNA sequence to become manifest, for example producing RNA (such as mRNA) or a protein by activating the cellular functions involved in transcription and translation of a corresponding gene or DNA sequence. A DNA sequence is expressed by a cell to form an “expression product” such as an RNA (e.g., an mRNA) or a protein. The expression product itself, e.g., the resulting RNA or protein, may also said to be “expressed” by the cell. As used herein, the term expression also refers to the amount or abundance of mRNA corresponding to a particular gene that is present in a cell.

“Amplification” of a nucleic acid, as used herein, denotes the use of an amplification synthetic process, such as polymerase chain reaction (PCR), to increase the concentration of a particular DNA or cDNA, or mRNA or cRNA sequence within a mixture of nucleic acid sequences. For a description of PCR see Saiki et al., Science 1988, 239:487.

The term “inhibitory RNA” can refer to an RNA species that can directly or indirectly inhibit expression of a gene or other nucleic acids by interfering with, or decreasing the process of transcription, and/or directly or indirectly increasing the degradation or cleavage of the targeted gene or nucleotide transcript, thus reducing the gene or nucleic acid's transcript levels or expression levels at the RNA and/or protein level. RNA molecules can be used to cause inhibition of expression of genes or other nucleotide sequences. RNA molecules utilized or employed for inhibition, can contain in whole or part, sequence that is at least similar to, or substantially identical to, or substantially complementary to (in whole or part), an RNA sequence produced from a gene or other nucleotide sequence being targeted (Shuey et al. Drug Discov Today. 2002 7(20):1040-6). Sequence-specific, or partically sequence specific inhibition of a gene or nucleotide transcript's expression, can be induced using several different methodologies and molecule types, including but not limited to: chemically modified antisense oligodeoxyribonucleic acids (ODNs), ribozymes and siRNAs, peptide nucleic acids (PNAs), morpholino phosphorodiamidates, DNAzymes and 5′-end-mutated U1 small nuclear RNAs (Dorsett et al. Nat Rev Drug Discov. 2004 3(4):318-29). Additionally, the introduction of single or double stranded RNA or RNA-like molecules that are preferably less than 30 nucleotides in length may be more useful for decreasing cell death and/or activation when the sequences are introduced. (Xu et al., Biochem Biophys Res Commun. 2004 316(3):680-7). The use of interference technologies such as RNAi for therapeutic approaches to physical states, diseases or disorders, can also include the introduction to cells, organs, tissues or organisms, of specific RNA molecules, either as uncomplexed oligonucleotides, and/or using viral or retroviral vectors, or other vectors such as plasmids or liposomes, containing small interfering RNA sequence (siRNA) or small hairpin RNA sequence (shRNA) or their precursor vector sequences (reviewed in Devroe et al., Expert Opin Biol Ther. 2004 4(3):319-27; Davidson et al., Lancet Neurol. 2004 (3):145-9).

A nucleic acid molecule is “hybridizable” to another nucleic acid molecule, such as a cDNA, oligo-DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. SCC is a 0.15M NaCl, 0.015M Na citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

Suitable hybridization conditions for oligonucleotides (e.g., for oligonucleotide probes or primers) are typically somewhat different than for full-length nucleic acids (e.g., full-length cDNA), because of the oligonucleotides' lower melting temperature. Because the melting temperature of oligonucleotides will depend on the length of the oligonucleotide sequences involved, suitable hybridization temperatures will vary depending upon the oligonucleotide molecules used. Exemplary temperatures may be 37° C. (for 14-base oligonucleotides), 48° C. (for 17-base oligoncucleotides), 55° C. (for 20-base oligonucleotides) and 60° C. (for 23-base oligonucleotides). Exemplary suitable hybridization conditions for oligonucleotides include washing in 6×SSC/0.05% sodium pyrophosphate, or other conditions that afford equivalent levels of hybridization.

Preferably, nucleic acid molecules in the present invention are detected by hybridization to probes of a microarray. Hybridization and wash conditions are therefore preferably chosen so that the probe “specifically binds” or “specifically hybridizes” to a specific target nucleic acid. In other words, the nucleic acid probe preferably hybridizes, duplexes or binds to a target nucleic acid molecules having a complementary nucleotide sequence, but does not hybridize to a nucleic acid molecules having a non-complementary sequence. As used herein, one oligonucleotide sequence is considered complementary to another when, if the shorter of the oligonucleotides is less than or equal to about 25 bases, there are no mismatches using standard base-pairing rules, or using mismatch analysis algorithms (Affymetrix Inc). If the shorter of the two polynucleotides is longer than about 25 bases, there is preferably no more than a 5% mismatch. Preferably, the two oligonucleotides are perfectly complementary (i.e., no mismatches). It can be easily demonstrated that particular hybridization conditions are suitable for specific hybridization by carrying out the assay using negative controls. See, for example, Shalon et al., Genome Research 1996, 639-645; and Chee et al., Science 1996, 274:610-614.

Optimal hybridization conditions for use with microarrays will depend on the length (e.g., oligonucleotide versus polynucleotide greater than about 200 bases) and type (e.g., RNA, DNA, PNA, etc.) of probe and target nucleic acid. General parameters for specific (i.e., stringent) hybridization conditions are described above. Hybridization conditions for use of Affymetrix commercial oligonucleotide arrays have been developed for standardized use (Affymetrix Inc.) For cDNA microarrays, such as those described by Schena et al. (Proc. Natl. Acad. Sci. USA; 1996, 93:10614), typical hybridization conditions comprise hybridizing in 5×SSC and 0.2% SDS at 65° C. for about four hours, followed by washes at 25° C. in a low stringency wash buffer (for example, 1×SSC and 0.2% SDS), and about 10 minutes washing at 25° C. in a high stringency wash buffer (for example, 0.1×SSC and 0.2% SDS). Useful hybridization conditions are also provided, e.g., in Tijessen, Hybridization with Nucleic Acid Probes, Elsevier Sciences Publishers (1996), and Kricka, Nonisotopic DNA Probe Techniques, Academic Press, San Diego Calif. (1992). Generally commercially available expression screening systems that use hybridization provide defined hybridization and wash conditions.

### Measuring Expression Profiles

Various commercial systems are available for profiling gene expression. These include the powerful single gene amplification processes such as reverse transcription-polymerase chain reaction (RT-PCR). Multigene profiling can be performed in single reaction mixtures using specific detection signals, such as dyes, in separate reaction mixtures, or on arrays. Various commercial systems are available for expression profiling as well.

eXpress Profiling (XP) by Althea (San Diego, Calif.) is useful in screening large numbers of compounds for effects on expression of a limited number of known target genes (approximately up to 20 per single well reaction). The assay employs discernible fluorescent dyes that can be reliably and simultaneously detected in a single reaction mixture. XP works by first amplifying the cDNA sources to be compared with a pair of gene-specific primers that each carry a universal sequence at their 5′ end. The resulting PCR amplicon is then further amplified with a pair of primers that hybridize to the universal sequences at both termini of the original PCR amplicon. One of the latter primer pair is fluorescently labeled, such that the final product can be quantified.

Assays-on-Demands by Applied Biosystems (Foster City, Calif.) can be used for validation of microarray hits. The assay provides a means of higher reliability and accuracy in the expression profiling of single genes. Each kit is custom tailored to a particular gene; kits can be combined for multigene profiles. It is useful for standardization purposes, due to better comparability of results between different experiments/laboratories. The assay uses random primers in the initial cDNA synthesis step, which enables higher quality signal detection along the transcript. The PCR amplification step is based on AB's TaqMan system which then allows one to quantify the amount of cDNA in the sample.

EnzyStart™ by GeneCopeia (Frederick, Md.) blocks the 3′ end of amplification primers with an enzymatically removable blocking group, which avoids non-specifically primed DNA polymerization that may otherwise occur due to primer hybridization at ambient temperature. A Terminal Blocker Group Remove Enzyme (TBGRE) present in the reaction is activated at temperatures above 55° C. to produce free hydroxyl-groups at the 3′ end of the primer, thus allowing the PCR reaction to start only after non-specifically hybridized primers are melted off the template. This is particularly useful when very low concentrations of cDNA are to be detected, when signal to noise ration is a problem.

Omega Beacon™ by Gorilla Genomics (Alameda, Calif.) provides a quantitative real-time PCR method useful for measurement of gene expression. These probes form stem-loop structures, where the loop sequence hybridizes specifically to the DNA target of interest. Upon hybridization the stem is destabilized and opens, which releases a fluorescence quencher from the proximity of the fluorophore, and thus allowing for fluorescence and the quantification thereof.

Black Hole Quenchers by Biosearch Technologies (Novato, Calif.) employs on a similar mechanism as Omega Beacons. Here fluorophore and quencher are kept in proximity in the unhybridized state due to the random coiling of the probe. Upon hybridization to the target sequence the probe is stretched out, which permits quantifiable fluorescence emission.

### Nucleic Acid Arrays

The terms “array” and “microarray” are used interchangeably and refer generally to any ordered arrangement (e.g., on a surface or substrate) or different molecules, referred to herein as “probes”. Each different probe of an array specifically recognizes and/or binds to a particular molecule, which is referred to herein as its “target”. Microarrays are therefore useful for simultaneously detecting the presence or absence of a plurality of different target molecules, e.g., in a sample. In preferred embodiments, arrays used in the present invention are “addressable arrays” where each different probe is associated with a particular “address”. For example, in preferred embodiments where the probes of are immobilized on a surface or a substrate, each different probe of the addressable array may be immobilized at a particular, known location on the surface or substrate. The presence or absence of that probe's target molecule in a sample may therefore be readily determined by simply determining whether a target has bound to that particular location on the surface or substrate.

The methods of the invention may be practiced using nucleic acid arrays (also referred to herein as “transcript arrays” or “hybridization arrays”) that comprise a plurality of nucleic acid probes immobilized on a surface or substrate. The different nucleic acid probes are complementary to, and therefore hybridize to, different target nucleic acid molecules, e.g., in a sample. Thus such probes may be used to simultaneously detect the presence and/or abundance of a plurality of different nucleic acid molecules in a sample, including the expression of a plurality of different genes; e.g., the presence and/or abundance of different tiRNA molecules, or of nucleic acid molecules derived therefrom (for example, cDNA or cRNA).

There are two major types of microarray technology; spotted cDNA arrays and manufactured oligonucleotide arrays. Examples 1 and 2 employ high density oligonucleotide Affymetrix® GeneChip arrays (reviewed in Schena at el., 1998).

Transcript Arrays Generally. In a preferred embodiment the present invention makes use of “transcript arrays” (also called herein “microarrays”) for determining the effect of a test compound on gene expression. Transcript arrays can be employed for analyzing the transcriptional state in a surrogate cell in comparison to a known cell (whether known to be normal or known to be from a subject with an abnormal physical state).

Microarrays can be made in a number of ways, of which several are described below. However produced, microarrays share certain characteristics. The arrays are preferably reproducible, allowing multiple copies of a given array to be produced and easily compared with each other. Preferably the microarrays are small, usually smaller than 5 cm2, and they are made from materials that are stable under binding (e.g., nucleic acid hybridization) conditions. A given binding site or unique set of binding sites in the microarray will specifically bind the product of a single gene in the cell. Although there may be more than one physical binding site (hereinafter “site”) per specific mRNA, for the sake of clarity the discussion below will assume that there is a single site. It will be appreciated that when cDNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (with a fluorophore) cDNA complementary to the total cellular mRNA is hybridized to a microarray, the site on the array corresponding to a gene (i.e., capable of specifically binding a nucleic acid product of the gene) that is not transcribed in the cell will have little or no signal, and a gene for which the encoded mRNA is prevalent will have a relatively strong signal.

The use of a two-color fluorescence labeling and detection scheme to define alterations in gene expression has been described (e.g., Shena et al., Science 1995, 270:467-470). An advantage of using cDNA labeled with two different fluorophores is that a direct and internally controlled comparison of the mRNA levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular mRNA in, e.g., a treated and untreated cell.

By way of example, GeneChip expression analysis (Affymetrix, Santa Clara, Calif.) generates data for the assessment of gene expression profiles and other biological assays. Oligonucleotide expression arrays simultaneously and quantitatively interrogate thousands of mRNA transcripts (genes or ESTs, via a cRNA synthesis step), simplifying large genomic studies. Each transcript can be represented on a probe array by multiple probe pairs, representing different regions of the genes or ESTs, to differentiate among closely related members of gene families. Each probe cell contains millions of copies of a specific oligonucleotide probe, permitting the accurate and sensitive detection of low-intensity mRNA hybridization patterns. After hybridization intensity data is captured, e.g., using a Hewlett-Packard GeneArray™ scanner, software can be used to automatically calculate intensity values for each probe cell. Probe cell intensities can be used to calculate an average intensity for each gene, which directly correlates with mRNA abundance levels. Expression data can be quickly sorted on any analysis parameter and displayed in a variety of graphical formats for any selected subset of genes. Other gene expression detection technologies include the research products manufactured and sold by Perkin-Elmer and Gene Logic. Additionally, software such as BRB Array Tools (NCI), GeneSpring (Silicon Genetics), GeneLinker Platinum (Predictive Patterns Software Inc.) can also be used to perform clustering, gene profiling, sample classification and statistical analyses of expression profiles.

Preparation of Microarrays. Microarrays are known in the art and preferably comprise a surface to which short or long oligonucleotide or cDNA probes, that correspond in sequence to gene products (e.g., cDNAs, mRNAs, cRNAs, polypeptides, and fragments thereof), can be specifically hybridized or bound at a known position within the microarray. In one embodiment, the microarray is an array in which each position represents a discrete binding site for a product encoded by a gene (e.g., a protein or RNA), and in which binding sites are present for products of most or almost all of the genes in the organism's genome. In a preferred embodiment, the “binding site” (hereinafter, “site”) is a nucleic acid or nucleic acid analogue to which a particular cognate cDNA or cRNA can specifically hybridize. The nucleic acid or analogue of the binding site can be, e.g., a synthetic oligomer, a full-length cDNA, a less-than full length cDNA, or a gene fragment.

Although in a preferred embodiment the microarray contains binding sites for products of all or almost all genes in the target organism's genome, such comprehensiveness is not necessarily required for diagnostic arrays with a defined set of genes that are differentially expressed (the expression profile genes).

Preparing Nucleic Acids for Microarrays. As noted above, the “binding site” to which a particular cognate cDNA or cRNA specifically hybridizes is usually a nucleic acid or nucleic acid analogue attached at that binding site. In one embodiment, the binding sites of the microarray are DNA polynucleotides corresponding to at least a portion of each gene in an organism's genome. These DNAs can be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments (i.e., fragments that do not share more than 10 bases of contiguous identical sequence with any other fragment on the microarray). Computer programs are useful in the design of primers with the required specificity and optimal amplification properties. See, e.g., Oligo version 5.0 (National Biosciences). In the case of binding sites corresponding to very long genes, it will sometimes be desirable to amplify segments near the 3′ end of the gene so that when oligo-dT primed cDNA probes are hybridized to the microarray, less-than-full length probes will bind efficiently. Typically each gene fragment on the microarray will be between about 50 bp and about 2000 bp, more typically between about 100 bp and about 1000 bp, and usually between about 300 bp and about 800 bp in length. PCR methods are well known and are described, for example, in Innis et al., eds., 1990, PCR Protocols: A Guide to Methods and Applications, Academic Press Inc. San Diego, Calif. It will be apparent that computer controlled robotic systems are useful for isolating and amplifying nucleic acids.

An alternative means for generating the nucleic acid for the microarray is by synthesis of synthetic polynucleotides or oligonucleotides, e.g., using N-phosphonate or phosphoramidite chemistries (Froehler et al., Nucleic Acid Res. 1986, 14:5399-5407; McBride et al., Tetrahedron Lett. 1983, 24:245-248). Synthetic sequences are between about 15 and about 500 bases in length, more typically between about 20 and about 50 bases. In some embodiments, synthetic nucleic acids include non-natural bases, e.g., inosine. As noted above, nucleic acid analogues may be used as binding sites for hybridization. An example of a suitable nucleic acid analogue is peptide nucleic acid (see, for example, Egholm et al., Nature 1993, 365:566-568. See, also, U.S. Pat. No. 5,539,083).

In an alternative embodiment, the binding (hybridization) sites are made from plasmid or phage clones of genes, cDNAs (e.g., expressed sequence tags), or inserts therefrom (Nguyen et al., Genomics 1995, 29:207-209). In yet another embodiment, the polynucleotide of the binding sites is RNA.

Attaching Nucleic Acids to the Solid Surface. The nucleic acids or analogues are attached to a solid support, which may be made from glass, plastic (e.g., polypropylene, nylon), polyacrylamide, nitrocellulose, or other materials. A preferred method for attaching the nucleic acids to a surface is by printing on glass plates, as is described generally by Schena et al., Science 1995, 270:467-470. This method is especially useful for preparing microarrays of cDNA. See also DeRisi et al., Nature Genetics 1996, 14:457-460; Shalon et al., Genome Res. 1996, 6:639-645; and Schena et al., Proc. Natl. Acad. Sci. USA 1995, 93:10539-11286.

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et al., Science 1991, 251:767-773; Pease et al., Proc. Natl. Acad. Sci. USA 1994, 91:5022-5026; Lockhart et al., Nature Biotech. 1996, 14:1675. See, also, U.S. Pat. Nos. 5,578,832; 5,556,752; and 5,510,270) or other methods for rapid synthesis and deposition of defined oligonucleotides (Blanchard et al., Biosensors & Bioelectronics 1996, 11:687-90). When these methods are used, oligonucleotides (e.g., 20-mers) of known sequence are synthesized directly on a surface such as a derivatized glass slide. Usually, the array produced is redundant, with several oligonucleotide molecules per RNA. Oligonucleotide probes can be chosen to detect alternatively spliced mRNAs.

Other methods for making microarrays, e.g., by masking (Maskos and Southern, Nuc. Acids Res. 1992, 20:1679-1684), may also be used. In principal, any type of array, for example, dot blots on a nylon hybridization membrane (see, Sambrook et al., Molecular Cloning—A Laboratory Manual (2nd Ed.), Vol. 1-3, Cold Spring Harbor Laboratory, Cold Spring Harbor, N.Y., 1989), could be used, although, as will be recognized by those of skill in the art, very small arrays will be preferred because hybridization volumes will be smaller.

Generating Labeled Probes. Methods for preparing total and poly(A)+ RNA are well known and are described generally in Sambrook et al., supra. In one embodiment, RNA is extracted from cells of the various types of interest in this invention using guanidinium thiocyanate lysis followed by CsCl centrifugation (Chirgwin et al., Biochemistry 1979, 18:5294-5299). Poly(A)+ RNA is selected by selection with oligo-dT cellulose (see Sambrook et al., supra). Cells of interest may include, but are not limited to, wild-type cells, surrogate cells, drug-exposed wild-type cells, modified cells, and drug-exposed modified cells.

Labeled cDNA is prepared from mRNA by oligo dT-primed or random-primed reverse transcription, both of which are well known in the art (see, for example, Klug and Berger, Methods Enzymol. 1987, 152:316-325). Reverse transcription may be carried out in the presence of a dNTP conjugated to a detectable label, most preferably a fluorescently labeled dNTP. Alternatively, isolated mRNA can be converted to labeled antisense RNA synthesized by in vitro transcription of double-stranded cDNA in the presence of labeled dNTPs (Lockhart et al., Nature Biotech. 1996, 14:1675). In alternative embodiments, the cDNA or RNA probe can be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

When fluorescently-labeled probes are used, many suitable fluorophores are known, including fluorescein, lissamine, phycoerythrin, rhodamine (Perkin Elmer Cetus), Cy2, Cy3, Cy3.5, Cy5, Cy5.5, Cy7, Fluor X (Amersham) and others (see, e.g., Kricka, 1992, Nonisotopic DNA Probe Techniques, Academic Press San Diego, Calif.). It will be appreciated that pairs of fluorophores are chosen that have distinct emission spectra so that they can be easily distinguished.

In another embodiment, a label other than a fluorescent label is used. For example, a radioactive label, or a pair of radioactive labels with distinct emission spectra, can be used (see Zhao et al., Gene 1995, 156:207; Pietu et al., Genome Res. 1996, 6:492). However, because of scattering of radioactive particles, and the consequent requirement for widely spaced binding sites, use of radioisotopes is a less-preferred embodiment.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM Rhodamine 110 UTP (Perken Elmer Cetus) or 0.1 mM Cy3 dUTP (Amersham)) with reverse transcriptase (e.g., SuperScript™ II, LTI Inc.) at 42° C. for 60 minutes.

Hybridization to Microarrays. Nucleic acid hybridization and wash conditions are chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It can easily be demonstrated that specific hybridization conditions result in specific hybridization by carrying out a hybridization assay including negative controls (see, e.g., Shalon et al., supra; and Chee et al., supra).

Optimal hybridization conditions will depend on the length (e.g., oligomer versus polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described above. When cDNA microarrays, such as those described by Schena et al. are used, typical hybridization conditions are hybridization in 5×SSC plus 0.2% SDS at 65 1C for 4 hours, followed by washes at 25° C. in low stringency wash buffer (e.g., 1×SSC plus 0.2% SDS) followed by 10 minutes at 25° C. in high stringency wash buffer (0.1×SSC plus 0.2% SDS). See, Shena et al., Proc. Natl. Acad. Sci. USA 1996, 93:10614). Useful hybridization conditions are also provided in, e.g., Tijessen, 1993, Hybridization With Nucleic Acid Probes, Elsevier Science Publishers B.V. See, also, Kricka, 1992, Nonisotopic DNA Probe Techniques, Academic Press, San Diego, Calif.

Signal Detection and Data Analysis. When fluorescently labeled probes are used, the fluorescence emissions at each site of a transcript array can be preferably detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see, Shalon et al., Genome Research 1996, 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 1996, 6:639-645 and in other references cited herein. Alternatively, the fiber-optic bundle described by Ferguson et al., Nature Biotech. 1996, 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated, e.g., by administering a drug, drug-candidate or other compound, or by any other tested event.

In one embodiment of the invention, the relative abundance of an mRNA in two cells or subjects or cell lines tested (e.g., in a treated verses untreated cell or subject) may be scored as perturbed (i.e., where the abundance is different in the two sources of mRNA tested) or as not perturbed (i.e., where the relative abundance in the two sources is the same or is unchanged). Preferably, the difference is scored as perturbed if the difference between the two sources of RNA of at least a factor of about 10% (i.e., RNA from one sources is about 10% more abundant than in the other source), or may be about 25% or about 50%. Still more preferably, the RNA may be scored as perturbed when the difference between the two sources of RNA is at least about a factor of 1.5. Indeed, the difference in abundance between the two sources may be by a factor of two, of five, or more.

In other embodiments, it may be advantageous to also determine the magnitude of the perturbation. This may be done, as noted above, by calculating the ratio of the emission of the two fluorophores used for differential labeling, or by analogous methods that will be readily apparent to those of skill in the art.

In a specific embodiment, exemplified below, Affymetrix® Microarray Suite software can be employed for image acquisition and normalization of the fluorescent signals using internal standards. Analysis of the resultant signal intensities over each oligonucleotide, or data point, within each experiment may then fall into two main categories: supervised learning algorithms (Golub et al., 1999; Slonim et al., 1999; Yeang et al., 2001; Ramaswamy et al., 2001), and Hierarchical Clustering (Eisen et al., 1998; Alizadeh et al., 2000; Perou et al., 2000) (see Example A for the full reference citations). Preferably any algorithms to be employed have the capacity to analyze the very large datasets, and allow comparisons of multiple experiments and multiple points within a single experiment, for determining expression profiles.

### EXAMPLES

The following Example(s) illustrate the invention, but are not limiting.

### Example 1

### Expression Profiling of Blood Cells Distinguishes Prostate Cancer Patients

Patient and Control Subject Recruitment and Study Procedure Institutional Review Board (IRB) approval of the study protocol was obtained.

Medical Exclusions. A list of medical exclusions was generated for both prostate cancer patients and control subjects. A blood count (CBC) was performed for all samples collected and subjects were excluded if their cell counts were outside of the normal range. Serum PSA tests were performed on all patient and control subjects. Any control subject with serum PSA >4 ng/ml was excluded from further analysis (n=0).

Prostate Cancer Subjects. Eleven subjects have been recruited for this study since the initiation of screening of men undergoing radical prostatectomy for treatment of prostate cancer. For all subjects, informed consent was obtained according to the regulations of the IRB. Subjects completed a questionnaire (with the assistance of the study coordinator), documenting current medication and general health status. Fifteen ml of blood was then drawn prior to surgery for prostate removal. Blood was processed immediately as described below. All patient records were screened and pertinent data entered into the subject database, such as serum PSA values (at the time of surgery, prior to surgery, and post surgery), date of biopsy, Gleason score at biopsy and post prostatectomy, TNM tumor stage. The IRB approved study protocol allows the study team to access all patient records following surgery. Ethnicity of prostate cancer subjects was as follows: Caucasian=5, Hispanic=3, African American=2, Asian=1.

Control Subjects. Seven control subjects, age-matched to the patient group, were recruited. Subjects completed a questionnaire documenting that neither they nor their first degree relatives had a history of prostate cancer, or any other tumor. Questionnaires were also completed listing current medication use and medical history. Subjects were seen at their place of work and an informed consent interview was conducted and consent obtained according to the regulations of the IRB. 15 ml blood was drawn from each control and processed immediately as described below. 3 ml blood was employed for PSA and CBC tests, 12 ml was employed for leukocyte extraction. Ethnicity of age-matched control subjects was as follows: Caucasian=4, Hispanic=2, African American=1, Asian=0.

Sample Processing and Microarray Hybridization. Immediately after collection, blood leukocytes were isolated by lysis of red cells, centrifugation and washing, according to standard protocols (Qiagen). Total purified leukocytes were split into two tubes and stored at −70° C. prior to RNA extraction. In studies performed for other projects it is possible to store leukocyte samples for up to 6 months with no effect to quality or quantity of the RNA extracted. Total RNA was extracted in duplicate from the two leukocytes samples, using an RNA preparation kit and accompanying protocol (Qiagen). RNA was quantified by UV spectrometry, using RNA standards for normalization. The quality of RNA was analyzed by electrophoresis through formaldehyde agarose gels. Only RNA samples with good quality ribosomal RNA were processed to completion. For samples employed for microarray analysis, 8 μg of total RNA was used as a template for cDNA synthesis, using an oligo-dT primer and Reverse Transcriptase enzyme, according to standard Affymetrix protocols. Purified cDNA was then employed as a template to generate biotin labeled cRNA, using Enzo Bioarray High Yield RNA Transcript labeling Kits (Enzo). cRNA samples were quantified and stored at −70° C. prior to fragmentation and hybridization.

Following fragmentation of the cRNA samples, 20 ng of each fragmented product was hybridized to an Affymetrix TEST3 array to check the quality of each sample. In each instance the cRNA sample was then hybridized to an HU95A GeneChip array. Patient and control samples were processed and hybridized in a random order.

Affymetrix® Microarray Suite Software. Following scanning of GeneChip arrays, data acquisition of each array was performed using the Affymetrix Microarray Software Suite V5. Briefly, this software initially quantifies the signal over every oligonucleotide probe set on the microarray, then normalizes against the intensity of the signal over the internal control oligonucleotides. The probe set for each gene is then queried by perfect match (PM) and mismatch (MM) oligonucleotide probes, each 25 bases in length. The MM probes have a single base change in the center of the oligonucleotide sequence. Comparison of the hybridization signals from the PM and MM probes permits a measurement of the specificity of signal intensity, and eliminates from the data analysis the majority of non-specific cross hybridization. Values of intensity difference, as well as ratios of each probe pair, are used to determine whether a gene is “present”, i.e. the sample that was hybridized to the array expresses that genes transcript, or “absent”—there is no expression of that gene in the sample used for RNA extraction. To normalize between arrays (to remove experimental noise, such as differences in final cRNA quantity), each array was scaled using a target intensity of 100.

The resultant data was converted to Excel spreadsheets, and collated. As described above, each sample was processed in duplicate. Therefore all data analysis was performed on both the original expression values for each subject duplicate sample, plus the mean expression values of the duplicate subject samples. All gene expression values that were given an “absent call” were removed from the data sets. Gene expression data was filtered by removing all genes with expression levels less than two standard deviation above background levels. All statistical tests and data analysis were performed in Excel, except those described in detail below.

Data analysis; Hierarchical Clustering. Following normalization and filtering, unsupervised and supervised hierarchical clustering was performed using the Cluster program (M. Eisen, discussed Example A). The gene expression data was log-transformed and then median centered over each patient and control sample. Log intensity values for each gene (row), within each subject (column), were then normalized to set all the magnitudes (sum of the squares of the values) to 1.0. Average-linked clustering was performed on this adjusted dataset, employing a correlation centered metric. In this experiment, all genes and subjects were given an equal weighting of 1.0. The results of the clustering run were visualized using the program TreeView (M. Eisen).

Real-Time Polymerase Chain Reaction. 200 ng of total RNA from all patients and controls was employed for first strand cDNA synthesis, using random hexamer primers and Superscript^{II} Reverse Transcriptase enzyme (Invitrogen). Primers were designed using the Primer3 program (Whitehead Institute), except for the 18S ribosomal RNA primers, which were purchased as an internal standard PCR kit (Ambion). For real-time PCR the SYBR Green assay, which measures the linear binding of florescent molecules to double-stranded DNA at each cycle of the PCR amplification, was performed using the Quantitech Kit (Qiagen), on an ABI PRISM 7700 apparatus. The resultant florescence data was imported into Sequence Detector, v1.7a software (ABI), and Cts were calculated. The Ct (the PCR threshold cycle where an increase in reporter fluorescence above a baseline signal can first be detected) has a direct correlation with template concentration. The Cts of samples with known copy numbers were employed to generate standard amplification curves for each set of specific gene primers. Final copy numbers of each patient and control RNA sample were determined from each standard curve, and compared with the control 18S standard results.

Standard PCR protocols were also employed to analyze genes expressed at very low levels in subject leukocytes. cDNA was prepared as described above, and then employed as a template for PCR, using Hotstar polymerase enzyme (Qiagen) and a Hybaid PCR apparatus. Products were analyzed by staining with ethidium bromide following agarose gel electrophoresis. DNA was visualized using a gel documentation system (Kodak).

### Results of the Preliminary Studies

Pair-wise Analysis of Microarray Results. To investigate total sample variability, a pair-wise comparison of expression levels was performed. It is expected that over 12,000 data points, samples should be highly correlated to allow meaningful comparison of the data. Correlation coefficients were within the range of 0.85-0.93 for each comparison (data not shown). In preliminary studies duplicate processing was performed, and pair-wise comparisons between duplicates showed high correlations between intra-subject samples. A scatter plot of expression data from patient A (sample A1-0 and A1-2) yielded an R² value of 0.967.

Analysis of gene expression from genes differentially regulated in peripheral blood. Expression level data for each of the genes previously found to be differentially regulated in peripheral blood were investigated. The mean expression levels were calculated across subjects processed to date from the two groups (mean expression values over duplicate samples). Decreased levels of MSH2 were observed (>20% lower in patients than controls), which although is not significantly different between subject groups (p>0.05), is consistent with the findings reported by Strom et al. (Strom et al., Prostate 2001; 47(4):269-75). Additionally, it was found that transcript levels of IFN gamma were decreased by >20% in the patient leukocytes compared to control subjects. Decreasing levels of serum IFN gamma protein were previously found to correlate with increasing tumor mass (Elsasser-Beile et al., J Cancer Res Clin Oncol. 1993; 119(7):430-3), and the present data suggests that this correlation is directly related to decreased expression in patient peripheral blood leukocytes.

Of interest to this Example 4 in this invention, transcript levels of HER2 were found to be increased in the blood of prostate cancer patients when compared to control subjects (>38% increased in patients versus control subjects). HER2, a proto-oncogenic member of the type 1 tyrosine kinase family is amplified in up to 30% of human breast cancers (Slamon et al., Science. 1987; 9; 235(4785):177-82), and serum levels of HER2, plus RT-PCR amplification of HER2 from circulating metastatic breast cancer cells are being explored as predictors of breast cancer patient survival (Willsher et al., Breast Cancer Res Treat. 1996; 40(3):251-5). Furthermore, many genes that were found to be altered to a much larger degree between the two subject groups than the genes described above, validating the experimental design of using a microarray approach to identify patterns of differentially regulated genes. Examples include the genes Megakaryocyte associated tyrosine kinase (116% decreased in patients versus controls, or >3 fold decrease), programmed cell death-like cDNA (72% decreased in patients versus controls, or >2.8 fold decrease) and MMP9 (40% increased in patients versus controls, or >2 fold increase).

Analysis of IL-8 Leukocyte Gene expression. Veltri et al., supra, reported a significant increase in IL-8 gene expression in leukocytes from patients with metastatic disease, when compared to 18 transcript levels from a pool of control subjects. Analysis of expression levels following microarray hybridization of cRNA transcribed from each patient and control sample showed that IL-8 expression, although quite low, was not different between the two subject groups. The microarray IL-8 gene expression was investigated further, using a PCR based approach. cDNA was transcribed from each RNA sample, and then employed in a real-time PCR assay. To standardize input cDNA and thus RNA levels, PCR amplification products were normalized to the 18S ribosomal RNA gene. Thus real-time PCR was performed, employing 18S primers at concentrations that have been optimized to be in the range of amplification consistent with genes expressed at low levels (Ambion).

Real-Time PCR of the 18S Ribosomal RNA gene. The normalized SYBR Green signal (log Rn; Y axis) is plotted against PCR cycle number (X axis) for each sample. In this experiment, an arbitrary Ct was set to intersect each sample within the linear amplification stage of the PCR, and is represented by the dotted horizontal line. The samples show the control amplifications of a known sample concentration, at no dilution (1.0), 10 fold dilution, 100 fold and 1000 fold. Six subject sample 18S PCR amplifications are performed in duplicate.

A standard curve for 18S was generated, using dilutions of the control sample. The standard curve can be employed to determine both the relative concentration of starting template in each of the subject samples, as well as the actual numbers of molecules employed for analysis. The Cts calculated for each of the subject samples by the Sequence Detector, v1.7a software (ABI), were thus employed to determine the concentration of starting template for each of the samples which were found to be consistent with each other.

In the IL-8 assay, no DNA product was detected in any of the samples after 25 cycles of amplification (which is similar to PCR protocols followed by Veltri et al., Urology 1999; 53(1):139-47). After 40 PCR cycles, product was observed with a clear difference in IL-8 amplification was detected among the samples (data not shown). In each instance, levels of amplification were correlated with those detected following microarray hybridization described above. These results suggest that IL-8 expression is not a marker of localized prostate cancer, but increased expression levels of IL-8 may be a marker of metastatic disease, as detected by Veltri et al. (Veltri et al., supra).

Hierarchical Clustering of Prostate Cancer Patients and Control Subjects. Following normalization and filtering of the data, an unsupervised hierarchical clustering was initially performed, where data is analyzed in the Cluster program, with no previous set constraints on the data. For this analysis, the gene expression data was log transformed and then median centered over each patient and control sample. Following filtering of the data, an initial analysis of genes found to be called “present” in at least two of the samples processed to date was performed; thus a total of 6834 genes remained for further investigation. An unsupervised hierarchical clustering algorithm was implemented, employing the expression intensity levels of genes from 18 subjects. An average-linked cluster was performed on both absolute intensity values of each sample (n=18×2), and the mean intensity levels over the duplicate samples (n=18). Results from both Cluster analysis were viewed in the TreeView program (data not shown), and indicated that using the expression level measurements of 6834 genes, 90% of the prostate cancer patients clustered into one node. However, the classification was not exact as two control subjects also clustered into this node (data not shown).

Supervised Hierarchical Clustering Prostate Cancer Patients and Control Subjects. It may prove useful to perform a supervised clustering experiment, as surrogate tissue in which differences in the patterns of gene expression of leukocytes from tumor patients may be more subtle than the differences obtained from analysis of the tumor tissue itself. Other researchers investigating diagnostic gene expression profiles have performed supervised clustering by manipulating the data before input into the algorithm, for example Dhanasekaran et al. computed t-statistics of prostate cancer versus benign sample for each gene, to create a more limited and also more informative set of genes for analysis (Dhanasekaran et al., Nature. 2001; 412(6849):822-6). Following this example a student two-tailed t-test across the 6834 genes expressed in the patient and control subjects leukocytes was performed. Of the original 6834 genes, 896 were found to have expression values significantly different between the patients and controls (p<0.05), and 1535 were found to have p<0.1 between the two groups. Also performed was an identical student T-test on different permutations of randomized data, where subject samples were randomly placed into one of the two groups (using an approach similar to a permutation method for analysis of non-random data; Draghici et al., Drug Discov Today 2002; 7(11):S55-S63). It was found that the average number of genes found to be significantly different between the randomized groups was 200 (p<0.05), while <500 genes were found to have p<0.1. A t-test performed on the p-values of the “real” group versus the random groups showed a significant difference between groups (p<0.0001). Therefore, randomizing the data results in nearly 80% less genes found to be significantly different between subject groups and may represent the noise of this experimental system.

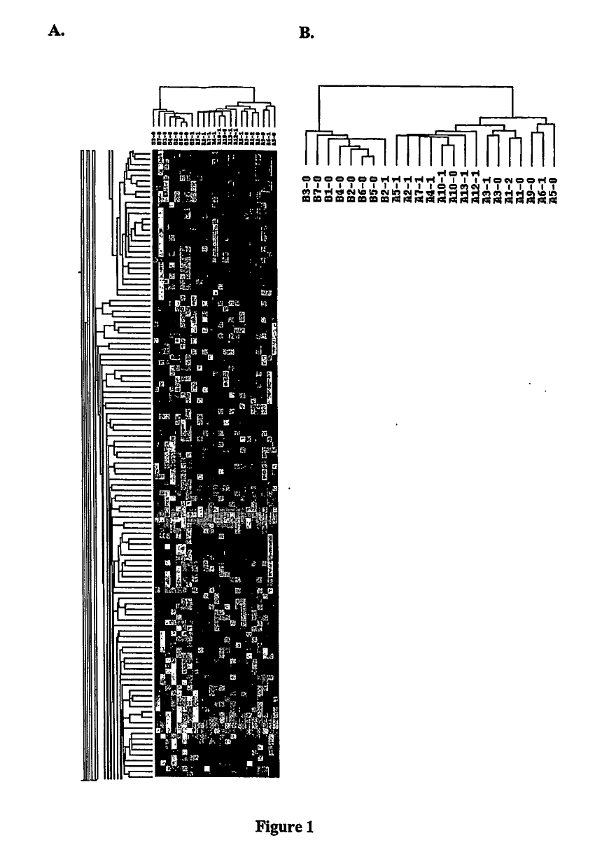

TreeView Representation of Cluster patterns of gene expression among men with prostate cancer and age-matched control subjects (FIG. 1). Data are represented in matrix format. Each row represents a single gene (for space gene names have been omitted). Each column represents an experimental leukocyte patient or control sample. For each sample the ratio of the abundance of transcripts of each gene, to the median abundance of the genes's transcript among the individuals leukocytes, is represented by the color of the corresponding matrix. Green means that transcript levels are less than median; black means the transcript levels are median; red means the transcript levels are greater than median. Grey is used to indicate that the gene is absent. Color saturation represents the magnitude of the ratio relative to the median for the total set of samples. A dendrogram along the horizontal axis indicates the clusters of most similar subjects, based on gene expression levels of 1535 genes. The dendrogram along the vertical axis represents sample nodes of the total Cluster results, where genes appear together on the branches of the tree if they have similar patterns of gene expression. Examples of Cluster nodes are taken from the total TreeView data, showing genes that are generally expressed at lower levels in the prostate cancer samples (A1 to A13), than control subject samples (B1 to B7). A scaled representation of the horizontal dendrogram showing patient and control cluster results can be shown.

The 1535 genes (p<0.1) were further analyzed employing the Cluster program with readout in TreeView. Again, this analysis was performed using both the mean of duplicate subject samples and the absolute intensity levels of each sample. FIG. 1 shows an example of this data analysis, where mean intensity levels were employed for all but three samples. The results of this supervised cluster analysis indicates that the overall leukocyte expression of 1535 genes from the 11 prostate cancer patients is different to the overall gene expression data of the seven control subjects. Specifically, the prostate cancer patients cluster in a node that is separate to the node of control subjects, and suggests that distinctive patterns of gene expression can be employed to differentiate between prostate cancer patients and control subjects. The use of duplicate samples permits a finding that experimental difference (as observed between B2-0 and B2-1), do not influence the final cluster results.

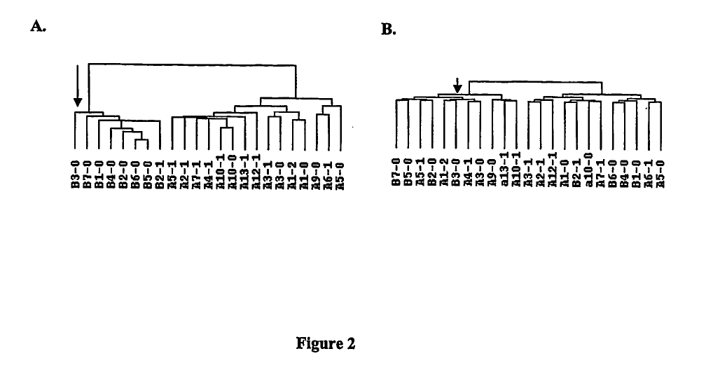

To perform an investigation on this clustering result, subject gene expression levels were randomized within the dataset and the resultant data were re-clustered. Five different re-iterations of randomizing the data were performed. A TreeView readout from the clustering of 1535 genes, where subjects have been classified into one of two nodes representing cancer patients or control subjects, and a TreeView readout generated following Cluster analysis of the randomized dataset was used to analyze the data (FIG. 2). Short branch lengths between each node of the dendrogram of random data show that following intra-subject randomization, patient samples have overall gene expression patterns very similar to each other. Furthermore, the dendrogram has not organized the samples into an order significantly different from the initial order of data input into the Cluster algorithm and duplicate samples are dispersed over the tree. The Cluster analysis of the other random data iterations resulted in TreeView readouts where either the samples remained in the order of input into Cluster, or alternatively branch lengths were observed to be vastly reduced, indicating very minor differences in overall gene expression between subjects. These results suggest that this supervised hierarchical clustering, which demonstrates a correct classification of prostate cancer patients and control subjects into their respective groups, is not due to random microarray data.

Table 1 shows a list of genes from PBLs up- or down-regulated in prostate cancer subjects.

TABLE 1
Prostate Cancer Gene Expression Results
This table includes gene expression profile data from 11 prostate cancer patients
versus 6 control subjects. The table includes the Affymetrix probe-set ID for the HU95Av2
GeneChip array, and also the EASE assignment. The EASE data were included because
there are instances where an unknown EST (as referenced to by the Affymetrix probeset
ID) has later been characterized by others. However, these curation methods are not 100%
accurate.
It is very important to note that the significance levels for the genes/ESTs can
change with increasing statistical power from comparing additional samples. Therefore, it
may be likely that some genes/ESTs may change in significance.

| | Mean levels | | |
|---|---|---|---|
| | expression in | two tailed | |
| Affymetrix | prostate cancer | Students | |
| HU95A version2 | patients compared | t-test | EASE Names |
| probe set ids | to healthy controls | significance | (david.niaid.nih.gov/david/ease.h |
| 37046_at | down | 1.95E−07 | proteasome (prosome, macropain) subunit, alpha type, 5 |
| 2010_at | down | 3.1E−07 | S-phase kinase-associated protein 1A (p19A) |
| 36546_r_at | up | 1.66E−06 | KIAA0542 gene product |
| 33602_at | up | 4.77E−06 | endothelial differentiation, G-protein-coupled receptor 6 |
| 36591_at | up | 4.9E−06 | tubulin, alpha 1 (testis specific) |
| 33190_g_at | up | 5.31E−06 | chromosome 10 open reading frame 6 |
| 32595_at | down | 6.5E−06 | G-rich RNA sequence binding factor 1 |
| 39030_at | up | 7.14E−06 | Rab acceptor 1 (prenylated) |
| 36567_at | up | 7.59E−06 | solute carrier family 17 (sodium-dependent inorganic |
| | | | phosphate cotransporter), member 7 |
| 32066_g_at | up | 9.85E−06 | cAMP responsive element modulator |
| 38964_r_at | up | 1.15E−05 | Wiskott-Aldrich syndrome (eczema-thrombocytopenia) |
| 35485_at | up | 1.35E−05 | glutamate receptor, metabotropic 4 |
| 32622_at | up | 1.53E−05 | dynamin 2 |
| 33126_at | down | 1.7E−05 | glycosyltransferase AD-017 |
| 38621_at | up | 1.9E−05 | dimethylarginine dimethylaminohydrolase 2 |
| 31620_at | up | 2.07E−05 | similar to transcription factor TBX10 |
| 330_s_at | up | 2.28E−05 | Tubulin, Alpha 1, isoform 44 |
| 32378_at | up | 2.76E−05 | pyruvate kinase, muscle |
| 36098_at | down | 2.87E−05 | splicing factor, arginine/serine-rich 1 (splicing factor 2, |
| | | | alternate splicing factor) |
| 1268_at | up | 3.43E−05 | ubiquitin-activating enzyme E1 (A1S9T and BN75 |
| | | | temperature sensitivity complementing) |
| 31391_at | up | 6.24E−05 | huntingtin-associated protein 1 (neuroan 1) |
| 39797_at | down | 6.39E−05 | ubiquitin ligase E3 alpha-II |
| 1660_at | down | 6.4E−05 | ubiquitin-conjugating enzyme E2N (UBC13 homolog, |
| | | | yeast) |
| 31901_at | up | 7.3E−05 | potassium voltage-gated channel, shaker-related |
| | | | subfamily, beta member 2 |
| 1772_s_at | down | 7.48E−05 | farnesyltransferase, CAAX box, alpha |
| 36994_at | up | 8.05E−05 | ATPase, H+ transporting, lysosomal 16 kDa, V0 subunit c |
| 32659_at | down | 8.47E−05 | eukaryotic translation initiation factor 2B, subunit 4 delta, |
| | | | 67 kDa |
| 41248_at | down | 9.53E−05 | likely ortholog of mouse variant polyadenylation protein |
| | | | CSTF-64 |
| 39709_at | up | 0.000116 | selenoprotein W, 1 |
| 31740_s_at | up | 0.000125 | paired box gene 4 |
| 40418_at | down | 0.000125 | retinoblastoma binding protein 4 |
| 39792_at | down | 0.000141 | heterogeneous nuclear ribonucleoprotein R |
| 41078_at | up | 0.000149 | KIAA0150 protein |
| 31341_at | up | 0.000154 | potassium voltage-gated channel, Shaw-related |
| | | | subfamily, member 3 |
| 32163_f_at | up | 0.000157 | chorionic somatomammotropin hormone 2 |
| 676_g_at | up | 0.000162 | interferon induced transmembrane protein 1 (9-27) |
| 34832_s_at | up | 0.000163 | KIAA0763 gene product |
| 924_s_at | down | 0.000166 | protein phosphatase 2 (formerly 2A), catalytic subunit, |
| | | | beta isoform |
| 34491_at | up | 0.000166 | 2′-5′-oligoadenylate synthetase-like |
| 1392_at | up | 0.000172 | G protein-coupled receptor kinase 6 |
| 39118_at | down | 0.000176 | DnaJ (Hsp40) homolog, subfamily A, member 1 |
| 34141_at | up | 0.000188 | |
| 31785_f_at | up | 0.000197 | unnamed HERV-H protein |
| 41219_at | down | 0.000227 | KIAA0570 gene product |
| 38105_at | down | 0.000234 | hypothetical protein FLJ11021 similar to splicing factor, |
| | | | arginine/serine-rich 4 |
| 37334_at | down | 0.000239 | heterogeneous nuclear ribonucleoprotein A0 |
| 40452_at | up | 0.000249 | copine I |
| 32784_at | down | 0.000259 | PRP4 pre-mRNA processing factor 4 homolog B (yeast) |
| 31968_at | up | 0.000266 | |
| 36907_at | up | 0.000271 | mevalonate kinase (mevalonic aciduria) |
| 35577_at | up | 0.000275 | serine (or cysteine) proteinase inhibitor, clade B |
| | | | (ovalbumin), member 7 |
| 32115_r_at | up | 0.000285 | adenosine A2a receptor |
| 1577_at | up | 0.000288 | androgen receptor (dihydrotestosterone receptor; |
| | | | testicular feminization; spinal and bulbar muscular |
| | | | atrophy; Kennedy disease) |
| 1830_s_at | up | 0.000295 | transforming growth factor, beta 1 (Camurati-Engelmann |
| | | | disease) |
| 33803_at | down | 0.000306 | thrombomodulin |
| 41159_at | down | 0.000311 | clathrin, heavy polypeptide (Hc) |
| 1158_s_at | up | 0.000315 | calmodulin 3 (phosphorylase kinase, delta) |
| 39162_at | down | 0.000328 | Arg/Abl-interacting protein ArgBP2 |
| 37201_at | up | 0.000335 | inter-alpha (globulin) inhibitor H4 (plasma Kallikrein- |
| | | | sensitive glycoprotein) |
| 37383_f_at | up | 0.000338 | major histocompatibility complex, class I, C |
| 41836_at | down | 0.000345 | calcium homeostasis endoplasmic reticulum protein |
| 38963_i_at | up | 0.000349 | Wiskott-Aldrich syndrome (eczema-thrombocytopenia) |
| 34827_at | up | 0.000362 | unc-51-like kinase 1 (C. elegans) |
| 37074_at | up | 0.00037 | syntrophin, beta 1 (dystrophin-associated protein A1, |
| | | | 59 kDa, basic component 1) |
| 37746_r_at | up | 0.000372 | suppression of tumorigenicity 5 |
| 37267_at | up | 0.000373 | thimet oligopeptidase 1 |
| 33779_at | up | 0.000386 | vesicle-associated membrane protein 1 (synaptobrevin 1) |
| 457_s_at | down | 0.000395 | ubiquitin-like 1 (sentrin) |
| 41745_at | up | 0.000404 | interferon induced transmembrane protein 3 (1-8U) |
| 37468_at | down | 0.000419 | Janus kinase 2 (a protein tyrosine kinase) |
| 35802_at | down | 0.000424 | formin binding protein 4 |
| 1698_at | down | 0.000429 | polymerase (DNA directed), beta |
| 38409_at | down | 0.000433 | sperm specific antigen 2 |
| 38093_at | down | 0.000442 | chromosome 14 open reading frame 32 |
| 36143_at | down | 0.000469 | caspase 3, apoptosis-related cysteine protease |
| 34151_at | up | 0.000471 | DKFZP586M1019 protein |
| 41033_at | down | 0.000475 | zinc finger protein 84 (HPF2) |
| 32053_at | down | 0.000477 | cyclin T2 |
| 38865_at | up | 0.000509 | GRB2-related adaptor protein 2 |
| 36377_at | up | 0.00052 | interleukin 18 receptor 1 |
| 37977_at | up | 0.00052 | deltex homolog 2 (Drosophila) |
| 32447_at | up | 0.000538 | nuclear receptor subfamily 5, group A, member 1 |
| 36926_at | down | 0.00055 | mitogen-activated protein kinase 6 |
| 869_at | down | 0.000554 | general transcription factor IIA, 2, 12 kDa |
| 34604_at | up | 0.000585 | solute carrier family 6 (neurotransmitter transporter, |
| | | | serotonin), member 4 |
| 41795_at | down | 0.000605 | NCK adaptor protein 1 |
| 33542_at | up | 0.00061 | |
| 40355_at | up | 0.000621 | AND-1 protein |
| 40585_at | down | 0.000638 | adenylate cyclase 7 |
| 34384_at | down | 0.000654 | ATP-binding cassette, sub-family C (CFTR/MRP), |
| | | | member 1 |
| 34907_at | up | 0.000654 | apoptosis-associated tyrosine kinase |
| 2058_s_at | up | 0.000655 | integrin, beta 5 |
| 35899_at | up | 0.000659 | artemin |
| 140_s_at | down | 0.000663 | splicing factor, arginine/serine-rich 10 (transformer 2. |
| | | | homolog, Drosophila) |
| 40976_at | up | 0.000674 | katanin p80 (WD repeat containing) subunit B 1 |
| 33180_at | down | 0.000681 | protein phosphatase 1, regulatory (inhibitor) subunit 2 |
| 34157_f_at | up | 0.000685 | histone 1, H2al |
| 32080_at | up | 0.000692 | tetracycline transporter-like protein |
| 39336_at | up | 0.000697 | ADP-ribosylation factor 3 |
| 36675_r_at | up | 0.000703 | profilin 1 |
| 36720_at | up | 0.000705 | pyruvate dehydrogenase kinase, isoenzyme 3 |
| 38223_at | down | 0.000723 | TBC1 domain family, member 8 (with GRAM domain) |
| 32198_at | up | 0.000738 | hypothetical protein FLJ20452 |
| 40007_at | up | 0.000744 | zinc finger protein, subfamily 1A, 1 (Ikaros) |
| 1351_at | up | 0.000757 | EphB4 |
| 1307_at | down | 0.000757 | xeroderma pigmentosum, complementation group A |
| 36258_at | up | 0.000769 | protein kinase, cGMP-dependent, type I |
| 37692_at | down | 0.000799 | diazepam binding inhibitor (GABA receptor modulator, |
| | | | acyl-Coenzyme A binding protein) |
| 32548_at | down | 0.000801 | unactive progesterone receptor, 23 kD |
| 38608_at | up | 0.000802 | lectin, galactoside-binding, soluble, 7 (galectin 7) |
| 37968_at | up | 0.000806 | natural cytotoxicity triggering receptor 3 |
| 39091_at | down | 0.000806 | vitamin A responsive; cytoskeleton related |
| 39057_at | up | 0.000808 | kinesin 2 60/70 kDa |
| 33226_at | up | 0.00082 | KIAA0876 protein |
| 40580_r_at | up | 0.000825 | parathymosin |
| 41428_at | up | 0.000843 | ATP-binding cassette, sub-family C (CFTR/MRP), |
| | | | member 5 |
| 354_s_at | down | 0.000847 | RecQ protein-like (DNA helicase Q1-like) |
| 34694_at | up | 0.000848 | SWI/SNF related, matrix associated, actin dependent |
| | | | regulator of chromatin, subfamily d, member 2 |
| 32433_at | down | 0.00085 | |
| 34098_f_at | up | 0.000893 | integrin cytoplasmic domain-associated protein 1 |
| 34062_at | up | 0.000902 | ets variant gene 2 |
| 38967_at | down | 0.000913 | chromosome 14 open reading frame 2 |
| 34330_at | down | 0.000915 | cytochrome c oxidase subunit VIIa polypeptide 2 like |
| 32201_at | up | 0.000925 | Sjogren's syndrome nuclear autoantigen 1 |
| 1127_at | up | 0.000936 | ribosomal protein S6 kinase, 90 kDa, polypeptide 1 |
| 40268_at | up | 0.000942 | FOS-like antigen 2 |
| 36023_at | down | 0.000951 | proline-rich protein HaeIII subfamily 1 |
| AFFX-CreX-3_st | up | 0.000965 | |
| 33913_at | up | 0.000995 | HLA-B associated transcript 2 |
| 37838_at | up | 0.001007 | coagulation factor XII (Hageman factor) |
| 37098_at | up | 0.001029 | protoporphyrinogen oxidase |
| 1333_f_at | up | 0.001043 | breakpoint cluster region |
| 32904_at | up | 0.001057 | perforin 1 (pore forming protein) |
| 33103_s_at | down | 0.001068 | adducin 3 (gamma) |
| 34811_at | down | 0.001072 | ATP synthase, H+ transporting, mitochondrial F0 complex, |
| | | | subunit c (subunit 9) isoform 3 |
| 40504_at | up | 0.001073 | paraoxonase 2 |
| 33764_at | up | 0.001075 | G protein-coupled receptor 51 |
| 35626_at | up | 0.001085 | N-sulfoglucosamine sulfohydrolase (sulfamidase) |
| 38726_at | up | 0.001085 | dolichyl-phosphate mannosyltransferase polypeptide 2, |
| | | | regulatory subunit |
| 1794_at | up | 0.001089 | cyclin D3 |
| 534_s_at | up | 0.001117 | folate receptor 1 (adult) |
| 34714_at | down | 0.001123 | SAM domain and HD domain 1 |
| 1452_at | down | 0.001145 | LIM domain only 4 |
| 35132_at | up | 0.001152 | myosin IF |
| 40947_at | up | 0.001185 | hypothetical protein FLJ12671 |
| 36343_at | up | 0.001189 | tolloid-like 2 |
| 35693_at | up | 0.001227 | hippocalcin-like 1 |
| 34486_at | up | 0.001262 | |
| 34702_f_at | up | 0.001285 | chorionic somatomammotropin hormone 2 |
| 35171_at | down | 0.00129 | spastic paraplegia 4 (autosomal dominant; spastin) |
| 38057_at | up | 0.001298 | dermatopontin |
| 41333_at | down | 0.001302 | centaurin, beta 2 |
| 34703_f_at | up | 0.001305 | chorionic somatomammotropin hormone 2 |
| 41821_at | down | 0.001307 | cell division cycle 2-like 5 (cholinesterase-related cell |
| | | | division controller) |
| 41788_i_at | down | 0.001308 | KIAA0669 gene product |
| 37604_at | down | 0.001315 | histamine N-methyltransferase |
| 921_s_at | up | 0.001335 | |
| 39444_at | down | 0.001337 | splicing factor 3b, subunit 1, 155 kDa |
| 38072_at | down | 0.001339 | hypothetical protein dJ465N24.2.1 |
| 39624_at | up | 0.001377 | leukotriene B4 receptor |
| AFFX- | up | 0.001382 | actin, beta |
| HSAC07/X00351_M_at | | | |
| 38449_at | up | 0.0014 | WD repeat domain 23 |
| 39353_at | down | 0.001411 | heat shock 10 kDa protein 1 (chaperonin 10) |
| 40260_g_at | up | 0.001416 | RNA binding motif protein 9 |
| 33372_at | up | 0.001431 | RAB31, member RAS oncogene family |
| 39166_s_at | up | 0.001476 | serine (or cysteine) proteinase inhibitor, clade H (heat |
| | | | shock protein 47), member 1, (collagen binding protein 1 |
| 40138_at | up | 0.00148 | COP9 subunit 6 (MOV34 homolog, 34 kD) |
| 35451_s_at | up | 0.001495 | SCAN domain containing 2 |
| 34802_at | up | 0.001519 | collagen, type VI, alpha 2 |
| 36654_s_at | down | 0.00153 | heterogeneous nuclear ribonucleoprotein A2/B1 |
| 33887_at | up | 0.00159 | hepatocyte growth factor-regulated tyrosine kinase |
| | | | substrate |
| 39748_at | down | 0.001591 | |
| 35753_at | up | 0.001648 | PRP8 pre-mRNA processing factor 8 homolog (yeast) |
| 41752_at | down | 0.001662 | growth hormone inducible transmembrane protein |
| 31926_at | up | 0.001667 | cytochrome P450, family 7, subfamily A, polypeptide 1 |
| 32407_f_at | up | 0.001671 | |
| 39909_g_at | up | 0.001683 | TAF6-like RNA polymerase II, p300/CBP-associated |
| | | | factor (PCAF)-associated factor, 65 kDa |
| 36270_at | down | 0.001685 | CD86 antigen (CD28 antigen ligand 2, B7-2 antigen) |
| 40359_at | up | 0.001688 | chromosome 11 open reading frame 13 |
| 39079_at | down | 0.001696 | enhancer of rudimentary homolog (Drosophila) |
| 650_s_at | down | 0.001755 | calcium/calmodulin-dependent protein kinase (CaM |
| | | | kinase) II gamma |
| 33783_at | up | 0.001762 | plexin B1 |
| 39119_s_at | up | 0.001784 | natural killer cell transcript 4 |
| 36814_at | down | 0.001798 | hypothetical protein KIAA1109 |
| 40518_at | down | 0.001806 | protein tyrosine phosphatase, receptor type, C |
| 34056_g_at | up | 0.001806 | activin A receptor, type IB |
| 40110_at | down | 0.001822 | isocitrate dehydrogenase 3 (NAD+) beta |
| 421_at | up | 0.001832 | translocated promoter region (to activated MET |
| | | | oncogene) |
| 37386_i_at | up | 0.001833 | KDEL (Lys-Asp-Glu-Leu) endoplasmic reticulum protein |
| | | | retention receptor 1 |
| 32803_at | down | 0.001853 | comichon homolog (Drosophila) |
| 38336_at | up | 0.001856 | GRP1-binding protein GRSP1 |
| 34263_s_at | down | 0.001864 | diaphanous homolog 2 (Drosophila) |
| 39949_at | up | 0.001866 | molybdenum cofactor synthesis 1 |
| 36715_at | up | 0.001899 | adrenergic, alpha-1A-, receptor |
| 38500_at | down | 0.001906 | CGI-109 protein |
| 31557_at | up | 0.001915 | thymosin, beta 4, X chromosome |
| 32206_at | up | 0.001916 | CDC42 binding protein kinase alpha (DMPK-like) |
| 34819_at | down | 0.001946 | CD164 antigen, sialomucin |
| 40988_at | down | 0.001949 | YME1-like 1 (S. cerevisiae) |
| 38982_at | down | 0.001952 | telomeric repeat binding factor 2, interacting protein |
| 31610_at | up | 0.001954 | membrane-associated protein 17 |
| 33378_at | down | 0.001956 | IDN3 protein |
| 34353_at | down | 0.00196 | KIAA0648 protein |
| 41529_g_at | down | 0.001972 | |
| 36895_at | down | 0.001975 | origin recognition complex, subunit 3-like (yeast) |
| 38814_at | down | 0.001995 | ATPase, H+ transporting, lysosomal 13 kDa, V1 subunit |
| | | | isoform 1 |
| 35986_at | up | 0.001998 | MYST histone acetyltransferase 1 |
| 37075_at | up | 0.002003 | syntrophin, beta 1 (dystrophin-associated protein A1, |
| | | | 59 kDa, basic component 1) |
| 37358_at | down | 0.002024 | ubiquitin-conjugating enzyme E2E 1 (UBC4/5 homolog, |
| | | | yeast) |
| 37995_s_at | down | 0.002036 | fragile X mental retardation 1 |
| 36694_at | up | 0.00204 | potassium voltage-gated channel, delayed-rectifier, |
| | | | subfamily S, member 3 |
| 35804_at | down | 0.002047 | ash2 (absent, small, or homeotic)-like (Drosophila) |
| 34409_at | up | 0.002066 | low density lipoprotein receptor-related protein 10 |
| 40360_at | up | 0.002068 | Protein P3 |
| 35600_at | down | 0.00209 | ROD1 regulator of differentiation 1 (S. pombe) |
| 32005_at | up | 0.002111 | pro-melanin-concentrating hormone |
| 40489_at | up | 0.002142 | dentatorubral-pallidoluysian atrophy (atrophin-1) |
| 38355_at | down | 0.002155 | DEAD/H (Asp-Glu-Ala-Asp/His) box polypeptide, Y |
| | | | chromosome |
| 41598_at | down | 0.002175 | SEC22 vesicle trafficking protein-like 1 (S. cerevisiae) |
| 193_at | down | 0.002179 | TAF9 RNA polymerase II, TATA box binding protein |
| | | | (TBP)-associated factor, 32 kDa |
| 41756_at | down | 0.002228 | XPA binding protein 1 |
| 40509_at | down | 0.00226 | electron-transfer-flavoprotein, alpha polypeptide (glutaric |
| | | | aciduria II) |
| 36981_at | down | 0.002271 | signal recognition particle 9 kDa |
| 400 _at | down | 0.002273 | mago-nashi homolog, proliferation-associated |
| | | | (Drosophila) |
| 40698_at | down | 0.002275 | C-type (calcium dependent, carbohydrate-recognition |
| | | | domain) lectin, superfamily member 2 (activation-induced |
| 33664_g_at | up | 0.002278 | |
| 37389_at | down | 0.002286 | small acidic protein |
| 640_at | up | 0.002306 | angiotensin II receptor-like 2 |
| 37031_at | down | 0.002324 | chromosome 9 open reading frame 10 |
| 40844_at | down | 0.00233 | SH2 domain binding protein 1 (tetratricopeptide repeat |
| | | | containing) |
| 35735_at | down | 0.002374 | guanylate binding protein 1, interferon-inducible, 67 kDa |
| 254_at | down | 0.002379 | H3 histone, family 3A |
| 32232_at | down | 0.002401 | NADH dehydrogenase (ubiquinone) 1 beta subcomplex, |
| | | | 5, 16 kDa |
| 39039_s_at | down | 0.002472 | ubiquitin-conjugating enzyme E2, J1 (UBC6 homolog, |
| | | | yeast) |
| 35768_at | up | 0.002473 | ring finger protein 40 |
| 37145_at | up | 0.002506 | granulysin |
| 41360_at | down | 0.002506 | CCR4-NOT transcription complex, subunit 8 |
| 1798_at | down | 0.002559 | LIV-1 protein, estrogen regulated |
| 322_at | up | 0.002593 | phosphoinositide-3-kinase, regulatory subunit, polypeptide |
| | | | 3 (p55, gamma) |
| 35035_at | up | 0.002603 | cholinergic receptor, nicotinic, beta polypeptide 3 |
| 34558_at | up | 0.002611 | opiate receptor-like 1 |
| 32789_at | down | 0.002639 | nuclear cap binding protein subunit 2, 20 kDa |
| 32422_at | up | 0.002657 | double C2-like domains, beta |
| 31388_at | up | 0.002677 | early lymphoid activation protein |
| 38880_at | up | 0.002701 | likely ortholog of mouse mitogen activated protein kinase |
| | | | binding proten 1 |
| 34611_at | up | 0.002705 | zinc finger protein 192 |
| 39629_at | up | 0.002729 | phospholipase A2, group V |
| 1827_s_at | up | 0.002732 | v-myc myelocytomatosis viral oncogene homolog (avian) |
| 34786_at | down | 0.002751 | jumonji domain containing 1 |
| 652_g_at | down | 0.002754 | replication protein A3, 14 kDa |
| 38480_s_at | up | 0.002774 | ubiquitin-conjugating enzyme E2I (UBC9 homolog, yeast) |
| 38860_at | up | 0.002779 | phosphodiesterase 4C, cAMP-specific |
| | | | (phosphodiesterase E1 dunce homolog, Drosophila) |
| 39083_at | down | 0.002817 | ubiquitin-conjugating enzyme E2D 3 (UBC4/5 homolog, |
| | | | yeast) |
| 38589_l_at | down | 0.00284 | prothymosin, alpha (gene sequence 28) |
| 38753_at | down | 0.002847 | exportin, tRNA (nuclear export receptor for tRNAs) |
| 41423_at | up | 0.002861 | calsyntenin 3 |
| 36474_at | down | 0.002868 | KIAA0776 protein |
| 34336_at | down | 0.00288 | lysyl-tRNA synthetase |
| 184_at | up | 0.002913 | angiotensin II receptor-like 1 |
| 33546_at | up | 0.002927 | |
| 40044_at | up | 0.002937 | ELL gene (11-19 lysine-rich leukemia gene) |
| 2031_s_at | up | 0.002964 | cyclin-dependent kinase inhibitor 1A (p21, Cip1) |
| 34178_at | up | 0.002967 | zinc finger protein 297 |
| 33098_at | down | 0.003008 | chemokine (C—C motif) receptor 3 |
| 955_at | up | 0.003035 | Calmodulin Type 1 |
| 33440_at | down | 0.00306 | transcription factor 8 (represses interleukin 2 expression |
| 31870_at | up | 0.003092 | CD37 antigen |
| 31861_at | up | 0.003094 | immunoglobulin mu binding protein 2 |
| 36949_at | up | 0.00313 | casein kinase 1, delta |
| 35681_r_at | down | 0.003133 | zinc finger homeobox 1b |
| 1848_at | down | 0.003145 | RAP1A, member of RAS oncogene family |
| 41612_at | up | 0.003146 | zinc finger protein 264 |
| 40038_at | up | 0.003148 | suppression of tumorigenicity 7 |
| 35749_at | up | 0.003152 | transcriptional adaptor 3 (NGG1 homolog, yeast)-like |
| 38370_at | down | 0.00316 | |
| 33848_r_at | down | 0.003175 | cyclin-dependent kinase inhibitor 1B (p27, Kip1) |
| 35426_at | up | 0.003201 | SPPL2b |
| 33417_at | up | 0.003215 | RAB3 GTPase-ACTIVATING PROTEIN |
| 36791_g_at | up | 0.003221 | tropomyosin 1 (alpha) |
| 38822_at | up | 0.003235 | serine/threonine kinase 17a (apoptosis-inducing) |
| 40481_r_at | up | 0.003244 | FYN oncogene related to SRC, FGR, YES |
| 36805_s_at | up | 0.003265 | neurotrophic tyrosine kinase, receptor, type 1 |
| 31519_f_at | down | 0.003267 | basic transcription factor 3, like 3 |
| 37844_at | down | 0.003277 | class I cytokine receptor |
| 39553_at | down | 0.003292 | phosphatase and tensin homolog (mutated in multiple |
| | | | advanced cancers 1) |
| 41386_i_at | up | 0.003297 | KIAA0346 protein |
| 41141_at | down | 0.003301 | protein-kinase, interferon-inducible double stranded RN |
| | | | dependent inhibitor, repressor of (P58 repressor) |
| 40957_at | down | 0.003302 | joined to JAZF1 |
| 33820_g_at | down | 0.00335 | lactate dehydrogenase B |
| 36688_at | down | 0.003368 | sterol carrier protein 2 |
| 1760_s_at | up | 0.003403 | protein tyrosine phosphatase, non-receptor type 7 |
| 31584_at | down | 0.003438 | tumor protein, translationally-controlled 1 |
| 40610_at | down | 0.003441 | zinc finger RNA binding protein |
| 108_g_at | up | 0.003453 | |
| 32590_at | down | 0.003459 | nucleolin |
| 38516_at | up | 0.003465 | sodium channel, voltage-gated, type I, beta |
| 33113_at | down | 0.003483 | Cbp/p300-interacting transactivator, with Glu/Asp-rich |
| | | | carboxy-terminal domain, 2 |
| 34337_s_at | down | 0.003512 | likely ortholog of mouse metal response element binding |
| | | | transcription factor 2 |
| 35976_at | up | 0.003514 | Cbp/p300-interacting transactivator, with Glu/Asp-rich |
| | | | carboxy-terminal domain, 1 |
| 33622_at | up | 0.003533 | calcium channel, voltage-dependent, L type, alpha 1C |
| | | | subunit |
| 552_at | up | 0.003551 | Rho GTPase activating protein 1 |
| 36571_at | down | 0.00356 | topoisomerase (DNA) II beta 180 kDa |
| 36887_f_at | up | 0.003595 | killer cell immunoglobulin-like receptor, three domains, |
| | | | long cytoplasmic tail, 1 |
| 1662_r_at | up | 0.003647 | Antigen, Prostate Specific, Alt. Splice Form 2 |
| 40555_at | down | 0.003673 | ras homolog gene family, member Q |
| 1389_at | up | 0.003688 | membrane metallo-endopeptidase (neutral |
| | | | endopeptidase, enkephalinase, CALLA, CD10) |
| 37729_at | down | 0.003702 | exportin 1 (CRM1 homolog, yeast) |
| 34485_r_at | up | 0.003769 | ADP-ribosylation factor guanine nucleotide-exchange |
| | | | factor 2 (brefeldin A-inhibited) |
| 582_g_at | down | 0.003786 | nuclear receptor subfamily 2, group C, member 1 |
| 38415_at | down | 0.003786 | protein tyrosine phosphatase type IVA, member 2 |
| 2070_i_at | up | 0.003801 | mitogen-activated protein kinase 8 |
| 40392_at | up | 0.003801 | caudal type homeo box transcription factor 2 |
| 35761_at | down | 0.003805 | aminoadipate-semialdehyde dehydrogenase- |
| | | | phosphopantetheinyl transferase |
| 36793_at | up | 0.003865 | hypothetical protein AY099107 |
| 31859_at | up | 0.003871 | matrix metalloproteinase 9 (gelatinase B, 92 kDa |
| | | | gelatinase, 92 kDa type IV collagenase) |
| 40928_at | down | 0.003876 | SOCS box-containing WD protein SWiP-1 |
| 41253_s_at | down | 0.003901 | chorionic somatomammotropin hormone 2 |
| 1724_at | up | 0.003958 | E2F transcription factor 4, p107/p130-binding |
| 37448_s_at | up | 0.003965 | GNAS complex locus |
| 34081_at | up | 0.004003 | |
| 40098_at | up | 0.004012 | EH-domain containing 1 |
| 38915_at | up | 0.004044 | KIAA0563 gene product |
| 37523_at | up | 0.004046 | acyl-Coenzyme A dehydrogenase, long chain |
| 36179_at | up | 0.004048 | mitogen-activated protein kinase-activated protein kinase 2 |
| 37481_at | down | 0.00405 | cell division cycle 40 homolog (yeast) |
| 40376_at | up | 0.004057 | arylsulfatase E (chondrodysplasia punctata 1) |
| 1862_at | up | 0.004074 | ataxia telangiectasia mutated (includes complementation |
| | | | groups A, C and D) |
| 40497_at | up | 0.004088 | homologous to yeast nitrogen permease (candidate tumor |
| | | | suppressor) |
| 35317_at | down | 0.004109 | meningioma expressed antigen 5 (hyaluronidase) |
| 2051_at | up | 0.004115 | O-6-methylguanine-DNA methyltransferase |
| 1759_f_at | up | 0.00415 | cytochrome P450, family 3, subfamily A, polypeptide 7 |
| 35842_at | down | 0.00418 | |
| 276_at | down | 0.004191 | DnaJ (Hsp40) homolog, subfamily A, member 1 |
| 38661_at | up | 0.004217 | RNA-binding region (RNP1, RRM) containing 1 |
| 39086_g_at | down | 0.004239 | single-stranded DNA binding protein |
| 35861_at | up | 0.004246 | sialyltransferase 4A (beta-galactoside alpha-2,3- |
| | | | sialyltransferase) |
| 38756_at | up | 0.004248 | RAP1A, member of RAS oncogene family |
| 34441_at | up | 0.004253 | |
| AFFX- | up | 0.004262 | actin, beta |
| HSAC07/X00351_5_at | | | |
| 39169_at | down | 0.004272 | Sec61 gamma |
| 1128_s_at | up | 0.004279 | chemokine (C—C motif) receptor 1 |
| 38882_r_at | up | 0.004292 | tripartite motif-containing 16 |
| 1929_at | down | 0.004324 | angiopoietin 1 |
| 32088_at | down | 0.004339 | basic leucine zipper nuclear factor 1 (JEM-1) |
| 38558_at | up | 0.004345 | myelin associated glycoprotein |
| 31385_at | up | 0.004347 | ribosomal protein L28 |
| 32011_g_at | up | 0.004354 | hypothetical protein EAN57 |
| 494_at | up | 0.004374 | interleukin 13 |
| 38326_at | up | 0.004396 | putative lymphocyte G0/G1 switch gene |
| 33351_at | down | 0.004415 | translation factor sui1 homolog |
| 34885_at | up | 0.004455 | synaptogyrin 2 |
| 38443_at | down | 0.004483 | protein tyrosine phosphatase, non-receptor type 11 |
| | | | (Noonan syndrome 1) |
| 40643_at | up | 0.004486 | integrin, alpha 2b (platelet glycoprotein IIb of IIb/IIIa |
| | | | complex, antigen CD41B) |
| 37769_at | up | 0.004498 | endothelial differentiation, lysophosphatidic acid G- |
| | | | protein-coupled receptor, 4 |
| 34307_at | down | 0.004507 | transmembrane 9 superfamily member 2 |
| 40083_at | down | 0.004509 | KIAA0625 protein |
| 40649_at | up | 0.004524 | proprotein convertase subtilisin/kexin type 1 |
| 32227_at | down | 0.004544 | proteoglycan 1, secretory granule |
| 36114_r_at | up | 0.004572 | troponin T1, skeletal, slow |
| 31549_at | up | 0.004598 | MAS1 oncogene |
| 39688_at | up | 0.004617 | requiem, apoptosis response zinc finger gene |
| 1620_at | up | 0.004646 | cadherin 6, type 2, K-cadherin (fetal kidney) |
| 40601_at | down | 0.004663 | beta-amyloid binding protein precursor |
| AFFX-BioDn- | up | 0.004695 | |
| 5_st | | | |
| 39145_at | up | 0.004699 | myosin, light polypeptide 9, regulatory |
| 1091_at | up | 0.004758 | protein kinase, cAMP-dependent, regulatory, type I, beta |
| 36029_at | up | 0.004763 | chromosome 11 open reading frame 8 |
| 41237_at | up | 0.004771 | major histocompatibility complex, class I, A |
| 1104_s_at | up | 0.004778 | heat shock 70 kDa protein 1A |
| 38590_r_at | down | 0.004787 | prothymosin, alpha (gene sequence 28) |
| 38280_s_at | up | 0.004802 | neurotrophic tyrosine kinase, receptor, type 2 |
| 40943_at | up | 0.004812 | ELOVL family member 6, elongation of long chain fatty |
| | | | acids (FEN1/Elo2, SUR4/Elo3-like, yeast) |
| 36557_at | up | 0.004814 | calcium channel, voltage-dependent, beta 1 subunit |
| 759_at | up | 0.004832 | prostaglandin I2 (prostacyclin) synthase |
| 201_s_at | up | 0.004856 | beta-2-microglobulin |
| 36581_at | down | 0.004873 | glycyl-tRNA synthetase |
| 39825_at | up | 0.00488 | solute carrier family 25 (mitochondrial carrier; citrate |
| | | | transporter), member 1 |
| 31471_at | up | 0.004912 | |
| 37511_at | up | 0.004922 | B9 protein |
| 33948_at | up | 0.004978 | corticotropin releasing hormone receptor 2 |
| 39594_f_at | up | 0.005046 | metallothionein 1H |
| 41154_r_at | down | 0.005048 | catenin (cadherin-associated protein), alpha 1, 102 kDa |
| 36173_r_at | down | 0.005061 | adaptor-related protein complex 3, delta 1 subunit |
| 41812_s_at | down | 0.005062 | nucleoporin 210 |
| 34879_at | down | 0.005073 | dolichyl-phosphate mannosyltransferase polypeptide 1, |
| | | | catalytic subunit |
| 33301_g_at | up | 0.005075 | cell division cycle 2-like 2 |
| 32701_at | up | 0.005095 | armadillo repeat gene deletes in velocardiofacial |
| | | | syndrome |
| 37450_r_at | up | 0.005122 | GNAS complex locus |
| 600_at | down | 0.005135 | RAB5A, member RAS oncogene family |
| 1064_at | down | 0.005246 | PTK9 protein tyrosine kinase 9 |
| 38562_g_at | up | 0.005247 | down-regulated in metastasis |
| 41850_s_at | up | 0.005324 | hepatitis delta antigen-interacting protein A |
| 35826_at | up | 0.005331 | suppressor of Ty 5 homolog (S. cerevisiae) |
| 38820_at | down | 0.005384 | 15 kDa selenoprotein |
| 1269_at | down | 0.005394 | phosphoinositide-3-kinase, regulatory subunit, polypeptic |
| | | | 1 (p85 alpha) |
| 37726_at | down | 0.005424 | mitochondrial ribosomal protein L3 |
| 34155_s_at | up | 0.005435 | tyrosinase (oculocutaneous albinism IA) |
| 37296_at | down | 0.005438 | ADP-ribosylation factor-like 1 |
| 38060_at | down | 0.005484 | NADH dehydrogenase (ubiquinone) Fe—S protein 5, |
| | | | 15 kDa (NADH-coenzyme Q reductase) |
| 36734_at | up | 0.005503 | small proline-rich protein 2A |
| 36107_at | down | 0.005549 | ATP synthase, H+ transporting, mitochondrial F0 comple |
| | | | subunit F6 |
| 36961_at | down | 0.00558 | cervical cancer 1 protooncogene |
| 31386_at | up | 0.005585 | immunoglobulin kappa variable 1/OR15-118 |
| 38939_r_at | up | 0.00562 | T-box, brain, 1 |
| 34824_at | down | 0.005649 | ubiquilin 2 |
| 41443_at | up | 0.005702 | SEC7 homolog |
| 411_i_at | up | 0.005711 | interferon induced transmembrane protein 2 (1-8D) |
| 32442_at | up | 0.005715 | |
| 31481_s_at | down | 0.005719 | thymosin, beta 10 |
| 32726_g_at | down | 0.005746 | BH3 interacting domain death agonist |
| 34326_at | down | 0.005796 | coatomer protein complex, subunit beta |
| 126_s_at | up | 0.005887 | synovial sarcoma, X breakpoint 2 |
| 40027_at | down | 0.005902 | ATP synthase, H+ transporting, mitochondrial F0 comple |
| | | | subunit s (factor B) |
| 324_f_at | down | 0.005902 | |
| 41292_at | down | 0.005909 | heterogeneous nuclear ribonucleoprotein H1 (H) |
| 32394_s_at | down | 0.005972 | ribosomal protein L23 |
| 37560_at | up | 0.006036 | FLJ00133 protein |
| 38398_at | up | 0.006052 | MAP-kinase activating death domain |
| 38448_at | up | 0.006057 | actinin, alpha 2 |
| 32859_at | down | 0.006069 | signal transducer and activator of transcription 1, 91 kDa |
| 33535_at | up | 0.006079 | purinergic receptor P2X, ligand-gated ion channel, 1 |
| 35886_at | up | 0.006119 | protein kinase C and casein kinase substrate in neurons |
| 1930_at | up | 0.00618 | ATP-binding cassette, sub-family C (CFTR/MRP), |
| | | | member 3 |
| 37970_at | up | 0.006192 | mitogen-activated protein kinase 8 interacting protein 3 |
| 41677_at | down | 0.006198 | interleukin 15 receptor, alpha |
| 38966_at | up | 0.006205 | glycoprotein, synaptic 2 |
| 40137_at | down | 0.006261 | protein tyrosine phosphatase, non-receptor type 1 |
| 32010_at | up | 0.006291 | hypothetical protein EAN57 |
| 34557_at | up | 0.006316 | melanocortin 1 receptor (alpha melanocyte stimulating |
| | | | hormone receptor) |
| 39310_at | up | 0.006345 | bradykinin receptor B2 |
| 38412_at | up | 0.006424 | protein phosphatase 1, regulatory (inhibitor) subunit 11 |
| 35266_at | down | 0.006494 | bladder cancer associated protein |
| 37693_at | down | 0.006535 | numb homolog (Drosophila) |
| 32802_at | down | 0.00654 | similar to S. cerevisiae SSM4 |
| 39099_at | down | 0.00654 | Sec23 homolog A (S. cerevisiae) |
| 41376_i_at | up | 0.006549 | UDP glycosyltransferase 2 family, polypeptide B7 |
| 38209_at | up | 0.006601 | prostaglandin E receptor 1 (subtype EP1), 42 kDa |
| 37337_at | down | 0.006676 | small nuclear ribonucleoprotein polypeptide G |
| 2036_s_at | down | 0.006697 | CD44 antigen (homing function and Indian blood group |
| | | | system) |
| 39168_at | up | 0.00673 | Ac-like transposable element |
| 36229_at | up | 0.006732 | interleukin 17 receptor |
| 39034_at | down | 0.006748 | DKFZP564O123 protein |
| 39428_at | down | 0.00676 | lymphocyte adaptor protein |
| 33181_at | down | 0.006774 | protein phosphatase 2 (formerly 2A), catalytic subunit, |
| | | | alpha isoform |
| 40225_at | up | 0.006775 | cyclin G associated kinase |
| 37939_at | up | 0.006831 | apolipoprotein B mRNA editing enzyme, catalytic |
| | | | polypeptide-like 3C |
| 35814_at | down | 0.006855 | dendritic cell protein |
| 40790_at | down | 0.006931 | basic helix-loop-helix domain containing, class B, 2 |
| 36779_at | up | 0.006977 | fatty acid binding protein 6, ileal (gastrotropin) |
| 1525_s_at | up | 0.006997 | fibroblast growth factor 8 (androgen-induced) |
| 34630_s_at | up | 0.007011 | dynein, axonemal, heavy polypeptide 9 |
| 40306_at | up | 0.007012 | v-raf murine sarcoma viral oncogene homolog B1 |
| 37731_at | down | 0.007018 | epidermal growth factor receptor pathway substrate 15 |
| 35512_at | up | 0.007135 | |
| 39926_at | down | 0.007144 | MAD, mothers against decapentaplegic homolog 5 |
| | | | (Drosophila) |
| 34397_at | down | 0.007213 | acid-inducible phosphoprotein |
| 39784_at | down | 0.007234 | eukaryotic translation initiation factor 2, subunit 1 alpha, |
| | | | 35 kDa |
| 39454_f_at | up | 0.007249 | T-cell leukemia, homeobox 2 |
| 35892_at | up | 0.00729 | complement component (3b/4b) receptor 1, including |
| | | | Knops blood group system |
| 38848_at | up | 0.007302 | zymogen granule protein 16 |
| 2094_s_at | up | 0.007347 | v-fos FBJ murine osteosarcoma viral oncogene homolog |
| 34559_at | up | 0.007367 | |
| 35643_at | down | 0.007384 | nucleobindin 2 |
| 40885_s_at | down | 0.007411 | syntaxin 16 |
| 40847_at | up | 0.007437 | flavoprotein oxidoreductase MICAL2 |
| 237_s_at | down | 0.007442 | protein phosphatase 2 (formerly 2A), catalytic subunit, |
| | | | alpha isoform |
| 35286_r_at | down | 0.007473 | putative nucleic acid binding protein RY-1 |
| 518_at | up | 0.007484 | nuclear receptor subfamily 1, group H, member 2 |
| 162_at | up | 0.007506 | ubiquitin specific protease 11 |
| 38226_at | down | 0.007549 | hypothetical protein FLJ10569 |
| 32134_at | down | 0.007574 | testis derived transcript (3 LIM domains) |
| 33385_g_at | down | 0.0076 | calpastatin |
| 35716_at | up | 0.007601 | sulfotransferase family, cytosolic, 1C, member 1 |
| 38447_at | up | 0.007604 | adrenergic, beta, receptor kinase 1 |
| 38992_at | down | 0.007658 | DEK oncogene (DNA binding) |
| 33889_s_at | up | 0.007667 | DiGeorge syndrome critical region gene 2 |
| 38162_at | up | 0.007683 | regulating synaptic membrane exocytosis 2 |
| 38707_r_at | up | 0.007701 | E2F transcription factor 4, p107/p130-binding |
| 41212_r_at | down | 0.007709 | Williams-Beuren syndrome chromosome region 1 |
| 32740_at | down | 0.007724 | KIAA0941 protein |
| 35246_at | up | 0.007732 | TYRO3 protein tyrosine kinase |
| 32090_at | up | 0.007747 | nicotinamide nucleotide adenylyltransferase 2 |
| 35411_at | up | 0.007824 | chromosome 16 open reading frame 7 |
| 31957_r_at | up | 0.007826 | ribosomal protein, large, P1 |
| 38084_at | down | 0.007858 | chromobox homolog 3 (HP1 gamma homolog, Drosophil |
| 39136_at | down | 0.007923 | oxidative-stress responsive 1 |
| 33727_r_at | up | 0.007975 | tumor necrosis factor receptor superfamily, member 6b, |
| | | | decoy |
| 39160_at | down | 0.007995 | pyruvate dehydrogenase (lipoamide) beta |
| 584_s_at | down | 0.008042 | X-ray repair complementing defective repair in Chinese |
| | | | hamster cells 5 (double-strand-break rejoining; Ku |
| | | | autoantigen, 80 kDa) |
| 36317_at | up | 0.008056 | coronin, actin binding protein, 2A |
| 32298_at | up | 0.008057 | a disintegrin and metalloproteinase domain 2 (fertilin bet |
| 39714_at | down | 0.008105 | SH3 domain binding glutamic acid-rich protein like |
| 36523_at | down | 0.00813 | ATPase, Cu++ transporting, alpha polypeptide (Menkes |
| | | | syndrome) |
| 37411_at | up | 0.008164 | centaurin, beta 1 |
| 33247_at | down | 0.008182 | proteasome (prosome, macropain) 26S subunit, non- |
| | | | ATPase, 14 |
| 32836_at | up | 0.008183 | 1-acylglycerol-3-phosphate O-acyltransferase 1 |
| | | | (lysophosphatidic acid acyltransferase, alpha) |
| 36473_at | up | 0.008198 | ubiquitin specific protease 20 |
| 1499_at | down | 0.0082 | farnesyltransferase, CAAX box, alpha |
| 33633_at | up | 0.008238 | purinergic receptor P2Y, G-protein coupled, 11 |
| 38736_at | down | 0.008238 | WD repeat domain 1 |
| 31796_at | up | 0.008255 | kinesin family member 1C |
| 36608_at | down | 0.008257 | malate dehydrogenase 1, NAD (soluble) |
| 32725_at | down | 0.008258 | BH3 interacting domain death agonist |
| 34615_at | up | 0.008286 | keratin 12 (Meesmann corneal dystrophy) |
| 39517_at | down | 0.008321 | HTGN29 protein |
| 34503_at | up | 0.008323 | |
| 37740_r_at | down | 0.008356 | solute carrier family 25 (mitochondrial carrier; adenine |
| | | | nucleotide translocator), member 5 |
| 39442_at | down | 0.008385 | unc-50 related |
| 38395_at | down | 0.00841 | NADH dehydrogenase (ubiquinone) Fe—S protein 1, |
| | | | 75 kDa (NADH-coenzyme Q reductase) |
| 33336_at | up | 0.008411 | solute carrier family 4, anion exchanger, member 1 |
| | | | (erythrocyte membrane protein band 3, Diego blood |
| | | | group) |
| 35738_at | down | 0.008416 | high mobility group nucleosomal binding domain 4 |
| 39473_r_at | up | 0.008426 | protein tyrosine phosphatase type IVA, member 3 |
| 32070_at | up | 0.008449 | protein tyrosine phosphatase, receptor type, C-associat |
| | | | protein |
| 36824_at | up | 0.008494 | astrotactin |
| 35492_at | up | 0.00852 | cytochrome P450, family 4, subfamily F, polypeptide 12 |
| 40146_at | down | 0.008529 | RAP1B, member of RAS oncogene family |
| 36660_at | down | 0.00856 | RAB11A, member RAS oncogene family |
| 33791_at | up | 0.008585 | deleted in lymphocytic leukemia, 1 |
| 37475_at | up | 0.008624 | DKFZP434J046 protein |
| 34480_at | up | 0.008635 | cadherin 16, KSP-cadherin |
| 35278_at | up | 0.008637 | ribosomal protein S29 |
| 37720_at | down | 0.008641 | heat shock 60 kDa protein 1 (chaperonin) |
| 35612_at | up | 0.00866 | DKFZP564P1916 protein |
| 36090_at | down | 0.0087 | transducin (beta)-like 2 |
| 41722_at | down | 0.008706 | nicotinamide nucleotide transhydrogenase |
| 1228_s_at | down | 0.008746 | meningioma expressed antigen 6 (coiled-coil proline-rich |
| 34323_at | down | 0.008748 | thyroid receptor interacting protein 15 |
| 36975_at | down | 0.00878 | hypothetical protein MGC8721 |
| 875_g_at | up | 0.008864 | chemokine (C—C motif) ligand 2 |
| 1908_at | up | 0.008924 | ets variant gene 3 |
| 33665_s_at | down | 0.00895 | colony stimulating factor 2 receptor, alpha, low-affinity |
| | | | (granulocyte-macrophage) |
| 37351_at | up | 0.008972 | uridine phosphorylase |
| 38656_s_at | down | 0.008989 | hypothetical protein MGC5576 |
| 33845_at | down | 0.00901 | heterogeneous nuclear ribonucleoprotein H1 (H) |
| 1187_at | up | 0.009027 | ligase III, DNA, ATP-dependent |
| 31700_at | up | 0.009079 | G protein-coupled receptor 35 |
| 37166_at | up | 0.009158 | 3-hydroxyanthranilate 3,4-dioxygenase |
| 35521_at | up | 0.009159 | claudin 9 |
| 39384_at | up | 0.009225 | ELAV (embryonic lethal, abnormal vision, Drosophila)-lik |
| | | | 1 (Hu antigen R) |
| 31495_at | up | 0.009226 | chemokine (C motif) ligand 2 |
| 1011_s_at | down | 0.009274 | tyrosine 3-monooxygenase/tryptophan 5-monooxygenas |
| | | | activation protein, epsilon polypeptide |
| 33150_at | down | 0.009294 | disrupter of silencing 10 |
| 41118_at | up | 0.009318 | hypothetical protein FLJ13639 |
| 34370_at | down | 0.009349 | archain 1 |
| AFFX- | down | 0.009365 | signal transducer and activator of transcription 1, 91 kDa |
| HUMISGF3A/M97935_3_at | | | |
| 32778_at | down | 0.009367 | inositol 1,4,5-triphosphate receptor, type 1 |
| 41223_at | down | 0.009391 | cytochrome c oxidase subunit Va |
| 32452_at | up | 0.009405 | cyclin-dependent kinase 3 |
| 39326_at | up | 0.009436 | ATPase, H+ transporting, lysosomal V0 subunit a isoform 1 |
| 36264_at | up | 0.00945 | megakaryocyte-associated tyrosine kinase |
| 35136_at | down | 0.009509 | nuclear transport factor 2-like export factor 2 |
| 34448_s_at | up | 0.009565 | caspase 2, apoptosis-related cysteine protease (neural |
| | | | precursor cell expressed, developmentally down-regulat |
| | | | 2) |
| 36012_at | down | 0.00959 | progesterone-induced blocking factor 1 |
| 39375_g_at | up | 0.009636 | G-2 and S-phase expressed 1 |
| 39023_at | down | 0.009731 | isocitrate dehydrogenase 1 (NADP+), soluble |
| 41771_g_at | up | 0.009755 | monoamine oxidase A |
| 37579_at | up | 0.009808 | cytoplasmic FMR1 interacting protein 2 |
| 36931_at | up | 0.009886 | transgelin |
| 37328_at | down | 0.009996 | pleckstrin |
| 38058_at | up | 0.010002 | dermatopontin |
| 40802_at | down | 0.010028 | DKFZP434C212 protein |
| 1675_at | down | 0.010041 | RAS p21 protein activator (GTPase activating protein) 1 |
| 35741_at | down | 0.01005 | phosphatidylinositol-4-phosphate 5-kinase, type II, beta |
| 38046_at | down | 0.01007 | IK cytokine, down-regulator of HLA II |
| 39686_g_at | down | 0.010084 | like mouse brain protein E46 |
| 850_r_at | up | 0.010147 | insulin receptor substrate 1 |
| 36152_at | up | 0.010166 | GDP dissociation inhibitor 1 |
| 40931_at | down | 0.010287 | CGI-100 protein |
| 38375_at | down | 0.010289 | esterase D/formylglutathione hydrolase |
| 31726_at | up | 0.010328 | gamma-aminobutyric acid (GABA) A receptor, alpha 3 |
| 33902_at | up | 0.01033 | glycerol-3-phosphate dehydrogenase 1 (soluble) |
| 32749_s_at | up | 0.010344 | filamin A, alpha (actin binding protein 280) |
| 33331_at | up | 0.010358 | BENE protein |
| 35276_at | up | 0.010366 | claudin 4 |
| 34196_at | down | 0.010443 | ocular development-associated gene |
| 1211_s_at | down | 0.010444 | CASP2 and RIPK1 domain containing adaptor with deat |
| | | | domain |
| 133_at | down | 0.010451 | cathepsin C |
| 41342_at | down | 0.010536 | RAN binding protein 1 |
| 39605_at | up | 0.010552 | forkhead box G1B |
| 35412_at | up | 0.010552 | cytochrome P450, family 4, subfamily A, polypeptide 11 |
| 33645_at | up | 0.01057 | GM2 ganglioside activator protein |
| 34778_at | up | 0.010585 | |
| 39281_at | up | 0.010599 | Rho guanine nucleotide exchange factor (GEF) 11 |
| 38676_at | down | 0.010653 | stress 70 protein chaperone, microsome-associated, |
| | | | 60 kDa |
| 37254_at | up | 0.010656 | zinc finger protein 133 (clone pHZ-13) |
| 38631_at | down | 0.010686 | tumor necrosis factor, alpha-induced protein 2 |
| 33373_at | down | 0.010724 | |
| 32816_at | down | 0.01074 | small glutamine-rich tetratricopeptide repeat (TPR)- |
| | | | containing |
| 567_s_at | up | 0.01077 | promyelocytic leukemia |
| 34707_at | up | 0.010772 | chromodomain helicase DNA binding protein 3 |
| 34785_at | down | 0.010772 | KIAA1025 protein |
| AFFX-BioDn- | up | 0.0108 | |
| 3_at | | | |
| 34894_r_at | up | 0.010828 | protease, serine, 22 |
| 1787_at | down | 0.010871 | cyclin-dependent kinase inhibitor 1C (p57, Kip2) |
| 35960_at | up | 0.010922 | inhibitor of kappa light polypeptide gene enhancer in B- |
| | | | cells, kinase beta |
| 33679_f_at | up | 0.010949 | tubulin, beta, 2 |
| 33458_r_at | down | 0.010981 | histone 1, H2bc |
| 32212_at | down | 0.011004 | programmed cell death 8 (apoptosis-inducing factor) |
| 32210_at | up | 0.011012 | phosphoglucomutase 1 |
| 38976_at | up | 0.011013 | coronin, actin binding protein, 1A |
| 41657_at | up | 0.011105 | serine/threonine kinase 11 (Peutz-Jeghers syndrome) |
| 39760_at | down | 0.011179 | quaking homolog, KH domain RNA binding (mouse) |
| 39592_r_at | down | 0.011186 | fibrinogen-like 2 |
| 38269_at | up | 0.011209 | protein kinase D2 |
| 39212_at | up | 0.011221 | hypothetical protein FLJ11191 |
| 41081_at | up | 0.0113 | BUB1 budding uninhibited by benzimidazoles 1 homolog |
| | | | (yeast) |
| 36272_r_at | up | 0.011309 | peripheral myelin protein 2 |
| 36669_at | up | 0.011328 | FBJ murine osteosarcoma viral oncogene homolog B |
| 40874_at | down | 0.011345 | endothelial differentiation-related factor 1 |
| 1877_g_at | down | 0.011363 | |
| 36886_f_at | up | 0.011466 | killer cell immunoglobulin-like receptor, two domains, Ion |
| | | | cytoplasmic tail, 3 |
| 36924_r_at | up | 0.011516 | secretogranin II (chromogranin C) |
| 38439_at | up | 0.011558 | nuclear factor (erythroid-derived 2)-like 1 |
| 39733_at | down | 0.011569 | homocysteine-inducible, endoplasmic reticulum stress- |
| | | | inducible, ubiquitin-like domain member 1 |
| 33034_at | up | 0.011597 | rhomboid, veinlet-like 1 (Drosophila) |
| 32380_at | up | 0.01161 | plakophilin 1 (ectodermal dysplasia/skin fragility |
| | | | syndrome) |
| 41819_at | down | 0.011617 | FYN binding protein (FYB-120/130) |
| 781_at | down | 0.011656 | Rab geranylgeranyltransferase, beta subunit |
| 37943_at | down | 0.011657 | zinc finger, FYVE domain containing 26 |
| 41641_at | up | 0.011665 | GPI-anchored metastasis-associated protein homolog |
| 273_g_at | up | 0.011718 | gastrin-releasing peptide |
| 36891_at | up | 0.01173 | putative acyltransferase |
| 32235_at | up | 0.011759 | mahogunin, ring finger 1 |
| 33750_at | up | 0.01177 | protein tyrosine phosphatase, receptor type, U |
| 2063_at | down | 0.011827 | excision repair cross-complementing rodent repair |
| | | | deficiency, complementation group 5 (xeroderma |
| | | | pigmentosum, complementation group G (Cockayne |
| | | | syndrome)) |
| 466_at | down | 0.011856 | general transcription factor II, i |
| 38755_at | up | 0.011889 | Fas (TNFRSF6)-associated via death domain |
| 37850_at | up | 0.011942 | hypothetical protein dJ462O23.2 |
| 36894_at | up | 0.011976 | plakophilin 4 |
| 38710_at | up | 0.012032 | ubiquitin-specific protease otubain 1 |
| 35165_at | down | 0.012038 | hypothetical protein MGC13033 |
| 37728_r_at | down | 0.012091 | reticulocalbin 2, EF-hand calcium binding domain |
| 32837_at | up | 0.0121 | 1-acylglycerol-3-phosphate O-acyltransferase 2 |
| | | | (lysophosphatidic acid acyltransferase, beta) |
| 1717_s_at | down | 0.012108 | baculoviral IAP repeat-containing 3 |
| 933_f_at | down | 0.012134 | zinc finger protein 91 (HPF7, HTF10) |
| 37919_at | up | 0.012214 | solute carrier family 21 (prostaglandin transporter), |
| | | | member 2 |
| 1196_at | up | 0.012219 | chromosome condensation 1 |
| 1285_at | up | 0.012233 | |
| 41490_at | down | 0.012323 | phosphoribosyl pyrophosphate synthetase 2 |
| 504_at | down | 0.012336 | ubiquitin-conjugating enzyme E2D 3 (UBC4/5 homolog, |
| | | | yeast) |
| 34146_at | up | 0.01236 | 8-oxoguanine DNA glycosylase |
| 36336_s_at | up | 0.012444 | KIAA0963 protein |
| 36198_at | down | 0.012492 | translocase of outer mitochondrial membrane 20 (yeast) |
| | | | homolog |
| 32000_g_at | up | 0.012498 | ATP-binding cassette, sub-family A (ABC1), member 1 |
| 41257_at | down | 0.012502 | calpastatin |
| 34768_at | down | 0.012514 | thioredoxin domain containing |
| 31977_at | up | 0.012525 | guanylate cyclase 2D, membrane (retina-specific) |
| 39628_at | up | 0.012571 | RAB9A, member RAS oncogene family |
| 36583_at | down | 0.012698 | sorting nexin 1 |
| 41179_at | down | 0.012738 | ring finger protein 44 |
| 36436_at | up | 0.012745 | leukocyte cell-derived chemotaxin 2 |
| 39327_at | up | 0.012808 | Melanoma associated gene |
| 31525_s_at | up | 0.012813 | hemoglobin, alpha 2 |
| 1815_g_at | down | 0.012835 | transforming growth factor, beta receptor II (70/80 kDa) |
| 40745_at | up | 0.012844 | adaptor-related protein complex 1, beta 1 subunit |
| 1795_g_at | up | 0.012848 | cyclin D3 |
| 1079_g_at | up | 0.012852 | prolactin receptor |
| 40837_at | up | 0.012862 | transducin-like enhancer of split 2 (E(sp1) homolog, |
| | | | Drosophila) |
| 34440_at | up | 0.012898 | DiGeorge syndrome critical region gene 9 |
| 33433_at | down | 0.012905 | DKFZP564F0522 protein |
| 40613_at | down | 0.012921 | chromosome 6 open reading frame 62 |
| 40182_s_at | up | 0.012959 | coactivator-associated arginine methyltransferase-1 |
| 36425_at | up | 0.012979 | nebulette |
| 31995_g_at | up | 0.012992 | ADP-ribosylation factor guanine nucleotide-exchange |
| | | | factor 2 (brefeldin A-inhibited) |
| 36338_at | up | 0.013004 | leucine zipper protein 1 |
| 34753_at | down | 0.013079 | synaptobrevin-like 1 |
| 41036_at | up | 0.01309 | hypothetical protein FLJ12242 |
| 33568_at | up | 0.013116 | cholinergic receptor, nicotinic, beta polypeptide 4 |
| 36550_at | down | 0.013154 | Ras and Rab interactor 2 |
| 35564_at | up | 0.013202 | |
| 34334_at | up | 0.013236 | ephrin-B2 |
| 35848_at | down | 0.013256 | retinoic acid induced 17 |
| 33264_at | up | 0.013299 | rTS beta protein |
| 41080_at | up | 0.013323 | H2A histone family, member B |
| 40130_at | up | 0.013335 | follistatin-like 1 |
| 32233_at | down | 0.013347 | torsin family 1, member B (torsin B) |
| 35209_at | down | 0.013359 | EPM2A (laforin) interacting protein 1 |
| 40644_g_at | up | 0.013371 | integrin, alpha 2b (platelet glycoprotein IIb of IIb/IIIa |
| | | | complex, antigen CD41B) |
| 41063_g_at | up | 0.013386 | likely ortholog of mouse nervous system polycomb 1 |
| 37747_at | down | 0.013389 | annexin A5 |
| 31599_f_at | up | 0.013395 | melanoma antigen, family A, 6 |
| 39140_at | down | 0.013426 | nucleic acid helicase DDXx |
| 41634_at | down | 0.013478 | KIAA0256 gene product |
| 1789_at | down | 0.013483 | COP9 constitutive photomorphogenic homolog subunit 5 |
| | | | (Arabidopsis) |
| 34385_at | down | 0.013542 | succinate dehydrogenase complex, subunit C, integral |
| | | | membrane protein, 15 kDa |
| 39955_at | up | 0.013562 | deleted in lymphocytic leukemia, 2 |
| 1147_at | up | 0.013573 | |
| 40539_at | up | 0.013587 | myosin IXB |
| 36472_at | down | 0.013625 | N-myc (and STAT) interactor |
| 40783_s_at | up | 0.013679 | phosphatidylinositol 4-kinase, catalytic, alpha polypeptid |
| 36171_at | down | 0.013705 | activated RNA polymerase II transcription cofactor 4 |
| 35868_at | up | 0.013726 | advanced glycosylation end product-specific receptor |
| 1245_i_at | down | 0.013758 | p21 (CDKN1A)-activated kinase 2 |
| 37793_r_at | up | 0.013825 | RAD51-like 3 (S. cerevisiae) |
| 35082_at | up | 0.013847 | Zic family member 3 heterotaxy 1 (odd-paired homolog, |
| | | | Drosophila) |
| 34762_at | up | 0.013858 | ring finger protein (C3HC4 type) 8 |
| 41187_at | down | 0.013902 | myosin regulatory light chain MRLC2 |
| 33879_at | up | 0.013908 | type I sigma receptor |
| 1652_at | up | 0.013938 | pim-2 oncogene |
| 40417_at | down | 0.013938 | chaperonin containing TCP1, subunit 5 (epsilon) |
| 41129_at | down | 0.013943 | KIAA0033 protein |
| 38420_at | up | 0.013954 | collagen, type V, alpha 2 |
| 34210_at | down | 0.014015 | CDW52 antigen (CAMPATH-1 antigen) |
| 39344_at | down | 0.014023 | transformer-2 alpha (htra-2 alpha) |
| 1706_at | up | 0.014079 | v-raf murine sarcoma 3611 viral oncogene homolog 1 |
| 34661_at | up | 0.014103 | KIAA0350 protein |
| 38993_r_at | down | 0.014129 | |
| 32184_at | down | 0.014139 | LIM domain only 2 (rhombotin-like 1) |
| 36631_at | down | 0.014147 | peroxiredoxin 3 |
| 35371_at | up | 0.014173 | LPS-responsive vesicle trafficking, beach and anchor |
| | | | containing |
| 39640_at | up | 0.014174 | glutamine-fructose-6-phosphate transaminase 2 |
| 36019_at | up | 0.014196 | serine/threonine kinase 19 |
| 37584_at | up | 0.014203 | Fanconi anemia, complementation group G |
| 36011_at | up | 0.014257 | syntaxin 10 |
| 36482_s_at | up | 0.014257 | ATPase, Ca++ transporting, ubiquitous |
| 31950_at | down | 0.014258 | poly(A) binding protein, cytoplasmic 1 |
| 37220_at | down | 0.014462 | Fc fragment of IgG, high affinity Ia, receptor for (CD64) |
| 37121_at | up | 0.014557 | natural killer cell group 7 sequence |
| 36538_at | up | 0.014685 | protein phosphatase 1, regulatory (inhibitor) subunit 13B |
| 38686_at | up | 0.014734 | ATPase, H+ transporting, lysosomal 38 kDa, V0 subunit |
| | | | isoform 1 |
| 32695_at | down | 0.014748 | HIV TAT specific factor 1 |
| 32121_at | down | 0.014803 | phosphoinositide-3-kinase, catalytic, delta polypeptide |
| 37374_at | down | 0.014809 | annexin A4 |
| 41273_at | up | 0.014835 | FK506 binding protein 12-rapamycin associated protein |
| 32464_at | up | 0.014865 | defensin, beta 4 |
| 34293_at | up | 0.014917 | kinesin family member C3 |
| 37035_at | down | 0.014931 | stress-associated endoplasmic reticulum protein 1 |
| 1318_at | down | 0.014941 | retinoblastoma binding protein 4 |
| 35215_at | down | 0.014941 | HDCMA18P protein |
| 38572_at | up | 0.01496 | FGFR1 oncogene partner |
| 32258_r_at | down | 0.014963 | telomeric repeat binding factor (NIMA-interacting) 1 |
| 34646_at | down | 0.014971 | ribosomal protein S7 |
| 33821_at | down | 0.014993 | homolog of yeast long chain polyunsaturated fatty acid |
| | | | elongation enzyme 2 |
| 37038_at | down | 0.015036 | ATP-binding cassette, sub-family D (ALD), member 3 |
| 38463_s_at | down | 0.015059 | adenosine monophosphate deaminase (isoform E) |
| 31668_f_at | up | 0.015097 | erythrocyte membrane protein band 4.1-like 2 |
| 34310_at | up | 0.015109 | adenine phosphoribosyltransferase |
| 1324_at | up | 0.015162 | RAD9 homolog (S. pombe) |
| 40989_at | up | 0.01517 | tetraspan 5 |
| 32493_at | up | 0.015183 | thyrotrophic embryonic factor |
| 39694_at | up | 0.015198 | hypothetical protein MGC5508 |
| 34763_at | down | 0.015201 | chondroitin sulfate proteoglycan 6 (bamacan) |
| 41134_at | up | 0.015209 | disks large-associated protein 4 |
| 36136_at | up | 0.015225 | tumor protein p53 inducible protein 11 |
| 35973_at | down | 0.015225 | huntingtin interacting protein 14 |
| 36004_at | up | 0.015262 | inhibitor of kappa light polypeptide gene enhancer in B- |
| | | | cells, kinase gamma |
| 37506_at | down | 0.01527 | formin binding protein 3 |
| 36795_at | up | 0.015294 | prosaposin (variant Gaucher disease and variant |
| | | | metachromatic leukodystrophy) |
| 31808_at | down | 0.015332 | inhibitor of growth family, member 3 |
| 38829_r_at | down | 0.015403 | KH-type splicing regulatory protein (FUSE binding protein |
| | | | 2) |
| 34301_r_at | up | 0.015458 | keratin 17 |
| 39392_at | down | 0.01557 | glyceronephosphate O-acyltransferase |
| 41132_r_at | down | 0.015577 | heterogeneous nuclear ribonucleoprotein H2 (H′) |
| 35952_at | up | 0.015587 | |
| 31882_at | up | 0.015605 | RNA, U3 small nucleolar interacting protein 2 |
| 40132_g_at | down | 0.015651 | follistatin-like 1 |
| 31999_at | up | 0.01568 | ATP-binding cassette, sub-family A (ABC1), member 1 |
| 32214_at | down | 0.015754 | thioredoxin-like, 32 kDa |
| 38244_at | up | 0.015774 | hypothetical protein FLJ10178 |
| 38841_at | down | 0.01586 | putative glialblastoma cell differentiation-related |
| 40615_at | down | 0.015863 | hypothetical protein FLJ21439 |
| 36932_at | down | 0.015868 | general transcription factor IIIC, polypeptide 2, beta |
| | | | 110 kDa |
| 40684_at | up | 0.015914 | GTP cyclohydrolase I feedback regulatory protein |
| 537_f_at | up | 0.015947 | breakpoint cluster region |
| 40246_at | down | 0.015992 | discs, large (Drosophila) homolog 1 |
| 31924_at | up | 0.016021 | testicular soluble adenylyl cyclase |
| 952_at | down | 0.016057 | |
| 33925_at | up | 0.016064 | neurogranin (protein kinase C substrate, RC3) |
| 41784_at | down | 0.016071 | SR rich protein |
| 32696_at | down | 0.016102 | pre-B-cell leukemia transcription factor 3 |
| 39857_at | down | 0.016137 | syntaxin 11 |
| 33186_i_at | up | 0.01614 | |
| 33297_at | down | 0.016149 | chromosome 6 open reading frame 130 |
| 872_i_at | up | 0.016203 | insulin receptor substrate 1 |
| 35080_at | up | 0.016227 | neurotensin receptor 1 (high affinity) |
| 40933_f_at | up | 0.016236 | zinc finger, DHHC domain containing 18 |
| 38380_at | down | 0.016279 | POP4 (processing of precursor, S. cerevisiae) homolog |
| 34962_at | up | 0.01628 | |
| 40203_at | down | 0.016313 | putative translation initiation factor |
| 31511_at | up | 0.016334 | ribosomal protein S9 |
| 821_s_at | down | 0.016432 | folate receptor 1 (adult) |
| 37973_at | down | 0.016448 | sorting nexin 13 |
| 229_at | down | 0.01645 | CCAAT-box-binding transcription factor |
| 1159_at | up | 0.016461 | interleukin 7 |
| 343_s_at | up | 0.016526 | ectonucleotide pyrophosphatase/phosphodiesterase 1 |
| 40315_at | up | 0.016527 | serine protease inhibitor, Kazal type, 5 |
| 34813_at | down | 0.016531 | eukaryotic translation initiation factor 1A |
| 38868_at | up | 0.01656 | Fc fragment of IgA, receptor for |
| 1601_s_at | down | 0.016625 | insulin-like growth factor binding protein 5 |
| 40189_at | down | 0.016702 | SET translocation (myeloid leukemia-associated) |
| 34679_at | up | 0.016704 | breakpoint cluster region |
| 35915_at | up | 0.01671 | inhibin, beta C |
| 40619_at | up | 0.016734 | ubiquitin carrier protein |
| 39740_g_at | down | 0.016741 | nascent-polypeptide-associated complex alpha |
| | | | polypeptide |
| 38016_at | down | 0.016744 | heterogeneous nuclear ribonucleoprotein D (AU-rich |
| | | | element RNA binding protein 1, 37 kDa) |
| 1707_g_at | up | 0.01675 | v-raf murine sarcoma 3611 viral oncogene homolog 1 |
| 41459_at | down | 0.016767 | tripeptidyl peptidase II |
| 41524_at | down | 0.01681 | inositol polyphosphate-1-phosphatase |
| 41085_at | up | 0.016818 | polymerase (DNA directed), epsilon 2 (p59 subunit) |
| 155_s_at | down | 0.016819 | ubiquitin-like 1 (sentrin) |
| 1650_g_at | up | 0.016871 | chromosome 20 open reading frame 16 |
| 41059_at | down | 0.016883 | leukocyte membrane antigen |
| 32700_at | down | 0.016939 | guanylate binding protein 2, interferon-inducible |
| 41749_at | down | 0.01696 | chromosome 21 open reading frame 33 |
| 33603_at | up | 0.016974 | ATP-binding cassette, sub-family D (ALD), member 1 |
| 36159_s_at | down | 0.017029 | prion protein (p27-30) (Creutzfeld-Jakob disease, |
| | | | Gerstmann-Strausler-Scheinker syndrome, fatal familial |
| | | | insomnia) |
| 37843_i_at | up | 0.017095 | class I cytokine receptor |
| 1555_f_at | up | 0.017101 | cytochrome P450, family 2, subfamily A, polypeptide 7 |
| 36445_at | up | 0.017109 | chemokine (C—C motif) ligand 23 |
| 37449_i_at | up | 0.017262 | GNAS complex locus |
| 31613_at | up | 0.017304 | laminin, beta 4 |
| 31746_at | up | 0.017309 | zinc finger protein 204 |
| 37962_r_at | down | 0.017344 | syntaxin binding protein 3 |
| 2044_s_at | down | 0.017376 | retinoblastoma 1 (including osteosarcoma) |
| 35327_at | down | 0.017394 | eukaryotic translation initiation factor 3, subunit 3 gamma |
| | | | 40 kDa |
| 34730_g_at | up | 0.017407 | trophinin |
| 31406_at | up | 0.017412 | G protein-coupled receptor 50 |
| 31932_f_at | down | 0.017423 | basic transcription factor 3 |
| 442_at | down | 0.017431 | tumor rejection antigen (gp96) 1 |
| 151_s_at | up | 0.017469 | hypothetical protein DKFZp434N0650 |
| 40817_at | up | 0.017498 | nucleobindin 1 |
| 34637_f_at | up | 0.017511 | alcohol dehydrogenase 1A (class I), alpha polypeptide |
| 32350_at | down | 0.017517 | mucosa associated lymphoid tissue lymphoma |
| | | | translocation gene 1 |
| 33778_at | up | 0.017561 | chromosome 22 open reading frame 4 |
| 31687_f_at | up | 0.017608 | hemoglobin, beta |
| AFFX-BioC-3_at | up | 0.017611 | |
| 1420_s_at | down | 0.017633 | eukaryotic translation initiation factor 4A, isoform 2 |
| 33441_at | up | 0.017697 | T-cell leukemia translocation altered gene |
| 37871_at | up | 0.017737 | islet amyloid polypeptide |
| 32971_at | up | 0.017788 | Friedreich ataxia region gene X123 |
| 39547_at | up | 0.017849 | RAN binding protein 9 |
| 37727_i_at | down | 0.017866 | reticulocalbin 2, EF-hand calcium binding domain |
| 33467_at | up | 0.017871 | CMRF35 leukocyte immunoglobulin-like receptor |
| 32287_s_at | up | 0.017884 | killer cell lectin-like receptor subfamily C, member 3 |
| 1903_at | down | 0.017927 | |
| 36195_at | down | 0.018011 | isocitrate dehydrogenase 3 (NAD+) alpha |
| 40609_at | up | 0.018045 | helicase with SNF2 domain 1 |
| 32599_at | down | 0.018087 | tuberous sclerosis 1 |
| 37015_at | down | 0.018105 | aldehyde dehydrogenase 1 family, member A1 |
| 36590_at | up | 0.018149 | solute carrier family 16 (monocarboxylic acid |
| | | | transporters), member 2 (putative transporter) |
| 38830_at | up | 0.018172 | hypothetical protein FLJ11198 |
| 1986_at | down | 0.018179 | retinoblastoma-like 2 (p130) |
| 192_at | down | 0.018213 | TAF7 RNA polymerase II, TATA box binding protein |
| | | | (TBP)-associated factor, 55 kDa |
| 35630_at | up | 0.018226 | lethal giant larvae homolog 2 (Drosophila) |
| 40637_at | down | 0.018269 | heat shock 70 kDa protein 8 |
| 31768_at | up | 0.018276 | histone 1, H2ai |
| 34253_at | down | 0.018328 | nucleoporin 160 kDa |
| 36783_f_at | down | 0.018333 | Krueppel-related zinc finger protein |
| 31914_at | up | 0.018338 | chromodomain helicase DNA binding protein 1-like |
| 41374_at | up | 0.0184 | ribosomal protein S6 kinase, 70 kDa, polypeptide 2 |
| 40754_at | up | 0.018431 | general transcription factor IIH, polypeptide 3, 34 kDa |
| 34857_at | down | 0.018445 | hypothetical protein FLJ20986 |
| 33534_at | up | 0.018485 | endothelial cell-specific molecule 1 |
| 41852_at | up | 0.018525 | rearranged L-myc fusion sequence |
| 32181_at | up | 0.018613 | fiotillin 2 |
| 37967_at | up | 0.01865 | leukocyte specific transcript 1 |
| 33084_at | up | 0.018756 | complexin 2 |
| 32345_at | up | 0.018764 | |
| 1295_at | down | 0.018786 | v-rel reticuloendotheliosis viral oncogene homolog A, |
| | | | nuclear factor of kappa light polypeptide gene enhancer |
| | | | B-cells 3, p65 (avian) |
| 40166_at | down | 0.01879 | likely ortholog of mouse WD-40-repeat-containing protein |
| | | | with a SOCS box 2 |
| 38068_at | down | 0.018828 | autocrine motility factor receptor |
| 1038_s_at | down | 0.018932 | interferon gamma receptor 1 |
| 41601_at | down | 0.018953 | a disintegrin and metalloproteinase domain 17 (tumor |
| | | | necrosis factor, alpha, converting enzyme) |
| 2057_g_at | up | 0.018958 | fibroblast growth factor receptor 1 (fms-related tyrosine |
| | | | kinase 2, Pfeiffer syndrome) |
| 226_at | down | 0.018995 | protein kinase, cAMP-dependent, regulatory, type I, alph |
| | | | (tissue specific extinguisher 1) |
| 32850_at | down | 0.018999 | nucleoporin 153 kDa |
| 36186_at | up | 0.01902 | RNA binding protein S1, serine-rich domain |
| 34644_at | up | 0.019096 | beta-2-microglobulin |
| 300_f_at | down | 0.019103 | |
| 825_at | down | 0.019195 | PRP4 pre-mRNA processing factor 4 homolog B (yeast) |
| 35938_at | down | 0.019217 | phospholipase A2, group IVA (cytosolic, calcium- |
| | | | dependent) |
| 36539_at | up | 0.019221 | immunoglobulin lambda locus |
| 34086_at | up | 0.019292 | endothelial differentiation, sphingolipid G-protein-couple |
| | | | receptor, 5 |
| 34525_at | up | 0.019298 | T-cell leukemia/lymphoma 1B |
| 38007_at | up | 0.019318 | neurofibromin 2 (bilateral acoustic neuroma) |
| 36463_at | down | 0.019332 | BCL2-associated athanogene 5 |
| 33109_f_at | up | 0.019346 | SRY (sex determining region Y)-box 2 |
| 40309_at | up | 0.019388 | carbonic anhydrase IX |
| 34349_at | down | 0.019479 | SEC63-like (S. cerevisiae) |
| 691_g_at | up | 0.019503 | procollagen-proline, 2-oxoglutarate 4-dioxygenase (proli |
| | | | 4-hydroxylase), beta polypeptide (protein disulfide |
| | | | isomerase; thyroid hormone binding protein p55) |
| 38657_s_at | up | 0.019523 | clathrin, light polypeptide (Lca) |
| 40365_at | up | 0.019556 | guanine nucleotide binding protein (G protein), alpha 15 |
| | | | (Gq class) |
| 36576_at | down | 0.019565 | H2A histone family, member Y |
| 2004_at | down | 0.019576 | mitogen-activated protein kinase kinase kinase 1 |
| 31890_s_at | down | 0.019638 | zinc finger protein 143 (clone pHZ-1) |
| 510_g_at | down | 0.019641 | MAD, mothers against decapentaplegic homolog 4 |
| | | | (Drosophila) |
| 32183_at | down | 0.01973 | splicing factor, arginine/serine-rich 11 |
| 37812_at | up | 0.019731 | cut-like 2 (Drosophila) |
| 41131_f_at | down | 0.019743 | heterogeneous nuclear ribonucleoprotein H2 (H′) |
| 32265_at | up | 0.019747 | nuclear receptor subfamily 4, group A, member 1 |
| 33771_at | up | 0.019763 | T-cell activation leucine repeat-rich protein |
| 531_at | down | 0.019841 | GLI pathogenesis-related 1 (glioma) |
| 34796_at | down | 0.01992 | translocation associated membrane protein 1 |
| 35312_at | up | 0.019979 | MCM2 minichromosome maintenance deficient 2, mitoti |
| | | | (S. cerevisiae) |
| 35303_at | down | 0.020035 | insulin induced gene 1 |
| 36547_r_at | up | 0.020045 | KIAA0542 gene product |
| 35650_at | down | 0.020047 | KIAA0356 gene product |
| 34387_at | down | 0.020082 | KIAA0205 gene product |
| 40208_at | up | 0.020123 | growth differentiation factor 11 |
| 41147_at | down | 0.020124 | hypothetical protein MGC4276 similar to CG8198 |
| 32171_at | down | 0.020139 | eukaryotic translation initiation factor 5 |
| 35798_at | up | 0.020169 | NS1-associated protein 1 |
| 39657_at | up | 0.02024 | keratin 4 |
| 34147_g_at | up | 0.02024 | 8-oxoguanine DNA glycosylase |
| 41195_at | down | 0.020265 | LIM domain containing preferred translocation partner in |
| | | | lipoma |
| 36514_at | down | 0.02029 | cell growth regulatory with ring finger domain |
| 39833_at | up | 0.020298 | misshapen/NIK-related kinase |
| 38717_at | down | 0.02043 | DKFZP586A0522 protein |
| 34884_at | up | 0.020442 | carbamoyl-phosphate synthetase 1, mitochondrial |
| 35674_at | down | 0.020444 | peptidyl arginine deiminase, type II |
| 930_at | up | 0.020469 | protein phosphatase 2 (formerly 2A), regulatory subunit |
| | | | B″, alpha |
| 40141_at | down | 0.020498 | cullin 4B |
| 35012_at | down | 0.020538 | myeloid cell nuclear differentiation antigen |
| 31326_at | up | 0.020632 | |
| 38088_r_at | up | 0.020721 | S100 calcium binding protein A4 (calcium protein, |
| | | | calvasculin, metastasin, murine placental homolog) |
| 41335_at | down | 0.020743 | DKFZP566O1646 protein |
| 35048_at | up | 0.020747 | glutamate receptor, ionotrophic, AMPA 3 |
| 37545_at | up | 0.020815 | secretory carrier membrane protein 5 |
| 39750_at | up | 0.020847 | zinc finger, DHHC domain containing 3 |
| 37811_at | up | 0.020897 | calcium channel, voltage-dependent, alpha 2/delta subu 2 |
| 31728_at | up | 0.02093 | major histocompatibility complex, class II, DO alpha |
| 39178_at | down | 0.020932 | reticulon 1 |
| 40636_at | up | 0.020943 | fiotillin 1 |
| 1062_g_at | down | 0.02097 | interleukin 10 receptor, alpha |
| 39400_at | up | 0.020978 | KIAA1055 protein |
| 34383_at | down | 0.021052 | ubiquitin specific protease 1 |
| 39793_at | down | 0.021068 | glioblastoma amplified sequence |
| 36934_at | down | 0.021189 | chromosome 20 open reading frame 111 |
| 38815_at | down | 0.021221 | actin related protein 2/3 complex, subunit 1A, 41 kDa |
| 38780_at | down | 0.021235 | aldo-keto reductase family 1, member A1 (aldehyde |
| | | | reductase) |
| 38685_at | down | 0.021249 | syntaxin 12 |
| 34751_at | down | 0.021264 | zinc finger and BTB domain containing 1 |
| 39108_at | up | 0.021289 | lanosterol synthase (2,3-oxidosqualene-lanosterol |
| | | | cyclase) |
| 33908_at | up | 0.02134 | calpain 1, (mu/l) large subunit |
| 32895_f_at | up | 0.021352 | HIV-1 Rev binding protein-like |
| 37409_at | down | 0.021533 | SFRS protein kinase 2 |
| 32575_at | up | 0.021556 | nucleosome assembly protein 1-like 4 |
| _at | down | 0.0216 | mucosa associated lymphoid tissue lymphoma |
| | | | translocation gene 1 |
| 36952_at | up | 0.021656 | hydroxyacyl-Coenzyme A dehydrogenase/3-ketoacyl- |
| | | | Coenzyme A thiolase/enoyl-Coenzyme A hydratase |
| | | | (trifunctional protein), alpha subunit |
| 34767_at | down | 0.021706 | modulator of apoptosis 1 |
| AFFX- | up | 0.021712 | glyceraldehyde-3-phosphate dehydrogenase |
| HUMGAPDH/M33197_M_st | | | |
| 352_at | up | 0.021712 | phosphotidylinositol transfer protein |
| 38383_at | down | 0.021745 | 5-methyltetrahydrofolate-homocysteine methyltransferas |
| 36648_at | down | 0.021783 | cofactor required for Sp1 transcriptional activation, subur |
| | | | 9, 33 kDa |
| 40431_at | down | 0.021788 | KIAA0431 protein |
| 36433_at | up | 0.021793 | glycine receptor, alpha 3 |
| 35918_at | up | 0.021822 | deleted in lung and esophageal cancer 1 |
| 37078_at | up | 0.021832 | CD3Z antigen, zeta polypeptide (TiT3 complex) |
| 33278_at | up | 0.021853 | SA hypertension-associated homolog (rat) |
| 39839_at | down | 0.021855 | cold shock domain protein A |
| 39088_at | up | 0.021896 | seven transmembrane domain protein |
| 35036_at | down | 0.02195 | complement component 1, q subcomponent, receptor 1 |
| 33382_at | down | 0.021991 | N-acylsphingosine amidohydrolase (acid ceramidase)-lik |
| 41520_at | up | 0.022029 | hypothetical protein LOC284352 |
| 32563_at | down | 0.022071 | ATPase, Na+/K+ transporting, beta 3 polypeptide |
| 40457_at | down | 0.022156 | splicing factor, arginine/serine-rich 3 |
| 36404_at | up | 0.022162 | glucagon-like peptide 1 receptor |
| 893_at | up | 0.022233 | ubiquitin carrier protein |
| 37691_at | up | 0.0223 | MADS box transcription enhancer factor 2, polypeptide B |
| | | | (myocyte enhancer factor 2B) |
| 36191_at | down | 0.022333 | transcription factor A, mitochondrial |
| 40801_at | down | 0.022358 | DKFZP434C212 protein |
| 32953_at | up | 0.022369 | CD5 antigen (p56-62) |
| 35450_s_at | up | 0.022389 | general transcription factor II, i |
| 41726_at | up | 0.022435 | endothelin converting enzyme 1 |
| 37463_r_at | up | 0.022459 | splicing factor 3a, subunit 2, 66 kDa |
| 37622_r_at | down | 0.022516 | PC4 and SFRS1 interacting protein 2 |
| 263_g_at | down | 0.022574 | adenosylmethionine decarboxylase 1 |
| 34400_at | down | 0.022648 | low molecular mass ubiquinone-binding protein (9.5 kD) |
| 39501_f_at | up | 0.022704 | amyloid beta (A4) precursor protein-binding, family A, |
| | | | member 2 binding protein |
| 2032_s_at | up | 0.022708 | integrin, alpha V (vitronectin receptor, alpha polypeptide, |
| | | | antigen CD51) |
| 31879_at | down | 0.022724 | far upstream element (FUSE) binding protein 3 |
| 20 _at | down | 0.022731 | integrin, alpha 4 (antigen CD49D, alpha 4 subunit of VLA· |
| | | | 4 receptor) |
| 41185_f_at | down | 0.022767 | SMT3 suppressor of mif two 3 homolog 2 (yeast) |
| 41829_at | up | 0.022808 | likely ortholog of mouse la related protein |
| 41388_at | up | 0.022831 | Meis1, myeloid ecotropic viral integration site 1 homolog |
| | | | (mouse) |
| 38401_s_at | up | 0.022852 | DKFZP434D1335 protein |
| 452_at | down | 0.022861 | SWI/SNF related, matrix associated, actin dependent |
| | | | regulator of chromatin, subfamily c, member 1 |
| 1649_at | up | 0.022875 | chromosome 20 open reading frame 16 |
| 38106_at | down | 0.022878 | TGF beta-inducible nuclear protein 1 |
| 34704_r_at | up | 0.022898 | chorionic somatomammotropin hormone 2 |
| 1388_g_at | down | 0.022922 | vitamin D (1,25-dihydroxyvitamin D3) receptor |
| 35432_at | down | 0.022924 | mediator of RNA polymerase II transcription, subunit 6 |
| | | | homolog (yeast) |
| AFFX- | up | 0.022931 | actin, beta |
| HSAC07/X00351_3_at | | | |
| 37493_at | down | 0.023078 | colony stimulating factor 2 receptor, beta, low-affinity |
| | | | (granulocyte-macrophage) |
| 32646_at | up | 0.023083 | KIAA0449 protein |
| 32001_s_at | up | 0.023099 | paired basic amino acid cleaving system 4 |
| 886_at | down | 0.023115 | deoxycytidine kinase |
| 32858_at | up | 0.023123 | ubinuclein 1 |
| 707_s_at | up | 0.023162 | |
| 38791_at | up | 0.02319 | dolichyl-diphosphooligosaccharide-protein |
| | | | glycosyltransferase |
| 40579_at | down | 0.023229 | HIV-1 Rev binding protein |
| 41214_at | down | 0.023253 | ribosomal protein S4, Y-linked |
| 37393_at | down | 0.02326 | hairy and enhancer of split 1, (Drosophila) |
| 35487_at | up | 0.023272 | bromodomain, testis-specific |
| 32866_at | up | 0.023293 | KIAA0605 gene product |
| 34773_at | down | 0.023349 | tubulin-specific chaperone a |
| 32669_at | down | 0.023384 | suppressor of cytokine signaling 5 |
| 37697_s_at | down | 0.023475 | voltage-dependent anion channel 2 |
| 39809_at | down | 0.023498 | HMG-box containing protein 1 |
| 33268_at | up | 0.023524 | Smcx homolog, X chromosome (mouse) |
| 33320_at | down | 0.023552 | MHC class I region ORF |
| 32297_s_at | up | 0.023584 | killer cell lectin-like receptor subfamily C, member 2 |
| 34059_at | up | 0.023596 | Pvt1 oncogene homolog, MYC activator (mouse) |
| 38732_at | down | 0.023596 | chloride channel, nucleotide-sensitive, 1A |
| 41716_at | down | 0.023639 | rabconnectin-3 |
| 172_at | up | 0.023662 | inositol polyphosphate-5-phosphatase, 145 kDa |
| 40361_at | up | 0.023669 | chaperonin containing TCP1, subunit 6B (zeta 2) |
| 38693_at | down | 0.02374 | ATP synthase, H+ transporting, mitochondrial F0 comple; |
| | | | subunit g |
| 40108_at | down | 0.02382 | basic leucine zipper and W2 domains 1 |
| 34306_at | down | 0.023824 | muscleblind-like (Drosophila) |
| 34967_at | up | 0.02384 | similar to RNA polymerase I transcription factor RRN3 |
| 40363_r_at | up | 0.023842 | nuclear factor of kappa light polypeptide gene enhancer |
| | | | B-cells 2 (p49/p100) |
| 32264_at | up | 0.023943 | granzyme M (lymphocyte met-ase 1) |
| 34084_at | up | 0.023985 | aldo-keto reductase family 1, member D1 (delta 4-3- |
| | | | ketosteroid-5-beta-reductase) |
| 39742_at | down | 0.024012 | TRAF family member-associated NFKB activator |
| 33298_at | down | 0.024013 | striatin, calmodulin binding protein |
| 37819_at | down | 0.024063 | nuclear protein double minute 1 |
| 33389_at | down | 0.024066 | cytochrome P450, family 51, subfamily A, polypeptide 1 |
| 39905_i_at | down | 0.024139 | ADP-ribosylation factor GTPase activating protein 3 |
| 32782_r_at | up | 0.024141 | bullous pemphigoid antigen 1, 230/240 kDa |
| 36369_at | up | 0.024193 | polymerase I and transcript release factor |
| 335_r_at | up | 0.024272 | |
| 37094_at | up | 0.024294 | X-ray repair complementing defective repair in Chinese |
| | | | hamster cells 3 |
| 33854_at | down | 0.0243 | ATPase, H+ transporting, lysosomal 34 kDa, V1 subunit D |
| 37651_at | down | 0.024336 | REST corepressor |
| 32989_at | up | 0.024341 | regulatory factor X, 1 (influences HLA class II expression) |
| 40536_f_at | up | 0.024357 | translation initiation factor IF2 |
| 38981_at | down | 0.024383 | NADH dehydrogenase (ubiquinone) 1 beta subcomplex, |
| | | | 3, 12 kDa |
| 325_s_at | up | 0.024407 | |
| 41574_at | down | 0.024445 | pinin, desmosome associated protein |
| 41865_at | down | 0.024447 | ATP synthase mitochondrial F1 complex assembly factor 2 |
| 40275_at | up | 0.024455 | karyopherin alpha 6 (importin alpha 7) |
| 40971_at | down | 0.024476 | ankyrin repeat and SAM domain containing 1 |
| 39868_at | up | 0.024556 | poly(rC) binding protein 3 |
| 37284_at | up | 0.024592 | sema domain, immunoglobulin domain (Ig), |
| | | | transmembrane domain (TM) and short cytoplasmic |
| | | | domain, (semaphorin) 4D |
| 33994_g_at | up | 0.024774 | myosin, light polypeptide 6, alkali, smooth muscle and |
| | | | non-muscle |
| 32705_at | up | 0.024783 | cytochrome P450, family 3, subfamily A, polypeptide 7 |
| 1757_i_at | up | 0.024825 | cytochrome P450, family 3, subfamily A, polypeptide 7 |
| 35199_at | down | 0.024877 | KIAA0982 protein |
| 31468_f_at | up | 0.024961 | glutamate receptor, metabotropic 1 |
| 36372_at | up | 0.02499 | hexokinase 3 (white cell) |
| 41082_at | up | 0.024994 | ras homolog gene family, member N |
| 32110_at | down | 0.025003 | KIAA0523 protein |
| 32162_r_at | up | 0.025004 | |
| 39746_at | down | 0.025006 | polymerase (RNA) II (DNA directed) polypeptide B, |
| | | | 140 kDa |
| 39598_at | up | 0.025046 | gap junction protein, beta 1, 32 kDa (connexin 32, |
| | | | Charcot-Marie-Tooth neuropathy, X-linked) |
| 33815_at | down | 0.025072 | uridine monophosphate synthetase (orotate |
| | | | phosphoribosyl transferase and orotidine-5′- |
| | | | decarboxylase) |
| 1315_at | up | 0.025082 | ornithine decarboxylase antizyme 1 |
| 33368_at | down | 0.025136 | protease, serine, 15 |
| 33312_at | down | 0.025138 | crystallin, alpha A |
| 922_at | up | 0.025139 | protein phosphatase 2 (formerly 2A), regulatory subunit, |
| | | | (PR 65), alpha isoform |
| 33234_at | down | 0.02522 | KIAA0117 protein |
| 39383_at | up | 0.025256 | adenylate cyclase 6 |
| 36375_at | up | 0.025313 | outer dense fiber of sperm tails 1 |
| 32039_at | down | 0.025325 | adaptor-related protein complex 3, beta 1 subunit |
| 1397_at | down | 0.025353 | mitogen-activated protein kinase kinase kinase 11 |
| 36599_at | down | 0.02539 | malic enzyme 2, NAD(+)-dependent, mitochondrial |
| 39316_at | up | 0.025404 | RAB40C, member RAS oncogene family |
| 41194_at | down | 0.025416 | signal recognition particle 14 kDa (homologous Alu RNA |
| | | | binding protein) |
| 41088_at | down | 0.02547 | abhydrolase domain containing 2 |
| 38869_at | up | 0.025484 | KIAA1069 protein |
| 33885_at | down | 0.025514 | KIAA0907 protein |
| 32955_at | down | 0.025531 | hypothetical protein HSPC132 |
| 36670_at | up | 0.025564 | autoantigen |
| 33630_s_at | up | 0.025599 | spectrin, beta, non-erythrocytic 2 |
| 36930_at | down | 0.02563 | nucleolar GTPase |
| 33459_at | up | 0.025662 | |
| 40121_at | down | 0.02568 | huntingtin interacting protein 2 |
| 37562_at | up | 0.025703 | protocadherin 1 (cadherin-like 1) |
| 37658_at | up | 0.025706 | growth arrest-specific 6 |
| 32880_at | up | 0.02574 | secretoglobin, family 1D, member 2 |
| 40094_r_at | up | 0.025791 | Lutheran blood group (Auberger b antigen included) |
| 1849_s_at | down | 0.025971 | retinoblastoma binding protein 1 |
| 34342_s_at | up | 0.025976 | secreted phosphoprotein 1 (osteopontin, bone sialoprote |
| | | | I, early T-lymphocyte activation 1) |
| 39679_at | up | 0.025985 | aquaporin 2 (collecting duct) |
| 41133_at | down | 0.02604 | Ras-GTPase-activating protein SH3-domain-binding |
| | | | protein |
| 40718_at | up | 0.026084 | cathepsin W (lymphopain) |
| _ _at | up | 0.0261 | galanin receptor 3 |
| 40029_at | up | 0.026123 | EGF-like-domain, multiple 3 |
| 41239_r_at | down | 0.026185 | cathepsin S |
| 33363_at | up | 0.026322 | JTV1 gene |
| 40980_at | up | 0.026323 | helicase with SNF2 domain 1 |
| 37280_at | up | 0.026356 | MAD, mothers against decapentaplegic homolog 1 |
| | | | (Drosophila) |
| 37059_at | up | 0.026359 | glucokinase (hexokinase 4) regulatory protein |
| 34304_s_at | down | 0.02639 | spermidine/spermine N1-acetyltransferase |
| 41750_at | down | 0.026432 | protein disulfide isomerase-related protein |
| 34026_at | up | 0.026444 | |
| 35828_at | up | 0.026456 | cysteine-rich protein 2 |
| 34655_at | up | 0.02651 | membrane protein, palmitoylated 2 (MAGUK p55 |
| | | | subfamily member 2) |
| 35955_at | up | 0.026544 | cytochrome c-like antigen |
| 33930_at | down | 0.026608 | chromosome 14 open reading frame 163 |
| 40664_at | up | 0.026615 | brain-specific angiogenesis inhibitor 3 |
| 34110_g_at | up | 0.026639 | proline dehydrogenase (oxidase) 1 |
| 36271_at | up | 0.026668 | KIAA1024 protein |
| 1498_at | up | 0.026683 | zeta-chain (TCR) associated protein kinase 70 kDa |
| 1647_at | down | 0.026835 | IQ motif containing GTPase activating protein 2 |
| 35069_at | up | 0.026876 | hypothetical protein similar to preferentially expressed |
| | | | antigen of melanoma |
| 35135_at | down | 0.026898 | hypothetical protein MGC10471 |
| 34099_f_at | down | 0.02691 | nucleosome assembly protein 1-like 1 |
| 33861_at | down | 0.02691 | CCR4-NOT transcription complex, subunit 2 |
| 39720_g_at | up | 0.026932 | zona pellucida glycoprotein 3 (sperm receptor) |
| 249_at | up | 0.026938 | nuclear factor of activated T-cells, cytoplasmic, |
| | | | calcineurin-dependent 4 |
| 35013_at | up | 0.026947 | lipopolysaccharide binding protein |
| 40164_at | up | 0.027001 | Rho GDP dissociation inhibitor (GDI) alpha |
| 41828_at | down | 0.027034 | methyl-CpG binding domain protein 1 |
| 33658_at | down | 0.027078 | zinc finger protein 124 (HZF-16) |
| 32665_at | down | 0.027087 | protein phosphatase 1B (formerly 2C), magnesium- |
| | | | dependent, beta isoform |
| 35734_at | down | 0.027114 | ARP2 actin-related protein 2 homolog (yeast) |
| 36753_at | down | 0.027141 | leukocyte immunoglobulin-like receptor, subfamily B (with |
| | | | TM and ITIM domains), member 4 |
| 35087_at | down | 0.027142 | |
| 34887_at | down | 0.027168 | radixin |
| 36048_at | down | 0.027245 | zinc finger protein 318 |
| 728_at | up | 0.027264 | |
| 33030_at | up | 0.027277 | histone 1, H1d |
| 35818_at | down | 0.027352 | cytochrome c, somatic |
| 1171_s_at | up | 0.027371 | |
| 32508_at | down | 0.027482 | HBxAg transactivated protein 2 |
| 37758_s_at | up | 0.027495 | transcription factor Dp-1 |
| 31474_r_at | down | 0.027515 | tankyrase, TRF1-interacting ankyrin-related ADP-ribose |
| | | | polymerase |
| 31716_at | up | 0.027701 | protocadherin 1 (cadherin-like 1) |
| 39377_at | down | 0.027712 | mitochondrial ribosomal protein S27 |
| 32833_at | down | 0.027752 | CDC-like kinase 1 |
| 36702_at | up | 0.027768 | T-box 19 |
| 222_at | up | 0.027825 | exostoses (multiple) 1 |
| 35378_at | up | 0.027861 | luteinizing hormone beta polypeptide |
| 37399_at | up | 0.027864 | aldo-keto reductase family 1, member C3 (3-alpha |
| | | | hydroxysteroid dehydrogenase, type II) |
| 41249_at | down | 0.027899 | NAD kinase |
| 37419_g_at | up | 0.027936 | POU domain, class 2, transcription factor 2 |
| 36598_s_at | up | 0.028084 | inositol polyphosphate phosphatase-like 1 |
| 36400_at | up | 0.028084 | |
| 34946_at | down | 0.028102 | immunoglobulin superfamily, member 6 |
| 39568_g_at | up | 0.028143 | aquaporin 7 |
| 33847_s_at | down | 0.028151 | cyclin-dependent kinase inhibitor 1B (p27, Kip1) |
| 33774_at | down | 0.028182 | caspase 8, apoptosis-related cysteine protease |
| 723_s_at | down | 0.028219 | |
| 40852_at | down | 0.028237 | tudor repeat associator with PCTAIRE 2 |
| 32315_at | down | 0.02827 | ribosomal protein S24 |
| 32017_at | up | 0.028338 | par-6 partitioning defective 6 homolog beta (C. elegans) |
| 32254_at | up | 0.02834 | vesicle-associated membrane protein 2 (synaptobrevin 2 |
| 32923_r_at | up | 0.028392 | synapsin I |
| 38515_at | up | 0.028404 | bone morphogenetic protein 7 (osteogenic protein 1) |
| 32012_at | up | 0.02841 | pecanex homolog (Drosophila) |
| 39510_r_at | down | 0.028439 | programmed cell death 4 (neoplastic transformation |
| | | | inhibitor) |
| 34077_at | up | 0.028472 | chemokine (C—X—C motif) receptor 3 |
| 41198_at | up | 0.028513 | granulin |
| 32649_at | down | 0.028531 | transcription factor 7 (T-cell specific, HMG-box) |
| 40968_at | up | 0.028537 | suppressor of cytokine signaling 3 |
| 1587_at | up | 0.028563 | retinoic acid receptor, gamma |
| 38583_at | down | 0.028622 | UDP-Gal:betaGlcNAc beta 1,3-galactosyltransferase, |
| | | | polypeptide 2 |
| 1859_s_at | up | 0.028629 | Mdm2, transformed 3T3 cell double minute 2, p53 bindin |
| | | | protein (mouse) |
| 36244_at | up | 0.028641 | zinc finger protein 239 |
| 39346_at | down | 0.028649 | KH domain containing, RNA binding, signal transduction |
| | | | associated 1 |
| 40607_at | down | 0.028666 | dihydropyrimidinase-like 2 |
| 40659_at | up | 0.02869 | nuclear receptor subfamily 4, group A, member 3 |
| 37112_at | down | 0.028745 | chromosome 6 open reading frame 32 |
| 812_at | down | 0.028758 | protein phosphatase 1, regulatory (inhibitor) subunit 2 |
| 35095_r_at | down | 0.028761 | leukocyte immunoglobulin-like receptor, subfamily A |
| | | | (without TM domain), member 3 |
| 35178_at | up | 0.028823 | WNT inhibitory factor 1 |
| 40655_at | up | 0.028827 | huntingtin-associated protein interacting protein (duo) |
| 38082_at | down | 0.02887 | KIAA0650 protein |
| 41465_at | down | 0.028906 | zinc finger protein 148 (pHZ-52) |
| 39932_at | down | 0.028952 | |
| 35417_at | up | 0.028976 | cubilin (intrinsic factor-cobalamin receptor) |
| 40893_at | down | 0.029072 | succinate-CoA ligase, ADP-forming, beta subunit |
| 40941_at | up | 0.029096 | VAMP (vesicle-associated membrane protein)-associate |
| | | | protein B and C |
| 40351_at | down | 0.029119 | guanine nucleotide binding protein (G protein), beta |
| | | | polypeptide 3 |
| 988_at | down | 0.02912 | carcinoembryonic antigen-related cell adhesion molecule |
| | | | 1 (biliary glycoprotein) |
| 34906_g_at | up | 0.029139 | glutamate receptor, ionotropic, kainate 5 |
| 35900_at | up | 0.029265 | artemin |
| 33138_at | up | 0.029266 | myeloid leukemia factor 1 |
| 36722_s_at | up | 0.029339 | hepatocyte nuclear factor 4, alpha |
| 38025_r_at | up | 0.029343 | rap2 interacting protein x |
| 1271_g_at | up | 0.029435 | v-rel reticuloendotheliosis viral oncogene homolog A, |
| | | | nuclear factor of kappa light polypeptide gene enhancer i |
| | | | B-cells 3, p65 (avian) |
| 39278_at | up | 0.029472 | transglutaminase 4 (prostate) |
| 766_at | up | 0.029553 | lectin, galactoside-binding, soluble, 9 (galectin 9) |
| 36335_at | up | 0.029597 | butyrophilin, subfamily 1, member A1 |
| 38270_at | down | 0.02961 | poly (ADP-ribose) glycohydrolase |
| 258_at | up | 0.029653 | lymphotoxin alpha (TNF superfamily, member 1) |
| 38289_r_at | up | 0.029684 | neurofibromin 1 (neurofibromatosis, von Recklinghausen |
| | | | disease, Watson disease) |
| 37336_at | down | 0.029708 | UBX domain containing 2 |
| 35252_at | down | 0.02973 | KIAA0528 gene product |
| 1102_s_at | down | 0.0298 | nuclear receptor subfamily 3, group C, member 1 |
| | | | (glucocorticoid receptor) |
| 38041_at | down | 0.029812 | UDP-N-acetyl-alpha-D-galactosamine:polypeptide N- |
| | | | acetylgalactosaminyltransferase 1 (GalNAc-T1) |
| 36330_at | up | 0.029865 | cysteine conjugate-beta lyase; cytoplasmic (glutamine |
| | | | transaminase K, kyneurenine aminotransferase) |
| 525_g_at | down | 0.029867 | PMS1 postmeiotic segregation increased 1 (S. cerevisiae |
| 41458_at | up | 0.029904 | KIAA0467 protein |
| 32466_at | up | 0.029925 | ribosomal protein L41 |
| 1659_s_at | down | 0.029946 | Ras homolog enriched in brain 2 |
| 35460_at | up | 0.029947 | G protein-coupled receptor 4 |
| 223_at | down | 0.02995 | ubiquitin-conjugating enzyme E2L 3 |
| 34411_at | down | 0.030124 | 3′-phosphoadenosine 5′-phosphosulfate synthase 1 |
| 1632_at | up | 0.030132 | |
| 33051_at | up | 0.030141 | gamma-aminobutyric acid (GABA) receptor, rho 1 |
| 1441_s_at | down | 0.030183 | tumor necrosis factor receptor superfamily, member 6 |
| 32946_r_at | up | 0.030222 | mannose-binding lectin (protein C) 2, soluble (opsonic |
| | | | defect) |
| 35258_f_at | down | 0.030223 | splicing factor, arginine/serine-rich 2, interacting protein |
| 36677_at | down | 0.030277 | coatomer protein complex, subunit beta 2 (beta prime) |
| 41463_at | up | 0.030285 | hypothetical protein FLJ38984 |
| 36636_at | down | 0.030335 | ornithine aminotransferase (gyrate atrophy) |
| 39778_at | up | 0.030338 | mannosyl (alpha-1,3-)-glycoprotein beta-1,2-N- |
| | | | acetylglucosaminyltransferase |
| 41000_at | down | 0.03037 | checkpoint suppressor 1 |
| 32065_at | up | 0.030391 | cAMP responsive element modulator |
| 34372_at | down | 0.030409 | upstream regulatory element binding protein 1 |
| 35424_g_at | up | 0.030417 | glutamate receptor, metabotropic 5 |
| 36835_at | down | 0.030439 | protein kinase C-like 2 |
| 39920_r_at | up | 0.030491 | C1q-related factor |
| 35408_i_at | down | 0.030498 | GIOT-2 for gonadotropin inducible transcription repress 2 |
| 31878_at | down | 0.030618 | ATP-binding cassette, sub-family F (GCN20), member 2 |
| 38613_at | up | 0.03062 | putative cyclin G1 interacting protein |
| 41780_at | down | 0.03067 | protein tyrosine phosphatase, receptor type, f polypeptid |
| | | | (PTPRF), interacting protein (liprin), alpha 1 |
| 33369_at | down | 0.030743 | sterol-C4-methyl oxidase-like |
| 34783_s_at | down | 0.030757 | BUB3 budding uninhibited by benzimidazoles 3 homolog |
| | | | (yeast) |
| 35212_at | down | 0.03079 | ring finger protein 139 |
| 38507_at | up | 0.030852 | cytochrome P450, family 2, subfamily D, polypeptide 6 |
| 33163_r_at | down | 0.030898 | glutamate-cysteine ligase, modifier subunit |
| 36798_g_at | up | 0.030948 | sialophorin (gpL115, leukosialin, CD43) |
| 39132_at | down | 0.031073 | SWI/SNF related, matrix associated, actin dependent |
| | | | regulator of chromatin, subfamily a, member 5 |
| 34534_at | up | 0.031122 | opioid receptor, mu 1 |
| 40074_at | down | 0.031215 | methylene tetrahydrofolate dehydrogenase (NAD+ |
| | | | dependent), methenyltetrahydrofolate cyclohydrolase |
| 38293_s_at | up | 0.031221 | homeo box D3 |
| 33237_at | down | 0.031222 | RNA helicase |
| 36233_at | up | 0.031254 | 3′-phosphoadenosine 5′-phosphosulfate synthase 2 |
| 32145_at | up | 0.031307 | adducin 1 (alpha) |
| 39499_s_at | up | 0.031346 | par-3 partitioning defective 3 homolog (C. elegans) |
| 36684_at | down | 0.031356 | adenosylmethionine decarboxylase 1 |
| 34760_at | down | 0.031481 | C-type lectin BIMLEC precursor |
| 40126_at | up | 0.031527 | paired mesoderm homeo box 1 |
| 31349_at | up | 0.031545 | DNA-binding protein amplifying expression of surfactant |
| | | | protein B |
| 1007_s_at | up | 0.031546 | discoidin domain receptor family, member 1 |
| 37185_at | up | 0.031584 | serine (or cysteine) proteinase inhibitor, clade B |
| | | | (ovalbumin), member 2 |
| 32100_r_at | up | 0.031585 | galactosamine (N-acetyl)-6-sulfate sulfatase (Morquio |
| | | | syndrome, mucopolysaccharidosis type IVA) |
| 34381_at | down | 0.031631 | cytochrome c oxidase subunit VIIc |
| 36285_at | up | 0.031708 | potassium inwardly-rectifying channel, subfamily J, |
| | | | member 4 |
| 171_at | down | 0.031713 | von Hippel-Lindau binding protein 1 |
| 39659_at | down | 0.031715 | Ts translation elongation factor, mitochondrial |
| 37289_at | up | 0.031818 | cadherin 8, type 2 |
| 36218_g_at | up | 0.031859 | serine/threonine kinase 38 |
| 31410_at | up | 0.031863 | tumor necrosis factor receptor superfamily, member 13B |
| 35387_r_at | up | 0.031917 | acetylcholinesterase (YT blood group) |
| 33598_r_at | down | 0.031933 | cold autoinflammatory syndrome 1 |
| 38435_at | down | 0.031934 | peroxiredoxin 4 |
| 36510_at | down | 0.031942 | general transcription factor IIF, polypeptide 2, 30 kDa |
| 41048_at | down | 0.031992 | phorbol-12-myristate-13-acetate-induced protein 1 |
| 38441_s_at | down | 0.03202 | membrane cofactor protein (CD46, trophoblast- |
| | | | lymphocyte cross-reactive antigen) |
| 39483_s_at | down | 0.032042 | integrin, beta 1 (fibronectin receptor, beta polypeptide, |
| | | | antigen CD29 includes MDF2, MSK12) |
| 31889_at | up | 0.032073 | melan-A |
| 39443_s_at | down | 0.032107 | cytochrome c oxidase subunit Vb |
| 40172_g_at | up | 0.032138 | frequently rearranged in advanced T-cell lymphomas 2 |
| 36580_at | down | 0.032164 | hypothetical protein FLJ13910 |
| 1140_at | down | 0.032164 | integrin, alpha E (antigen CD103, human mucosal |
| | | | lymphocyte antigen 1; alpha polypeptide) |
| 37877_at | up | 0.032166 | DKFZP564C103 protein |
| 38402_at | down | 0.032188 | lysosomal-associated membrane protein 2 |
| 39037_at | down | 0.032197 | myeloid/lymphoid or mixed-lineage leukemia (trithorax |
| | | | homolog, Drosophila); translocated to, 2 |
| 34830_at | up | 0.032228 | hypothetical protein DKFZp564K0822 |
| 32313_at | up | 0.032235 | tropomyosin 2 (beta) |
| 36332_at | up | 0.032309 | arylalkylamine N-acetyltransferase |
| 36979_at | up | 0.032388 | solute carrier family 2 (facilitated glucose transporter), |
| | | | member 3 |
| 32892_at | up | 0.032417 | ribosomal protein S6 kinase, 90 kDa, polypeptide 2 |
| 1818_at | down | 0.032454 | |
| 38040_at | down | 0.032494 | splicing factor 30, survival of motor neuron-related |
| 31771_at | up | 0.032514 | |
| 37006_at | down | 0.032531 | immunoglobulin J polypeptide, linker protein for |
| | | | immunoglobulin alpha and mu polypeptides |
| 35310_at | up | 0.032585 | |
| 2090_i_at | up | 0.032631 | |
| 36647_at | down | 0.032661 | hypothetical protein FLJ10326 |
| 35259_s_at | down | 0.032688 | splicing factor, arginine/serine-rich 2, interacting protein |
| 1105_s_at | up | 0.032709 | T cell receptor beta locus |
| 31904_at | up | 0.03274 | phosphodiesterase 2A, cGMP-stimulated |
| 40207_g_at | down | 0.032759 | chymotrypsin-like |
| 38397_at | down | 0.03281 | polymerase (DNA-directed), delta 4 |
| 38681_at | down | 0.03289 | eukaryotic translation initiation factor 3, subunit 6 48 kDa |
| 37164_at | up | 0.032937 | Rhesus blood group, D antigen |
| 37944_at | down | 0.032989 | GTP cyclohydrolase 1 (dopa-responsive dystonia) |
| 34091_s_at | up | 0.033014 | vimentin |
| 34008_at | up | 0.033133 | RAS (RAD and GEM)-like GTP-binding |
| 39879_s_at | up | 0.033144 | hypothetical protein FLJ10120 |
| 37178_at | up | 0.03329 | hypothetical protein BC017169 |
| 36444_s_at | down | 0.033356 | chemokine (C—C motif) ligand 23 |
| 33574_at | up | 0.033458 | chromosome 6 open reading frame 10 |
| 39725_at | down | 0.033516 | RNA-binding region (RNP1, RRM) containing 2 |
| 33871_s_at | up | 0.033547 | folate receptor 2 (fetal) |
| 891_at | down | 0.033587 | YY1 transcription factor |
| 37514_s_at | up | 0.033701 | mannan-binding lectin serine protease 2 |
| 32666_at | up | 0.033716 | chemokine (C—X—C motif) ligand 12 (stromal cell-derived |
| | | | factor 1) |
| 32498_at | up | 0.033761 | glutamate receptor, metabotropic 2 |
| 33967_at | up | 0.033788 | major histocompatibility complex, class II, DO alpha |
| 34280_at | up | 0.033821 | gamma-aminobutyric acid (GABA) A receptor, epsilon |
| 32178_r_at | down | 0.033864 | synaptosomal-associated protein, 23 kDa |
| 39701_at | up | 0.033899 | paternally expressed 3 |
| 38715_at | up | 0.033904 | glycophorin B (includes Ss blood group) |
| 40144_at | down | 0.033918 | protein tyrosine phosphatase, non-receptor type substr 1 |
| 41830_at | down | 0.033922 | KIAA0494 gene product |
| 35211_at | up | 0.034035 | protein phosphatase 2 (formerly 2A), regulatory subunit |
| | | | B″, alpha |
| 38123_at | down | 0.034045 | chromosome 10 open reading frame 7 |
| 39341_at | up | 0.034163 | thyroid hormone receptor interactor 6 |
| 33651_at | up | 0.034184 | aquaporin 8 |
| 41549_s_at | down | 0.034205 | adaptor-related protein complex 1, sigma 2 subunit |
| 40897_at | down | 0.034209 | phosphodiesterase 6A, cGMP-specific, rod, alpha |
| 37586_at | up | 0.034298 | zinc finger protein 142 (clone pHZ-49) |
| 32175_at | down | 0.034333 | CDC10 cell division cycle 10 homolog (S. cerevisiae) |
| 34368_at | down | 0.034371 | histone deacetylase 2 |
| 40859_at | down | 0.034403 | nuclear protein UKp68 |
| 35588_at | down | 0.034495 | zinc finger protein 443 |
| 33977_at | up | 0.034609 | growth factor independent 1 |
| 623_s_at | down | 0.034643 | RAB2, member RAS oncogene family |
| 41747_s_at | down | 0.034656 | MADS box transcription enhancer factor 2, polypeptide |
| | | | (myocyte enhancer factor 2A) |
| 33381_at | down | 0.034676 | nuclear receptor coactivator 3 |
| 35367_at | down | 0.03477 | lectin, galactoside-binding, soluble, 3 (galectin 3) |
| 1964_g_at | up | 0.034805 | fms-related tyrosine kinase 1 (vascular endothelial growt |
| | | | factor/vascular permeability factor receptor) |
| 41637_at | up | 0.034911 | dexamethasone-induced transcript |
| 39888_at | up | 0.034927 | UDP-Gal:betaGlcNAc beta 1,4-galactosyltransferase, |
| | | | polypeptide 5 |
| 38847_at | up | 0.034942 | maternal embryonic leucine zipper kinase |
| 36897_at | up | 0.034982 | megalencephalic leukoencephalopathy with subcortical |
| | | | cysts 1 |
| 37984_s_at | down | 0.035019 | ADP-ribosylation factor 6 |
| 41505_r_at | down | 0.035043 | v-maf musculoaponeurotic fibrosarcoma oncogene |
| | | | homolog (avian) |
| 1292_at | up | 0.035215 | dual specificity phosphatase 2 |
| 35505_at | up | 0.035231 | SWI/SNF related, matrix associated, actin dependent |
| | | | regulator of chromatin, subfamily f, member 1 |
| 38664_at | down | 0.035232 | craniofacial development protein 1 |
| 35572_f_at | down | 0.035287 | zinc finger protein 253 |
| 32281_at | up | 0.035303 | sorting nexin 15 |
| 32478_f_at | down | 0.035339 | |
| 32094_at | up | 0.035363 | carbohydrate (chondroitin 6) sulfotransferase 3 |
| 38597_f_at | up | 0.035398 | solute carrier family 11 (proton-coupled divalent metal io |
| | | | transporters), member 1 |
| 41446_f_at | up | 0.035415 | RNA helicase-related protein |
| 38711_at | down | 0.035439 | cytoplasmic linker associated protein 2 |
| 1017_at | down | 0.035486 | |
| 39288_at | up | 0.035547 | nectin-like protein 1 |
| 710_at | up | 0.035599 | procollagen-proline, 2-oxoglutarate 4-dioxygenase (proli |
| | | | 4-hydroxylase), beta polypeptide (protein disulfide |
| | | | isomerase; thyroid hormone binding protein p55) |
| 36690_at | down | 0.03562 | nuclear receptor subfamily 3, group C, member 1 |
| | | | (glucocorticoid receptor) |
| 33424_at | down | 0.035644 | ribophorin I |
| 943_at | down | 0.035681 | runt-related transcription factor 1 (acute myeloid leukem |
| | | | 1; aml1 oncogene) |
| 39822_s_at | down | 0.035691 | growth arrest and DNA-damage-inducible, beta |
| 41768_at | down | 0.035716 | protein kinase, cAMP-dependent, regulatory, type I, alp |
| | | | (tissue specific extinguisher 1) |
| 40773_at | up | 0.03575 | myosin, light polypeptide 5, regulatory |
| 35720_at | down | 0.035787 | KIAA0893 protein |
| 40151_s_at | down | 0.035808 | peroxisome receptor 1 |
| 1640_at | down | 0.035835 | suppression of tumorigenicity 13 (colon carcinoma) |
| | | | (Hsp70 interacting protein) |
| 41054_at | up | 0.035852 | KIAA0290 protein |
| 37724_at | down | 0.0359 | v-myc myelocytomatosis viral oncogene homolog (avian |
| 38654_at | down | 0.035915 | heterogeneous nuclear ribonucleoprotein U (scaffold |
| | | | attachment factor A) |
| 34251_at | up | 0.035965 | homeo box B5 |
| 36974_at | down | 0.036018 | proteasome (prosome, macropain) inhibitor subunit 1 |
| | | | (PI31) |
| 34197_at | up | 0.036025 | phosphoinositide-3-kinase, regulatory subunit, polypepti |
| | | | 2 (p85 beta) |
| 39092_at | down | 0.036029 | histone H2A.F/Z variant |
| 38972_at | down | 0.036149 | hypothetical protein BC013764 |
| 34060_g_at | up | 0.036265 | Pvt1 oncogene homolog, MYC activator (mouse) |
| 32898_at | up | 0.036315 | actin like protein |
| 40945_at | up | 0.03632 | TGFB inducible early growth response 2 |
| AFFX-BioDn- | up | 0.036336 | |
| 3_st | | | |
| 37568_at | up | 0.036344 | |
| 37591_at | up | 0.036353 | uncoupling protein 2 (mitochondrial, proton carrier) |
| 35991_at | down | 0.036357 | LSM6 homolog, U6 small nuclear RNA associated (S. cerevisiae) |
| 40193_at | up | 0.036372 | enolase 2, (gamma, neuronal) |
| 39885_at | down | 0.036385 | putative dimethyladenosine transferase |
| 239_at | up | 0.036478 | cathepsin D (lysosomal aspartyl protease) |
| 41279_f_at | up | 0.03657 | mitogen-activated protein kinase 8 interacting protein 1 |
| 32630_f_at | down | 0.03659 | butyrophilin, subfamily 3, member A1 |
| 38985_at | down | 0.036653 | leptin receptor overlapping transcript-like 1 |
| 41510_s_at | down | 0.036688 | heat shock 70 kDa protein 9B (mortalin-2) |
| 39744_at | down | 0.036695 | DEAD/H (Asp-Glu-Ala-Asp/His) box polypeptide 3 |
| 41561_s_at | down | 0.03671 | intraflagellar transport protein IFT20 |
| 37482_at | up | 0.036796 | aldo-keto reductase family 1, member B10 (aldose |
| | | | reductase) |
| 36968_s_at | down | 0.036823 | Opa-interacting protein 2 |
| 34750_r_at | down | 0.036851 | zinc finger and BTB domain containing 1 |
| 1274_s_at | up | 0.036896 | cell division cycle 34 |
| 35713_at | up | 0.036927 | Fanconi anemia, complementation group C |
| 37643_at | down | 0.036948 | tumor necrosis factor receptor superfamily, member 6 |
| 34364_at | up | 0.036957 | peptidylprolyl isomerase E (cyclophilin E) |
| 760_at | down | 0.037036 | dual-specificity tyrosine-(Y)-phosphorylation regulated |
| | | | kinase 2 |
| 34965_at | up | 0.037053 | cystatin F (leukocystatin) |
| 32503_at | down | 0.037099 | sorting nexin 27 |
| 37192_at | up | 0.037113 | erythrocyte membrane protein band 4.9 (dematin) |
| 39613_at | down | 0.037219 | mannosidase, alpha, class 1A, member 1 |
| 975_at | up | 0.03724 | serine/threonine kinase 18 |
| 37673_at | down | 0.037261 | neutral sphingomyelinase (N-SMase) activation |
| | | | associated factor |
| 34191_at | down | 0.037275 | |
| 1450_g_at | down | 0.03728 | proteasome (prosome, macropain) subunit, alpha type, |
| 33154_at | down | 0.037299 | proteasome (prosome, macropain) subunit, beta type, 4 |
| 39637_at | down | 0.037323 | solute carrier family 26 (sulfate transporter), member 2 |
| 33781_s_at | up | 0.037374 | ubiquitin-conjugating enzyme E2M (UBC12 homolog, |
| | | | yeast) |
| 38364_at | down | 0.037386 | transducin-like enhancer of split 4 (E(sp1) homolog, |
| | | | Drosophila) |
| 34902_at | up | 0.03739 | KIAA0492 protein |
| 36516_at | up | 0.037461 | zinc finger protein ZFP100 |
| 949_s_at | down | 0.037476 | proteasome (prosome, macropain) 26S subunit, ATPas 6 |
| 36777_at | up | 0.037482 | DNA segment on chromosome 12 (unique) 2489 |
| | | | expressed sequence |
| 38764_at | down | 0.037533 | |
| 1231_at | down | 0.037551 | ubiquitin-conjugating enzyme E2B (RAD6 homolog) |
| 33765_at | up | 0.037599 | chemokine (C—X—C motif) ligand 2 |
| 34089_at | up | 0.037666 | KIAA1030 protein |
| 39104_at | up | 0.037676 | |
| 36311_at | up | 0.037683 | phosphodiesterase 1A, calmodulin-dependent |
| 31636_s_at | up | 0.037708 | solute carrier family 18 (vesicular acetylcholine), membe 3 |
| 32029_at | up | 0.037719 | 3-phosphoinositide dependent protein kinase-1 |
| 37107_at | down | 0.037821 | protein phosphatase ID magnesium-dependent, delta |
| | | | isoform |
| 36780_at | up | 0.037836 | clusterin (complement lysis inhibitor, SP-40, 40, sulfated |
| | | | glycoprotein 2, testosterone-repressed prostate messag |
| | | | 2, apolipoprotein J) |
| 39915_at | up | 0.037879 | transient receptor potential cation channel, subfamily M, |
| | | | member 2 |
| 38895_i_at | up | 0.038008 | neutrophil cytosolic factor 4, 40 kDa |
| 31689_at | down | 0.038009 | DKFZP564G092 protein |
| 35652_g_at | down | 0.038037 | mitogen-activated protein kinase kinase kinase 4 |
| 37483_at | down | 0.038043 | histone deacetylase 9 |
| 36016_at | up | 0.038044 | cortistatin |
| 36568_at | up | 0.03806 | solute carrier family 17 (sodium-dependent inorganic |
| | | | phosphate cotransporter), member 7 |
| 31411_at | up | 0.038106 | variable charge, Y chromosome, 2 |
| 314_at | down | 0.038139 | phosphatidylinositol glycan, class B |
| 33761_s_at | up | 0.038262 | KIAA0493 protein |
| 32983_at | up | 0.038292 | adrenergic, alpha-1B-, receptor |
| 40143_at | down | 0.038345 | KIAA0140 gene product |
| 37671_at | down | 0.038399 | laminin, alpha 4 |
| 38727_at | down | 0.03847 | multiple coagulation factor deficiency protein 2 |
| 35535_f_at | up | 0.038485 | KIAA0565 gene product |
| 35762_at | down | 0.038526 | KIAA0483 protein |
| 1260_s_at | up | 0.038541 | glutathione S-transferase A2 |
| 35539_at | up | 0.038541 | interphotoreceptor matrix proteoglycan 1 |
| 33067_at | up | 0.038554 | histone 1, H1a |
| 33450_at | up | 0.038556 | actin-like 6 |
| 34795_at | up | 0.038587 | procollagen-lysine, 2-oxoglutarate 5-dioxygenase (lysine |
| | | | hydroxylase) 2 |
| 35353_at | down | 0.038672 | proteasome (prosome, macropain) 26S subunit, ATPas 2 |
| 390_at | up | 0.038847 | chemokine (C—C motif) receptor 4 |
| 38160_at | down | 0.038867 | lymphocyte antigen 75 |
| 962_at | up | 0.038972 | BMX non-receptor tyrosine kinase |
| 36347_f_at | up | 0.039084 | histone 1, H2bn |
| 35221_at | down | 0.039152 | purine-rich element binding protein A |
| 32043_at | up | 0.039176 | stanniocalcin 2 |
| 1940_at | down | 0.039252 | v-Ki-ras2 Kirsten rat sarcoma 2 viral oncogene homolog |
| 39338_at | down | 0.039273 | S100 calcium binding protein A10 (annexin II ligand, |
| | | | calpactin I, light polypeptide (p11)) |
| 40840_at | up | 0.039302 | peptidylprolyl isomerase F (cyclophilin F) |
| 422_s_at | up | 0.03932 | MAX protein |
| 32196_at | up | 0.039333 | TBP-interacting protein |
| 41699_f_at | down | 0.039349 | bromodomain containing 1 |
| 31826_at | up | 0.039468 | KIAA0674 protein |
| 37309_at | up | 0.03947 | ras homolog gene family, member A |
| 35262_at | up | 0.039475 | integrin beta 4 binding protein |
| 2089_s_at | up | 0.039507 | v-erb-b2 erythroblastic leukemia viral oncogene homolog |
| | | | 3 (avian) |
| 31798_at | up | 0.039562 | trefoil factor 1 (breast cancer, estrogen-inducible |
| | | | sequence expressed in) |
| 39068_at | up | 0.039709 | protein phosphatase 2, regulatory subunit B (B56), delta |
| | | | isoform |
| 40668_s_at | up | 0.039712 | CD6 antigen |
| 38405_at | down | 0.03972 | fragile X mental retardation, autosomal homolog 1 |
| 37664_at | up | 0.039744 | developmentally regulated GTP binding protein 2 |
| 39908_at | up | 0.039777 | TAF6-like RNA polymerase II, p300/CBP-associated |
| | | | factor (PCAF)-associated factor, 65 kDa |
| 32209_at | up | 0.03978 | Mouse Mammary Turmor Virus Receptor homolog 1 |
| 37885_at | up | 0.039796 | hypothetical protein AF038169 |
| 38454_g_at | down | 0.039796 | intercellular adhesion molecule 2 |
| 37007_at | down | 0.039799 | tumor differentially expressed 1 |
| 697_f_at | up | 0.039946 | |
| 34643_at | up | 0.039976 | ribosomal protein S4, X-linked |
| 31331_at | up | 0.040002 | surfactant protein A binding protein |
| 1726_at | up | 0.040012 | |
| 33208_at | down | 0.040098 | DnaJ (Hsp40) homolog, subfamily C, member 3 |
| 38220_at | down | 0.040129 | dihydropyrimidine dehydrogenase |
| 36986_at | up | 0.040158 | lysophospholipase II |
| 41429_at | up | 0.040222 | protein phosphatase 2 (formerly 2A), regulatory subunit |
| | | | (PR 65), beta isoform |
| 34460_at | up | 0.040229 | benzodiazapine receptor (peripheral) associated protein |
| 762_f_at | up | 0.040254 | histone 1, H4i |
| 31860_at | down | 0.040289 | chromosome 17 open reading frame 35 |
| 39111_s_at | down | 0.040343 | peptidylprolyl isomerase (cyclophilin)-like 2 |
| 40102_at | down | 0.040375 | oxysterol binding protein-like 2 |
| 35719_at | down | 0.040521 | pleckstrin homology domain containing, family E (with |
| | | | leucine rich repeats) member 1 |
| 34605_at | up | 0.040586 | activating transcription factor 7 |
| 37324_at | down | 0.040601 | transferrin receptor (p90, CD71) |
| 41692_at | down | 0.040613 | synaptojanin 1 |
| 39345_at | down | 0.040629 | Niemann-Pick disease, type C2 |
| 40612_at | down | 0.040677 | KIAA1117 protein |
| 38690_at | down | 0.040763 | chromosome 3 open reading frame 4 |
| 41046_s_at | up | 0.040853 | zinc finger protein 261 |
| 38450_at | down | 0.040905 | Sjogren syndrome antigen B (autoantigen La) |
| 31461_at | up | 0.041106 | proteasome (prosome, macropain) 26S subunit, non- |
| | | | ATPase, 4, pseudogene |
| 32087_at | down | 0.041149 | heat shock transcription factor 2 |
| 317_at | up | 0.041156 | legumain |
| 32204_at | up | 0.04122 | phosphodiesterase 6G, cGMP-specific, rod, gamma |
| 37242_at | down | 0.041221 | hypothetical protein MGC5149 |
| 35163_at | down | 0.041239 | KIAA1041 protein |
| 34047_at | up | 0.041244 | ovo-like 1(Drosophila) |
| 292_s_at | down | 0.041287 | |
| 37889_at | up | 0.041327 | CD47 antigen (Rh-related antigen, integrin-associated |
| | | | signal transducer) |
| 706_at | down | 0.04142 | |
| 1946_at | up | 0.041472 | Wilms tumor associated protein |
| 34067_at | up | 0.041485 | type II transmembrane serine protease 6 |
| 41698_at | up | 0.041503 | solute carrier family 9 (sodium/hydrogen exchanger), |
| | | | isoform 8 |
| 36325_at | up | 0.041595 | crystallin, beta A1 |
| 39103_s_at | up | 0.041671 | |
| 39319_at | down | 0.041707 | lymphocyte cytosolic protein 2 (SH2 domain containing |
| | | | leukocyte protein of 76 kDa) |
| 39621_at | up | 0.04171 | KIAA0459 protein |
| 32830_g_at | down | 0.041825 | translocase of inner mitochondrial membrane 17 homolo |
| | | | A (yeast) |
| 34816_at | down | 0.041866 | E1A binding protein p400 |
| 32736_at | up | 0.041867 | ras-related C3 botulinum toxin substrate 2 (rho family, |
| | | | small GTP binding protein Rac2) |
| 33553_r_at | down | 0.041915 | chemokine (C—C motif) receptor 6 |
| 1521_at | down | 0.041957 | non-metastatic cells 1, protein (NM23A) expressed in |
| 35290_at | down | 0.041996 | hypothetical protein FLJ31657 |
| 34430_at | up | 0.042007 | glutamic-pyruvate transaminase (alanine |
| | | | aminotransferase) |
| 34376_at | up | 0.042019 | protein kinase (cAMP-dependent, catalytic) inhibitor |
| | | | gamma |
| 1323_at | down | 0.042034 | ubiquitin B |
| 1537_at | up | 0.042061 | epidermal growth factor receptor (erythroblastic leukemia |
| | | | viral (v-erb-b) oncogene homolog, avian) |
| 33008_at | up | 0.042078 | olfactory receptor, family 7, subfamily E, member 24 |
| | | | pseudogene |
| 32918_at | up | 0.0422 | |
| 39027_at | down | 0.042311 | cytochrome c oxidase subunit IV isoform 1 |
| 41436_at | down | 0.042331 | zinc finger protein 198 |
| 34864_at | up | 0.042446 | hypothetical protein CGI-57 |
| 39503_s_at | up | 0.042509 | dihydropyrimidinase-like 4 |
| 35709_at | down | 0.042524 | hypothetical protein FLJ11149 |
| 37011_at | down | 0.04259 | allograft inflammatory factor 1 |
| 31439_f_at | up | 0.042596 | Rhesus blood group, CcEe antigens |
| 39557_at | down | 0.042673 | |
| 40019_at | down | 0.042779 | ecotropic viral integration site 2B |
| 32499_at | up | 0.04278 | Rho GDP dissociation inhibitor (GDI) gamma |
| 35854_at | down | 0.042925 | solute carrier family 18 (vesicular monoamine), member 2 |
| 34840_at | down | 0.042926 | |
| 37936_at | down | 0.043001 | PRP4 pre-mRNA processing factor 4 homolog (yeast) |
| 36099_at | down | 0.043002 | splicing factor, arginine/serine-rich 1 (splicing factor 2, |
| | | | alternate splicing factor) |
| 39530_at | up | 0.043068 | enigma (LIM domain protein) |
| 40926_at | up | 0.043095 | solute carrier family 6 (neurotransmitter transporter, |
| | | | creatine), member 8 |
| AFFX-BioB-3_at | up | 0.043174 | |
| 34135_at | up | 0.043181 | |
| 31544_at | up | 0.043193 | forkhead box I1 |
| 38276_at | down | 0.043244 | nuclear factor of kappa light polypeptide gene enhancer |
| | | | B-cells inhibitor, epsilon |
| 35166_at | down | 0.043337 | Down syndrome critical region gene 3 |
| 41394_at | up | 0.043359 | phospholipase D2 |
| 33339_g_at | down | 0.043366 | signal transducer and activator of transcription 1, 91 kDa |
| 33317_at | down | 0.043374 | cyclin-dependent kinase 7 (MO15 homolog, Xenopus |
| | | | laevis, cdk-activating kinase) |
| 33920_at | up | 0.043398 | diaphanous homolog 1 (Drosophila) |
| 39897_at | down | 0.043457 | splicing factor YT521-B |
| 39739_at | down | 0.043459 | nascent-polypeptide-associated complex alpha |
| | | | polypeptide |
| 40596_at | up | 0.04355 | Treacher Collins-Franceschetti syndrome 1 |
| 39794_at | down | 0.043575 | ubiquitin specific protease 8 |
| 41153_f_at | down | 0.043582 | catenin (cadherin-associated protein), alpha 1, 102 kDa |
| 38503_at | up | 0.043583 | aldehyde dehydrogenase 1 family, member B1 |
| 40220_at | down | 0.043623 | HMBA-inducible |
| 31752_at | up | 0.043695 | hypothetical protein FLJ23142 |
| 36174_at | down | 0.043725 | MARCKS-like protein |
| 36197_at | down | 0.043728 | chitinase 3-like 1 (cartilage glycoprotein-39) |
| 1692_s_at | up | 0.043818 | GDNF family receptor alpha 2 |
| 35513_r_at | up | 0.043851 | Rho family guanine-nucleotide exchange factor |
| 31566_at | up | 0.043873 | |
| 32161_at | down | 0.043989 | seven in absentia homolog 1 (Drosophila) |
| 34277_at | up | 0.044051 | carbonic anhydrase XI |
| 38808_at | up | 0.044055 | adhesion regulating molecule 1 |
| 40864_at | down | 0.044105 | ras-related C3 botulinum toxin substrate 1 (rho family, |
| | | | small GTP binding protein Rac1) |
| 35388_at | up | 0.044116 | LIM homeobox protein 1 |
| 251_at | down | 0.04425 | calcium/calmodulin-dependent protein kinase I |
| 34792_at | down | 0.044292 | S-adenosylhomocysteine hydrolase-like 1 |
| 32007_at | up | 0.044325 | |
| 39163_at | down | 0.04434 | likely homolog of rat kinase D-interacting substance of |
| | | | 220 kDa |
| 31676_at | up | 0.044351 | zinc finger protein 208 |
| 38323_at | down | 0.044372 | carboxypeptidase, vitellogenic-like |
| 32028_at | up | 0.044373 | phosphomannomutase 2 |
| 34959_at | up | 0.044378 | Fc fragment of IgE, low affinity II, receptor for (CD23A) |
| 35193_at | down | 0.044416 | chromosome condensation 1-like |
| 33415_at | down | 0.044429 | non-metastatic cells 2, protein (NM23B) expressed in |
| 37356_r_at | down | 0.044508 | vesicle docking protein p115 |
| 33950_g_at | up | 0.044544 | corticotropin releasing hormone receptor 2 |
| 36913_at | down | 0.044573 | stem-loop (histone) binding protein |
| 41559_at | down | 0.044597 | KIAA1201 protein |
| 38187_at | down | 0.04461 | N-acetyltransferase 1 (arylamine N-acetyltransferase) |
| 40972_at | down | 0.044616 | v-akt murine thymoma viral oncogene homolog 2 |
| 595_at | down | 0.04463 | tumor necrosis factor, alpha-induced protein 3 |
| 34693_at | down | 0.044662 | sialyltransferase |
| 34870_at | up | 0.044673 | LIM domain binding 3 |
| 1116_at | up | 0.044741 | CD19 antigen |
| 36490_s_at | up | 0.044883 | phosphoribosyl pyrophosphate synthetase 1 |
| 37696_at | down | 0.044938 | voltage-dependent anion channel 2 |
| 32369_at | up | 0.044951 | serum amyloid A4, constitutive |
| 36537_at | down | 0.044967 | Rho-specific guanine nucleotide exchange factor p114 |
| 279_at | down | 0.044968 | nuclear receptor subfamily 4, group A, member 1 |
| 35379_at | up | 0.045025 | collagen, type IX, alpha 1 |
| 1573_at | up | 0.045053 | platelet-derived growth factor beta polypeptide (simian |
| | | | sarcoma viral (v-sis) oncogene homolog) |
| 38247_at | down | 0.045143 | coagulation factor II (thrombin) receptor-like 1 |
| 39763_at | up | 0.045196 | hemopexin |
| 1093_at | down | 0.045205 | protein phosphatase 2 (formerly 2A), regulatory subunit |
| | | | (PR 65), beta isoform |
| 36238_at | up | 0.045459 | myeloid/lymphoid or mixed-lineage leukemia (trithorax |
| | | | homolog, Drosophila); translocated to, 7 |
| 31434_at | up | 0.045471 | |
| 36177_at | down | 0.04549 | translin |
| 39270_at | up | 0.045516 | CD209 antigen-like |
| 32547_at | down | 0.045552 | mannose-6-phosphate receptor (cation dependent) |
| 32948_at | up | 0.045614 | Usher syndrome 2A (autosomal recessive, mild) |
| 39028_at | down | 0.045662 | karyopherin (importin) beta 3 |
| 34634_s_at | up | 0.045678 | 5-hydroxytryptamine (serotonin) receptor 7 (adenylate |
| | | | cyclase-coupled) |
| 38544_at | up | 0.04577 | inhibin, alpha |
| 41597_s_at | down | 0.045846 | SEC22 vesicle trafficking protein-like 1 (S. cerevisiae) |
| 38144_at | up | 0.045854 | hypothetical protein DKFZp667B1218 |
| 39758_f_at | up | 0.045944 | lysosomal-associated membrane protein 1 |
| 38436_at | down | 0.045972 | KIAA0252 protein |
| 32143_at | up | 0.045998 | odd-skipped-related 2A protein |
| 37391_at | down | 0.046076 | cathepsin L |
| 41860_at | down | 0.046107 | hypothetical protein BC015148 |
| 1913_at | down | 0.046111 | cyclin G2 |
| 244_at | down | 0.046131 | heat shock transcription factor 1 |
| 38746_at | up | 0.046186 | integrin, beta 4 |
| 35896_at | up | 0.046222 | DKFZp434P211 protein |
| 40115_at | down | 0.046251 | ATP synthase, H+ transporting, mitochondrial F1 comple |
| | | | gamma polypeptide 1 |
| 35251_at | down | 0.046264 | human immunodeficiency virus type I enhancer binding |
| | | | protein 1 |
| 1678_g_at | up | 0.046272 | insulin-like growth factor binding protein 5 |
| 32389_at | up | 0.046313 | RNA, U2 small nuclear |
| 40951_at | up | 0.04634 | BTG3 associated nuclear protein |
| 33708_at | up | 0.046346 | prostate cancer overexpressed gene 1 |
| 41530_at | down | 0.046412 | acetyl-Coenzyme A acyltransferase 2 (mitochondrial 3- |
| | | | oxoacyl-Coenzyme A thiolase) |
| 40049_at | down | 0.046435 | death-associated protein kinase 1 |
| 37276_at | down | 0.04647 | IQ motif containing GTPase activating protein 2 |
| 37512_at | up | 0.046491 | 3-hydroxysteroid epimerase |
| 33862_at | up | 0.046504 | phosphatidic acid phosphatase type 2B |
| 1427_g_at | down | 0.046519 | Src-like-adaptor |
| 34367_at | up | 0.04653 | phosphoglycerate dehydrogenase |
| 38823_s_at | down | 0.0466 | serine/threonine kinase 17a (apoptosis-inducing) |
| 1257_s_at | up | 0.046621 | quiescin Q6 |
| 37237_at | up | 0.046666 | adaptor-related protein complex 3, mu 2 subunit |
| 38747_at | up | 0.046695 | CD34 antigen |
| 35038_at | up | 0.046799 | myosin binding protein C, cardiac |
| 39046_at | down | 0.046853 | histone H2A.F/Z variant |
| 35109_at | up | 0.046897 | neurofascin |
| 34586_s_at | up | 0.046906 | distal-less homeo box 2 |
| 37522_r_at | down | 0.046935 | nucleolar cysteine-rich protein |
| 40160_at | down | 0.046944 | POM121 membrane glycoprotein (rat) |
| 40791_at | up | 0.046947 | polymerase (RNA) II (DNA directed) polypeptide A, |
| | | | 220 kDa |
| 31357_at | up | 0.046987 | |
| 33808_at | up | 0.046995 | TEA domain family member 3 |
| 40831_at | down | 0.047033 | DKFZP586B0923 protein |
| 37938_at | down | 0.047074 | acidic 82 kDa protein mRNA |
| 35858_at | up | 0.047084 | postmeiotic segregation increased 2-like 9 |
| 33466_at | up | 0.047098 | hypothetical gene supported by AF038182; BC009203 |
| 783_at | down | 0.047127 | WW domain-containing protein 1 |
| 36232_at | up | 0.047127 | fibroblast growth factor 13 |
| 33947_at | up | 0.047147 | G protein-coupled receptor 3 |
| 40317_at | up | 0.047216 | amiloride-sensitive cation channel 1, neuronal (degener |
| 39782_at | down | 0.047224 | nuclear DNA-binding protein |
| 35829_at | up | 0.04723 | immunoglobulin superfamily, member 4 |
| 38193_at | up | 0.047282 | immunoglobulin kappa constant |
| 31510_s_at | down | 0.047301 | H3 histone, family 3B (H3.3B) |
| 41303_r_at | down | 0.047306 | hypothetical protein dJ465N24.2.1 |
| 37831_at | up | 0.047318 | KIAA0545 protein |
| 38138_at | up | 0.047335 | S100 calcium binding protein A11 (calgizzarin) |
| 32852_at | up | 0.047348 | thioredoxin 2 |
| 31408_at | up | 0.047356 | retinal pigment epithelium-derived rhodopsin homolog |
| 38758_at | up | 0.047434 | PDGFA associated protein 1 |
| 37905_r_at | up | 0.047444 | |
| 41572_r_at | down | 0.047457 | v-rel reticuloendotheliosis viral oncogene homolog (avian |
| 35294_at | down | 0.047527 | Sjogren syndrome antigen A2 (60 kDa, ribonucleoprotein |
| | | | autoantigen SS-A/Ro) |
| 40494_at | up | 0.047561 | death effector domain containing |
| 37510_at | down | 0.047611 | syntaxin 8 |
| 39056_at | down | 0.047621 | phosphoribosylaminoimidazole carboxylase, |
| | | | phosphoribosylaminoimidazole succinocarboxamide |
| | | | synthetase |
| 38527_at | down | 0.047648 | non-POU domain containing, octamer-binding |
| 37713_at | up | 0.047659 | aminoacylase 1 |
| 39755_at | up | 0.047681 | X-box binding protein 1 |
| 31816_at | up | 0.047754 | glucosidase, alpha; acid (Pompe disease, glycogen |
| | | | storage disease type II) |
| 31381_at | up | 0.048043 | peptidoglycan recognition protein |
| 32240_at | up | 0.048048 | proteasome (prosome, macropain) 26S subunit, non- |
| | | | ATPase, 5 |
| 40105_at | down | 0.048138 | methylmalonyl Coenzyme A mutase |
| 35311_at | down | 0.048157 | cellular repressor of E1A-stimulated genes |
| 39122_at | up | 0.048174 | glucose phosphate isomerase |
| 37640_at | down | 0.048183 | hypoxanthine phosphoribosyltransferase 1 (Lesch-Nyhar |
| | | | syndrome) |
| 36313_at | down | 0.048202 | ecotropic viral integration site 2A |
| 32052_at | up | 0.048233 | hemoglobin, beta |
| 32928_at | up | 0.048347 | POU domain, class 2, transcription factor 3 |
| 40519_at | down | 0.048417 | protein tyrosine phosphatase, receptor type, C |
| 40910_at | down | 0.048437 | capping protein (actin filament) muscle Z-line, alpha 1 |
| 39139_at | down | 0.048449 | signal peptidase complex (18 kD) |
| 35184_at | down | 0.048653 | hypothetical protein MGC23401 |
| 38319_at | down | 0.048706 | CD3D antigen, delta polypeptide (TiT3 complex) |
| 32716_at | up | 0.048729 | diacylglycerol kinase, alpha 80 kDa |
| 37931_at | up | 0.048751 | centromere protein B, 80 kDa |
| 41435_at | up | 0.048872 | protein tyrosine phosphatase, receptor type, f polypeptid |
| | | | (PTPRF), interacting protein (liprin), alpha 3 |
| 40833_r_at | down | 0.048945 | lamina-associated polypeptide 1B |
| 34292_at | down | 0.048964 | chromosome X open reading frame 12 |
| 38368_at | down | 0.048974 | dUTP pyrophosphatase |
| 33395_at | up | 0.048977 | DKFZP566C0424 protein |
| 36432_at | up | 0.049021 | methylcrotonoyl-Coenzyme A carboxylase 2 (beta) |
| 36920_at | down | 0.049082 | myotubular myopathy 1 |
| 35830_at | up | 0.049138 | exportin 6 |
| 39531_at | up | 0.049147 | microtubule-associated protein 1B |
| 31710_at | up | 0.049156 | |
| 35550_at | down | 0.04917 | phosphate cytidylyltransferase 1, choline, beta isoform |
| 32643_at | up | 0.049176 | glucan (1,4-alpha-), branching enzyme 1 (glycogen |
| | | | branching enzyme, Andersen disease, glycogen storage |
| | | | disease type IV) |
| 1213_at | down | 0.049284 | SFRS protein kinase 2 |
| 38765_at | down | 0.049409 | Dicer1, Dcr-1 homolog (Drosophila) |
| 777_at | down | 0.049529 | GDP dissociation inhibitor 2 |
| 39854_r_at | up | 0.049529 | transport-secretion protein 2.2 |
| 36118_at | down | 0.049576 | nuclear receptor coactivator 1 |
| 39009_at | down | 0.049585 | LSM3 homolog, U6 small nuclear RNA associated (S. cerevisiae) |
| 40106_at | up | 0.049587 | E1B-55 kDa-associated protein 5 |
| 32597_at | down | 0.049613 | retinoblastoma-like 2 (p130) |
| 37423_at | up | 0.049689 | solute carrier family 12 (sodium/potassium/chloride |
| | | | transporters), member 2 |
| 32792_at | down | 0.049898 | GCIP-interacting protein p29 |
| 37900_at | down | 0.049937 | peroxisomal biogenesis factor 11B |
| 218_at | down | 0.050114 | IK cytokine, down-regulator of HLA II |
| 1983_at | up | 0.050135 | cyclin D2 |
| 38199_at | up | 0.050157 | similar to RIKEN cDNA 2610307I21 |
| 35370_at | down | 0.050158 | SPTF-associated factor 65 gamma |
| 36509_at | down | 0.050193 | ribosomal protein L35a |
| 41855_at | down | 0.050238 | histone acetyltransferase 1 |
| 33921_at | up | 0.050257 | glucose-6-phosphatase, transport (glucose-6-phosphate) |
| | | | protein 1 |
| 32338_at | up | 0.050322 | DKFZP564C152 protein |
| 31573_at | down | 0.050336 | ribosomal protein S25 |
| 35686_s_at | up | 0.05038 | mature T-cell proliferation 1 |
| 34508_r_at | up | 0.050407 | kinase phosphatase inhibitor 2 |
| 39264_at | up | 0.050425 | 2′-5′-oligoadenylate synthetase 2, 69/71 kDa |
| 937_at | down | 0.050456 | |
| 39507_at | down | 0.050466 | O-linked N-acetylglucosamine (GlcNAc) transferase |
| | | | (UDP-N-acetylglucosamine:polypeptide-N- |
| | | | acetylglucosaminyl transferase) |
| 36460_at | down | 0.05048 | polymerase (RNA) I polypeptide C, 30 kDa |
| 1449_at | down | 0.050486 | proteasome (prosome, macropain) subunit, alpha type, 4 |
| 32982_at | down | 0.050635 | myelin transcription factor 2 |
| 41400_at | down | 0.050664 | thymidine kinase 1, soluble |
| 41266_at | down | 0.050685 | integrin, alpha 6 |
| 336_at | up | 0.050688 | thromboxane A2 receptor |
| 35083_at | up | 0.050695 | ferritin, light polypeptide |
| 34563_at | up | 0.050709 | kinesin family member 14 |
| 39951_at | up | 0.050726 | plastin 1 (I isoform) |
| 469_at | up | 0.05075 | ephrin-B3 |
| 1589_s_at | down | 0.050778 | interferon (alpha, beta and omega) receptor 2 |
| 32915_at | up | 0.050787 | |
| 32319_at | up | 0.050795 | tumor necrosis factor (ligand) superfamily, member 4 (ta |
| | | | transcriptionally activated glycoprotein 1, 34 kDa) |
| 40516_at | down | 0.050823 | aryl hydrocarbon receptor |
| 40762_g_at | down | 0.050837 | solute carrier family 16 (monocarboxylic acid |
| | | | transporters), member 5 |
| 32872_at | up | 0.050892 | |
| 32598_at | down | 0.050917 | NEL-like 2 (chicken) |
| 40066_at | down | 0.050921 | ubiquitin-activating enzyme E1C (UBA3 homolog, yeast) |
| 37191_at | up | 0.050937 | phytanoyl-CoA hydroxylase interacting protein |
| 36731_g_at | up | 0.050945 | ATP-binding cassette, sub-family C (CFTR/MRP), |
| | | | member 10 |
| 38032_at | up | 0.050977 | synaptic vesicle glycoprotein 2A |
| 39891_at | down | 0.051005 | |
| 40727_at | down | 0.051151 | anaphase-promoting complex subunit 10 |
| 34573_at | up | 0.051166 | ephrin-A3 |
| 37575_at | down | 0.051171 | |
| 41837_at | up | 0.051172 | chromosome 14 open reading frame 132 |
| 39706_at | down | 0.051192 | copine III |
| 39796_at | up | 0.051208 | proteasome (prosome, macropain) activator subunit 3 |
| | | | (PA28 gamma; Ki) |
| 37205_at | up | 0.051225 | F-box and leucine-rich repeat protein 7 |
| 33517_f_at | up | 0.051227 | melanoma antigen, family A, 3 |
| 37089_at | up | 0.051376 | a disintegrin and metalloproteinase domain 3a (cyritestir |
| | | | 1) |
| 41525_at | up | 0.051469 | high-mobility group 20B |
| 37387_r_at | down | 0.05148 | KDEL (Lys-Asp-Glu-Leu) endoplasmic reticulum protein |
| | | | retention receptor 1 |
| 41753_at | up | 0.051513 | actinin, alpha 4 |
| 40561_at | up | 0.05152 | T-cell leukemia, homeobox 2 |
| 33523_at | up | 0.051666 | alkaline phosphatase, intestinal |
| 36041_at | up | 0.051715 | exonuclease 1 |
| 39789_at | up | 0.051771 | sarcolipin |
| 36799_at | up | 0.051797 | frizzled homolog 2 (Drosophila) |
| 41493_at | down | 0.051813 | ATPase, Class VI, type 11A |
| 34820_at | up | 0.051886 | pleiotrophin (heparin binding growth factor 8, neurite |
| | | | growth-promoting factor 1) |
| 36484_at | down | 0.051889 | UDP-N-acetyl-alpha-D-galactosamine:polypeptide N- |
| | | | acetylgalactosaminyltransferase 3 (GalNAc-T3) |
| 40381_at | up | 0.051999 | KIAA0972 protein |
| 38230_at | up | 0.052097 | endothelial PAS domain protein 1 |
| 33961_at | up | 0.052099 | |
| 40371_at | up | 0.052175 | dopamine receptor D2 |
| 36494_at | down | 0.052182 | spondyloepiphyseal dysplasia, late, pseudogene |
| 33198_at | down | 0.052191 | binder of Arl Two |
| 35743_at | up | 0.052209 | cleavage and polyadenylation specific factor 4, 30 kDa |
| 34791_at | down | 0.052211 | t-complex 1 |
| 32811_at | up | 0.05223 | myosin IC |
| 37381_g_at | down | 0.052289 | general transcription factor IIB |
| 379_at | down | 0.052302 | ATP binding protein associated with cell differentiation |
| 39519_at | down | 0.052312 | KIAA0692 protein |
| 34167_s_at | up | 0.052385 | solute carrier family 6 (neurotransmitter transporter, L- |
| | | | proline), member 7 |
| 31367_at | up | 0.052421 | KIAA0998 protein |
| 36971_at | down | 0.052422 | likely ortholog of mouse Rw1 |
| 37021_at | down | 0.052436 | cathepsin H |
| 37620_at | down | 0.052482 | TAF12 RNA polymerase II, TATA box binding protein |
| | | | (TBP)-associated factor, 20 kDa |
| 765_s_at | up | 0.052582 | lectin, galactoside-binding, soluble, 4 (galectin 4) |
| 39003_at | down | 0.052675 | pituitary tumor-transforming 1 interacting protein |
| 31962_at | up | 0.052686 | ribosomal protein L37a |
| 551_at | down | 0.052715 | E1A binding protein p300 |
| 40491_at | up | 0.052889 | retinoblastoma binding protein 1-like 1 |
| 39692_at | down | 0.052957 | hypothetical protein DKFZp586F2423 |
| 32533_s_at | down | 0.053017 | vesicle-associated membrane protein 5 (myobrevin) |
| 39064_at | down | 0.053053 | 5,10-methenyltetrahydrofolate synthetase (5- |
| | | | formyltetrahydrofolate cyclo-ligase) |
| 35957_at | up | 0.053064 | stannin |
| 36440_at | up | 0.053117 | pre T-cell antigen receptor alpha |
| 39707_at | down | 0.053118 | myotubularin related protein 4 |
| 32335_r_at | down | 0.053145 | ubiquitin C |
| 867_s_at | up | 0.053202 | thrombospondin 1 |
| 33545_at | up | 0.053202 | sodium channel, voltage-gated, type IV, alpha |
| 716_at | up | 0.053248 | gamma-glutamyltransferase-like activity 1 |
| 37490_at | up | 0.053251 | solute carrier family 4, anion exchanger, member 3 |
| 34963_at | up | 0.053255 | collagen, type XIV, alpha 1 (undulin) |
| 1878_g_at | down | 0.053278 | excision repair cross-complementing rodent repair |
| | | | deficiency, complementation group 1 (includes |
| | | | overlapping antisense sequence) |
| 41218_at | down | 0.053298 | KIAA0570 gene product |
| 37306_at | down | 0.053313 | cytoplasmic FMR1 interacting protein 1 |
| 32654_g_at | down | 0.053395 | bromodomain containing 8 |
| 1180_g_at | down | 0.053406 | |
| 738_at | down | 0.053443 | 5′-nucleotidase, cytosolic II |
| 32843_s_at | up | 0.05356 | casein kinase 2, beta polypeptide |
| 39818_at | down | 0.053596 | putative c-Myc-responsive |
| 1984_s_at | up | 0.05365 | Rho GDP dissociation inhibitor (GDI) beta |
| 34705_at | up | 0.053743 | similar to yeast BET3 (S. cerevisiae) |
| 35147_at | up | 0.053798 | MCF.2 cell line derived transforming sequence-like |
| 36237_at | up | 0.053829 | solute carrier family 22 (organic anion transporter), |
| | | | member 6 |
| 39070_at | down | 0.053862 | fascin homolog 1, actin-bundling protein |
| | | | (Strongylocentrotus purpuratus) |
| 39061_at | up | 0.053904 | bone marrow stromal cell antigen 2 |
| 39457_r_at | up | 0.053929 | sorting nexin 4 |
| 40466_at | up | 0.053949 | nuclear transcription factor Y, gamma |
| 38485_at | down | 0.054018 | NADH dehydrogenase (ubiquinone) 1, subcomplex |
| | | | unknown, 1, 6 kDa |
| 37307_at | up | 0.054022 | guanine nucleotide binding protein (G protein), alpha |
| | | | inhibiting activity polypeptide 2 |
| 633_s_at | up | 0.054102 | transcription factor Dp-2 (E2F dimerization partner 2) |
| 883_s_at | up | 0.054292 | pim-1 oncogene |
| 815_at | up | 0.054384 | docking protein 1, 62 kDa (downstream of tyrosine kinase |
| | | | 1) |
| 39549_at | down | 0.054465 | neuronal PAS domain protein 2 |
| 35748_at | down | 0.054501 | eukaryotic translation elongation factor 1 beta 2 |
| 32488_at | up | 0.054503 | collagen, type III, alpha 1 (Ehlers-Danlos syndrome type |
| | | | IV, autosomal dominant) |
| 1125_s_at | up | 0.054566 | CD44 antigen (homing function and Indian blood group |
| | | | system) |
| 38931_at | down | 0.054596 | zinc finger protein, X-linked |
| 35047_at | up | 0.054596 | regulatory factor X, 2 (influences HLA class II expression |
| 36807_at | up | 0.054671 | TED protein |
| 32745_at | down | 0.054805 | mitochondrial ribosomal protein L40 |
| 33079_at | down | 0.054837 | syntaxin 6 |
| 38195_at | up | 0.054849 | KIAA0783 gene product |
| 40348_s_at | up | 0.054929 | acidic (leucine-rich) nuclear phosphoprotein 32 family, |
| | | | member E |
| 35675_at | up | 0.055021 | vinexin beta (SH3-containing adaptor molecule-1) |
| 41655_at | up | 0.055041 | midline 2 |
| 35502_at | up | 0.055045 | anti-Mullerian hormone receptor, type II |
| 39590_at | up | 0.055058 | amyloid beta (A4) precursor protein-binding, family A, |
| | | | member 2 (X11-like) |
| 39921_at | down | 0.05506 | cytochrome c oxidase subunit Vb |
| 1904_at | down | 0.055112 | c-myc binding protein |
| 40326_at | up | 0.055127 | cerebellin 1 precursor |
| 39364_s_at | up | 0.055204 | protein phosphatase 1, regulatory (inhibitor) subunit 3C |
| 32860_g_at | down | 0.055251 | signal transducer and activator of transcription 1, 91 kDa |
| 39899_at | up | 0.055292 | TSLC1-like 2 |
| 38850_at | up | 0.055469 | |
| 37725_at | down | 0.055509 | protein phosphatase 1, catalytic subunit, gamma isoform |
| 32106_at | up | 0.055553 | serine (or cysteine) proteinase inhibitor, clade A (alpha-1 |
| | | | antiproteinase, antitrypsin), member 4 |
| 409_at | down | 0.055578 | tyrosine 3-monooxygenase/tryptophan 5-monooxygenas |
| | | | activation protein, theta polypeptide |
| 33715_r_at | down | 0.055635 | general transcription factor IIH, polypeptide 2, 44 kDa |
| 32587_at | down | 0.055636 | zinc finger protein 36, C3H type-like 2 |
| 38172_at | down | 0.055664 | carbonyl reductase 3 |
| 37436_at | up | 0.055701 | mitochondrial capsule selenoprotein |
| 41070_r_at | down | 0.055707 | transcription termination factor, mitochondrial |
| 39841_at | down | 0.055715 | solute carrier family 16 (monocarboxylic acid |
| | | | transporters), member 6 |
| 35354_at | up | 0.055782 | synaptogyrin 1 |
| 38526_at | up | 0.055797 | phosphodiesterase 4D, cAMP-specific |
| | | | (phosphodiesterase E3 dunce homolog, Drosophila) |
| 1412_g_at | up | 0.055798 | cytochrome P450, family 11, subfamily B, polypeptide 1 |
| 37840_at | up | 0.055809 | cyclic nucleotide gated channel alpha 1 |
| 36606_at | up | 0.05594 | carboxypeptidase E |
| 36163_at | down | 0.055944 | dihydrolipoamide dehydrogenase (E3 component of |
| | | | pyruvate dehydrogenase complex, 2-oxo-glutarate |
| | | | complex, branched chain keto acid dehydrogenase |
| | | | complex) |
| 34843_at | down | 0.056006 | KIAA0222 gene product |
| 41418_at | up | 0.056134 | latrophilin 1 |
| 38416_at | down | 0.056171 | chaperonin containing TCP1, subunit 6A (zeta 1) |
| 36998_s_at | down | 0.056286 | spinocerebellar ataxia 2 (olivopontocerebellar ataxia 2, |
| | | | autosomal dominant, ataxin 2) |
| 34587_at | up | 0.0563 | eosinophil peroxidase |
| 38627_at | up | 0.056394 | hepatic leukemia factor |
| 1076_at | down | 0.056444 | interleukin 1, alpha |
| 1337_s_at | up | 0.056459 | retinoic acid receptor, alpha |
| AFFX-BioDn- | up | 0.056496 | |
| 5_at | | | |
| 37694_at | down | 0.056501 | PHD finger protein 3 |
| 39337_at | down | 0.056519 | H2A histone family, member Z |
| 37077_at | up | 0.056579 | pyruvate kinase, liver and RBC |
| 38371_at | down | 0.056601 | proteasome (prosome, macropain) subunit, alpha type, 1 |
| 33205_at | up | 0.056644 | suppressor of Ty 3 homolog (S. cerevisiae) |
| 33281_at | up | 0.056699 | inhibitor of kappa light polypeptide gene enhancer in B- |
| | | | cells, kinase epsilon |
| 32303_at | up | 0.05673 | ets variant gene 3 |
| 32546_at | down | 0.056793 | fumarate hydratase |
| 734_at | up | 0.0568 | |
| 35022_at | up | 0.056829 | SRY (sex determining region Y)-box 5 |
| 32224_at | down | 0.056859 | KIAA0769 gene product |
| 37001_at | down | 0.056907 | calpain 2, (m/II) large subunit |
| 39991_at | up | 0.056999 | corneodesmosin |
| 41496_at | up | 0.057011 | HCF-binding transcription factor Zhangfei |
| 39894_f_at | down | 0.057027 | bromodomain containing 1 |
| 35824_at | down | 0.057055 | zinc finger protein 238 |
| 37499_at | up | 0.057095 | KIAA0408 gene product |
| 36446_s_at | down | 0.057135 | hepatoma-derived growth factor (high-mobility group |
| | | | protein 1-like) |
| 37994_at | down | 0.057304 | fragile X mental retardation 1 |
| 33670_at | up | 0.057341 | |
| 41622_r_at | down | 0.05735 | zinc finger protein 266 |
| 41294_at | up | 0.057454 | keratin 7 |
| 39747_at | down | 0.057465 | polymerase (RNA) II (DNA directed) polypeptide G |
| 2068_s_at | down | 0.057538 | E2F transcription factor 2 |
| 37751_at | down | 0.057572 | KIAA0255 gene product |
| 33039_at | down | 0.057696 | T-cell receptor interacting molecule |
| 1621_at | down | 0.057779 | CDC5 cell division cycle 5-like (S. pombe) |
| 182_at | up | 0.057817 | inositol 1,4,5-triphosphate receptor, type 3 |
| 31587_at | up | 0.057841 | solute carrier family 14 (urea transporter), member 2 |
| 34402_at | down | 0.057844 | unr-interacting protein |
| 36620_at | down | 0.057858 | superoxide dismutase 1, soluble (amyotrophic lateral |
| | | | sclerosis 1 (adult)) |
| 39439_at | up | 0.057864 | |
| 40738_at | up | 0.057874 | CD2 antigen (p50), sheep red blood cell receptor |
| 36165_at | down | 0.057904 | cytochrome c oxidase subunit VIc |
| 35267_g_at | down | 0.057925 | bladder cancer associated protein |
| 41858_at | up | 0.057932 | FGF receptor activating protein 1 |
| 32013_at | down | 0.057996 | zinc finger protein 409 |
| 35958_at | up | 0.058051 | ADP-ribosylation factor-like 7 |
| 31851_at | down | 0.058101 | ret finger protein 2 |
| 33069_f_at | up | 0.058119 | UDP glycosyltransferase 2 family, polypeptide B15 |
| 32392_s_at | up | 0.058127 | UDP glycosyltransferase 1 family, polypeptide A4 |
| 39150_at | down | 0.058159 | ring finger protein 11 |
| 35831_at | up | 0.058337 | ATPase, Class II, type 9A |
| 35434_at | up | 0.058387 | MADS box transcription enhancer factor 2, polypeptide D |
| | | | (myocyte enhancer factor 2D) |
| 39412_at | up | 0.058422 | tripartite motif-containing 26 |
| 39294_at | up | 0.058457 | nuclear receptor subfamily 2, group F, member 1 |
| 41638_at | down | 0.058458 | KIAA0073 protein |
| 34404_at | down | 0.05849 | COP9 constitutive photomorphogenic homolog subunit 7 |
| | | | (Arabidopsis) |
| 32733_at | down | 0.05852 | mitochondrial ribosomal protein S14 |
| 33706_at | up | 0.058622 | squamous cell carcinoma antigen recognised by T cells |
| 40578_s_at | up | 0.058655 | tropomodulin 1 |
| 37165_f_at | up | 0.058858 | Rhesus blood group, CcEe antigens |
| 35172_at | down | 0.058889 | tyrosylprotein sulfotransferase 2 |
| AFFX-DapX-3_at | up | 0.05895 | |
| 41705_at | up | 0.059029 | radical fringe homolog (Drosophila) |
| 1735_g_at | up | 0.059035 | transforming growth factor, beta 3 |
| 36687_at | down | 0.059039 | cytochrome c oxidase subunit VIIb |
| 33403_at | down | 0.059201 | DKFZP547E1010 protein |
| 553_g_at | down | 0.059352 | Rho GTPase activating protein 1 |
| 31869_at | down | 0.059449 | SWAP-70 protein |
| 715_s_at | up | 0.059541 | gamma-glutamyltransferase 1 |
| 40688_at | up | 0.059541 | linker for activation of T cells |
| 33514_at | down | 0.059565 | calcium/calmodulin-dependent protein kinase IV |
| 38019_at | up | 0.059588 | casein kinase 1, epsilon |
| 36570_at | up | 0.059666 | calbindin 1, 28 kDa |
| 1680_at | up | 0.059714 | growth factor receptor-bound protein 7 |
| 2015_s_at | down | 0.059737 | PMS2 postmeiotic segregation increased 2 (S. cerevisiae |
| 331_at | up | 0.059749 | |
| 32117_at | up | 0.059788 | apoptosis antagonizing transcription factor |
| 32606_at | down | 0.059843 | brain abundant, membrane attached signal protein 1 |
| 38256_s_at | down | 0.059857 | DKFZP564O092 protein |
| 1657_at | up | 0.059889 | protein tyrosine phosphatase, receptor type, R |
| 38414_at | down | 0.060003 | CDC20 cell division cycle 20 homolog (S. cerevisiae) |
| 40710_at | up | 0.060006 | calmegin |
| 36506_at | down | 0.060098 | A kinase (PRKA) anchor protein (yotiao) 9 |
| 36517_at | down | 0.060144 | U2(RNU2) small nuclear RNA auxillary factor 1 |
| 38314_at | up | 0.060166 | capicua homolog (Drosophila) |
| 38031_at | down | 0.06017 | KIAA0111 gene product |
| 1915_s_at | up | 0.060242 | v-fos FBJ murine osteosarcoma viral oncogene homolog |
| 1250_at | down | 0.060379 | protein kinase, DNA-activated, catalytic polypeptide |
| 31309_r_at | up | 0.060453 | |
| 31318_at | up | 0.060492 | |
| 38183_at | down | 0.060518 | forkhead box F1 |
| 35953_at | up | 0.060518 | carboxypeptidase N, polypeptide 1, 50 kD |
| 40482_s_at | up | 0.060522 | transcriptional activator of the c-fos promoter |
| 34073_s_at | up | 0.060534 | guanine nucleotide binding protein (G protein), alpha |
| | | | transducing activity polypeptide 1 |
| 499_at | up | 0.060624 | MAD1 mitotic arrest deficient-like 1 (yeast) |
| 34096_at | up | 0.060635 | KIAA0912 protein |
| 40453_s_at | down | 0.060669 | splicing factor, arginine/serine-rich 5 |
| 35554_f_at | up | 0.060751 | Zic family member 2 (odd-paired homolog, Drosophila) |
| 38281_at | down | 0.060874 | caspase 7, apoptosis-related cysteine protease |
| 41807_at | down | 0.060901 | sin3-associated polypeptide, 18 kDa |
| 35374_at | up | 0.060921 | rootletin |
| 41506_at | down | 0.060926 | mitogen-activated protein kinase-activated protein kinase 5 |
| 32194_at | down | 0.06103 | CCAAT-box-binding transcription factor |
| 386_g_at | up | 0.0611 | |
| 36572_r_at | down | 0.06115 | ADP-ribosylation factor-like 6 interacting protein |
| 32965_f_at | up | 0.061162 | heat shock 70 kDa protein 1B |
| 34814_at | down | 0.06127 | SUMO-1 activating enzyme subunit 2 |
| 39399_at | up | 0.061337 | tubulin-specific chaperone d |
| 38872_at | up | 0.061352 | zinc finger protein 230 |
| 31539_r_at | up | 0.061358 | |
| 38483_at | up | 0.061407 | dullard homolog (Xenopus laevis) |
| 838_s_at | up | 0.061516 | ubiquitin-conjugating enzyme E2I (UBC9 homolog, yeast |
| 41833_at | up | 0.061566 | jumping translocation breakpoint |
| 33004_g_at | up | 0.06165 | NCK adaptor protein 2 |
| 39151_at | up | 0.061669 | astrotactin |
| 33952_at | up | 0.06176 | zinc finger protein 306 |
| 35575_f_at | up | 0.061928 | zinc finger protein 253 |
| 40567_at | down | 0.061963 | tubulin, alpha 3 |
| 905_at | down | 0.061983 | guanylate kinase 1 |
| 39133_at | down | 0.061983 | GCN5 general control of amino-acid synthesis 5-like 1 |
| | | | (yeast) |
| 41669_at | down | 0.062016 | KIAA0191 protein |
| 39048_at | up | 0.06202 | Notch homolog 4 (Drosophila) |
| 40134_at | down | 0.062031 | ATP synthase, H+ transporting, mitochondrial F0 comple |
| | | | subunit f, isoform 2 |
| 610_at | down | 0.062036 | adrenergic, beta-2-, receptor, surface |
| 445_at | up | 0.062045 | NK3 transcription factor related, locus 1 (Drosophila) |
| 36353_at | up | 0.062046 | hairy and enhancer of split (Drosophila) homolog 2 |
| 40180_at | up | 0.062046 | insulin receptor substrate 2 |
| 32963_s_at | down | 0.062118 | Rag D protein |
| 1458_at | up | 0.062196 | protein tyrosine phosphatase, non-receptor type 3 |
| 40667_at | up | 0.062229 | CD6 antigen |
| 34821_at | down | 0.062272 | chromosome 6 open reading frame 80 |
| 38114_at | down | 0.062321 | RAD21 homolog (S. pombe) |
| 40409_at | down | 0.06235 | aldehyde dehydrogenase 3 family, member A2 |
| 36900_at | up | 0.062386 | stromal interaction molecule 1 |
| 40604_at | down | 0.062441 | dual-specificity tyrosine-(Y)-phosphorylation regulated |
| | | | kinase 2 |
| 37608_g_at | up | 0.062657 | ketohexokinase (fructokinase) |
| 38607_at | up | 0.062688 | transmembrane 4 superfamily member 5 |
| 31496_g_at | up | 0.062707 | chemokine (C motif) ligand 2 |
| 39005_s_at | down | 0.062761 | zinc finger protein 294 |
| 41038_at | down | 0.062773 | neutrophil cytosolic factor 2 (65 kDa, chronic |
| | | | granulomatous disease, autosomal 2) |
| 731_f_at | up | 0.062914 | |
| 776_at | down | 0.062923 | phosphatidylinositol glycan, class F |
| 2050_s_at | down | 0.06298 | ras-related C3 botulinum toxin substrate 1 (rho family, |
| | | | small GTP binding protein Rac1) |
| 37892_at | up | 0.062998 | collagen, type XI, alpha 1 |
| 34237_at | down | 0.063043 | HBS1-like (S. cerevisiae) |
| AFFX- | up | 0.063076 | glyceraldehyde-3-phosphate dehydrogenase |
| HUMGAPDH/M3 | | | |
| 3197_M_at | | | |
| 41023_at | up | 0.063109 | complement component 8, alpha polypeptide |
| 38408_at | up | 0.063134 | transmembrane 4 superfamily member 2 |
| 35552_at | up | 0.063161 | phosphate cytidylyltransferase 1, choline, beta isoform |
| 36058_at | up | 0.063188 | ASC-1 complex subunit P100 |
| 32592_at | up | 0.063295 | KIAA0323 protein |
| 32559_s_at | down | 0.063304 | LSM4 homolog, U6 small nuclear RNA associated (S. cerevisiae) |
| 31675_s_at | down | 0.063337 | phosphatase and tensin homolog (mutated in multiple |
| | | | advanced cancers 1), pseudogene 1 |
| 34003_at | down | 0.06334 | triosephosphate isomerase 1 |
| 34412_s_at | up | 0.06344 | peanut-like 1 (Drosophila) |
| 37853_at | up | 0.063455 | urocortin |
| 40125_at | down | 0.063554 | calnexin |
| 36148_at | up | 0.063582 | amyloid beta (A4) precursor-like protein 1 |
| 35078_at | up | 0.063583 | intercellular adhesion molecule 4, Landsteiner-Wiener |
| | | | blood group |
| 32967_at | up | 0.06359 | regulator of Fas-induced apoptosis |
| 35939_s_at | up | 0.063597 | POU domain, class 4, transcription factor 1 |
| 40777_at | down | 0.063706 | catenin (cadherin-associated protein), beta 1, 88 kDa |
| 33448_at | up | 0.063706 | serine protease inhibitor, Kunitz type 1 |
| 36845_at | down | 0.063755 | nuclear matrix protein NXP2 |
| 33678_i_at | up | 0.063774 | tubulin, beta, 2 |
| 1956_s_at | up | 0.063778 | |
| 34993_at | up | 0.063791 | sarcoglycan, delta (35 kDa dystrophin-associated |
| | | | glycoprotein) |
| 39017_at | down | 0.063812 | LSM1 homolog, U6 small nuclear RNA associated (S. cerevisiae) |
| 199_s_at | down | 0.063818 | protein kinase C-like 2 |
| 37779_at | up | 0.063874 | acid sphingomyelinase-like phosphodiesterase |
| 522_s_at | up | 0.063911 | folate receptor 3 (gamma) |
| 33822_at | up | 0.063948 | nuclear mitotic apparatus protein 1 |
| 34019_at | up | 0.063949 | cholinergic receptor, nicotinic, alpha polypeptide 3 |
| 1453_at | down | 0.064036 | MAD, mothers against decapentaplegic homolog 2 |
| | | | (Drosophila) |
| 31733_at | up | 0.064146 | purinergic receptor P2X, ligand-gated ion channel, 3 |
| 38306_at | down | 0.06416 | brefeldin A-inhibited guanine nucleotide-exchange protein 1 |
| 36276_at | up | 0.06418 | contactin 2 (axonal) |
| 851_s_at | up | 0.064186 | insulin receptor substrate 1 |
| 556_s_at | up | 0.064191 | glutathione S-transferase M4 |
| 35661_g_at | up | 0.064238 | S-antigen; retina and pineal gland (arrestin) |
| 39339_at | up | 0.064293 | KIAA0792 gene product |
| 36916_at | up | 0.064357 | sialyltransferase 4C (beta-galactoside alpha-2,3- |
| | | | sialyltransferase) |
| 31559_at | up | 0.064387 | solute carrier family 13 (sodium-dependent dicarboxylate |
| | | | transporter), member 2 |
| 35610_at | up | 0.06442 | matrilin 1, cartilage matrix protein |
| 41145_at | down | 0.064477 | family with sequence similarity 13, member A1 |
| 41681_at | down | 0.064546 | ATP-binding cassette, sub-family B (MDR/TAP), member 7 |
| 32954_at | up | 0.064582 | DKFZP434D193 protein |
| 32571_at | up | 0.064689 | methionine adenosyltransferase II, alpha |
| 33684_at | up | 0.064701 | wingless-type MMTV integration site family, member 2B |
| 38164_at | down | 0.064739 | retinitis pigmentosa GTPase regulator |
| 32681_at | up | 0.064752 | solute carrier family 9 (sodium/hydrogen exchanger), |
| | | | isoform 1 (antiporter, Na+/H+, amiloride sensitive) |
| 40623_at | down | 0.064767 | ubiquitin protein ligase |
| 36763_at | up | 0.064769 | wingless-type MMTV integration site family, member 7A |
| 40838_at | down | 0.064777 | zinc finger protein 292 |
| 40419_at | up | 0.064956 | stomatin |
| 39195_s_at | up | 0.064983 | leucine-rich repeats and immunoglobulin-like domains 1 |
| 40506_s_at | down | 0.065005 | poly(A) binding protein, cytoplasmic 4 (inducible form) |
| 38186_g_at | up | 0.065027 | paired box gene 2 |
| 32697_at | down | 0.065062 | inositol(myo)-1(or 4)-monophosphatase 1 |
| 35813_at | up | 0.065103 | transportin-SR |
| 33895_at | down | 0.065122 | likely ortholog of mouse Sh3 domain YSC-like 1 |
| 40406_at | up | 0.065199 | macrophage stimulating, pseudogene 9 |
| 41162_at | up | 0.065234 | protein phosphatase 1G (formerly 2C), magnesium- |
| | | | dependent, gamma isoform |
| 35368_at | down | 0.06526 | zinc finger protein 207 |
| 35019_at | up | 0.065337 | zinc finger protein 254 |
| 39460_g_at | up | 0.065365 | ribosomal protein S13 |
| 39647_s_at | up | 0.065434 | calcium channel, voltage-dependent, beta 2 subunit |
| 31801_at | up | 0.065498 | |
| 37081_at | up | 0.065507 | dynein, axonemal, heavy polypeptide 7 |
| 31765_at | up | 0.065614 | hypothetical protein FLJ20220 |
| 38668_at | down | 0.065811 | KIAA0553 protein |
| 32050_r_at | down | 0.065884 | |
| 35116_at | down | 0.065906 | KIAA0874 protein |
| 36808_at | up | 0.065972 | protein tyrosine phosphatase, non-receptor type 22 |
| | | | (lymphoid) |
| 40854_at | down | 0.066118 | ubiquinol-cytochrome c reductase core protein II |
| 35673_at | up | 0.066212 | Rho guanine nucleotide exchange factor (GEF) 5 |
| 40269_at | down | 0.066219 | PRP18 pre-mRNA processing factor 18 homolog (yeast) |
| 1403_s_at | up | 0.066241 | chemokine (C—C motif) ligand 5 |
| 31436_s_at | up | 0.066263 | estrogen receptor 2 (ER beta) |
| 308_f_at | up | 0.066282 | growth hormone 2 |
| 41476_at | up | 0.066339 | guanine nucleotide binding protein (G protein), alpha 11 |
| | | | (Gq class) |
| 1195_s_at | down | 0.066371 | integrin cytoplasmic domain-associated protein 1 |
| 39096_at | down | 0.066452 | SON DNA binding protein |
| 36945_at | up | 0.066499 | chromosome 12 open reading frame 8 |
| 39036_g_at | down | 0.066538 | progestin induced protein |
| 41029_at | up | 0.066712 | U1-snRNP binding protein homolog |
| 35721_at | down | 0.0668 | hydroxy-delta-5-steroid dehydrogenase, 3 beta- and |
| | | | steroid delta-isomerase 1 |
| 38949_at | up | 0.066925 | protein kinase C, theta |
| 38945_at | up | 0.067053 | metal-regulatory transcription factor 1 |
| 34626_at | up | 0.06707 | hypermethylated in cancer 1 |
| 744_at | down | 0.067118 | DEAD/H (Asp-Glu-Ala-Asp/His) box polypeptide 8 (RNA |
| | | | helicase) |
| 34596_at | up | 0.067122 | casein kappa |
| 36935_at | down | 0.067265 | RAS p21 protein activator (GTPase activating protein) 1 |
| 1952_s_at | down | 0.067351 | MAD, mothers against decapentaplegic homolog 5 |
| | | | (Drosophila) |
| 41278_at | down | 0.067459 | BAF53 |
| 643_at | up | 0.067476 | nuclear receptor subfamily 0, group B, member 2 |
| 1399_at | down | 0.067506 | transcription elongation factor B (SIII), polypeptide 1 |
| | | | (15 kDa, elongin C) |
| 32545_r_at | up | 0.067532 | Ras suppressor protein 1 |
| 34649_at | down | 0.067605 | decorin |
| 37966_at | up | 0.06761 | parvin, beta |
| 40832_s_at | down | 0.067654 | lamina-associated polypeptide 1B |
| 38672_at | up | 0.067756 | protein phosphatase 1, regulatory subunit 10 |
| 35576_f_at | up | 0.067819 | histone 1, H2bl |
| 33438_at | up | 0.067968 | WW domain binding protein 2 |
| 35481_at | up | 0.068025 | myosin heavy chain Myr 8 |
| 32217_at | down | 0.068042 | chromosome 12 open reading frame 22 |
| 228_at | down | 0.068127 | v-ral simian leukemia viral oncogene homolog B (ras |
| | | | related; GTP binding protein) |
| 464_s_at | down | 0.06822 | interferon-induced protein 35 |
| 39552_at | down | 0.068223 | phosphatase and tensin homolog (mutated in multiple |
| | | | advanced cancers 1) |
| 41110_at | down | 0.068425 | cullin 5 |
| 37023_at | down | 0.068458 | lymphocyte cytosolic protein 1 (L-plastin) |
| 36948_at | down | 0.068499 | CREBBP/EP300 inhibitory protein 1 |
| 40963_at | up | 0.068522 | ATP-binding cassette, sub-family A (ABC1), member 4 |
| 38633_at | down | 0.068526 | metastasis associated 1 |
| 31339_at | up | 0.068694 | protease inhibitor 15 |
| AFFX-CreX-5_st | up | 0.068736 | |
| 41051_at | down | 0.068762 | translin-associated factor X |
| 33842_at | up | 0.06881 | hypothetical protein FLJ11560 |
| 36597_at | down | 0.068981 | nucleolar and coiled-body phosphoprotein 1 |
| 31523_f_at | up | 0.068994 | histone 1, H2be |
| 41460_at | down | 0.069064 | RNA binding motif protein 14 |
| 36958_at | up | 0.069077 | zyxin |
| 36483_at | down | 0.069179 | UDP-N-acetyl-alpha-D-galactosamine:polypeptide N- |
| | | | acetylgalactosaminyltransferase 3 (GalNAc-T3) |
| 32274_r_at | up | 0.069303 | |
| 36616_at | down | 0.069311 | DAZ associated protein 2 |
| 39721_at | up | 0.069372 | ephrin-B1 |
| 950_at | down | 0.06943 | translocation protein 1 |
| 33354_at | down | 0.069464 | E3 ubiquitin ligase SMURF2 |
| 40571_at | down | 0.0695 | myosin VA (heavy polypeptide 12, myoxin) |
| 41343_at | down | 0.06951 | CDP-diacylglycerol synthase (phosphatidate |
| | | | cytidylyltransferase) 2 |
| 38159_at | up | 0.069545 | |
| 33989_f_at | up | 0.069619 | testis enhanced gene transcript (BAX inhibitor 1) |
| 39646_at | up | 0.06966 | calcium channel, voltage-dependent, beta 2 subunit |
| 39141_at | up | 0.069763 | ATP-binding cassette, sub-family F (GCN20), member 1 |
| 966_at | up | 0.069776 | RAD54-like (S. cerevisiae) |
| 40660_at | up | 0.069809 | nuclear receptor subfamily 4, group A, member 3 |
| 35037_at | up | 0.069895 | solute carrier family 28 (sodium-coupled nucleoside |
| | | | transporter), member 1 |
| 36978_at | down | 0.069901 | proteasome (prosome, macropain) activator subunit 4 |
| 1446_at | down | 0.069924 | proteasome (prosome, macropain) subunit, alpha type, 2 |
| 41296_s_at | down | 0.069939 | START domain containing 7 |
| 36104_at | down | 0.06998 | ubiquinol-cytochrome c reductase hinge protein |
| 238_at | down | 0.07003 | ribosomal protein S6 kinase, 70 kDa, polypeptide 1 |
| 36667_at | up | 0.070033 | phosphorylase, glycogen; brain |
| 39992_at | up | 0.070076 | solute carrier family 22 (organic cation transporter), |
| | | | member 1-like |
| 35864_at | up | 0.070114 | acrosin |
| 39295_s_at | up | 0.070249 | Arg/Abl-interacting protein ArgBP2 |
| 38522_s_at | down | 0.070415 | CD22 antigen |
| 40916_at | down | 0.070458 | hypothetical protein FLJ10097 |
| 37230_at | down | 0.070461 | KIAA0469 gene product |
| 39717_g_at | down | 0.07051 | mitochondrial ribosomal protein L33 |
| 35793_at | down | 0.07062 | Ras-GTPase activating protein SH3 domain-binding |
| | | | protein 2 |
| 39633_at | up | 0.070855 | S100 calcium binding protein A3 |
| 35923_at | up | 0.070865 | cholecystokinin B receptor |
| 32615_at | down | 0.070887 | aspartyl-tRNA synthetase |
| 40761_at | down | 0.071214 | solute carrier family 16 (monocarboxylic acid |
| | | | transporters), member 5 |
| 32108_at | up | 0.071219 | sepiapterin reductase (7,8-dihydrobiopterin:NADP+ |
| | | | oxidoreductase) |
| 38392_at | down | 0.071227 | actin related protein 2/3 complex, subunit 5, 16 kDa |
| 35275_at | up | 0.071315 | adaptor-related protein complex 1, gamma 1 subunit |
| AFFX- | up | 0.07143 | |
| hum_alu_at | | | |
| 35527_at | up | 0.071458 | calcium channel, voltage-dependent, alpha 2/delta subur 1 |
| 34752_at | down | 0.071628 | NIMA (never in mitosis gene a)-related kinase 7 |
| 35634_at | down | 0.071647 | mitogen-activated protein kinase kinase kinase 7 |
| | | | interacting protein 1 |
| 36452_at | up | 0.071789 | synaptopodin |
| 35975_at | down | 0.071826 | myeloid/lymphoid or mixed-lineage leukemia (trithorax |
| | | | homolog, Drosophila); translocated to, 3 |
| 33707_at | up | 0.071894 | phospholipase A2, group IVC (cytosolic, calcium- |
| | | | independent) |
| 34274_at | down | 0.071954 | RNA binding motif protein 16 |
| 40282_s_at | up | 0.071967 | D component of complement (adipsin) |
| 40929_at | down | 0.072007 | SOCS box-containing WD protein SWiP-1 |
| 37024_at | down | 0.072145 | lipopolysaccharide-induced TNF factor |
| 1005_at | up | 0.072186 | dual specificity phosphatase 1 |
| 38968_at | up | 0.072257 | SH3-domain binding protein 5 (BTK-associated) |
| 34013_f_at | up | 0.072278 | POU domain, class 1, transcription factor 1 (Pit1, growth |
| | | | hormone factor 1) |
| 33867_s_at | down | 0.072333 | RNA binding motif, single stranded interacting protein 1 |
| 38096_f_at | down | 0.072339 | major histocompatibility complex, class II, DP beta 1 |
| 37657_at | up | 0.072576 | paralemmin |
| 33388_at | up | 0.072624 | testis expressed gene 261 |
| 39561_at | up | 0.072667 | chromobox homolog 6 |
| 34578_at | up | 0.072676 | sarcoglycan, gamma (35 kDa dystrophin-associated |
| | | | glycoprotein) |
| 40458_at | down | 0.072706 | signal transducer and activator of transcription 5A |
| 934_at | up | 0.072711 | glycosylphosphatidylinositol specific phospholipase D1 |
| 36875_at | down | 0.072713 | inhibitor of Bruton's tyrsoine kinase |
| 35526_at | up | 0.07279 | complement component 9 |
| 38359_at | up | 0.072822 | RAS guanyl releasing protein 2 (calcium and DAG- |
| | | | regulated) |
| 41156_g_at | down | 0.072839 | catenin (cadherin-associated protein), alpha 1, 102 kDa |
| 37000_at | down | 0.072839 | DKFZP564B167 protein |
| 450_g_at | down | 0.072846 | cell growth regulatory with ring finger domain |
| 38892_at | down | 0.072861 | KIAA0240 protein |
| 33224_at | up | 0.072965 | cysteine and histidine rich 1 |
| 33646_g_at | up | 0.073003 | GM2 ganglioside activator protein |
| 31667_r_at | up | 0.073068 | nuclear receptor subfamily 2, group E, member 3 |
| 32853_at | down | 0.073154 | translocase of outer mitochondrial membrane 70 homolo |
| | | | A (yeast) |
| 34193_at | up | 0.073157 | cell adhesion molecule with homology to L1CAM (close |
| | | | homolog of L1) |
| 36017_at | up | 0.073176 | chromosome 13 open reading frame 1 |
| 36110_at | down | 0.073203 | RAB5A, member RAS oncogene family |
| 37861_at | up | 0.073269 | CD1E antigen, e polypeptide |
| 41826_at | up | 0.073366 | KIAA1467 protein |
| 31682_s_at | up | 0.073398 | chondroitin sulfate proteoglycan 2 (versican) |
| 34389_at | up | 0.073463 | collagen, type XIV, alpha 1 (undulin) |
| 33627_at | up | 0.073511 | phosphoinositide-3-kinase, catalytic, delta polypeptide |
| 37238_s_at | up | 0.07355 | membrane-associated tyrosine- and threonine-specific |
| | | | cdc2-inhibitory kinase |
| 39167_r_at | down | 0.073633 | serine (or cysteine) proteinase inhibitor, clade H (heat |
| | | | shock protein 47), member 1, (collagen binding protein 1 |
| 38735_at | up | 0.073757 | KIAA0513 gene product |
| 38477_at | down | 0.073758 | diptheria toxin resistance protein required for diphthamid |
| | | | biosynthesis-like 1 (S. cerevisiae) |
| 36399_at | up | 0.073791 | pre-mRNA splicing SR protein rA4 |
| 40150_at | down | 0.074001 | small nuclear ribonucleoprotein D3 polypeptide 18 kDa |
| 40276_at | down | 0.074021 | proteasome (prosome, macropain) 26S subunit, non- |
| | | | ATPase, 7 (Mov34 homolog) |
| 31948_at | down | 0.07403 | ribosomal protein S21 |
| 34269_at | down | 0.074059 | erythroid differentiation-related factor 1 |
| 1568_s_at | down | 0.074061 | interferon (alpha, beta and omega) receptor 2 |
| 33242_at | down | 0.074072 | hypothetical protein DT1P1A10 |
| 31967_at | up | 0.074076 | nephrosis 1, congenital, Finnish type (nephrin) |
| 40167_s_at | down | 0.074152 | likely ortholog of mouse WD-40-repeat-containing protei |
| | | | with a SOCS box 2 |
| 35766_at | down | 0.074163 | keratin 18 |
| 41553_at | up | 0.074209 | chromosome 8 open reading frame 1 |
| 755_at | down | 0.07422 | inositol 1,4,5-triphosphate receptor, type 1 |
| 34697_at | up | 0.074223 | low density lipoprotein receptor-related protein 6 |
| 38271_at | down | 0.074257 | histone deacetylase 4 |
| 32629_f_at | up | 0.074409 | butyrophilin, subfamily 3, member A1 |
| 40263_at | up | 0.074575 | zinc finger protein-like 1 |
| 34357_g_at | down | 0.074581 | SRB7 suppressor of RNA polymerase B homolog (yeast) |
| AFFX-BioB-M_at | up | 0.074593 | |
| 36507_at | up | 0.074761 | zinc finger protein 282 |
| 37377_i_at | up | 0.074824 | lamin A/C |
| 39381_at | down | 0.074885 | PHD finger protein 10 |
| 38374_at | down | 0.074924 | TGFB inducible early growth response |
| 32961_at | down | 0.075047 | c-myc promoter-binding protein |
| 38219_at | down | 0.075065 | v-crk sarcoma virus CT10 oncogene homolog (avian) |
| 40517_at | down | 0.075102 | KIAA0372 gene product |
| 40825_at | up | 0.075117 | microtubule-associated protein, RP/EB family, member 3 |
| 35208_at | down | 0.075192 | KIAA0874 protein |
| 37521_s_at | down | 0.075228 | nucleolar cysteine-rich protein |
| 37173_at | up | 0.07526 | centromere protein E, 312 kDa |
| 31738_at | up | 0.075288 | |
| 38071_at | down | 0.075384 | heterogeneous nuclear ribonucleoprotein F |
| 33285_i_at | down | 0.075391 | hypothetical protein FLJ21168 |
| 153_f_at | up | 0.075466 | histone 1, H2bj |
| 32734_at | down | 0.075516 | protein phosphatase 2, regulatory subunit B (B56), epsilo |
| | | | isoform |
| 39360_at | down | 0.07553 | sorting nexin 3 |
| 39509_at | down | 0.07559 | programmed cell death 4 (neoplastic transformation |
| | | | inhibitor) |
| 39852_at | down | 0.075598 | spastic paraplegia 20, spartin (Troyer syndrome) |
| 32333_at | up | 0.075602 | |
| 36535_at | down | 0.075653 | microfibrillar-associated protein 1 |
| 39780_at | down | 0.075735 | protein phosphatase 3 (formerly 2B), catalytic subunit, |
| | | | beta isoform (calcineurin A beta) |
| 35488_at | down | 0.075738 | small nuclear RNA activating complex, polypeptide 1, |
| | | | 43 kDa |
| 545_g_at | down | 0.075781 | nuclear factor of kappa light polypeptide gene enhancer |
| | | | B-cells 2 (p49/p100) |
| 41711_at | up | 0.075904 | thioredoxin reductase 2 |
| 34613_at | up | 0.075912 | KIAA1086 protein |
| 33831_at | down | 0.07592 | CREB binding protein (Rubinstein-Taybi syndrome) |
| 31797_at | down | 0.075936 | TBP-like 1 |
| 34548_at | up | 0.075953 | cytochrome P450, family 11, subfamily B, polypeptide 1 |
| 37030_at | down | 0.076042 | expressed in T-cells and eosinophils in atopic dermatitis |
| 845_at | up | 0.076077 | signal transducer and activator of transcription 6, |
| | | | interleukin-4 induced |
| 39253_s_at | down | 0.076112 | v-ral simian leukemia viral oncogene homolog A (ras |
| | | | related) |
| 1821_at | down | 0.076123 | |
| 41777_at | down | 0.07617 | ATPase, H+ transporting, lysosomal interacting protein 2 |
| 34457_at | up | 0.076243 | solute carrier family 30 (zinc transporter), member 3 |
| 41061_at | down | 0.076272 | huntingtin interacting protein 1 |
| 34829_at | up | 0.076322 | dyskeratosis congenita 1, dyskerin |
| 35751_at | down | 0.076335 | succinate dehydrogenase complex, subunit B, iron sulfur |
| | | | (Ip) |
| 35396_at | up | 0.076518 | hyaluronan synthase 2 |
| 39420_at | down | 0.076789 | DNA-damage-inducible transcript 3 |
| 100_g_at | up | 0.076854 | Rab geranylgeranyltransferase, alpha subunit |
| 33863_at | up | 0.076937 | hypoxia up-regulated 1 |
| 36980_at | down | 0.076989 | proline rich 2 |
| 41736_g_at | up | 0.077002 | KIAA0870 protein |
| 40148_at | down | 0.077005 | amyloid beta (A4) precursor protein-binding, family B, |
| | | | member 2 (Fe65-like) |
| 36216_at | down | 0.077027 | sorting nexin 4 |
| 33656_at | down | 0.077078 | ribosomal protein L37 |
| 135_g_at | down | 0.077114 | abl-interactor 2 |
| 41512_at | down | 0.077118 | BRCA1 associated protein |
| 1451_s_at | up | 0.07736 | osteoblast specific factor 2 (fasciclin I-like) |
| 36224_g_at | down | 0.077412 | splicing factor proline/glutamine rich (polypyrimidine trac |
| | | | binding protein associated) |
| 31903_at | up | 0.077415 | synovial sarcoma translocation gene on chromosome 18 |
| | | | like 1 |
| 33632_g_at | down | 0.077589 | similar to S. pombe dim1+ |
| 40625_f_at | up | 0.07767 | metaxin 1 |
| 37541_at | up | 0.077734 | selectin P ligand |
| 692_s_at | up | 0.077795 | superoxide dismutase 3, extracellular |
| 34259_at | up | 0.07782 | KIAA0664 protein |
| 32871_at | up | 0.077851 | |
| 41806_at | up | 0.077878 | fibroblast growth factor 2 (basic) |
| 40961_at | down | 0.077885 | SWI/SNF related, matrix associated, actin dependent |
| | | | regulator of chromatin, subfamily a, member 2 |
| 35671_at | up | 0.077932 | general transcription factor IIIC, polypeptide 1, alpha |
| | | | 220 kDa |
| 40379_at | up | 0.077985 | cytochrome P450, family 2, subfamily E, polypeptide 1 |
| 37163_at | down | 0.077986 | DKFZP586C1619 protein |
| 33619_at | down | 0.078048 | ribosomal protein S13 |
| 424_s_at | up | 0.07813 | fibroblast growth factor receptor 1 (fms-related tyrosine |
| | | | kinase 2, Pfeiffer syndrome) |
| 31758_at | up | 0.07818 | |
| 38779_r_at | up | 0.078277 | hepatoma-derived growth factor (high-mobility group |
| | | | protein 1-like) |
| 38102_at | down | 0.078302 | hypothetical protein FLJ34588 |
| 32768_at | up | 0.078331 | tudor domain containing 3 |
| 33659_at | up | 0.078352 | cofilin 1 (non-muscle) |
| 36374_at | up | 0.078482 | |
| 38006_at | down | 0.078527 | CD48 antigen (B-cell membrane protein) |
| 38927_i_at | up | 0.078558 | tyrosinase (oculocutaneous albinism IA) |
| 36094_at | up | 0.078582 | troponin T3, skeletal, fast |
| 350_at | down | 0.078582 | zinc finger protein 161 |
| 33155_at | up | 0.078626 | iduronidase, alpha-L- |
| 39315_at | up | 0.078642 | angiopoietin 1 |
| 33162_at | down | 0.078765 | insulin receptor |
| 1440_s_at | down | 0.078833 | tumor necrosis factor receptor superfamily, member 6 |
| 37566_at | up | 0.078937 | KIAA1045 protein |
| 40467_at | down | 0.079211 | succinate dehydrogenase complex, subunit D, integral |
| | | | membrane protein |
| 211_at | down | 0.079258 | down-regulator of transcription 1, TBP-binding (negative |
| | | | cofactor 2) |
| 36282_at | up | 0.079286 | |
| 39427_at | down | 0.079371 | ubiquinol-cytochrome c reductase binding protein |
| 39810_at | down | 0.079388 | hypothetical protein MGC2749 |
| 41824_at | down | 0.079408 | CGI-48 protein |
| 41508_at | up | 0.079432 | early growth response 4 |
| 1614_s_at | down | 0.079473 | ubiquitin specific protease 6 (Tre-2 oncogene) |
| 1072_g_at | up | 0.079479 | GATA binding protein 2 |
| 36582_g_at | down | 0.079698 | glycyl-tRNA synthetase |
| 35105_at | up | 0.079724 | sciellin |
| 34598_at | up | 0.079789 | tenascin R (restrictin, janusin) |
| 35154_at | up | 0.079876 | BTB (POZ) domain containing 2 |
| 40290_f_at | down | 0.080058 | sialyltransferase 4A (beta-galactoside alpha-2,3- |
| | | | sialyltransferase) |
| 34201_at | down | 0.080202 | DnaJ (Hsp40) homolog, subfamily A, member 2 |
| 40870_g_at | down | 0.08021 | RNA binding motif protein 6 |
| 36610_at | down | 0.080302 | R3H domain (binds single-stranded nucleic acids) |
| | | | containing |
| 36155_at | up | 0.080485 | sparc/osteonectin, cwcv and kazal-like domains |
| | | | proteoglycan (testican) 2 |
| 33209_at | down | 0.080629 | YY1 transcription factor |
| 40887_g_at | up | 0.080665 | eukaryotic translation elongation factor 1 alpha 1 |
| 617_at | down | 0.080689 | acid phosphatase, prostate |
| 35214_at | up | 0.080736 | UDP-glucose dehydrogenase |
| 916_at | up | 0.080801 | protein tyrosine phosphatase, receptor type, N |
| 40279_at | up | 0.080847 | KIAA0121 gene product |
| 38712_at | down | 0.080849 | chromosome 1 open reading frame 9 |
| 41120_at | up | 0.080863 | aminomethyltransferase (glycine cleavage system prote |
| | | | T) |
| 37813_at | up | 0.08087 | |
| 1486_at | up | 0.080961 | polymerase (RNA) II (DNA directed) polypeptide J, |
| | | | 13.3 kDa |
| 34351_at | up | 0.081014 | phospholipase C, gamma 1 (formerly subtype 148) |
| 34809_at | down | 0.081037 | KIAA0999 protein |
| 39040_at | down | 0.081117 | ubiquitin-conjugating enzyme E2, J1 (UBC6 homolog, |
| | | | yeast) |
| 40689_at | down | 0.081186 | sel-1 suppressor of lin-12-like (C. elegans) |
| 931_at | down | 0.081247 | Epstein-Barr virus induced gene 2 (lymphocyte-specific |
| | | | protein-coupled receptor) |
| 39147_g_at | up | 0.081251 | alpha thalassemia/mental retardation syndrome X-linked |
| | | | (RAD54 homolog, S. cerevisiae) |
| 38116_at | down | 0.08134 | KIAA0101 gene product |
| 1682_s_at | up | 0.081428 | ATP-binding cassette, sub-family B (MDR/TAP), membe 1 |
| 36519_at | down | 0.081516 | excision repair cross-complementing rodent repair |
| | | | deficiency, complementation group 1 (includes |
| | | | overlapping antisense sequence) |
| 40294_at | up | 0.081521 | ATP-binding cassette, sub-family B (MDR/TAP), membe 9 |
| 38567_at | down | 0.081526 | CD1D antigen, d polypeptide |
| 160029_at | down | 0.081576 | protein kinase C, beta 1 |
| 195_s_at | down | 0.081607 | caspase 4, apoptosis-related cysteine protease |
| 36748_at | up | 0.081853 | synapsin II |
| 475_at | up | 0.081884 | receptor (TNFRSF)-interacting serine-threonine kinase 1 |
| 31423_at | up | 0.081988 | |
| 35907_at | up | 0.081994 | cyclin F |
| 39033_at | down | 0.082005 | chromosome 1 open reading frame 8 |
| 31734_at | up | 0.082019 | homeo box C11 |
| 32640_at | up | 0.082173 | intercellular adhesion molecule 1 (CD54), human |
| | | | rhinovirus receptor |
| 39498_at | up | 0.082268 | FXYD domain containing ion transport regulator 2 |
| 1253_at | down | 0.082282 | glycogen synthase kinase 3 beta |
| 33435_r_at | down | 0.082325 | BET1 homolog (S. cerevisiae) |
| 35810_at | down | 0.082362 | actin related protein 2/3 complex, subunit 3, 21 kDa |
| 33964_at | up | 0.082441 | crystallin, gamma C |
| 40866_at | down | 0.082452 | nipsnap homolog 1 (C. elegans) |
| 37045_at | down | 0.082479 | sorting nexin 19 |
| 39431_at | down | 0.082604 | aminopeptidase puromycin sensitive |
| 38514_at | down | 0.082742 | immunoglobulin lambda-like polypeptide 1 |
| 33876_at | up | 0.082748 | transcriptional co-activator with PDZ-binding motif (TAZ) |
| 882_at | up | 0.082785 | colony stimulating factor 1 (macrophage) |
| 34123_at | up | 0.082836 | |
| 34533_at | up | 0.082873 | hypothetical protein FLJ32746 |
| 33543_s_at | down | 0.082933 | pinin, desmosome associated protein |
| 37980_at | down | 0.082971 | CBF1 interacting corepressor |
| 574_s_at | down | 0.083007 | caspase 1, apoptosis-related cysteine protease |
| | | | (interleukin 1, beta, convertase) |
| 789_at | up | 0.083148 | early growth response 1 |
| 34163_g_at | down | 0.083192 | RNA binding protein with multiple splicing |
| 39718_r_at | down | 0.083273 | mitochondrial ribosomal protein L33 |
| 1136_at | down | 0.083343 | deoxythymidylate kinase (thymidylate kinase) |
| 33893_r_at | down | 0.08337 | KARP-1-binding protein |
| 40673_at | up | 0.083476 | acyl-Coenzyme A dehydrogenase, short/branched chain |
| 36872_at | down | 0.08356 | cyclic AMP phosphoprotein, 19 kD |
| 31930_f_at | up | 0.083573 | Rhesus blood group, CcEe antigens |
| 40119_at | down | 0.083591 | cartilage associated protein |
| 241_g_at | up | 0.083623 | spermidine synthase |
| 519_g_at | up | 0.083723 | nuclear receptor subfamily 1, group H, member 2 |
| 37827_r_at | down | 0.083832 | chromosome 21 open reading frame 5 |
| 37679_at | down | 0.083856 | interferon-related developmental regulator 1 |
| 37385_at | down | 0.08386 | peptidyl-prolyl isomerase G (cyclophilin G) |
| 33760_at | down | 0.083864 | peroxisomal biogenesis factor 14 |
| 671_at | up | 0.083879 | secreted protein, acidic, cysteine-rich (osteonectin) |
| 35143_at | down | 0.083893 | hypothetical protein DKFZp566A1524 |
| 35946_at | up | 0.083985 | NEL-like 1 (chicken) |
| 34678_at | down | 0.084027 | fer-1-like 3, myoferlin (C. elegans) |
| 36121_at | up | 0.084131 | epsin 2 |
| 35413_s_at | up | 0.084141 | zinc finger protein 22 (KOX 15) |
| 41197_at | down | 0.084209 | RAD23 homolog A (S. cerevisiae) |
| 32173_at | up | 0.084294 | translational inhibitor protein p14.5 |
| 39791_at | down | 0.084465 | ATPase, Ca++ transporting, cardiac muscle, slow twitch |
| 41124_r_at | down | 0.084468 | ectonucleotide pyrophosphatase/phosphodiesterase 2 |
| | | | (autotaxin) |
| 41573_at | down | 0.08456 | Sp3 transcription factor |
| 31805_at | up | 0.084593 | fibroblast growth factor receptor 3 (achondroplasia, |
| | | | thanatophoric dwarfism) |
| 33080_s_at | up | 0.084607 | RAP1, GTPase activating protein 1 |
| 109_at | down | 0.084611 | Rab9 effector p40 |
| 34270_at | up | 0.084688 | LSM5 homolog, U6 small nuclear RNA associated (S. cerevisiae) |
| 37406_at | down | 0.084705 | microtubule-associated protein, RP/EB family, member 2 |
| 664_at | down | 0.08478 | interleukin 8 receptor, beta |
| 36350_at | up | 0.084859 | |
| 32883_at | down | 0.084885 | KRAB zinc finger protein KR18 |
| 35901_at | up | 0.08489 | piwi-like 1 (Drosophila) |
| 1885_at | up | 0.084893 | excision repair cross-complementing rodent repair |
| | | | deficiency, complementation group 3 (xeroderma |
| | | | pigmentosum group B complementing) |
| 40701_at | up | 0.085053 | ubiquitin specific protease 13 (isopeptidase T-3) |
| 268_at | down | 0.085065 | platelet/endothelial cell adhesion molecule (CD31 antige |
| 1845_at | down | 0.085084 | mitogen-activated protein kinase kinase 4 |
| 41813_at | up | 0.085175 | nucleoporin 210 |
| 37282_at | down | 0.085322 | MAD2 mitotic arrest deficient-like 1 (yeast) |
| 39883_at | down | 0.085337 | putative dimethyladenosine transferase |
| 39581_at | down | 0.085337 | cystatin A (stefin A) |
| 40558_at | up | 0.085339 | guanylate cyclase activator 1B (retina) |
| 1143_s_at | down | 0.085382 | |
| 37796_at | up | 0.085405 | leucine rich repeat neuronal 4 |
| 40758_at | down | 0.085415 | immature colon carcinoma transcript 1 |
| AFFX- | up | 0.0855 | |
| M27830_3_at | | | |
| 36462_at | up | 0.085538 | SMYD family member 5 |
| 31853_at | down | 0.08559 | embryonic ectoderm development |
| 37293_at | down | 0.085666 | KIAA0097 gene product |
| 38705_at | down | 0.085669 | ubiquitin-conjugating enzyme E2D 2 (UBC4/5 homolog, |
| | | | yeast) |
| 37588_s_at | up | 0.085678 | mitogen-activated protein kinase 8 interacting protein 2 |
| 40211_at | down | 0.08573 | heterogeneous nuclear ribonucleoprotein A1 |
| 467_at | down | 0.08595 | osteoclast stimulating factor 1 |
| 38530_at | up | 0.086109 | hypothetical protein FLJ22709 |
| 40048_at | down | 0.086249 | pumilio homolog 1 (Drosophila) |
| 34223_at | up | 0.086252 | colony stimulating factor 3 receptor (granulocyte) |
| 33240_at | up | 0.0863 | likely ortholog of mouse semaF cytoplasmic domain |
| | | | associated protein 3 |
| 33886_at | down | 0.086302 | spectrin SH3 domain binding protein 1 |
| 39832_at | up | 0.086357 | arsenate resistance protein ARS2 |
| 31638_at | up | 0.086423 | |
| 41814_at | down | 0.086559 | fucosidase, alpha-L-1, tissue |
| 31800_at | up | 0.086561 | |
| 31979_at | up | 0.086615 | 6-phosphofructo-2-kinase/fructose-2,6-biphosphatase 4 |
| 34961_at | up | 0.086616 | T cell activation, increased late expression |
| 35597_at | up | 0.086723 | phosphoprotein regulated by mitogenic pathways |
| 33529_at | up | 0.086755 | alcohol dehydrogenase 7 (class IV), mu or sigma |
| | | | polypeptide |
| 38889_at | up | 0.086863 | MAD, mothers against decapentaplegic homolog |
| | | | (Drosophila) interacting protein, receptor activation anc |
| 39221_at | down | 0.086888 | leukocyte immunoglobulin-like receptor, subfamily B (wi |
| | | | TM and ITIM domains), member 2 |
| 32443_at | up | 0.087 | zinc finger protein 157 (HZF22) |
| 34011_at | up | 0.08701 | harakiri, BCL2 interacting protein (contains only BH3 |
| | | | domain) |
| 39643_at | up | 0.087011 | polymerase (DNA directed), gamma 2, accessory subur |
| 37935_at | up | 0.08708 | PRP4 pre-mRNA processing factor 4 homolog (yeast) |
| 39749_at | down | 0.087136 | proteasome (prosome, macropain) 26S subunit, non- |
| | | | ATPase, 4 |
| 36865_at | down | 0.08718 | KIAA0759 protein |
| 36081_s_at | down | 0.087181 | chromosome 21 open reading frame 18 |
| 38347_at | down | 0.087211 | elongation protein 4 homolog (S. cerevisiae) |
| 485_at | up | 0.087219 | |
| 1001_at | up | 0.087263 | tyrosine kinase with immunoglobulin and epidermal |
| | | | growth factor homology domains |
| 35293_at | down | 0.087479 | Sjogren syndrome antigen A2 (60 kDa, ribonucleoprotein |
| | | | autoantigen SS-A/Ro) |
| 40733_f_at | up | 0.087601 | msh homeo box homolog 2 (Drosophila) |
| 34474_at | down | 0.087639 | |
| 41420_at | down | 0.087753 | insulin-like growth factor binding protein 5 |
| 210_at | up | 0.08778 | phospholipase C, beta 2 |
| 40901_at | down | 0.087859 | striatin, calmodulin binding protein 3 |
| 34708_at | up | 0.087884 | ficolin (collagen/fibrinogen domain containing) 3 (Hakata |
| | | | antigen) |
| 1671_s_at | up | 0.087925 | mitogen-activated protein kinase 14 |
| 33165_at | down | 0.087974 | target of EGR1, member 1 (nuclear) |
| 31679_at | up | 0.087997 | |
| 39290_f_at | up | 0.088024 | PAI-1 mRNA-binding protein |
| 40480_s_at | down | 0.088156 | FYN oncogene related to SRC, FGR, YES |
| 953_g_at | down | 0.088165 | |
| 35845_at | down | 0.088184 | SEC24 related gene family, member B (S. cerevisiae) |
| 41125_r_at | up | 0.088253 | ectonucleotide pyrophosphatase/phosphodiesterase 2 |
| | | | (autotaxin) |
| 32190_at | down | 0.088305 | fatty acid desaturase 2 |
| 41117_s_at | up | 0.088317 | solute carrier family 9 (sodium/hydrogen exchanger), |
| | | | isoform 3 regulatory factor 2 |
| 39824_at | up | 0.088333 | protein tyrosine phosphatase type IVA, member 3 |
| 38237_at | up | 0.088414 | gamma-glutamyltransferase-like activity 1 |
| 31713_s_at | up | 0.088448 | discs, large (Drosophila) homolog-associated protein 2 |
| 37323_r_at | up | 0.088637 | hydroxyprostaglandin dehydrogenase 15-(NAD) |
| 39862_at | up | 0.088689 | KIAA0296 gene product |
| 31594_at | up | 0.088804 | keratin, hair, acidic, 3A |
| 33272_at | up | 0.088835 | serum amyloid A1 |
| 38907_at | up | 0.088895 | |
| 32927_at | down | 0.088975 | |
| 41425_at | down | 0.089008 | Friend leukemia virus integration 1 |
| 39396_at | down | 0.089017 | lysophospholipase I |
| 34858_at | up | 0.089029 | potassium channel tetramerisation domain containing 2 |
| 32067_at | up | 0.089124 | cAMP responsive element modulator |
| 35473_at | up | 0.08921 | collagen, type I, alpha 1 |
| 38073_at | down | 0.089213 | RNA (guanine-7-) methyltransferase |
| 34859_at | up | 0.089268 | melanoma antigen, family D, 2 |
| 38048_at | up | 0.089393 | RNA binding protein with multiple splicing |
| 38061_at | up | 0.08946 | ribosomal protein S16 |
| 34671_at | down | 0.08955 | polymerase (RNA) III (DNA directed) (62 kD) |
| 32813_s_at | up | 0.089623 | katanin p80 (WD repeat containing) subunit B 1 |
| 39829_at | up | 0.089659 | ADP-ribosylation factor-like 7 |
| 34736_at | up | 0.089693 | cyclin B1 |
| 40515_at | down | 0.08972 | eukaryotic translation initiation factor 2B, subunit 2 beta, |
| | | | 39 kDa |
| 37763_at | up | 0.089731 | retinoid X receptor, beta |
| 33984_at | up | 0.089752 | heat shock 90 kDa protein 1, beta |
| 32025_at | down | 0.089771 | transcription factor 7-like 2 (T-cell specific, HMG-box) |
| 38990_at | down | 0.089788 | F-box only protein 9 |
| 38117_at | up | 0.089832 | SEC24 related gene family, member C (S. cerevisiae) |
| 36784_at | down | 0.090005 | chorionic somatomammotropin hormone-like 1 |
| 32662_at | up | 0.090014 | mediator of DNA damage checkpoint 1 |
| 41535_at | down | 0.090077 | CDK2-associated protein 1 |
| 39152_f_at | down | 0.0901 | coilin |
| 2069_s_at | down | 0.090137 | catenin (cadherin-associated protein), alpha 1, 102 kDa |
| 35307_at | down | 0.090206 | GDP dissociation inhibitor 2 |
| 35783_at | down | 0.090268 | vesicle-associated membrane protein 3 (cellubrevin) |
| 909_g_at | down | 0.090304 | interferon-induced protein with tetratricopeptide repeats: |
| 34038_at | up | 0.090364 | solute carrier family 6 (neurotransmitter transporter, |
| | | | GABA), member 13 |
| 37723_at | down | 0.090364 | cyclin G2 |
| 39913_at | up | 0.090436 | heparan sulfate 6-O-sulfotransferase 1 |
| 36511_at | down | 0.09055 | SAC1 suppressor of actin mutations 1-like (yeast) |
| 31846_at | up | 0.090573 | ras homolog gene family, member D |
| 38419_at | down | 0.090574 | KIAA0196 gene product |
| 1745_at | down | 0.090606 | |
| 34680_s_at | down | 0.090628 | KIAA0107 gene product |
| 31833_at | up | 0.090658 | phosphatidylinositol-4-phosphate 5-kinase, type I, alpha |
| 39385_at | up | 0.090659 | alanyl (membrane) aminopeptidase (aminopeptidase N, |
| | | | aminopeptidase M, microsomal aminopeptidase, CD13, |
| | | | p150) |
| 37768_at | up | 0.0908 | N-methylpurine-DNA glycosylase |
| 34161_at | up | 0.090915 | lactoperoxidase |
| 41868_at | up | 0.090976 | gamma-glutamyltransferase 1 |
| 37940_f_at | up | 0.090998 | apolipoprotein B mRNA editing enzyme, catalytic |
| | | | polypeptide-like 3C |
| 35230_at | up | 0.091025 | TIR domain containing adaptor inducing interferon-beta |
| 40107_at | up | 0.091064 | aldolase C, fructose-bisphosphate |
| 40764_at | up | 0.091092 | glutamic-oxaloacetic transaminase 2, mitochondrial |
| | | | (aspartate aminotransferase 2) |
| 37685_at | down | 0.091314 | phosphatidylinositol binding clathrin assembly protein |
| 1288_s_at | up | 0.091336 | eukaryotic translation elongation factor 1 alpha 1 |
| 33379_at | up | 0.09136 | synovial sarcoma, X breakpoint 2 interacting protein |
| 39641_at | up | 0.091498 | uracil-DNA glycosylase 2 |
| 38739_at | down | 0.091509 | v-ets erythroblastosis virus E26 oncogene homolog 2 |
| | | | (avian) |
| 35807_at | up | 0.091617 | cytochrome b-245, alpha polypeptide |
| 36213_at | up | 0.09167 | malignant fibrous histiocytoma amplified sequence 1 |
| 31522_f_at | up | 0.091671 | histone 1, H2bf |
| 36184_at | up | 0.091691 | procollagen-lysine, 2-oxoglutarate 5-dioxygenase (lysine |
| | | | hydroxylase, Ehlers-Danlos syndrome type VI) |
| 806_at | up | 0.091752 | cytokine-inducible kinase |
| 2081_s_at | down | 0.091827 | protein kinase C, theta |
| 37621_at | down | 0.091829 | interleukin 6 signal transducer (gp130, oncostatin M |
| | | | receptor) |
| 41334_r_at | down | 0.091888 | |
| 36613_at | up | 0.092021 | interferon-related developmental regulator 2 |
| 32878_f_at | down | 0.09204 | |
| 34908_at | up | 0.092066 | hypothetical protein FLJ13946 |
| 37212_at | up | 0.0921 | Sp2 transcription factor |
| 2084_s_at | up | 0.092203 | ets variant gene 4 (E1A enhancer binding protein, E1AF |
| 32856_at | up | 0.092236 | KIAA0819 protein |
| 39219_at | down | 0.092383 | CCAAT/enhancer binding protein (C/EBP), gamma |
| 37053_at | up | 0.092455 | ATPase, Ca++ transporting, plasma membrane 2 |
| 38962_at | up | 0.092537 | KIAA0298 gene product |
| 36624_at | up | 0.092613 | IMP (inosine monophosphate) dehydrogenase 2 |
| 40757_at | up | 0.092646 | granzyme A (granzyme 1, cytotoxic T-lymphocyte- |
| | | | associated serine esterase 3) |
| 31780_f_at | up | 0.092646 | |
| 37438_at | up | 0.092667 | KIAA0419 gene product |
| 35844_at | up | 0.092675 | syndecan 4 (amphiglycan, ryudocan) |
| AFFX-BioB-M_st | up | 0.092696 | |
| 38695_at | down | 0.092773 | NADH dehydrogenase (ubiquinone) Fe—S protein 4, |
| | | | 18 kDa (NADH-coenzyme Q reductase) |
| 31446_s_at | up | 0.092903 | proline rich 5 (salivary) |
| 32747_at | down | 0.092991 | aldehyde dehydrogenase 2 family (mitochondrial) |
| 32129_at | down | 0.093034 | zinc finger protein 364 |
| 37998_at | up | 0.093081 | superkiller viralicidic activity 2-like (S. cerevisiae) |
| 40944_at | down | 0.09309 | TGFB inducible early growth response 2 |
| 31512_at | up | 0.093117 | immunoglobulin kappa variable 1-13 |
| 41268_g_at | down | 0.093129 | KIAA1049 protein |
| 31960_f_at | up | 0.093132 | G antigen 2 |
| 2009_at | up | 0.093184 | PTK2B protein tyrosine kinase 2 beta |
| 37520_at | down | 0.093259 | nucleolar cysteine-rich protein |
| 32300_s_at | up | 0.09328 | tyrosine hydroxylase |
| 36837_at | up | 0.093336 | kinesin family member 2C |
| 39834_at | up | 0.093358 | cholinergic receptor, nicotinic, epsilon polypeptide |
| 40465_at | down | 0.09338 | prp28, U5 snRNP 100 kd protein |
| 33965_at | up | 0.093408 | chemokine (C—C motif) ligand 1 |
| 41068_at | down | 0.09347 | outer dense fiber of sperm tails 2 |
| 35100_at | up | 0.093484 | sialyltransferase 8C (alpha2,3Galbeta1,4GlcNAcalpha |
| | | | 2,8-sialyltransferase) |
| 38171_at | up | 0.093582 | WD-repeat protein |
| 34101_at | up | 0.093618 | |
| 35279_at | down | 0.09364 | Tax1 (human T-cell leukemia virus type I) binding protein 1 |
| 34129_at | up | 0.093741 | tomosyn-like |
| 38081_at | down | 0.093785 | leukotriene A4 hydrolase |
| 33923_s_at | down | 0.09381 | PR domain containing 2, with ZNF domain |
| 39965_at | up | 0.093868 | ras-related C3 botulinum toxin substrate 3 (rho family, |
| | | | small GTP binding protein Rac3) |
| 40691_at | down | 0.093908 | zinc finger protein 274 |
| 35562_at | up | 0.093917 | histone 1, H2bj |
| 40853_at | down | 0.093992 | ATPase, Class V, type 10D |
| 34112_r_at | up | 0.094039 | |
| 1073_at | down | 0.094039 | transcription elongation factor A (SII), 1 |
| 37535_at | down | 0.094144 | cAMP responsive element binding protein 1 |
| 32140_at | up | 0.094211 | sortilin-related receptor, L(DLR class) A repeats- |
| | | | containing |
| 33077_at | up | 0.094215 | |
| 34028_at | down | 0.094218 | G protein-coupled receptor 19 |
| 35356_at | down | 0.094405 | hypothetical protein MGC9651 |
| 40797_at | down | 0.094519 | a disintegrin and metalloproteinase domain 10 |
| 38569_at | up | 0.094591 | nuclear respiratory factor 1 |
| 1715_at | down | 0.094642 | tumor necrosis factor (ligand) superfamily, member 10 |
| 36277_at | up | 0.094818 | CD3E antigen, epsilon polypeptide (TiT3 complex) |
| 34529_at | up | 0.094849 | |
| 35445_at | up | 0.0949 | sorting nexin 26 |
| 32879_at | down | 0.094954 | |
| 41635_at | down | 0.094971 | Wilms tumor 1 associated protein |
| 38673_s_at | down | 0.094985 | cyclin-dependent kinase inhibitor 1C (p57, Kip2) |
| 37118_at | up | 0.095049 | ret finger protein-like 1 antisense |
| 35306_at | down | 0.095212 | DEAD/H (Asp-Glu-Ala-Asp/His) box polypeptide 15 |
| 40992_s_at | down | 0.09541 | sin3-associated polypeptide, 30 kDa |
| 37891_at | up | 0.095425 | |
| 39448_r_at | up | 0.095584 | B7 gene |
| 38843_at | down | 0.095688 | high-mobility group protein 2-like 1 |
| 39224_at | down | 0.0957 | centaurin, delta 1 |
| 39143_at | down | 0.095718 | nuclear factor of activated T-cells, cytoplasmic, |
| | | | calcineurin-dependent 1 |
| 39985_r_at | up | 0.095724 | FKBP-associated protein |
| 38592_s_at | up | 0.095754 | KIAA0284 protein |
| 1518_at | up | 0.095784 | v-ets erythroblastosis virus E26 oncogene homolog 1 |
| | | | (avian) |
| 35605_at | up | 0.095808 | angiopoietin-like factor |
| 41712_at | down | 0.095851 | likely ortholog of mouse aquarius |
| 35999_r_at | down | 0.095865 | KIAA0781 protein |
| 36735_f_at | up | 0.09588 | killer cell immunoglobulin-like receptor, three domains, |
| | | | long cytoplasmic tail, 2 |
| 33710_at | down | 0.095883 | putative protein similar to nessy (Drosophila) |
| 32888_at | up | 0.095889 | leukocyte tyrosine kinase |
| 37397_at | down | 0.095893 | platelet/endothelial cell adhesion molecule (CD31 antige |
| 41528_at | up | 0.095958 | |
| 40766_at | up | 0.096077 | complement component 4A |
| 36389_at | down | 0.096078 | class-I MHC-restricted T cell associated molecule |
| 39666_at | up | 0.096165 | guanine nucleotide binding protein (G protein), gamma 4 |
| 34755_at | up | 0.096176 | ADP-ribosyltransferase (NAD+; poly(ADP-ribose) |
| | | | polymerase)-like 2 |
| 32708_g_at | down | 0.096177 | katanin p60 (ATPase-containing) subunit A 1 |
| 32228_at | up | 0.096212 | adaptor-related protein complex 2, alpha 2 subunit |
| 40886_at | down | 0.09626 | eukaryotic translation elongation factor 1 alpha 1 |
| 32285_g_at | up | 0.096383 | T-box 1 |
| 39734_at | down | 0.096467 | small inducible cytokine subfamily E, member 1 |
| | | | (endothelial monocyte-activating) |
| 31337_at | up | 0.096538 | gonadotropin-releasing hormone 2 |
| 41488_at | down | 0.096667 | hypothetical protein A-211C6.1 |
| 34438_at | up | 0.096753 | serine (or cysteine) proteinase inhibitor, clade B |
| | | | (ovalbumin), member 9 |
| 31478_at | up | 0.096764 | pancreatic elastase IIB |
| 39867_at | up | 0.09692 | Tu translation elongation factor, mitochondrial |
| 41324_g_at | up | 0.096963 | forkhead box M1 |
| 1775_at | up | 0.097235 | polymerase (DNA-directed), alpha (70 kD) |
| 32391_g_at | down | 0.097269 | |
| 40803_at | down | 0.097361 | pro-oncosis receptor inducing membrane injury gene |
| 34677_f_at | down | 0.097379 | TL132 protein |
| 493_at | up | 0.097484 | casein kinase 1, delta |
| 37870_at | up | 0.097488 | transcription termination factor, RNA polymerase II |
| 41859_at | up | 0.097578 | uronyl-2-sulfotransferase |
| 37618_at | up | 0.097661 | homeo box B7 |
| 33453_at | up | 0.097774 | ATPase, H+ transporting, lysosomal interacting protein 1 |
| 32909_at | up | 0.097796 | aquaporin 5 |
| 38622_at | up | 0.097851 | hypothetical protein BC004409 |
| 40845_at | down | 0.097859 | interleukin enhancer binding factor 3, 90 kDa |
| 39351_at | down | 0.097885 | CD59 antigen p18-20 (antigen identified by monoclonal |
| | | | antibodies 16.3A5, EJ16, EJ30, EL32 and G344) |
| 33341_at | down | 0.097943 | guanine nucleotide binding protein (G protein), beta |
| | | | polypeptide 1 |
| 38279_at | up | 0.098024 | guanine nucleotide binding protein (G protein), alpha z |
| | | | polypeptide |
| 41543_at | up | 0.098055 | lymphoid nuclear protein related to AF4 |
| 41163_at | down | 0.09819 | integral type I protein |
| 32101_at | down | 0.098215 | galactosamine (N-acetyl)-6-sulfate sulfatase (Morquio |
| | | | syndrome, mucopolysaccharidosis type IVA) |
| 33172_at | up | 0.098326 | hypothetical protein FLJ10849 |
| 36168_at | up | 0.098482 | fibroblast growth factor receptor 1 (fms-related tyrosine |
| | | | kinase 2, Pfeiffer syndrome) |
| 41385_at | down | 0.098533 | erythrocyte membrane protein band 4.1-like 3 |
| 31537_at | up | 0.098538 | ADP-ribosyltransferase 1 |
| 32564_at | down | 0.098647 | protein translocation complex beta |
| 1922_g_at | up | 0.098667 | |
| 158_at | down | 0.098748 | DnaJ (Hsp40) homolog, subfamily B, member 4 |
| 857_at | down | 0.099054 | protein phosphatase 1A (formerly 2C), magnesium- |
| | | | dependent, alpha isoform |
| 34029_at | up | 0.099115 | dentin matrix acidic phosphoprotein |
| 34544_at | down | 0.099119 | zinc finger protein 267 |
| 35348_at | down | 0.099122 | protein kinase, AMP-activated, beta 1 non-catalytic |
| | | | subunit |
| 1075_f_at | up | 0.099205 | interferon, alpha 16 |
| 960_g_at | down | 0.099331 | |
| 35091_at | up | 0.099335 | neuregulin 2 |
| 40785_g_at | down | 0.099423 | protein phosphatase 2, regulatory subunit B (B56), |
| | | | gamma isoform |
| 1921_at | up | 0.099462 | |
| 666_at | up | 0.099504 | phosphodiesterase 4A, cAMP-specific |
| | | | (phosphodiesterase E2 dunce homolog, Drosophila) |
| 33340_at | down | 0.099814 | praja 2, RING-H2 motif containing |
| 31759_at | up | 0.099864 | alpha-2 macroglobulin family protein VIP |

### Example 2

### Psychiatric Illness Diagnosis with Multigene Expression Classification

### Patient and Control Subject Recruitment and Study Procedure

All subject recruitment was performed according to IRB regulations.

Medicated Schizophrenia (SZ) Subjects. Seven White SZ men between the ages of 25-65 were recruited from the residents of a psychiatric center and four community residential facilities. SZ patients were screened for inclusion based on SZ diagnosis. Patient records from previous admissions and from other facilities were collected for each subject. Informed consent was obtained on the patient's resident ward. Charts were screened for neuroleptic history and in addition for medical history and other medication use. The seven SZ patients who were analyzed in the preliminary study had medication profiles that were diverse and included several different classes of atypical and typical neuroleptic medications: Subject 493: Olanzapine, Depakote, Risperidone., Subject 494: Chloral Hydrate, Zyprexa., Subject 495: Loxapine, Benztropine, Seroquel, Vistaril., Subject 535: Clozapine, Artane., Subject 588: Haloperidol, Haloperidol Decanoate, Cogentin, Depakote., Subject 630: Olanzapine, Risperidone., Subject 631: Haloperidol, Clozapine. One patient (ID 494) had been neuroleptic drug-free (Clozapine) a short time (5 days).

Non-Medication SZ Subject. One never-medicated 39-year-old White male SZ subject was recruited into the study, Subject 964. Increasing delusions and paranoia precipitated the subject's admission to a local community hospital. He was hospitalized for 37 days but refused all medications. He was assessed for court-mandated treatment but did not fulfill the criteria of dangerousness and this avenue was not pursued. At no time during his hospitalization was any emergency or stat medication administered. The patient was given an Axis I paranoid schizophrenia diagnosis. His global assessment of functioning score was 28%. The patient's physical examination found no medical conditions or abnormalities, and his SMAC, CBC and urinalysis results were all within the normal ranges. At admission a urine drug toxicology screen proved negative.

Informed consent was obtained, and a blood draw was performed. The subject was questioned about his general health, his treatment history and any drug, alcohol and smoking histories (of which all were negative). A brief psychiatric rating scale (BPRS) was administered and his BPRS score was 43.

Control Subjects. Five age-matched controls were recruited from the staff. Subjects completed a form (with the study team assistance) documenting that neither they nor their first degree relatives had a history of SZ, other psychotic disorders, mood disorders or of paranoid, schizoid, or schizotype personality disorder. Subjects were also questioned about their smoking history any current use of, or history of alcohol or illicit drugs. Forms were also completed listing current medications and medical history. Subjects were seen at their place of work and informed consent obtained. Control subjects were given the study ID nos. 401, 492, 536, 634, and 641).

BPD Subjects. Two White male subjects with a diagnosis of BPD (both aged 41), were recruited into the study. Patient records from previous admissions and from other facilities were collected for each subject. Informed consent was obtained on the patient's resident ward. Charts were screened for present and past neuroleptic use and in addition for medical history exclusions and other medication or drug use and smoker status (as described above). The BPD subjects had medication profiles as follows: Subject 767: Depakote, Quietapine and Zoloft., Subject 846: Fluoxetine and Remeron.

Medical Exclusions. A list of medical exclusions was generated. A complete blood count (CBC) with differentials was performed for all samples collected 7.

Sample Processing and Microarray Hybridization. Immediately after blood collection, leukocytes were isolated by lysis of red cells, centrifugation and washing (Qiagen). Purified leukocytes were stored at −70° C. prior to RNA extraction. 8 μg of total RNA was employed as a cDNA synthesis template, using an oligo-dT primer and Reverse Transcriptase (RT) enzyme, according to standard Affymetrix protocols. Purified cDNA, quantified by gel electrophoresis, was then used as a template to generate biotin labeled cRNA, using an in-vitro transcription kit (Enzo). cRNA samples were quantified by UV spectrometry and stored at −70° C. prior to fragmentation. Following fragmentation, 20 ng of each cRNA product was hybridized to an Affymetrix TEST3 array to check the quality of each sample. Each cRNA sample was then hybridized to an HU95A array.

Real-Time RT-PCR. 200 ng of total RNA from each subject was employed for first strand cDNA synthesis, using random hexamer primers and Superscripe^{II} RT enzyme (Invitrogen). Primers were designed using the Primer3 program (Whitehead Institute), except for the 18S ribosomal RNA primers, which were purchased as an internal standard PCR kit (Ambion). For real-time PCR the SYBR Green assay, which measures the linear binding of florescent molecules to double-stranded DNA molecules at each cycle of the amplification, was performed using the Quantitech Kit (Qiagen), on an ABI PRISM 7700 apparatus. The resultant data was imported into Sequence Detector, v1.7a software (ABI), and Cts were calculated. The Ct (the PCR threshold cycle where an increase in reporter fluorescence above a baseline signal can first be detected) has a direct correlation with template concentration. The Ct's of samples with known copy numbers were employed to generate standard amplification curves for each set of specific gene primers. Final copy numbers of each sample RNA were determined from a standard curve, and compared with the 18S standard results.

### Gene Expression Data Acquisition and Analysis

Affymetrix® Microarray Suite Software (v5.0) Data acquisition was performed as described for Example 1. The resultant data was converted to Excel spreadsheets, and collated. All gene expression values given an “absent call” were removed from the datasets. Gene expression data was then filtered by removing all genes from analysis if they were not found to be “present” in at least two subjects. All statistical tests on the data were performed in Excel, except those described in detail below.

Data analysis and Hierarchical Clustering. Hierarchical clustering was performed as described for Example 1, above, using the Cluster program.

### Results of the Preliminary Studies

Pair-wise Analysis of microarray results. To investigate total sample variability, a pair-wise comparison of expression levels was performed. It is expected that over 12,000 data points, samples should be highly correlated to allow meaningful comparison of the data. Correlation coefficients were within the range of 0.85-0.93 for each comparison (data not shown). Two samples were processed in duplicate by multiple hybridizations to HU95A arrays. The reproducibility of the Affymetrix system was illustrated by the r² values of 0.97 and 0.99. For

Analysis of gene expression from genes differentially regulated in peripheral blood leukocytes. Genes or protein products previously found to be differentially regulated in blood were investigated. The mean and variance of expression levels were calculated across the SZ and Control groups. Altered expression levels (SZ v Controls for each gene) for the dopamine D₃ receptor (+20%), IL-1 receptor antagonist (+30%), IL-2 (−16%), CD3 (+44%), CD4 (+49%), CD8 (+66%), VLA-4 (+33%) and TNF-α(+185%) were found to concur with previously published findings (Ilani et al., Proc Natl Acad Sci USA 2001; 98(2):625-628; Akiyama. Schizophr Res 1999; 37(1), 97-106; Kim, et al., Biol Psychiatry 1998; 43(9):701-4; Sperner-Unterweger et al., Schizophr Res 1999; 38(1):61-70; Muller et al., Am J Psychiatry 1999; 156(4):634-6; Cazzullo et al., Schizophr Res 1998 31(1):49-55; Naudin et al., Schizophr Res 1997 26(2-3); 227-33). Interestingly, found many groups of genes were found that were more significantly altered between the two subject groups, showing the power of this microarray approach to identify patterns of differentially regulated genes. A few examples of genes that have previously been implicated in studies of SZ or other psychiatric disorders are; neural cell adhesion molecule (N-CAM), +112%, p=0.008, GABA-A receptor, +247%, p=0.0003, L-1 type, calcium channel, +39%, p=0.03, 14-3-3 protein eta chain, −30%, p=0.008, and Ciliary neurotrophic factor, +144%, p=0.005.

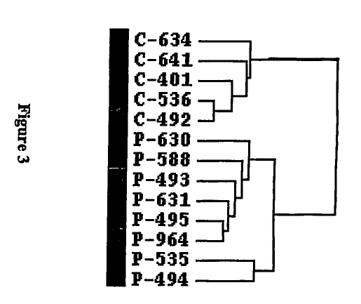

Hierarchical Clustering of SZ Subjects from Control Subjects. Following filtering of the data, a total of 2635 genes remained for further investigation. It may prove useful to perform a supervised clustering experiment, as surrogate tissue (blood leukocytes) is employed in which differences in the patterns of gene expression from SZ patients compared to control subjects may be more subtle than in tissues such as brain. A two-tailed t-test across the 2695 genes expressed in the subject's leukocytes was performed, however, for this analysis the non-medicated subject (Subject 964) was not included. Of the original 2695 genes, 513 were found to have expression values significantly different between the SZ subject group and control group (p<0.05), and 948 were found to have p<0.1 between the two groups. Interestingly, an identical t-test on randomized data was performed, where subject samples were randomly placed into one of two groups. This was repeated for multiple permutated datasets, and the mean numbers of differentially regulated genes calculated. 52 genes were found to be significantly different between the randomized groups (p<0.05), while only 122 genes were found to have p<0.1. Thus, randomizing the data results in a vast decrease in the number of genes found to be differentially expressed between subject groups, and may represent the noise of this experimental system. A clustering experiment was implemented on the 948 genes that differentiated between the subject groups (p<0.1, medicated SZ and controls), with the inclusion of subject 964 in this analysis. Expression levels of the 948 genes for each subject (n=13), were input into the cluster program and the results visualized in TreeView. FIG. 3 shows a partial TreeView figure of the subject cluster results. Two interesting observations were noted, 1) SZ subjects do not appear to cluster based on medication profile, for example, the three SZ subjects receiving Clozapine, (P-494, 535, and 631), do not appear within the same cluster subgroup, while subject 964, a never medicated SZ subject clusters with the SZ group, away from the control subjects, and 2) The smoking status of subjects does not appear to influence the segregation of subjects within the clusters (C-401, 641 and 492 smoke, as do all medicated SZ subjects, but not SZ subject 964). The results of multiple permutations of intra-subject randomization within the data-sets suggesting that these cluster results are not directed by random expression levels in the microarray datasets (data not shown). Preliminary analysis for these studies was performed, and we expect that use of larger subject numbers for each group and a more conservative analysis (p<0.05), will allow further investigation of factors affecting classification of subjects, prior to input into Cluster.

Concordance of expression of the Never-Medicated SZ Subject with Medicated SZ Subjects. When subject 964 was added to the SZ patient subject group for significance testing (two-tailed t-tests), versus the healthy control group, there was a 33% increase in the total number of genes that were differentially expressed (p<0.05) between the 2 subject groups, further indicating the concordance of the neuroleptic naive subject with the remainder of the SZ subject group. Additionally, t-testing between the SZ and control subject group, resulted in decreases in p-value (increased significance) for over 79.5% of the genes previously found to be differentially expressed between subject groups prior to the inclusion of subject 964.

Analysis of Leukocyte Gene Expression in SZ and Bipolar Disorder (BPD) Subjects. For additional data to support this application to investigate leukocyte gene expression profiles for classification of SZ and BPD, two further subjects were recruited and analyzed with a diagnosis of BPD (using the last of the B/START funds). In addition, we have also recruited one subject with major depression into the study. Although that these numbers are very small, this data supports the hypothesis presented herein, and therefore illustrates the value of continuing this investigation.

Analysis of Gene Expression From Genes Differentially Regulated in Peripheral Blood Leukocytes. Expression level data for genes previously found to be differentially regulated in SZ and BPD were investigated. The mean and variance of expression levels were calculated between the groups. Although the data is not statistically significant due to the small subject numbers, transcript levels of TNF-α were ˜100% increased in the SZ versus BPD. Other genes found to be differentially regulated include (SZ v BPD for each gene): IL-1 receptor antagonist (+82%), IL-1 beta (+47%) and dopamine D₃ receptor, (−83%). Many other genes that have also been implicated in psychiatric disorders were found to be differentially expressed between SZ and BPD subjects. For comparison, relative expression of those described above is listed as follows: IL-2 (+92%), CD3 (+42%), CD4 (−25%), CD8 (+36%), N-CAM (+56%), GABA-A receptor (+192%), L-1 type, calcium channel (+32%), 14-3-3 protein eta chain (−79%), and Ciliary neurotrophic factor, (+62%).

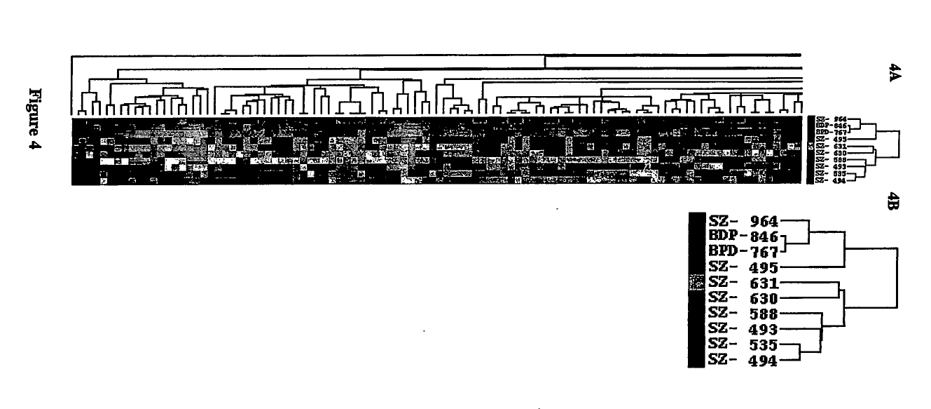

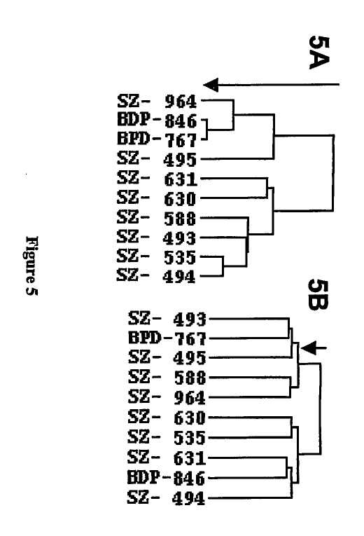

Hierarchical Clustering of SZ and BPD Subjects. A supervised analysis was then performed using all genes found to be differentially expressed between the SZ and BPD subjects (p<01, n=1002). While this result is not significant, it provides some indication into the likelihood of generating classification profiles when larger subject numbers are employed. The TreeView readout in FIG. 4A shows representative samples nodes of similar gene expression (vertical axis), ordered by the total gene expression among the 10 subjects (horizontal axis), where in this example expression levels in the SZ subject samples are lower than in both patients with BPD. The scaled horizontal cluster of subjects (FIG. 4B) indicates that distinctive patterns of gene expression can classify subjects into groups as shown by the sub-nodes within the tree diagram. It was observed that based on the overall gene expression of 1002 genes the two BPD patients (BPD-767, 846) clustered into one discrete sub-node away from the SZ patients. Subjects SZ-964 and 495 appears to cluster into a separate branch of the tree when compared to the other SZ subjects, and suggests that the use of additional subjects should allow further investigation of the actual sub-groupings within the subject clusters. To perform a preliminary investigation on this clustering result, subject gene expression levels were randomized within the dataset and the resultant data were re-clustered. One example readout is shown in FIG. 4, where intra-subject randomization of the data was performed. FIG. 5A shows the TreeView readout from the initial clustering of 1002 genes, as described above. FIG. 5B shows the TreeView readout generated following analysis of the randomized dataset. The short branch lengths between each node of the dendrogram imply that following randomization, subjects have overall gene expression patterns very similar to each other. The Cluster analysis of the other random data iterations, resulted in TreeView readouts where either the samples remained in the order of input into Cluster, or alternatively branch lengths were observed to be vastly reduced, indicating very minor differences in overall gene expression between subjects. These results may suggest that the separation of subjects into nodes within the TreeView diagram is not due to random gene expression levels in the microarray datasets.

Table 2 shows a list of up- or down-regulated genes from PBLs of the eight schizophrenia subjects.

TABLE 2
Schizophrenia Gene Expression Results
This table includes gene expression profile data from 8 schizophrenic subjects
versus 5 control subjects. The table includes the Affymetrix probe-set ID for the HU95Av2
GeneChip array, and also the EASE assignment. The EASE data were included because
there are instances where an unknown EST (as referenced to by the Affymetrix probeset
ID) has later been characterized by others. However, these curation methods are not 100%
accurate.
It is very important to note that the significance levels for the genes/ESTs can
change with increasing statistical power from comparing additional samples. Therefore, it
may be likely that some genes/ESTs may change in significance.

| | mean | | |
|---|---|---|---|
| | expression | | |
| | in | | |
| | schizophrenic | | |
| | patients | | |
| Affymetrix HU95A | compared to | two tailed | |
| version2 probe set | healthy | Students t-test | |
| ids | controls | significance | EASE Names (david.niaid.nih.gov/david/ease.htm) |
| 37444_at | up | 5.39844E−05 | par-6 partitioning defective 6 homolog alpha (C. elegans) |
| 37830_at | up | 6.10988E−05 | transmembrane 4 superfamily member tetraspan NET-5 |
| 1112_g_at | up | 6.37745E−05 | neural cell adhesion molecule 1 |
| 34480_at | up | 7.1664E−05 | cadherin 16, KSP-cadherin |
| 38736_at | down | 0.000100789 | WD repeat domain 1 |
| 1390_s_at | up | 0.000104589 | growth hormone releasing hormone |
| 37294_at | down | 0.000120464 | B-cell translocation gene 1, anti-proliferative |
| 34035_at | up | 0.000133975 | solute carrier family 10 (sodium/bile acid cotransporter family), |
| | | | member 1 |
| 33123_at | down | 0.000140899 | HRIHFB2206 protein |
| 35527_at | up | 0.000163856 | calcium channel, voltage-dependent, alpha 2/delta subunit 1 |
| 32206_at | up | 0.000172847 | CDC42 binding protein kinase alpha (DMPK-like) |
| 39428_at | down | 0.000182565 | lymphocyte adaptor protein |
| 41026_f_at | up | 0.000185284 | glycophorin B (includes Ss blood group) |
| 37388_at | up | 0.000226625 | tissue factor pathway inhibitor 2 |
| 38691_s_at | up | 0.000249289 | surfactant, pulmonary-associated protein C |
| 31700_at | up | 0.000257409 | G protein-coupled receptor 35 |
| 40107_at | up | 0.000263914 | aldolase C, fructose-bisphosphate |
| 35541_r_at | up | 0.000282816 | KIAA0506 protein |
| 36441_at | up | 0.000286506 | |
| 33177_at | up | 0.000290145 | hypothetical protein MGC4293 |
| 1836_at | down | 0.000307175 | cyclin I |
| 37059_at | up | 0.000311251 | glucokinase (hexokinase 4) regulatory protein |
| 34178_at | up | 0.000352425 | zinc finger protein 297 |
| 37631_at | up | 0.000379094 | myosin IE |
| 34011_at | up | 0.000383332 | harakiri, BCL2 interacting protein (contains only BH3 domain) |
| 31924_at | up | 0.000406589 | testicular soluble adenylyl cyclase |
| 40354_at | up | 0.000434143 | heat shock protein (hsp110 family) |
| 39016_r_at | up | 0.00044483 | keratin 6A |
| 34213_at | up | 0.000460936 | KIBRA protein |
| 480_at | up | 0.000466565 | membrane-associated tyrosine- and threonine-specific cdc2- |
| | | | inhibitory kinase |
| 35952_at | up | 0.00048402 | |
| 33727_r_at | up | 0.000532272 | tumor necrosis factor receptor superfamily, member 6b, decoy |
| 32671_at | up | 0.000563318 | KIAA0173 gene product |
| 41714_at | up | 0.000580592 | KIAA0455 gene product |
| 36000_at | up | 0.000583128 | cAMP responsive element binding protein-like 1 |
| 37473_at | up | 0.000586685 | keratin 16 (focal non-epidermolytic palmoplantar keratoderma) |
| 934_at | up | 0.00059989 | glycosylphosphatidylinositol specific phospholipase D1 |
| 35996_at | up | 0.00060253 | ZW10 interactor anti-sense |
| 38007_at | up | 0.000616603 | neurofibromin 2 (bilateral acoustic neuroma) |
| 1187_at | up | 0.000619655 | ligase III, DNA, ATP-dependent |
| 32701_at | up | 0.000630146 | armadillo repeat gene deletes in velocardiofacial syndrome |
| 33960_s_at | up | 0.00065576 | calcium channel, voltage-dependent, L type, alpha 1B subunit |
| 37584_at | up | 0.000662109 | Fanconi anemia, complementation group G |
| 37551_at | up | 0.000674595 | KIAA0211 gene product |
| 1937_at | up | 0.00067796 | |
| 33277_at | up | 0.000693507 | myotubularin related protein 2 |
| 36237_at | up | 0.000693967 | solute carrier family 22 (organic anion transporter), member 6 |
| 41377_f_at | up | 0.000700545 | UDP glycosyltransferase 2 family, polypeptide B7 |
| 35858_at | up | 0.000706501 | postmeiotic segregation increased 2-like 9 |
| 31495_at | up | 0.000713236 | chemokine (C motif) ligand 2 |
| 37413_at | up | 0.000724544 | dipeptidase 1 (renal) |
| 36222_at | up | 0.000729569 | lymphocyte antigen 6 complex, locus G6C |
| 39279_at | down | 0.000742449 | bone morphogenetic protein 6 |
| 37658_at | up | 0.000769593 | growth arrest-specific 6 |
| 34209_at | up | 0.000774852 | inositol 1,4,5-trisphosphate 3-kinase C |
| 34963_at | up | 0.000798158 | collagen, type XIV, alpha 1 (undulin) |
| 41081_at | up | 0.00080125 | BUB1 budding uninhibited by benzimidazoles 1 homolog (yeast) |
| 40997_at | up | 0.000802266 | mitogen-activated protein kinase kinase kinase 12 |
| 35384_at | up | 0.000812766 | histamine receptor H1 |
| 268_at | down | 0.000882562 | platelet/endothelial cell adhesion molecule (CD31 antigen) |
| 35890_at | up | 0.000883719 | sema domain, immunoglobulin domain (Ig), short basic domain, |
| | | | secreted, (semaphorin) 3D |
| 33778_at | up | 0.000883921 | chromosome 22 open reading frame 4 |
| 258_at | up | 0.000904043 | lymphotoxin alpha (TNF superfamily, member 1) |
| 35219_at | up | 0.000908143 | hypothetical protein MGC3047 |
| 35176_at | up | 0.000955593 | dolichyl-phosphate (UDP-N-acetylglucosamine) N- |
| | | | acetylglucosaminephosphotransferase 1 |
| | | | (GlcNAc-1-P transferase) |
| 32162_r_at | up | 0.000971421 | |
| 31391_at | up | 0.001022205 | huntingtin-associated protein 1 (neuroan 1) |
| 34479_at | up | 0.001039336 | phosphoinositide-3-kinase, regulatory subunit, polypeptide 3 |
| | | | (p55, gamma) |
| 738_at | down | 0.001062791 | 5′-nucleotidase, cytosolic II |
| 35719_at | down | 0.001077778 | pleckstrin homology domain containing, family E |
| | | | (with leucine rich repeats) member 1 |
| 236_at | up | 0.001119267 | guanine nucleotide binding protein (G protein), alpha activating |
| | | | activity polypeptide O |
| 33779_at | up | 0.001119359 | vesicle-associated membrane protein 1 (synaptobrevin 1) |
| 31653_at | up | 0.001120702 | peter pan homolog (Drosophila) |
| 41644_at | up | 0.001148448 | KIAA0790 protein |
| 35312_at | up | 0.001149439 | MCM2 minichromosome maintenance deficient 2, mitotin (S. cerevisiae) |
| 38202_at | up | 0.001170423 | FAT tumor suppressor homolog 2 (Drosophila) |
| 1943_at | up | 0.001242703 | cyclin A2 |
| 34894_r_at | up | 0.001244784 | protease, serine, 22 |
| 38162_at | up | 0.001251007 | regulating synaptic membrane exocytosis 2 |
| 689_at | up | 0.001251663 | paraneoplastic antigen |
| 41694_at | up | 0.001259985 | polymerase (RNA) III (DNA directed) polypeptide D, 44 kDa |
| 31991_at | up | 0.0012619 | |
| 41507_at | up | 0.001276543 | mitogen-activated protein kinase-activated protein kinase 5 |
| 34949_at | up | 0.001318033 | adaptor-associated kinase 1 |
| 33517_f_at | up | 0.001327311 | melanoma antigen, family A, 3 |
| 41483_s_at | down | 0.001346791 | jun D proto-oncogene |
| 41641_at | up | 0.001347939 | GPI-anchored metastasis-associated protein homolog |
| 35313_at | down | 0.001365937 | KIAA0310 gene product |
| 37779_at | up | 0.001386546 | acid sphingomyelinase-like phosphodiesterase |
| 388 _at | up | 0.001392133 | loricrin |
| 1499_at | down | 0.001398505 | farnesyltransferase, CAAX box, alpha |
| 35197_at | up | 0.001406638 | |
| 35853_at | up | 0.001414183 | protein kinase C, alpha binding protein |
| 35932_at | up | 0.001424663 | left-right determination, factor B |
| 39568_g_at | up | 0.001432152 | aquaporin 7 |
| 32000_g_at | up | 0.001434142 | ATP-binding cassette, sub-family A (ABC1), member 1 |
| 37436_at | up | 0.001447929 | mitochondrial capsule selenoprotein |
| 34235_at | up | 0.001481689 | G protein-coupled receptor 116 |
| 36907_at | up | 0.001501258 | mevalonate kinase (mevalonic aciduria) |
| 31882_at | up | 0.001503925 | RNA, U3 small nucleolar interacting protein 2 |
| 36535_at | down | 0.001515459 | microfibrillar-associated protein 1 |
| 1196_at | up | 0.001528997 | chromosome condensation 1 |
| 35505_at | up | 0.00152955 | SWI/SNF related, matrix associated, actin dependent |
| | | | regulator of chromatin, subfamily f, member 1 |
| 41118_at | up | 0.001529915 | hypothetical protein FLJ13639 |
| 34770_at | down | 0.001530656 | mitogen-activated protein kinase kinase kinase 8 |
| 37525_at | up | 0.001547367 | serine protease inhibitor-like, with Kunitz and WAP domains 1 |
| | | | (eppin) |
| 1285_at | up | 0.001552251 | |
| 643_at | up | 0.001552712 | nuclear receptor subfamily 0, group B, member 2 |
| 33031_at | up | 0.001558992 | |
| 37415_at | up | 0.001572633 | ATPase, Class V, type 10B |
| 38353_at | down | 0.001599369 | tubulin, gamma complex associated protein 3 |
| 32106_at | up | 0.001599378 | serine (or cysteine) proteinase inhibitor, clade A (alpha-1 |
| | | | antiproteinase, antitrypsin), member 4 |
| 31726_at | up | 0.001621904 | gamma-aminobutyric acid (GABA) A receptor, alpha 3 |
| 38027_at | up | 0.001641281 | fibulin 1 |
| 32420_at | up | 0.001677644 | G protein-coupled receptor 6 |
| 33854_at | down | 0.00173595 | ATPase, H+ transporting, lysosomal 34 kDa, V1 subunit D |
| 39101_at | up | 0.001763484 | myosin, heavy polypeptide 2, skeletal muscle, adult |
| 41502_at | up | 0.001776846 | homeodomain interacting protein kinase 3 |
| 39354_at | down | 0.001786265 | peroxiredoxin 6 |
| 39862_at | up | 0.00179467 | KIAA0296 gene product |
| 38982_at | down | 0.001798352 | telomeric repeat binding factor 2, interacting protein |
| 33640_at | up | 0.001802226 | allograft inflammatory factor 1 |
| 34131_at | up | 0.001817414 | solute carrier family 7, (cationic amino acid transporter, |
| | | | y+ system) member 11 |
| 31686_at | up | 0.001831044 | tubulin, beta polypeptide 4, member Q |
| 33648_at | up | 0.001850125 | |
| 35035_at | up | 0.001852417 | cholinergic receptor, nicotinic, beta polypeptide 3 |
| 39570_at | up | 0.001878741 | hypothetical protein DKFZp434G2311 |
| 38125_at | up | 0.001882819 | serine (or cysteine) proteinase inhibitor, clade E (nexin, |
| | | | plasminogen activator inhibitor type 1), member 1 |
| 37978_at | up | 0.001896075 | quinolinate phosphoribosyltransferase (nicotinate-nucleotide |
| | | | pyrophosphorylase (carboxylating)) |
| 32010_at | up | 0.001904026 | hypothetical protein EAN57 |
| 39609_at | up | 0.00193915 | single-minded homolog 2 (Drosophila) |
| 39622_at | up | 0.001950092 | glial cells missing homolog 1 (Drosophila) |
| 38707_r_at | up | 0.001970767 | E2F transcription factor 4, p107/p130-binding |
| 39520_at | up | 0.001987141 | KIAA0692 protein |
| 34506_at | up | 0.001990895 | aminolevulinate, delta-, dehydratase |
| 41771_g_at | up | 0.001997028 | monoamine oxidase A |
| 36281_at | up | 0.002006192 | nescient helix loop helix 1 |
| 39899_at | up | 0.002018086 | TSLC1-like 2 |
| 262_at | down | 0.002038292 | adenosylmethionine decarboxylase 1 |
| 33281_at | up | 0.002042034 | inhibitor of kappa light polypeptide gene enhancer in B-cells, |
| | | | kinase epsilon |
| 36730_at | up | 0.002098173 | ATP-binding cassette, sub-family C (CFTR/MRP), member 10 |
| 40236_at | up | 0.002111687 | solute carrier family 7 (cationic amino acid transporter, |
| | | | y+ system), member 2 |
| 36731_g_at | up | 0.002115082 | ATP-binding cassette, sub-family C (CFTR/MRP), member 10 |
| 282_at | up | 0.002123585 | M-phase phosphoprotein 1 |
| 41647_at | up | 0.002129848 | STE20-like kinase |
| 33080_s_at | up | 0.002156909 | RAP1, GTPase activating protein 1 |
| 558_at | up | 0.002159946 | keratin 1 (epidermolytic hyperkeratosis) |
| 39714_at | down | 0.002160768 | SH3 domain binding glutamic acid-rich protein like |
| 34630_s_at | up | 0.002160768 | dynein, axonemal, heavy polypeptide 9 |
| 41200_at | up | 0.002177956 | scavenger receptor class B, member 1 |
| 40020_at | up | 0.002182404 | cadherin, EGF LAG seven-pass G-type receptor 3 (flamingo |
| | | | homolog, Drosophila) |
| 37953_s_at | up | 0.002214218 | amiloride-sensitive cation channel 2, neuronal |
| 34425_at | up | 0.002221153 | major histocompatibility complex, class I-related |
| 40957_at | down | 0.002223327 | joined to JAZF1 |
| 2046_at | up | 0.002238232 | |
| 31628_at | up | 0.00224761 | solute carrier family 15 (oligopeptide transporter), member 1 |
| 34590_at | up | 0.002247733 | ciliary neurotrophic factor |
| 40219_at | up | 0.002257582 | HMBA-inducible |
| 35502_at | up | 0.00228274 | anti-Mullerian hormone receptor, type II |
| 32640_at | up | 0.00229347 | intercellular adhesion molecule 1 (CD54), human rhinovirus |
| | | | receptor |
| 31596_f_at | up | 0.0023025 | immunoglobulin lambda-like polypeptide 2 |
| 35920_at | up | 0.00234203 | hemoglobin, beta pseudogene 1 |
| 41013_at | up | 0.002357083 | |
| 36084_at | up | 0.00236503 | KIAA0076 gene product |
| 38171_at | up | 0.00238626 | WD-repeat protein |
| 41209_at | up | 0.002391825 | lipoprotein lipase |
| 38858_at | up | 0.002394103 | potassium voltage-gated channel, subfamily H (eag-related), |
| | | | member 2 |
| 40399_r_at | up | 0.002401232 | mesenchyme homeo box 2 (growth arrest-specific homeo box) |
| 32141_at | up | 0.002413611 | protein phosphatase 1E (PP2C domain containing) |
| 1681_at | up | 0.002427249 | estrogen receptor 1 |
| 32681_at | up | 0.002434539 | solute carrier family 9 (sodium/hydrogen exchanger), isoform 1 |
| | | | (antiporter, Na+/H+, amiloride sensitive) |
| 39097_at | down | 0.002438561 | SON DNA binding protein |
| 435_g_at | up | 0.002444462 | H1 histone family, member 0 |
| 40400_at | up | 0.002448507 | adenosine A1 receptor |
| 34704_r_at | up | 0.002448849 | chorionic somatomammotropin hormone 2 |
| 1662_r_at | up | 0.002482635 | |
| 35448_at | up | 0.002497094 | peptidylprolyl isomerase (cyclophilin)-like 2 |
| 32498_at | up | 0.002516445 | glutamate receptor, metabotropic 2 |
| 34370_at | down | 0.002528889 | archain 1 |
| 396_f_at | up | 0.002531111 | erythropoietin receptor |
| 1035_g_at | up | 0.002533413 | tissue inhibitor of metalloproteinase 3 (Sorsby fundus dystrophy, |
| | | | pseudoinflammatory) |
| 38957_at | up | 0.00254188 | doublecortin and CaM kinase-like 1 |
| 35921_at | up | 0.002559242 | hemoglobin, beta pseudogene 1 |
| 873_at | up | 0.002587161 | homeo box A5 |
| 545_g_at | down | 0.002588546 | nuclear factor of kappa light polypeptide gene enhancer in |
| | | | B-cells 2 (p49/p100) |
| 309_f_at | up | 0.002594278 | growth hormone 2 |
| 1442_at | up | 0.002597237 | estrogen receptor 2 (ER beta) |
| 1814_at | down | 0.002601426 | transforming growth factor, beta receptor II (70/80 kDa) |
| 37153_at | up | 0.002626944 | nephronophthisis 4 |
| 41807_at | down | 0.002629111 | sin3-associated polypeptide, 18 kDa |
| 38513_at | up | 0.00264673 | processing of precursors 1 |
| 33403_at | down | 0.002651277 | DKFZP547E1010 protein |
| 786_at | up | 0.002669364 | polymerase (DNA directed), alpha |
| 31975_at | up | 0.002694514 | |
| 38495_s_at | up | 0.00271851 | fucosyltransferase 3 (galactoside 3(4)-L-fucosyltransferase, |
| | | | Lewis blood group included) |
| 714_at | up | 0.002720175 | |
| 38482_at | up | 0.00272614 | claudin 7 |
| 1967_f_at | up | 0.002739683 | |
| 39240_at | up | 0.002744223 | neurexin 1 |
| 37575_at | down | 0.002760815 | |
| 1063_s_at | up | 0.0027749 | TYRO3 protein tyrosine kinase |
| 31590_g_at | up | 0.002776136 | |
| 37898_r_at | up | 0.002781402 | trefoil factor 3 (intestinal) |
| 37983_at | up | 0.002787601 | angiotensin II receptor, type 1 |
| 37273_at | up | 0.002809352 | |
| 926_at | up | 0.002825805 | metallothionein 1G |
| 34293_at | up | 0.002829536 | kinesin family member C3 |
| 33821_at | down | 0.00283335 | homolog of yeast long chain polyunsaturated fatty acid |
| | | | elongation enzyme 2 |
| 36840_at | up | 0.002837181 | hypothetical protein FLJ10737 |
| 31889_at | up | 0.002846254 | melan-A |
| 37151_at | up | 0.002860716 | |
| 32201_at | up | 0.002868158 | Sjogren's syndrome nuclear autoantigen 1 |
| 414_at | up | 0.002884861 | homeo box D10 |
| 35520_at | up | 0.002889864 | claudin 9 |
| 39666_at | up | 0.002894488 | guanine nucleotide binding protein (G protein), gamma 4 |
| 38621_at | up | 0.002895693 | dimethylarginine dimethylaminohydrolase 2 |
| 970_r_at | up | 0.002920493 | ubiquitin specific protease 9, X chromosome (fat facets-like |
| | | | Drosophila) |
| 41247_at | up | 0.002926933 | |
| 1022_f_at | up | 0.002930839 | interferon, alpha 14 |
| 41500_at | up | 0.002932033 | v-ski sarcoma viral oncogene homolog (avian) |
| 34679_at | up | 0.002938559 | breakpoint cluster region |
| 33942_s_at | up | 0.002939961 | syntaxin binding protein 1 |
| 33454_at | up | 0.002954371 | agrin |
| 32048_at | up | 0.002956969 | |
| 39567_at | up | 0.002960606 | aquaporin 7 |
| 734_at | up | 0.003021185 | |
| 40473_at | down | 0.003051481 | serine/threonine kinase 24 (STE20 homolog, yeast) |
| 160029_at | down | 0.003081574 | protein kinase C, beta 1 |
| 37097_at | up | 0.003086294 | solute carrier family 17 (sodium phosphate), member 1 |
| 32172_at | down | 0.003088013 | SMART/HDAC1 associated repressor protein |
| 38604_at | up | 0.003091956 | neuropeptide Y |
| 34621_at | up | 0.003098553 | keratin 2A (epidermal ichthyosis bullosa of Siemens) |
| 32349_at | up | 0.003130675 | annexin A10 |
| 38928_r_at | up | 0.003143861 | tyrosinase (oculocutaneous albinism IA) |
| 1988_at | up | 0.003144002 | platelet-derived growth factor receptor, alpha polypeptide |
| 1494_f_at | up | 0.003151857 | cytochrome P450, family 2, subfamily A, polypeptide 6 |
| 32156_at | up | 0.003158238 | poliovirus receptor-related 2 (herpesvirus entry mediator B) |
| 34440_at | up | 0.00317265 | DiGeorge syndrome critical region gene 9 |
| 37853_at | up | 0.003195503 | urocortin |
| 39839_at | down | 0.003202074 | cold shock domain protein A |
| 38747_at | up | 0.003205975 | CD34 antigen |
| 40565_at | up | 0.003225285 | apolipoprotein E |
| 31326_at | up | 0.003229141 | |
| 40668_s_at | up | 0.003236501 | CD6 antigen |
| 32923_r_at | up | 0.003262936 | synapsin I |
| 33972_r_at | up | 0.003267304 | deleted in azoospermia-like |
| 2027_at | up | 0.003272896 | S100 calcium binding protein A2 |
| 38038_at | up | 0.003272965 | lumican |
| 34820_at | up | 0.003338528 | pleiotrophin (heparin binding growth factor 8, neurite growth- |
| | | | promoting factor 1) |
| 34197_at | up | 0.003357164 | phosphoinositide-3-kinase, regulatory subunit, polypeptide 2 |
| | | | (p85 beta) |
| 41427_at | up | 0.003380281 | wingless-type MMTV integration site family, member 11 |
| _at | up | 0.003400348 | potassium voltage-gated channel, shaker-related subfamily, |
| | | | beta member 1 |
| 192_at | down | 0.003400695 | TAF7 RNA polymerase II, TATA box binding protein (TBP)- |
| | | | associated factor, 55 kDa |
| 34821_at | down | 0.003403862 | chromosome 6 open reading frame 80 |
| 33712_at | up | 0.003430688 | sulfotransferase family 4A, member 1 |
| 37588_s_at | up | 0.003433268 | mitogen-activated protein kinase 8 interacting protein 2 |
| 37372_at | up | 0.003438532 | sphingomyelin phosphodiesterase 1, acid lysosomal (acid |
| | | | sphingomyelinase) |
| 40372_at | up | 0.003447837 | pancreatic lipase-related protein 1 |
| 441_s_at | up | 0.003458549 | leukemia inhibitory factor (cholinergic differentiation factor) |
| 1849_s_at | down | 0.003460555 | retinoblastoma binding protein 1 |
| 541_g_at | up | 0.003461606 | heat shock 27 kDa protein 2 |
| 32443_at | up | 0.003463616 | zinc finger protein 157 (HZF22) |
| 121_at | up | 0.003467949 | paired box gene 8 |
| 41817_g_at | up | 0.00349005 | caspase recruitment domain family, member 10 |
| 39242_at | up | 0.003510672 | synaptotagmin V |
| 37687_i_at | up | 0.003516194 | Fc fragment of IgG, low affinity IIa, receptor for (CD32) |
| 40302_at | up | 0.003521173 | emilin and multimerin-domain containing protein 1 |
| 39192_at | up | 0.003526575 | tumor necrosis factor receptor superfamily, member 17 |
| 39722_at | up | 0.003536214 | nuclear receptor co-repressor 1 |
| 31936_s_at | down | 0.003552058 | limkain b1 |
| 32407_f_at | up | 0.003585808 | |
| 36304_at | up | 0.00359153 | complement component 8, beta polypeptide |
| 37270_at | up | 0.003593677 | ATPase, Na+/K+ transporting, beta 2 polypeptide |
| 40171_at | down | 0.003618084 | frequently rearranged in advanced T-cell lymphomas 2 |
| 39495_at | up | 0.003639204 | hypothetical protein FLJ20719 |
| 37139_at | up | 0.003644433 | ectodermal dysplasia 1, anhidrotic |
| 31681_at | up | 0.003648646 | erythropoietin receptor |
| 41276_at | up | 0.003680784 | sin3-associated polypeptide, 18 kDa |
| 36469_at | up | 0.003682865 | dystrobrevin, alpha |
| 32810_at | up | 0.003706452 | thiopurine S-methyltransferase |
| 34069_s_at | up | 0.003710418 | synovial sarcoma translocation, chromosome 18 |
| 37087_at | up | 0.003723312 | A kinase (PRKA) anchor protein 4 |
| 32513_at | up | 0.003730609 | neurofilament 3 (150 kDa medium) |
| 614_at | up | 0.003737015 | phospholipase A2, group IIA (platelets, synovial fluid) |
| 1019_g_at | up | 0.003737542 | wingless-type MMTV integration site family, member 10B |
| 36123_at | down | 0.003760113 | thiosulfate sulfurtransferase (rhodanese) |
| 33211_at | up | 0.003774765 | ribosome binding protein 1 homolog 180 kDa (dog) |
| 38541_at | up | 0.003778747 | cytochrome P450, family 21, subfamily A, polypeptide 2 |
| 39343_at | up | 0.003780079 | transformer-2 alpha (htra-2 alpha) |
| 36888_at | up | 0.00379196 | KIAA0841 protein |
| 37312_at | down | 0.003803597 | transcriptional regulator interacting with the PHS-bromodomain 2 |
| 37785_at | up | 0.003817423 | GTP-binding protein |
| 33323_r_at | up | 0.003818801 | stratifin |
| 35633_at | down | 0.003823077 | engulfment and cell motility 1 (ced-12 homolog, C. elegans) |
| 34273_at | up | 0.003831402 | regulator of G-protein signalling 4 |
| 35545_at | up | 0.003835274 | solute carrier family 4, sodium bicarbonate cotransporter, |
| | | | member 8 |
| 33661_at | up | 0.003844513 | ribosomal protein L5 |
| 40359_at | up | 0.003849677 | chromosome 11 open reading frame 13 |
| 37056_at | up | 0.003860515 | tec protein tyrosine kinase |
| 33268_at | up | 0.003860581 | Smcx homolog, X chromosome (mouse) |
| 37618_at | up | 0.003865292 | homeo box B7 |
| 36323_at | up | 0.003868425 | gamma-aminobutyric acid (GABA) A receptor, alpha 1 |
| 31654_at | up | 0.003872787 | VPS10 domain receptor protein SORCS 3 |
| 39990_at | up | 0.003883048 | ISL1 transcription factor, LIM/homeodomain, (islet-1) |
| 38608_at | up | 0.003891136 | lectin, galactoside-binding, soluble, 7 (galectin 7) |
| 35746_r_at | down | 0.003899767 | poly(rC) binding protein 2 |
| 259_s_at | up | 0.003922965 | tumor necrosis factor (TNF superfamily, member 2) |
| 34558_at | up | 0.00393898 | opiate receptor-like 1 |
| 34457_at | up | 0.003943871 | solute carrier family 30 (zinc transporter), member 3 |
| 31771_at | up | 0.003954233 | |
| 32292_at | up | 0.003968658 | collectin sub-family member 10 (C-type lectin) |
| 32171_at | down | 0.003976408 | eukaryotic translation initiation factor 5 |
| 37166_at | up | 0.004008678 | 3-hydroxyanthranilate 3,4-dioxygenase |
| 1612_s_at | down | 0.004008843 | jun D proto-oncogene |
| 38636_at | up | 0.004009285 | immunoglobulin superfamily containing leucine-rich repeat |
| 39939_at | up | 0.004024305 | collagen, type IV, alpha 6 |
| 39459_at | up | 0.004034943 | ribosomal protein S13 |
| 41437_at | down | 0.004042248 | chromosome 14 open reading frame 109 |
| 872_i_at | up | 0.00408932 | insulin receptor substrate 1 |
| 39091_at | down | 0.004093417 | vitamin A responsive; cytoskeleton related |
| 35319_at | down | 0.004100726 | CCCTC-binding factor (zinc finger protein) |
| 33967_at | up | 0.004109676 | major histocompatibility complex, class II, DO alpha |
| 333_s_at | down | 0.004110914 | |
| 39400_at | up | 0.00412939 | KIAA1055 protein |
| 39304_g_at | up | 0.004129597 | beta-transducin repeat containing |
| 37838_at | up | 0.004143313 | coagulation factor XII (Hageman factor) |
| 35970_g_at | down | 0.00414378 | M-phase phosphoprotein 9 |
| 1669_at | up | 0.004147458 | wingless-type MMTV integration site family, member 5A |
| 38822_at | up | 0.004163563 | serine/threonine kinase 17a (apoptosis-inducing) |
| 145_s_at | up | 0.004167288 | T-box 5 |
| 38883_at | up | 0.004187484 | |
| 39917_at | up | 0.004214466 | tubulin, gamma complex associated protein 2 |
| 32650_at | up | 0.004216408 | neuronal protein |
| 35007_at | down | 0.004227977 | |
| 41655_at | up | 0.004234936 | midline 2 |
| 37731_at | down | 0.004243561 | epidermal growth factor receptor pathway substrate 15 |
| 34066_at | up | 0.004275892 | hexose-6-phosphate dehydrogenase (glucose 1-dehydrogenase) |
| 33885_at | down | 0.004286872 | KIAA0907 protein |
| 37025_at | down | 0.004312041 | lipopolysaccharide-induced TNF factor |
| 35699_at | up | 0.004318144 | BUB1 budding uninhibited by benzimidazoles 1 homolog beta |
| | | | (yeast) |
| 36129_at | up | 0.004340344 | KIAA0397 gene product |
| 32629_f_at | up | 0.004351049 | butyrophilin, subfamily 3, member A1 |
| 40625_f_at | up | 0.004352482 | metaxin 1 |
| 37465_at | up | 0.004357604 | brain-specific protein p25 alpha |
| 1700_at | up | 0.00436827 | BCL2 binding component 3 |
| 32345_at | up | 0.00437274 | |
| 35281_at | up | 0.004383388 | laminin, gamma 2 |
| 32358_at | up | 0.004391543 | WNT1 inducible signaling pathway protein 3 |
| 32007_at | up | 0.004446678 | |
| 35803_at | up | 0.004448806 | ras homolog gene family, member E |
| 35630_at | up | 0.004449475 | lethal giant larvae homolog 2 (Drosophila) |
| 33467_at | up | 0.004454387 | CMRF35 leukocyte immunoglobulin-like receptor |
| 41449_at | up | 0.004456743 | sarcoglycan, epsilon |
| 1075_f_at | up | 0.004457857 | interferon, alpha 16 |
| 1567_at | up | 0.004477979 | fms-related tyrosine kinase 1 (vascular endothelial growth |
| | | | factor/vascular permeability factor receptor) |
| 32223_at | up | 0.004484703 | splicing factor, arginine/serine-rich 14 |
| 35745_f_at | down | 0.004489339 | poly(rC) binding protein 2 |
| 32888_at | up | 0.00451066 | leukocyte tyrosine kinase |
| 1777_at | up | 0.004521209 | Ras and Rab interactor 1 |
| 40042_r_at | up | 0.004524196 | proline dehydrogenase (oxidase) 1 |
| 35896_at | up | 0.004592292 | DKFZp434P211 protein |
| 34702_f_at | up | 0.004610914 | chorionic somatomammotropin hormone 2 |
| 1339_s_at | up | 0.004621845 | breakpoint cluster region |
| 1799_at | up | 0.004625646 | excision repair cross-complementing rodent repair deficiency, |
| | | | complementation group 4 |
| 35320_at | up | 0.004625842 | solute carrier family 11 (proton-coupled divalent metal ion |
| | | | transporters), member 2 |
| 37191_at | up | 0.00462609 | phytanoyl-CoA hydroxylase interacting protein |
| 33521_at | up | 0.004640954 | ATPase, H+/K+ exchanging, alpha polypeptide |
| 34527_r_at | up | 0.004649009 | |
| 34467_g_at | up | 0.004656371 | 5-hydroxytryptamine (serotonin) receptor 4 |
| 37760_at | up | 0.004663199 | BAI1-associated protein 2 |
| 33418_at | down | 0.004726336 | |
| 39720_g_at | up | 0.00473358 | zona pellucida glycoprotein 3 (sperm receptor) |
| 32028_at | up | 0.004738157 | phosphomannomutase 2 |
| 35666_at | up | 0.004741066 | sema domain, immunoglobulin domain (Ig), short basic domain, |
| | | | secreted, (semaphorin) 3F |
| 31591_s_at | up | 0.004769075 | complement factor H-related 4 |
| 39009_at | down | 0.004782089 | LSM3 homolog, U6 small nuclear RNA associated (S. cerevisiae) |
| 38847_at | up | 0.004785318 | maternal embryonic leucine zipper kinase |
| 37793_r_at | up | 0.004797672 | RAD51-like 3 (S. cerevisiae) |
| 32837_at | up | 0.004799361 | 1-acylglycerol-3-phosphate O-acyltransferase 2 |
| | | | (lysophosphatidic acid acyltransferase, beta) |
| 32092_at | up | 0.004804199 | syndecan 3 (N-syndecan) |
| 35307_at | down | 0.004819968 | GDP dissociation inhibitor 2 |
| 34141_at | up | 0.004824997 | |
| 40321_at | up | 0.004840659 | interleukin 1 receptor-like 1 |
| 40622_r_at | up | 0.004881409 | ubiquitin protein ligase |
| 39128_r_at | up | 0.004896276 | protein phosphatase 2A, regulatory subunit B′ (PR 53) |
| 36819_at | up | 0.004902066 | Machado-Joseph disease (spinocerebellar ataxia 3, |
| | | | olivopontocerebellar ataxia 3, autosomal dominant, ataxin 3) |
| 36702_at | up | 0.004916649 | T-box 19 |
| 1828_s_at | up | 0.004940536 | fibroblast growth factor 2 (basic) |
| 33047_at | up | 0.004989688 | |
| 41192_at | down | 0.004996772 | hypothetical protein 669 |
| 33134_at | up | 0.005003649 | adenylate cyclase 3 |
| 564_at | up | 0.005026874 | guanine nucleotide binding protein (G protein), alpha 11 (Gq |
| | | | class) |
| 38797_at | up | 0.005038875 | KIAA0062 protein |
| 40276_at | down | 0.005046073 | proteasome (prosome, macropain) 26S subunit, non- |
| | | | ATPase, 7 (Mov34 homolog) |
| 40199_at | up | 0.005054057 | msh homeo box homolog 1 (Drosophila) |
| 31818_at | up | 0.005058057 | |
| 1832_at | up | 0.00506241 | mutated in colorectal cancers |
| 39051_at | up | 0.005064802 | neuronatin |
| 31676_at | up | 0.005081672 | zinc finger protein 208 |
| 32479_at | up | 0.005105492 | tumor necrosis factor receptor superfamily, member 11a, |
| | | | activator of NFKB |
| 39197_s_at | up | 0.005111726 | |
| 39750_at | up | 0.005142581 | zinc finger, DHHC domain containing 3 |
| 39986_at | up | 0.005154791 | hepatocellularcarcinoma-associated antigen HCA557a |
| 37053_at | up | 0.005195125 | ATPase, Ca++ transporting, plasma membrane 2 |
| 32389_at | up | 0.005199094 | RNA, U2 small nuclear |
| 40376_at | up | 0.005209375 | arylsulfatase E (chondrodysplasia punctata 1) |
| 1379_at | up | 0.005219489 | EphA2 |
| 38440_s_at | down | 0.005255831 | hypothetical protein FLJ20811 |
| 33520_at | up | 0.005264186 | coagulation factor VII (serum prothrombin conversion |
| | | | accelerator) |
| 39688_at | up | 0.00527689 | requiem, apoptosis response zinc finger gene |
| 31829_r_at | up | 0.005284494 | trans-golgi network protein 2 |
| 2066_at | up | 0.005318536 | BCL2-associated X protein |
| 38294_at | up | 0.005318622 | homeo box D4 |
| 32971_at | up | 0.00533295 | Friedreich ataxia region gene X123 |
| 32509_at | up | 0.005359441 | HBxAg transactivated protein 2 |
| 41227_at | up | 0.00537468 | oculocerebrorenal syndrome of Lowe |
| 41840_r_at | up | 0.005385137 | |
| 1804_at | up | 0.005407113 | kallikrein 3, (prostate specific antigen) |
| 34703_f_at | up | 0.005425622 | chorionic somatomammotropin hormone 2 |
| 34060_g_at | up | 0.005447522 | Pvt1 oncogene homolog, MYC activator (mouse) |
| 39499_s_at | up | 0.005466238 | par-3 partitioning defective 3 homolog (C. elegans) |
| 32240_at | up | 0.005479107 | proteasome (prosome, macropain) 26S subunit, non-ATPase, 5 |
| 31817_at | up | 0.005494234 | gamma-aminobutyric acid (GABA) A receptor, beta 3 |
| 32077_s_at | up | 0.005543575 | potassium voltage-gated channel, KQT-like subfamily, member 1 |
| 33762_r_at | up | 0.005564914 | KIAA0493 protein |
| 37459_at | up | 0.005565205 | collagen, type VIII, alpha 1 |
| 1240_at | up | 0.00556878 | caspase 2, apoptosis-related cysteine protease (neural precursor |
| | | | cell expressed, developmentally down-regulated 2) |
| 33711_at | up | 0.00557075 | prooplomelanocortin (adrenocorticotropin/beta-lipotropin/alpha- |
| | | | melanocyte stimulating hormone/beta-melanocyte stimulating |
| | | | hormone/beta-endorphin) |
| 31411_at | up | 0.005579666 | variable charge, Y chromosome, 2 |
| 36337_at | up | 0.005593633 | KIAA0963 protein |
| 40340_at | up | 0.005596156 | hypothetical protein DKFZp586E1923 |
| 35536_at | up | 0.005620504 | endothelin converting enzyme 2 |
| 33000_at | up | 0.005629213 | hepatitis A virus cellular receptor 1 |
| 34906_g_at | up | 0.005635263 | glutamate receptor, ionotropic, kainate 5 |
| 37721_at | up | 0.005646504 | deoxyhypusine synthase |
| 32642_at | up | 0.005651069 | chondroitin sulfate proteoglycan 3 (neurocan) |
| 39160_at | down | 0.005660176 | pyruvate dehydrogenase (lipoamide) beta |
| 41264_at | up | 0.005670964 | |
| 34655_at | up | 0.0057165 | membrane protein, palmitoylated 2 (MAGUK p55 subfamily |
| | | | member 2) |
| 31861_at | up | 0.005718235 | immunoglobulin mu binding protein 2 |
| 36734_at | up | 0.005753457 | small proline-rich protein 2A |
| 39310_at | up | 0.005754564 | bradykinin receptor B2 |
| 770_at | up | 0.005756245 | glutathione peroxidase 3 (plasma) |
| 764_s_at | up | 0.005789206 | clock homolog (mouse) |
| 31350_at | up | 0.005792228 | olfactory receptor, family 10, subfamily H, member 3 |
| 40615_at | down | 0.005797033 | hypothetical protein FLJ21439 |
| 38180_f_at | up | 0.005826003 | pregnancy specific beta-1-glycoprotein 9 |
| 33574_at | up | 0.005842528 | chromosome 6 open reading frame 10 |
| 33986_r_at | up | 0.005846995 | heat shock 90 kDa protein 1, beta |
| 31542_at | up | 0.005886628 | filaggrin |
| 36578_at | down | 0.005897302 | baculoviral IAP repeat-containing 2 |
| 31442_at | up | 0.005902455 | |
| 37839_at | up | 0.005906266 | |
| 39445_at | up | 0.005922975 | UDP-Gal:betaGlcNAc beta 1,4-galactosyltransferase, polypeptide 3 |
| 41046_s_at | up | 0.00592598 | zinc finger protein 261 |
| 36546_r_at | up | 0.005940762 | KIAA0542 gene product |
| 40907_at | up | 0.005948234 | hypothetical protein FLJ32130 |
| 160020_at | up | 0.005967887 | matrix metalloproteinase 14 (membrane-inserted) |
| 41793_at | up | 0.005969246 | ATP-binding cassette, sub-family C (CFTR/MRP), member 8 |
| 35899_at | up | 0.00598163 | artemin |
| 41354_at | up | 0.005996623 | stanniocalcin 1 |
| 731_f_at | up | 0.006023842 | |
| 41051_at | down | 0.006083685 | translin-associated factor X |
| 39598_at | up | 0.006099426 | gap junction protein, beta 1, 32 kDa (connexin 32, Charcot-Marie- |
| | | | Tooth neuropathy, X-linked) |
| 40877_s_at | up | 0.006100957 | D15F37 (pseudogene) |
| 39619_at | up | 0.006107927 | SGC32445 protein |
| 40554_at | up | 0.006116743 | golgi phosphoprotein 4 |
| 548_s_at | down | 0.006131531 | spleen tyrosine kinase |
| 230_s_at | up | 0.00614997 | follicle stimulating hormone, beta polypeptide |
| 35637_at | up | 0.006154739 | hypothetical protein PRO2325 |
| 32241_at | down | 0.006160593 | TAR DNA binding protein |
| 2077_at | up | 0.006189769 | integrin, alpha 6 |
| 40825_at | up | 0.00622426 | microtubule-associated protein, RP/EB family, member 3 |
| 39472_s_at | up | 0.006239579 | BRAF35/HDAC2 complex (80 kDa) |
| 38320_s_at | up | 0.006246922 | lipase, hormone-sensitive |
| 37972_at | up | 0.006252496 | deoxyribonuclease I-like 3 |
| 31410_at | up | 0.006253494 | tumor necrosis factor receptor superfamily, member 13B |
| 33594_at | up | 0.006264788 | dickkopf homolog 4 (Xenopus laevis) |
| 40081_at | up | 0.00627529 | phospholipid transfer protein |
| 34301_r_at | up | 0.006279415 | keratin 17 |
| 35329_at | down | 0.006296693 | cytochrome b5 reductase 1 (B5R.1) |
| 31709_at | up | 0.006341656 | nuclear receptor co-repressor 2 |
| 34415_at | up | 0.006342999 | activin A receptor, type IB |
| 31406_at | up | 0.006344356 | G protein-coupled receptor 50 |
| 35254_at | up | 0.006348896 | FLN29 gene product |
| 31930_f_at | up | 0.006378393 | Rhesus blood group, CcEe antigens |
| 38460_at | up | 0.006382402 | cytochrome b-5 |
| 31537_at | up | 0.006385628 | ADP-ribosyltransferase 1 |
| 33538_at | up | 0.006387439 | myelin expression factor 2 |
| 916_at | up | 0.006393536 | protein tyrosine phosphatase, receptor type, N |
| 35599_at | up | 0.006406575 | glycine N-methyltransferase |
| 35950_at | up | 0.006461703 | synovial sarcoma, X breakpoint 4 |
| 38468_at | up | 0.006474053 | Hermansky-Pudlak syndrome 1 |
| 1792_g_at | up | 0.006476549 | cyclin-dependent kinase 2 |
| 33064_at | up | 0.006495075 | calcium channel, voltage-dependent, gamma subunit 1 |
| 1680_at | up | 0.006503388 | growth factor receptor-bound protein 7 |
| 34429_at | up | 0.006513609 | mucosal vascular addressin cell adhesion molecule 1 |
| 36600_at | down | 0.006518042 | proteasome (prosome, macropain) activator subunit 1 (PA28 |
| | | | alpha) |
| 34108_g_at | up | 0.006518826 | 6-phosphofructo-2-kinase/fructose-2,6 biphosphatase 2 |
| 36727_at | up | 0.006526838 | |
| 732_f_at | up | 0.006547759 | |
| 31668_f_at | up | 0.006549214 | erythrocyte membrane protein band 4.1-like 2 |
| 33784_at | up | 0.006564337 | TNF receptor-associated factor 2 |
| 40570_at | down | 0.006567141 | forkhead box O1A (rhabdomyosarcoma) |
| 34853_at | up | 0.006610556 | fibronectin leucine rich transmembrane protein 2 |
| 571_at | down | 0.006623427 | nucleosome assembly protein 1-like 1 |
| 33532_at | up | 0.006633032 | cartilage paired-class homeoprotein 1 |
| 38597_f_at | up | 0.00663979 | solute carrier family 11 (proton-coupled divalent metal ion |
| | | | transporters), member 1 |
| 31534_at | up | 0.006657321 | zinc finger protein, Y-linked |
| 35248_at | up | 0.006666707 | solute carrier family 19 (thiamine transporter), member 2 |
| 35938_at | down | 0.006675236 | phospholipase A2, group IVA (cytosolic, calcium-dependent) |
| 32430_at | up | 0.006677844 | gastrin-releasing peptide receptor |
| 35923_at | up | 0.006705555 | cholecystokinin B receptor |
| 40145_at | up | 0.006774519 | topoisomerase (DNA) II alpha 170 kDa |
| 35013_at | up | 0.006804943 | lipopolysaccharide binding protein |
| 35898_at | up | 0.00681255 | WNT1 inducible signaling pathway protein 2 |
| 41331_at | up | 0.006837963 | leucine-rich repeats and immunoglobulin-like domains 2 |
| 34308_at | down | 0.006842048 | histone 1, H2ac |
| 34795_at | up | 0.006845198 | procollagen-lysine, 2-oxoglutarate 5-dioxygenase (lysine |
| | | | hydroxylase) 2 |
| 38205_at | up | 0.006846391 | neurogenic differentiation 2 |
| 739_at | up | 0.006849272 | neurofibromin 1 (neurofibromatosis, von Recklinghausen disease, |
| | | | Watson disease) |
| 1208_at | up | 0.006860724 | PTK6 protein tyrosine kinase 6 |
| 38237_at | up | 0.006873965 | gamma-glutamyltransferase-like activity 1 |
| 37061_at | up | 0.006878942 | chitinase 1 (chitotriosidase) |
| 31897_at | up | 0.006912079 | downregulated in ovarian cancer 1 |
| 34259_at | up | 0.006921783 | KIAA0664 protein |
| 33510_s_at | up | 0.006943429 | glutamate receptor, metabotropic 1 |
| 37547_at | up | 0.006943934 | PTH-responsive osteosarcoma B1 protein |
| 38032_at | up | 0.006964957 | synaptic vesicle glycoprotein 2A |
| 41799_at | up | 0.006993973 | DnaJ (Hsp40) homolog, subfamily C, member 7 |
| 775_at | up | 0.00699531 | 5-hydroxytryptamine (serotonin) receptor 1B |
| 34175_r_at | up | 0.00700068 | |
| 36833_at | down | 0.007015057 | galactosidase, alpha |
| 34405_at | up | 0.007020147 | ubiquitin specific protease 5 (isopeptidase T) |
| 31745_at | up | 0.007034964 | mucin 3A, intestinal |
| 38507_at | up | 0.007046638 | cytochrome P450, family 2, subfamily D, polypeptide 6 |
| 38504_at | up | 0.00705255 | calpain 5 |
| 31921_at | up | 0.007070315 | olfactory receptor, family 2, subfamily F, member 1 |
| 715_s_at | up | 0.007073157 | gamma-glutamyltransferase 1 |
| 214_at | up | 0.007077911 | msh homeo box homolog 1 (Drosophila) |
| 41833_at | up | 0.007130788 | jumping translocation breakpoint |
| 36883_at | up | 0.007136738 | keratin 13 |
| 36404_at | up | 0.007145174 | glucagon-like peptide 1 receptor |
| 799_at | up | 0.007165141 | cyclin-dependent kinase 5, regulatory subunit 1 (p35) |
| 39805_at | up | 0.00717185 | ATP-binding cassette, sub-family B (MDR/TAP), member 6 |
| 38136_at | up | 0.007176078 | Werner syndrome |
| 38193_at | up | 0.007185711 | immunoglobulin kappa constant |
| 37939_at | up | 0.007225072 | apolipoprotein B mRNA editing enzyme, catalytic polypeptide-like |
| | | | 3C |
| 265_s_at | up | 0.00723378 | selectin E (endothelial adhesion molecule 1) |
| 39196_i_at | up | 0.007240769 | leucine-rich repeats and immunoglobulin-like domains 1 |
| 37129_at | up | 0.007242263 | neuropeptide FF-amide peptide precursor |
| 35243_at | up | 0.007262299 | PCTAIRE protein kinase 3 |
| 34167_s_at | up | 0.007272108 | solute carrier family 6 (neurotransmitter transporter, L-proline), |
| | | | member 7 |
| 40523_at | up | 0.007280257 | forkhead box A2 |
| 1925_at | up | 0.007284827 | cyclin F |
| 41340_at | up | 0.00729114 | sema domain, immunoglobulin domain (Ig), and GPI membrane |
| | | | anchor, (semaphorin) 7A |
| 911_s_at | down | 0.007294306 | calmodulin 2 (phosphorylase kinase, delta) |
| 1463_at | down | 0.007297165 | protein tyrosine phosphatase, non-receptor type 12 |
| 35816_at | down | 0.007313124 | cystatin B (stefin B) |
| 34061_at | up | 0.00731512 | Pvt1 oncogene homolog, MYC activator (mouse) |
| 363_at | up | 0.007332831 | protein kinase C, gamma |
| AFFX-BioC-3_at | up | 0.007343487 | |
| 37638_at | up | 0.007365403 | dedicator of cyto-kinesis 1 |
| 37778_at | down | 0.007365593 | KIN, antigenic determinant of recA protein homolog (mouse) |
| 36252_at | up | 0.007369173 | cardiotrophin 1 |
| 33568_at | up | 0.007372628 | cholinergic receptor, nicotinic, beta polypeptide 4 |
| 37432_g_at | up | 0.007374815 | Msx-interacting-zinc finger |
| 35317_at | down | 0.007374906 | meningioma expressed antigen 5 (hyaluronidase) |
| 1100_at | down | 0.007398264 | interleukin-1 receptor-associated kinase 1 |
| 31841_at | up | 0.007402655 | catenin (cadherin-associated protein), alpha 2 |
| 34754_at | up | 0.007404094 | ezrin-binding partner PACE-1 |
| 921_s_at | up | 0.007411006 | |
| 39099_at | down | 0.007442413 | Sec23 homolog A (S. cerevisiae) |
| 32998_at | up | 0.007466948 | cholecystokinin A receptor |
| 34752_at | down | 0.007502681 | NIMA (never in mitosis gene a)-related kinase 7 |
| 37906_at | up | 0.007506317 | latent transforming growth factor beta binding protein 2 |
| 32305_at | up | 0.007531192 | collagen, type I, alpha 2 |
| 32222_at | up | 0.007545106 | hypothetical protein FLJ14639 |
| 33067_at | up | 0.007558906 | histone 1, H1a |
| 34680_s_at | down | 0.007566871 | KIAA0107 gene product |
| 602_s_at | up | 0.00758131 | hydroxysteroid (17-beta) dehydrogenase 1 |
| 34466_at | up | 0.007587165 | 5-hydroxytryptamine (serotonin) receptor 4 |
| 36790_at | down | 0.007671431 | tropomyosin 1 (alpha) |
| 35000_at | up | 0.007695054 | tumor necrosis factor (ligand) superfamily, member 9 |
| 41619_at | up | 0.007696786 | interferon regulatory factor 6 |
| 33351_at | down | 0.007707386 | translation factor sui1 homolog |
| 34406_at | up | 0.007730668 | KIAA0602 protein |
| 118_at | up | 0.007740152 | inositol 1,4,5-trisphosphate 3-kinase A |
| 34315_at | up | 0.007744077 | AFG3 ATPase family gene 3-like 2 (yeast) |
| 39241_at | up | 0.007758585 | carbonic anhydrase I |
| 1475_s_at | up | 0.007800059 | v-myb myeloblastosis viral oncogene homolog (avian) |
| 39694_at | up | 0.00783975 | hypothetical protein MGC5508 |
| 272_at | up | 0.007842252 | gastrin-releasing peptide |
| 1827_s_at | up | 0.007852219 | v-myc myelocytomatosis viral oncogene homolog (avian) |
| 32592_at | up | 0.007864197 | KIAA0323 protein |
| 40844_at | down | 0.007876275 | SH2 domain binding protein 1 (tetratricopeptide repeat containing) |
| 35445_at | up | 0.00787895 | sorting nexin 26 |
| 35738_at | down | 0.007881499 | high mobility group nucleosomal binding domain 4 |
| 35306_at | down | 0.007886283 | DEAD/H (Asp-Glu-Ala-Asp/His) box polypeptide 15 |
| 33016_at | up | 0.007891585 | |
| 34359_at | down | 0.007895012 | CGI-130 protein |
| 37683_at | down | 0.007902912 | ubiquitin specific protease 10 |
| 40943_at | up | 0.007973159 | long-chain fatty-acyl elongase |
| 882_at | up | 0.007997396 | colony stimulating factor 1 (macrophage) |
| 40160_at | down | 0.007999975 | POM121 membrane glycoprotein (rat) |
| 34845_at | up | 0.008026503 | CGI-51 protein |
| 41076_at | up | 0.008027637 | gap junction protein, beta 3, 31 kDa (connexin 31) |
| 40406_at | up | 0.00804141 | macrophage stimulating, pseudogene 9 |
| 34296_at | up | 0.008078528 | midline 1 (Opitz/BBB syndrome) |
| 33866_at | down | 0.008090063 | tropomyosin 4 |
| 33493_at | up | 0.008093623 | erythroid differentiation and denucleation factor 1 |
| 37407_s_at | up | 0.008123032 | myosin, heavy polypeptide 11, smooth muscle |
| 33707_at | up | 0.008136505 | phospholipase A2, group IVC (cytosolic, calcium-independent) |
| 31609_s_at | up | 0.00813732 | procollagen C-endopeptidase enhancer |
| 38991_at | up | 0.008149715 | KIAA0220 protein |
| 34773_at | down | 0.008173332 | tubulin-specific chaperone a |
| 39262_at | up | 0.008173678 | protein predicted by clone 23627 |
| 1116_at | up | 0.008206557 | CD19 antigen |
| 863_g_at | up | 0.008219712 | serine (or cysteine) proteinase inhibitor, clade B (ovalbumin), |
| | | | member 5 |
| 35340_at | down | 0.008224703 | mel transforming oncogene (derived from cell line NK14)-RAB8 |
| | | | homolog |
| 34367_at | up | 0.00822972 | phosphoglycerate dehydrogenase |
| 36585_at | down | 0.008245871 | ADP-ribosylation factor 4 |
| 33105_at | up | 0.008266479 | pleckstrin homology domain containing, family B (evectins) |
| | | | member 1 |
| 33169_at | up | 0.008284475 | neogenin homolog 1 (chicken) |
| 40161_at | up | 0.008318429 | cartilage oligomeric matrix protein (pseudoachondroplasia, |
| | | | epiphyseal dysplasia 1, multiple) |
| 38816_at | up | 0.008319784 | transforming, acidic coiled-coil containing protein 2 |
| 38687_at | down | 0.008331445 | DKFZP566D193 protein |
| 34662_at | up | 0.008344705 | myc-induced nuclear antigen, 53 kDa |
| 38400_at | down | 0.008348744 | DKFZP434D1335 protein |
| 38052_at | down | 0.008362106 | coagulation factor XIII, A1 polypeptide |
| 34556_at | up | 0.008416122 | keratin 9 (epidermolytic palmoplantar keratoderma) |
| 39662_s_at | up | 0.008428792 | G protein-coupled receptor kinase 2-like (Drosophila) |
| 32188_at | up | 0.008428989 | myelin transcription factor 1 |
| 36684_at | down | 0.008431467 | adenosylmethionine decarboxylase 1 |
| 34436_at | up | 0.008455809 | solute carrier family 17 (sodium phosphate), member 3 |
| 38607_at | up | 0.008486615 | transmembrane 4 superfamily member 5 |
| 41428_at | up | 0.008492173 | ATP-binding cassette, sub-family C (CFTR/MRP), member 5 |
| 31923_f_at | up | 0.008506636 | ubiquilin 2 |
| 34365_at | up | 0.008508922 | peptidylprolyl isomerase E (cyclophilin E) |
| 36242_at | up | 0.008513005 | small proline-rich protein 2C |
| 38132_at | up | 0.008552125 | CDC42 effector protein (Rho GTPase binding) 1 |
| 1177_at | up | 0.008558202 | |
| 36706_at | up | 0.008564806 | cyclin-dependent kinase-like 5 |
| 41021_s_at | up | 0.0085664 | glycerol-3-phosphate dehydrogenase 2 (mitochondrial) |
| 38067_at | up | 0.008583304 | likely ortholog of mouse septin 8 |
| 33740_at | up | 0.00860968 | chromosome 1 open reading frame 2 |
| 1167_s_at | up | 0.008638816 | matrix metalloproteinase 15 (membrane-inserted) |
| 1726_at | up | 0.008668644 | |
| 40847_at | up | 0.008669583 | flavoprotein oxidoreductase MICAL2 |
| 37368_at | up | 0.008705091 | nuclear factor of activated T-cells, cytoplasmic, calcineurin- |
| | | | dependent 4 |
| 40277_at | up | 0.008712834 | golgi associated, gamma adaptin ear containing, ARF binding |
| | | | protein 2 |
| 35955_at | up | 0.008714371 | cytochrome c-like antigen |
| 41233_at | down | 0.008722182 | DnaJ (Hsp40) homolog, subfamily B, member 6 |
| 40649_at | up | 0.008763293 | proprotein convertase subtilisin/kexin type 1 |
| 36338_at | up | 0.008782835 | leucine zipper protein 1 |
| 35194_at | up | 0.008788982 | glutathione peroxidase 2 (gastrointestinal) |
| 40304_at | up | 0.008813094 | bullous pemphigoid antigen 1, 230/240 kDa |
| 34559_at | up | 0.008828164 | |
| 34753_at | down | 0.008836314 | synaptobrevin-like 1 |
| 40834_at | up | 0.008856926 | PDZ domain containing 3 |
| 1025_g_at | up | 0.008860423 | cytochrome P450, family 1, subfamily A, polypeptide 1 |
| 31352_at | up | 0.008862037 | |
| 38050_at | down | 0.008880693 | Bcl-2-associated transcription factor |
| 33072_at | up | 0.008883812 | hypocretin (orexin) receptor 2 |
| 41792_at | up | 0.00888827 | ATP-binding cassette, sub-family C (CFTR/MRP), member 8 |
| 35492_at | up | 0.008900509 | cytochrome P450, family 4, subfamily F, polypeptide 12 |
| 707_s_at | up | 0.008954052 | |
| 678_at | up | 0.00896 | alkaline phosphatase, placental-like 2 |
| 39633_at | up | 0.008965521 | S100 calcium binding protein A3 |
| 35379_at | up | 0.008977482 | collagen, type IX, alpha 1 |
| 38217_at | up | 0.00898785 | carboxyl ester lipase (bile salt-stimulated lipase) |
| 33788_at | down | 0.00901526 | lysosomal apyrase-like 1 |
| 454_at | up | 0.009032927 | SWI/SNF related, matrix associated, actin dependent regulator of |
| | | | chromatin, subfamily d, member 1 |
| 466_at | down | 0.00903801 | general transcription factor II, i |
| 1030_s_at | down | 0.009041555 | topoisomerase (DNA) I |
| 39198_s_at | up | 0.00906008 | CGI-87 protein |
| 40529_at | up | 0.009060975 | LIM homeobox protein 2 |
| 32098_at | up | 0.009061067 | collagen, type VI, alpha 2 |
| 37981_at | up | 0.009065909 | drebrin 1 |
| 36103_at | up | 0.009068444 | chemokine (C—C motif) ligand 3 |
| 40389_at | up | 0.009068618 | solute carrier family 38, member 3 |
| 40423_at | up | 0.009110888 | KIAA0903 protein |
| 37885_at | up | 0.009118294 | hypothetical protein AF038169 |
| 39308_r_at | up | 0.009130611 | clathrin, light polypeptide (Lcb) |
| 39468_r_at | up | 0.009165844 | |
| 34458_at | up | 0.009174722 | S100 calcium binding protein A7 (psoriasin 1) |
| 39583_at | up | 0.009175879 | glioma amplified on chromosome 1 protein (leucine-rich) |
| 34816_at | down | 0.009179229 | E1A binding protein p400 |
| 35275_at | up | 0.009197426 | adaptor-related protein complex 1, gamma 1 subunit |
| 170_at | up | 0.009214034 | caudal type homeo box transcription factor 2 |
| 38059_g_at | up | 0.009215265 | dermatopontin |
| 40501_s_at | up | 0.00921917 | myosin binding protein C, slow type |
| 32372_at | up | 0.00922417 | cathepsin B |
| 1957_s_at | up | 0.009230348 | transforming growth factor, beta receptor I (activin A receptor type |
| | | | II-like kinase, 53 kDa) |
| 123_at | up | 0.009235174 | protein kinase C, mu |
| 39150_at | down | 0.009258766 | ring finger protein 11 |
| 36813_at | up | 0.009266999 | thyroid hormone receptor interactor 13 |
| 38758_at | up | 0.009277164 | PDGFA associated protein 1 |
| 32198_at | up | 0.009286085 | hypothetical protein FLJ20452 |
| 39667_at | up | 0.009305838 | neuro-oncological ventral antigen 2 |
| 1796_s_at | up | 0.009311292 | B-cell CLL/lymphoma 3 |
| 35279_at | down | 0.009332113 | Tax1 (human T-cell leukemia virus type I) binding protein 1 |
| 32405_at | up | 0.009349281 | thioesterase, adipose associated |
| 31785_f_at | up | 0.009376814 | unnamed HERV-H protein |
| 41458_at | up | 0.009388812 | KIAA0467 protein |
| 34443_at | up | 0.009406186 | |
| 31324_at | up | 0.009411243 | |
| 32303_at | up | 0.00943436 | ets variant gene 3 |
| 32915_at | up | 0.00943445 | |
| 37004_at | up | 0.009438749 | surfactant, pulmonary-associated protein B |
| 35481_at | up | 0.009450411 | myosin heavy chain Myr 8 |
| 33282_at | up | 0.009471432 | ladinin 1 |
| 41307_at | up | 0.009526397 | CCR4-NOT transcription complex, subunit 2 |
| 39238_at | up | 0.009533529 | putative tumor suppressor |
| 41114_at | up | 0.009572837 | microtubule associated testis specific serine/threonine protein |
| | | | kinase |
| 39635_at | up | 0.009598631 | KIAA0960 protein |
| 867_s_at | up | 0.009646891 | thrombospondin 1 |
| 1289_at | up | 0.009657092 | glutathione S-transferase M5 |
| 39132_at | down | 0.009658304 | SWI/SNF related, matrix associated, actin dependent regulator of |
| | | | chromatin, subfamily a, member 5 |
| 31980_at | up | 0.009659298 | winged-helix nude |
| 33075_at | up | 0.009675595 | p21 (CDKN1A)-activated kinase 3 |
| 38448_at | up | 0.009681742 | actinin, alpha 2 |
| 34089_at | up | 0.009695291 | KIAA1030 protein |
| 31587_at | up | 0.009704155 | solute carrier family 14 (urea transporter), member 2 |
| 31390_at | up | 0.00970876 | zinc finger protein 154 (pHZ-92) |
| 31644_at | up | 0.009719178 | chemokine (C—C motif) ligand 27 |
| 1760_s_at | up | 0.009726568 | protein tyrosine phosphatase, non-receptor type 7 |
| 40491_at | up | 0.009743005 | retinoblastoma binding protein 1-like 1 |
| 36815_at | up | 0.009778133 | |
| 33969_at | up | 0.009780589 | interferon, omega 1 |
| 32784_at | down | 0.009784866 | PRP4 pre-mRNA processing factor 4 homolog B (yeast) |
| 40861_at | down | 0.009811631 | mortality factor 4 like 2 |
| 39473_r_at | up | 0.0099094 | protein tyrosine phosphatase type IVA, member 3 |
| 40058_s_at | up | 0.009913258 | LIM protein (similar to rat protein kinase C-binding enigma) |
| 1722_at | up | 0.009928793 | mitogen-activated protein kinase kinase 5 |
| 41074_at | up | 0.009948477 | G protein-coupled receptor 49 |
| 1488_at | up | 0.009953543 | protein tyrosine phosphatase, receptor type, K |
| 38850_at | up | 0.009961288 | |
| 1746_s_at | up | 0.009966628 | |
| 39059_at | up | 0.009967884 | 7-dehydrocholesterol reductase |
| 40252_g_at | up | 0.009972964 | HIV-1 rev binding protein 2 |
| 35695_at | down | 0.009979132 | Chediak-Higashi syndrome 1 |
| 34952_at | up | 0.010018587 | Dombrock blood group |
| 38734_at | up | 0.010025605 | phospholamban |
| 36737_at | up | 0.010027033 | crystallin, beta A4 |
| 41025_r_at | up | 0.010038866 | glycophorin E |
| 36020_at | up | 0.010041904 | KIAA1641 protein |
| 39281_at | up | 0.010049567 | Rho guanine nucleotide exchange factor (GEF) 11 |
| 31931_f_at | up | 0.010060411 | Rhesus blood group, CcEe antigens |
| 380_at | up | 0.010096649 | T-box 5 |
| 31471_at | up | 0.010173071 | |
| 32699_s_at | up | 0.010181466 | poliovirus receptor |
| 36869_at | up | 0.010188243 | paired box gene 8 |
| 1241_at | down | 0.010202166 | protein tyrosine phosphatase type IVA, member 2 |
| 833_at | up | 0.010217675 | integrin, alpha X (antigen CD11C (p150), alpha polypeptide) |
| 1296_at | up | 0.01026762 | cadherin 15, M-cadherin (myotubule) |
| 40142_at | up | 0.010274697 | DEAD/H (Asp-Glu-Ala-Asp/His) box polypeptide 24 |
| 41783_at | up | 0.010279779 | cellular retinoic acid binding protein 2 |
| 40294_at | up | 0.010290318 | ATP-binding cassette, sub-family B (MDR/TAP), member 9 |
| 38170_at | up | 0.010301634 | |
| 36398_at | up | 0.010305459 | RNA, U2 small nuclear |
| 41127_at | up | 0.010310422 | solute carrier family 1 (glutamate/neutral amino acid transporter), |
| | | | member 4 |
| 41445_at | up | 0.010315079 | transforming growth factor, beta 1 (Camurati-Engelmann disease) |
| 37184_at | up | 0.010336804 | syntaxin 1A (brain) |
| 37515_at | up | 0.010347685 | mannan-binding lectin serine protease 2 |
| 39640_at | up | 0.010360104 | glutamine-fructose-6-phosphate transaminase 2 |
| 33823_at | up | 0.010369528 | scavenger receptor class B, member 2 |
| 34448_s_at | up | 0.010370222 | caspase 2, apoptosis-related cysteine protease (neural precursor |
| | | | cell expressed, developmentally down-regulated 2) |
| 36774_f_at | up | 0.01037535 | proline-rich protein BstNI subfamily 1 |
| 35764_at | down | 0.0104032 | oral-facial-digital syndrome 1 |
| 35939_s_at | up | 0.01040841 | POU domain, class 4, transcription factor 1 |
| 36807_at | up | 0.010412052 | TED protein |
| 33034_at | up | 0.01042984 | rhomboid, veinlet-like 1 (Drosophila) |
| 36660_at | down | 0.010439681 | RAB11A, member RAS oncogene family |
| 37031_at | down | 0.010449766 | chromosome 9 open reading frame 10 |
| 39165_at | down | 0.010454441 | nitrogen fixation cluster-like |
| 32339_at | up | 0.010466418 | pancreatic polypeptide |
| 540_at | up | 0.010490446 | heat shock 27 kDa protein 2 |
| 31671_at | down | 0.010490552 | RNA binding motif, single stranded interacting protein 1 |
| 40792_s_at | up | 0.010508924 | triple functional domain (PTPRF interacting) |
| 31423_at | up | 0.010540363 | |
| 34786_at | down | 0.010555311 | jumonji domain containing 1 |
| 32217_at | down | 0.010581595 | chromosome 12 open reading frame 22 |
| 41290_at | up | 0.010587378 | neural cell adhesion molecule 1 |
| 33470_at | up | 0.010596187 | KIAA1719 protein |
| 39229_at | up | 0.010609138 | serologically defined colon cancer antigen 1 |
| 38209_at | up | 0.010647629 | prostaglandin E receptor 1 (subtype EP1), 42 kDa |
| 1138_at | up | 0.010681017 | solute carrier family 20 (phosphate transporter), member 1 |
| 41351_at | up | 0.010681598 | collagen, type VI, alpha 1 |
| 38530_at | up | 0.010725893 | hypothetical protein FLJ22709 |
| 32815_at | up | 0.010728637 | |
| 41223_at | down | 0.010758437 | cytochrome c oxidase subunit Va |
| 39448_r_at | up | 0.010786654 | B7 gene |
| 38208_at | up | 0.010791254 | solute carrier family 35 (UDP-N-acetylglucosamine (UDP-GlcNAc) |
| | | | transporter), member A3 |
| 33902_at | up | 0.010802466 | glycerol-3-phosphate dehydrogenase 1 (soluble) |
| 32885_f_at | up | 0.010812006 | proline-rich protein BstNI subfamily 2 |
| 41253_s_at | down | 0.010819631 | chorionic somatomammotropin hormone 2 |
| 32031_at | up | 0.010819727 | carbamoyl-phosphate synthetase 2, aspartate transcarbamylase, |
| | | | and dihydroorotase |
| 38025_r_at | up | 0.010821372 | rap2 interacting protein x |
| 35510_at | up | 0.010831878 | sodium channel, voltage gated, type VIII, alpha |
| 32956_at | up | 0.010838375 | G protein-coupled receptor, family C, group 5, member B |
| 41033_at | down | 0.01084462 | zinc finger protein 84 (HPF2) |
| 33768_at | up | 0.010848796 | dystrophia myotonica-containing WD repeat motif |
| 34449_at | up | 0.010856436 | caspase 2, apoptosis-related cysteine protease (neural precursor |
| | | | cell expressed, developmentally down-regulated 2) |
| 31674_s_at | up | 0.010905397 | bromodomain containing 3 |
| 32856_at | up | 0.010925156 | KIAA0819 protein |
| 1401_g_at | up | 0.010933721 | colony stimulating factor 2 (granulocyte-macrophage) |
| 35147_at | up | 0.010946156 | MCF.2 cell line derived transforming sequence-like |
| 924_s_at | down | 0.010998105 | protein phosphatase 2 (formerly 2A), catalytic subunit, beta |
| | | | isoform |
| 40912_s_at | up | 0.01100562 | biphenyl hydrolase-like (serine hydrolase; breast epithelial mucin- |
| | | | associated antigen) |
| 31457_at | up | 0.011007906 | forkhead box D2 |
| 40653_at | up | 0.011010145 | regulator of G-protein signalling 7 |
| 35815_at | down | 0.011012092 | huntingtin interacting protein B |
| 33690_at | up | 0.011014125 | |
| 32271_at | up | 0.011029711 | FOS-like antigen 1 |
| 35164_at | up | 0.011029719 | Wolfram syndrome 1 (wolframin) |
| 868_at | down | 0.011059405 | TAF10 RNA polymerase II, TATA box binding protein (TBP)- |
| | | | associated factor, 30 kDa |
| 31949_at | up | 0.011111935 | Ras protein-specific guanine nucleotide-releasing factor 1 |
| 34485_r_at | up | 0.011126448 | ADP-ribosylation factor guanine nucleotide-exchange factor 2 |
| | | | (brefeldin A-inhibited) |
| 40189_at | down | 0.011156147 | SET translocation (myeloid leukemia-associated) |
| 38026_at | up | 0.011184842 | fibulin 1 |
| 32789_at | down | 0.01118756 | nuclear cap binding protein subunit 2, 20 kDa |
| 2087_s_at | up | 0.011265547 | cadherin 11, type 2, OB-cadherin (osteoblast) |
| 477_at | up | 0.011266133 | interferon regulatory factor 5 |
| 1619_g_at | up | 0.011283533 | cytochrome P450, family 19, subfamily A, polypeptide 1 |
| 31368_at | up | 0.011303022 | zinc finger protein 291 |
| 35484_at | up | 0.011329287 | |
| 37221_at | down | 0.0113471 | protein kinase, cAMP-dependent, regulatory, type II, beta |
| 38479_at | down | 0.011383491 | acidic (leucine-rich) nuclear phosphoprotein 32 family, member B |
| AFFX- | up | 0.011438611 | |
| YEL024w/RIP1_at | | | |
| 36061_at | up | 0.011458942 | |
| 1800_g_at | up | 0.011481816 | excision repair cross-complementing rodent repair deficiency, |
| | | | complementation group 4 |
| 36052_at | up | 0.011487914 | adducin 2 (beta) |
| 36201_at | up | 0.011488798 | glyoxalase I |
| 40032_at | up | 0.011543083 | KIAA0133 gene product |
| 33539_at | up | 0.011546061 | myelin expression factor 2 |
| 35860_r_at | up | 0.011555174 | |
| 36601_at | down | 0.011556615 | vinculin |
| 34901_at | up | 0.011562757 | ubiquitin specific protease 2 |
| 31856_at | up | 0.011566968 | glycoprotein A repetitions predominant |
| 39376_at | down | 0.01157243 | homeodomain interacting protein kinase 1 |
| 37860_at | up | 0.011582858 | zinc finger protein 337 |
| AFFX-BioDn-5_at | up | 0.011589001 | |
| 34084_at | up | 0.011592343 | aldo-keto reductase family 1, member D1 (delta 4-3-ketosteroid-5- |
| | | | beta-reductase) |
| 32880_at | up | 0.01161328 | secretoglobin, family 1D, member 2 |
| 160038_s_at | up | 0.011619181 | insulin |
| 37007_at | down | 0.011628359 | tumor differentially expressed 1 |
| 36747_at | up | 0.011652598 | |
| 38882_r_at | up | 0.011660597 | tripartite motif-containing 16 |
| 41724_at | down | 0.011675116 | accessory protein BAP31 |
| 34042_at | up | 0.011693218 | chondroadherin |
| 37088_at | up | 0.011693649 | serine/threonine kinase 13 (aurora/IPL1-like) |
| 36452_at | up | 0.011707524 | synaptopodin |
| 39290_f_at | up | 0.011738795 | PAI-1 mRNA-binding protein |
| 853_at | down | 0.011747537 | nuclear factor (erythroid-derived 2)-like 2 |
| 36332_at | up | 0.011793078 | arylalkylamine N-acetyltransferase |
| 32415_at | up | 0.011799437 | interferon, alpha 5 |
| 35659_at | down | 0.011837246 | interleukin 10 receptor, alpha |
| 38126_at | up | 0.011859592 | biglycan |
| 37475_at | up | 0.011876768 | DKFZP434J046 protein |
| 40468_at | down | 0.011880956 | formin binding protein 1 |
| 1218_at | up | 0.011899656 | nuclear receptor subfamily 2, group F, member 6 |
| 34644_at | up | 0.011971442 | beta-2-microglobulin |
| 40036_at | down | 0.011989436 | mago-nashi homolog, proliferation-associated (Drosophila) |
| 33922_at | up | 0.012007232 | PR domain containing 2, with ZNF domain |
| 34803_at | up | 0.012039368 | ubiquitin specific protease 12 |
| 1235_at | down | 0.012045788 | tyrosine 3-monooxygenase/tryptophan 5-monooxygenase |
| | | | activation protein, zeta polypeptide |
| 40736_at | up | 0.012066991 | cadherin 17, LI cadherin (liver-intestine) |
| 36967_g_at | up | 0.012072207 | ankyrin 3, node of Ranvier (ankyrin G) |
| 36829_at | up | 0.01208281 | period homolog 1 (Drosophila) |
| 41802_at | up | 0.012147295 | hypothetical protein FLJ22531 |
| 34307_at | down | 0.012151793 | transmembrane 9 superfamily member 2 |
| 1631_at | up | 0.012168955 | |
| 40385_at | up | 0.012170497 | chemokine (C—C motif) ligand 20 |
| 31694_at | up | 0.012212301 | regulatory solute carrier protein, family 1, member 1 |
| 40299_at | up | 0.012242936 | G-protein coupled receptor |
| 35169_at | up | 0.01227325 | collagen, type XVI, alpha 1 |
| 31474_r_at | down | 0.012310633 | tankyrase, TRF1-interacting ankyrin-related ADP-ribose |
| | | | polymerase |
| 36557_at | up | 0.012312191 | calcium channel, voltage-dependent, beta 1 subunit |
| 37147_at | up | 0.012334348 | stem cell growth factor; lymphocyte secreted C-type lectin |
| 36544_at | up | 0.012341199 | |
| 40604_at | down | 0.012357898 | dual-specificity tyrosine-(Y)-phosphorylation regulated kinase 2 |
| 37746_r_at | up | 0.012358556 | suppression of tumorigenicity 5 |
| 41101_at | up | 0.012390473 | Sac domain-containing inositol phosphatase 3 |
| 37310_at | up | 0.01240215 | plasminogen activator, urokinase |
| 32232_at | down | 0.012415251 | NADH dehydrogenase (ubiquinone) 1 beta subcomplex, 5, 16 kDa |
| AFFX-BioC-5_at | up | 0.012424633 | |
| 32101_at | up | 0.01242678 | galactosamine (N-acetyl)-6-sulfate sulfatase (Morquio syndrome, |
| | | | mucopolysaccharidosis type IVA) |
| 32079_at | up | 0.012428316 | kinesin family member 13B |
| 38710_at | up | 0.01246202 | ubiquitin-specific protease otubain 1 |
| 40961_at | down | 0.012462865 | SWI/SNF related, matrix associated, actin dependent regulator of |
| | | | chromatin, subfamily a, member 2 |
| 32192_g_at | up | 0.012473564 | ring finger protein 110 |
| 38441_s_at | down | 0.012487948 | membrane cofactor protein (CD46, trophoblast-lymphocyte cross- |
| | | | reactive antigen) |
| 36016_at | up | 0.012489251 | cortistatin |
| 41305_at | up | 0.01250619 | solute carrier family 5 (sodium/glucose cotransporter), member 2 |
| 41289_at | up | 0.012530504 | neural cell adhesion molecule 1 |
| 32613_at | up | 0.012539347 | synaptic vesicle glycoprotein 2B |
| 31734_at | up | 0.012541867 | homeo box C11 |
| 35163_at | down | 0.012560197 | KIAA1041 protein |
| 41759_at | down | 0.012605139 | transcription elongation factor B (SIII), polypeptide 1 pseudogene |
| 37561_at | down | 0.012613195 | nuclear transcription factor Y, alpha |
| 39627_at | up | 0.012650879 | early endosome antigen 1, 162 kD |
| 38413_at | down | 0.012716582 | defender against cell death 1 |
| 35473_at | up | 0.012718357 | collagen, type I, alpha 1 |
| 33724_at | up | 0.012747577 | breast cancer 1, early onset |
| 572_at | up | 0.01277828 | TTK protein kinase |
| 33937_at | up | 0.012832989 | |
| 35229_at | up | 0.012850884 | carnitine palmitoyltransferase 1A (liver) |
| 39780_at | down | 0.01285708 | protein phosphatase 3 (formerly 2B), catalytic subunit, beta |
| | | | isoform (calcineurin A beta) |
| 41098_at | up | 0.012886931 | dishevelled associated activator of morphogenesis 2 |
| 38128_at | up | 0.012911811 | N-acetyltransferase 8 (camello like) |
| 35911_r_at | up | 0.012913888 | matrix metalloproteinase-like 1 |
| 34870_at | up | 0.012919562 | LIM domain binding 3 |
| 31705_at | up | 0.012932212 | ARS component B |
| 32469_at | down | 0.012937069 | carcinoembryonic antigen-related cell adhesion molecule 3 |
| 33808_at | up | 0.012942298 | TEA domain family member 3 |
| 36652_at | up | 0.012968039 | uroporphyrinogen III synthase (congenital erythropoietic porphyria |
| 35172_at | down | 0.012992358 | tyrosylprotein sulfotransferase 2 |
| 40186_at | up | 0.013034664 | dual specificity phosphatase 9 |
| 39454_f_at | up | 0.013098402 | T-cell leukemia, homeobox 2 |
| 36922_at | up | 0.013121302 | ribonucleotide reductase M2 polypeptide |
| 39840_at | up | 0.013143318 | cysteine knot superfamily 1, BMP antagonist 1 |
| 39031_at | up | 0.013178811 | cytochrome c oxidase subunit VIIa polypeptide 1 (muscle) |
| 1032_at | up | 0.013182233 | dihydropyrimidine dehydrogenase |
| 37314_at | up | 0.013203795 | chromosome 14 open reading frame 11 |
| 36814_at | down | 0.013248989 | hypothetical protein KIAA1109 |
| 31434_at | up | 0.013251593 | |
| 1523_g_at | up | 0.013266534 | tyrosine kinase, non-receptor, 1 |
| 210_at | up | 0.013282218 | phospholipase C, beta 2 |
| 31450_s_at | up | 0.013347747 | Ras-like without CAAX 2 |
| 35303_at | down | 0.013354891 | insulin induced gene 1 |
| 37042_at | up | 0.013358442 | hyaluronoglucosaminidase 2 |
| 37675_at | down | 0.013393808 | solute carrier family 25 (mitochondrial carrier; phosphate carrier), |
| | | | member 3 |
| 40363_r_at | up | 0.013426669 | nuclear factor of kappa light polypeptide gene enhancer in B-cells |
| | | | 2 (p49/p100) |
| 745_at | up | 0.013436936 | transcription elongation factor A (SII), 2 |
| 39427_at | down | 0.013456646 | ubiquinol-cytochrome c reductase binding protein |
| 33267_at | down | 0.013514384 | |
| 32381_at | up | 0.013553578 | RAR-related orphan receptor B |
| 31626_i_at | up | 0.013564285 | amine oxidase pseudogene |
| 39469_s_at | up | 0.013613609 | ATPase, aminophospholipid transporter-like, Class I, type 8A, |
| | | | member 2 |
| 1950_s_at | up | 0.013677073 | MAD, mothers against decapentaplegic homolog 3 (Drosophila) |
| 40512_at | up | 0.01367746 | chimerin (chimaerin) 1 |
| 37729_at | down | 0.013682129 | exportin 1 (CRM1 homolog, yeast) |
| 40402_at | up | 0.013695808 | solute carrier family 6 (neurotransmitter transporter, noradrenalin) |
| | | | member 2 |
| 40595_at | up | 0.01373059 | Treacher Collins-Franceschetti syndrome 1 |
| 37756_at | up | 0.01375692 | RYK receptor-like tyrosine kinase |
| 1664_at | up | 0.01381471 | |
| 31636_s_at | up | 0.013826343 | solute carrier family 18 (vesicular acetylcholine), member 3 |
| 41293_at | up | 0.013828112 | keratin 7 |
| 33546_at | up | 0.013836831 | |
| 31386_at | up | 0.013843476 | immunoglobulin kappa variable 1/OR15-118 |
| 33495_at | up | 0.013909579 | protein tyrosine phosphatase, receptor type, f polypeptide |
| | | | (PTPRF), interacting protein (liprin), alpha 2 |
| 37274_at | up | 0.013914109 | biotinidase |
| 41488_at | down | 0.013928912 | hypothetical protein A-211C6.1 |
| 31999_at | up | 0.013935691 | ATP-binding cassette, sub-family A (ABC1), member 1 |
| 39360_at | down | 0.013945505 | sorting nexin 3 |
| 34791_at | down | 0.013953726 | t-complex 1 |
| 34324_at | down | 0.01395827 | ceroid-lipofuscinosis, neuronal 5 |
| 40819_at | up | 0.013958858 | RNA binding motif protein 8A |
| 41529_g_at | down | 0.013984956 | |
| 35793_at | down | 0.014000217 | Ras-GTPase activating protein SH3 domain-binding protein 2 |
| 36075_at | up | 0.014076733 | RAB, member of RAS oncogene family-like 4 |
| 33322_i_at | up | 0.014082655 | stratifin |
| 35978_at | up | 0.014105216 | proline-rich Gla (G-carboxyglutamic acid) polypeptide 1 |
| 37961_at | up | 0.014113688 | phosphoinositide-3-kinase, regulatory subunit, polypeptide 3 (p55, |
| | | | gamma) |
| 35521_at | up | 0.01411609 | claudin 9 |
| 33266_at | up | 0.014136389 | serine/threonine kinase 12 |
| 31325_at | up | 0.014137098 | |
| 41001_at | up | 0.014148684 | likely ortholog of mouse rabphilin 3A |
| 41123_s_at | up | 0.014166567 | ectonucleotide pyrophosphatase/phosphodiesterase 2 (autotaxin) |
| 41079_at | up | 0.014179586 | amiloride-sensitive cation channel 3, testis |
| 33221_at | up | 0.014203535 | PAX transcription activation domain interacting protein 1 like |
| 806_at | up | 0.014209175 | cytokine-inducible kinase |
| 39657_at | up | 0.014284559 | keratin 4 |
| 36625_at | up | 0.014320189 | peroxisomal long-chain acyl-coA thioesterase |
| 38538_at | up | 0.014353625 | solute carrier family 24 (sodium/potassium/calcium exchanger), |
| | | | member 1 |
| 37189_at | up | 0.014377897 | phosphomannomutase 1 |
| 33670_at | up | 0.014395478 | |
| 41602_at | up | 0.014427132 | hippocalcin |
| 39490_f_at | up | 0.014434597 | ADP-ribosylation factor GTPase activating protein 3 |
| 39137_at | up | 0.014438261 | nuclear factor related to kappa B binding protein |
| 32104_i_at | up | 0.014440307 | calcium/calmodulin-dependent protein kinase (CaM kinase) II |
| | | | gamma |
| 37976_at | up | 0.014454216 | Ig superfamily protein |
| 36420_at | up | 0.014470534 | intersectin 2 |
| 37204_at | up | 0.014470631 | pre-alpha (globulin) inhibitor, H3 polypeptide |
| 742_at | up | 0.014501825 | hyaluronan binding protein 2 |
| 277_at | down | 0.014527659 | myeloid cell leukemia sequence 1 (BCL2-related) |
| 40945_at | up | 0.014552217 | TGFB inducible early growth response 2 |
| 31833_at | up | 0.014573625 | phosphatidylinositol-4-phosphate 5-kinase, type I, alpha |
| 36913_at | down | 0.014582499 | stem-loop (histone) binding protein |
| 33783_at | up | 0.014601433 | plexin B1 |
| 34764_at | up | 0.014611105 | leucyl-tRNA synthetase, mitochondrial |
| 32269_at | up | 0.014621549 | BAI1-associated protein 1 |
| 912_s_at | up | 0.014641028 | phospholipase A2, group IB (pancreas) |
| 37227_at | up | 0.014654821 | apoptotic protease activating factor |
| 33440_at | down | 0.014672964 | transcription factor 8 (represses interleukin 2 expression) |
| 38943_at | down | 0.014708654 | holocytochrome c synthase (cytochrome c heme-lyase) |
| 35113_at | up | 0.014808127 | solute carrier family 22 (organic cation transporter), member 1 |
| 38181_at | up | 0.014820162 | matrix metalloproteinase 11 (stromelysin 3) |
| 41018_at | up | 0.014823451 | DKFZP564O243 protein |
| 35266_at | down | 0.014833914 | bladder cancer associated protein |
| 463_g_at | up | 0.014919402 | nuclear factor I/B |
| 33545_at | up | 0.014947629 | sodium channel, voltage-gated, type IV, alpha |
| 41785_at | down | 0.014955497 | eukaryotic translation initiation factor 4 gamma, 2 |
| 37905_r_at | up | 0.015015551 | |
| 39564_s_at | up | 0.015051374 | ATP-binding cassette, sub-family B (MDR/TAP), member 6 |
| 35342_at | down | 0.015052012 | |
| 36378_at | up | 0.015073855 | uroplakin 1A |
| 35434_at | up | 0.015123449 | MADS box transcription enhancer factor 2, polypeptide D |
| | | | (myocyte enhancer factor 2D) |
| 41525_at | up | 0.015136461 | high-mobility group 20B |
| 37276_at | down | 0.015149254 | IQ motif containing GTPase activating protein 2 |
| 1424_s_at | down | 0.015163256 | tyrosine 3-monooxygenase/tryptophan 5-monooxygenase |
| | | | activation protein, eta polypeptide |
| 37442_at | down | 0.015178917 | hypothetical protein DKFZp586I1420 |
| 39214_at | up | 0.015195618 | plexin B3 |
| 36720_at | up | 0.015223928 | pyruvate dehydrogenase kinase, isoenzyme 3 |
| 40941_at | up | 0.015261702 | VAMP (vesicle-associated membrane protein)-associated protein |
| | | | B and C |
| 33374_at | up | 0.015278411 | complement component 2 |
| 40027_at | down | 0.015289109 | ATP synthase, H+ transporting, mitochondrial F0 complex, subun |
| | | | s (factor B) |
| 31738_at | up | 0.015316801 | |
| 33642_s_at | up | 0.015319584 | solute carrier family 6 (neurotransmitter transporter, creatine), |
| | | | member 8 |
| 34525_at | up | 0.015328652 | T-cell leukemia/lymphoma 1B |
| 34923_at | up | 0.015331205 | KIAA0522 protein |
| 36718_s_at | down | 0.015337288 | pyruvate dehydrogenase kinase, isoenzyme 3 |
| 33666_at | down | 0.015344184 | heterogeneous nuclear ribonucleoprotein C (C1/C2) |
| 39383_at | up | 0.015367512 | adenylate cyclase 6 |
| 37248_at | up | 0.015368335 | carboxypeptidase Z |
| 33807_at | up | 0.015395268 | phosphoinositol 3-phosphate-binding protein-3 |
| 1971_g_at | up | 0.015433856 | fragile histidine triad gene |
| 32522_f_at | up | 0.015452907 | clathrin, light polypeptide (Lcb) |
| 40237_at | up | 0.015487652 | tumor suppressing subtransferable candidate 3 |
| 35295_g_at | down | 0.01549105 | Sjogren syndrome antigen A2 (60 kDa, ribonucleoprotein |
| | | | autoantigen SS-A/Ro) |
| 218_at | down | 0.015538652 | IK cytokine, down-regulator of HLA II |
| 39639_s_at | up | 0.015540109 | transition protein 1 (during histone to protamine replacement) |
| 33144_at | up | 0.015542466 | solute carrier family 16 (monocarboxylic acid transporters), |
| | | | member 3 |
| 34697_at | up | 0.015588844 | low density lipoprotein receptor-related protein 6 |
| 39868_at | up | 0.015598787 | poly(rC) binding protein 3 |
| 35091_at | up | 0.015609298 | neuregulin 2 |
| 1096_g_at | up | 0.015618275 | CD19 antigen |
| 31452_at | up | 0.015629582 | survival motor neuron pseudogene |
| 39511_at | up | 0.015634398 | myeloid/lymphoid or mixed-lineage leukemia (trithorax homolog, |
| | | | Drosophila); translocated to, 4 |
| 36826_at | up | 0.015634839 | general transcription factor IIF, polypeptide 1, 74 kDa |
| 1415_at | up | 0.015636698 | embryonal Fyn-associated substrate |
| 41279_f_at | up | 0.015657636 | mitogen-activated protein kinase 8 interacting protein 1 |
| 36326_at | up | 0.015683908 | nescient helix loop helix 2 |
| 1555_f_at | up | 0.015707626 | cytochrome P450, family 2, subfamily A, polypeptide 7 |
| 37133_at | up | 0.015716843 | serine/threonine kinase 23 |
| 33610_at | up | 0.015729266 | claudin 8 |
| 40674_s_at | up | 0.015769271 | homeo box C6 |
| 39765_at | up | 0.015769941 | talin 2 |
| 35352_at | up | 0.015775962 | aryl-hydrocarbon receptor nuclear translocator 2 |
| 35974_at | down | 0.015787229 | lymphoid-restricted membrane protein |
| 34802_at | up | 0.015795589 | collagen, type VI, alpha 2 |
| 34902_at | up | 0.015815292 | KIAA0492 protein |
| 984_g_at | up | 0.015817247 | mitogen-activated protein kinase 12 |
| 37267_at | up | 0.015828594 | thimet oligopeptidase 1 |
| 34002_at | up | 0.0158432 | hydroxy-delta-5-steroid dehydrogenase, 3 beta- and steroid delta- |
| | | | isomerase 2 |
| 38946_at | up | 0.015843586 | protein phosphatase, EF hand calcium-binding domain 1 |
| 31704_at | up | 0.015883818 | deoxyribonuclease I-like 2 |
| 41042_r_at | up | 0.01592032 | myosin VIIA (Usher syndrome 1B (autosomal recessive, severe)) |
| 31926_at | up | 0.01593408 | cytochrome P450, family 7, subfamily A, polypeptide 1 |
| 37158_at | up | 0.015948773 | |
| 1896_s_at | up | 0.015958981 | ATP-binding cassette, sub-family C (CFTR/MRP), member 1 |
| 41416_at | up | 0.015961067 | fibrinogen-like 1 |
| 35910_f_at | up | 0.015964906 | matrix metalloproteinase-like 1 |
| 38582_at | up | 0.015969003 | serine protease inhibitor, Kazal type 1 |
| 38114_at | down | 0.016076891 | RAD21 homolog (S. pombe) |
| 40926_at | up | 0.016084766 | solute carrier family 6 (neurotransmitter transporter, creatine), |
| | | | member 8 |
| 34394_at | down | 0.01609586 | activity-dependent neuroprotector |
| 31556_at | up | 0.016136864 | |
| 32103_at | up | 0.016177838 | serine (or cysteine) proteinase inhibitor, clade F (alpha-2 |
| | | | antiplasmin, pigment epithelium derived factor), member 2 |
| 38572_at | up | 0.016177861 | FGFR1 oncogene partner |
| 34864_at | up | 0.016199781 | hypothetical protein CGI-57 |
| 35095_r_at | down | 0.016220568 | leukocyte immunoglobulin-like receptor, subfamily A (without TM |
| | | | domain), member 3 |
| 1391_s_at | up | 0.016223701 | cytochrome P450, family 4, subfamily A, polypeptide 11 |
| 31902_at | up | 0.016232561 | deiodinase, iodothyronine, type II |
| 37303_at | down | 0.016339359 | ADP-ribosyltransferase (NAD+; poly (ADP-ribose) polymerase)- |
| | | | like 1 |
| 38841_at | down | 0.016345714 | putative glialblastoma cell differentiation-related |
| 32204_at | up | 0.016360734 | phosphodiesterase 6G, cGMP-specific, rod, gamma |
| 37060_at | up | 0.016378817 | |
| 31577_at | up | 0.016382282 | collagen, type XIX, alpha 1 |
| 40180_at | up | 0.016385547 | insulin receptor substrate 2 |
| 39775_at | up | 0.016407009 | serine (or cysteine) proteinase inhibitor, clade G (C1 inhibitor), |
| | | | member 1, (angioedema, hereditary) |
| 41143_at | down | 0.016409508 | calmodulin 1 (phosphorylase kinase, delta) |
| 36583_at | down | 0.016418131 | sorting nexin 1 |
| 35828_at | up | 0.016423982 | cysteine-rich protein 2 |
| 37573_at | up | 0.016431244 | angiopoietin-like 2 |
| 39784_at | down | 0.016449639 | eukaryotic translation initiation factor 2, subunit 1 alpha, 35 kDa |
| 40745_at | up | 0.016531089 | adaptor-related protein complex 1, beta 1 subunit |
| 36987_at | up | 0.016535539 | lamin B2 |
| 35565_at | up | 0.016616751 | LanC lantibiotic synthetase component C-like 2 (bacterial) |
| 37672_at | down | 0.016674897 | ubiquitin specific protease 7 (herpes virus-associated) |
| 39103_s_at | up | 0.016676884 | |
| 37554_at | up | 0.016679024 | kallikrein 6 (neurosin, zyme) |
| 38657_s_at | up | 0.016700888 | clathrin, light polypeptide (Lca) |
| 36019_at | up | 0.016718156 | serine/threonine kinase 19 |
| 34214_at | up | 0.016732911 | KIAA0644 gene product |
| 36844_at | up | 0.016756145 | dedicator of cyto-kinesis 3 |
| 41711_at | up | 0.016773547 | thioredoxin reductase 2 |
| 939_at | up | 0.016782506 | |
| 35396_at | up | 0.016794613 | hyaluronan synthase 2 |
| 35198_at | up | 0.016796595 | promethin |
| 33947_at | up | 0.016809014 | G protein-coupled receptor 3 |
| 38840_s_at | down | 0.01682395 | profilin 2 |
| 31890_s_at | down | 0.016843461 | zinc finger protein 143 (clone pHZ-1) |
| 34024_at | up | 0.016873841 | chloride channel 5 (nephrolithiasis 2, X-linked, Dent disease) |
| 37146_at | down | 0.016887348 | KIAA0404 protein |
| 39801_at | up | 0.01689521 | procollagen-lysine, 2-oxoglutarate 5-dioxygenase 3 |
| 40879_at | down | 0.016929255 | coiled-coil protein BICD2 |
| 33595_r_at | up | 0.016932374 | recombination activating gene 2 |
| 706_at | down | 0.016934279 | |
| 35798_at | up | 0.01694392 | NS1-associated protein 1 |
| 1713_s_at | down | 0.016952268 | cyclin-dependent kinase inhibitor 2A (melanoma, p16, inhibits |
| | | | CDK4) |
| 35112_at | up | 0.016979898 | regulator of G-protein signalling 9 |
| 32163_f_at | up | 0.016998487 | chorionic somatomammotropin hormone 2 |
| 31881_at | down | 0.017004901 | mob protein |
| 31775_at | up | 0.017053345 | surfactant, pulmonary-associated protein D |
| 37995_s_at | down | 0.017056726 | fragile X mental retardation 1 |
| 35915_at | up | 0.017078706 | inhibin, beta C |
| 36896_s_at | down | 0.017114074 | aryl hydrocarbon receptor nuclear translocator-like |
| 34222_at | up | 0.017137337 | hypothetical protein from clone 24828 |
| 39194_at | up | 0.017213039 | glutathione peroxidase 2 (gastrointestinal) |
| 33879_at | up | 0.017232449 | type I sigma receptor |
| 36152_at | up | 0.017242899 | GDP dissociation inhibitor 1 |
| 263_g_at | down | 0.017262681 | adenosylmethionine decarboxylase 1 |
| 36690_at | down | 0.017262777 | nuclear receptor subfamily 3, group C, member 1 (glucocorticoid |
| | | | receptor) |
| 35398_at | up | 0.017273339 | |
| 38046_at | down | 0.017297027 | IK cytokine, down-regulator of HLA II |
| 1483_at | up | 0.017308878 | cadherin 4, type 1, R-cadherin (retinal) |
| 34567_at | up | 0.017357311 | cylicin, basic protein of sperm head cytoskeleton 2 |
| 33122_at | up | 0.017373574 | regulator of G-protein signalling 10 |
| 884_at | up | 0.017392757 | integrin, alpha 3 (antigen CD49C, alpha 3 subunit of VLA-3 |
| | | | receptor) |
| 35080_at | up | 0.017429659 | neurotensin receptor 1 (high affinity) |
| 2022_at | up | 0.017431884 | v-akt murine thymoma viral oncogene homolog 2 |
| 40535_i_at | up | 0.017441987 | translation initiation factor IF2 |
| 32587_at | down | 0.017445795 | zinc finger protein 36, C3H type-like 2 |
| 1399_at | down | 0.017450684 | transcription elongation factor B (SIII), polypeptide 1 (15 kDa, |
| | | | elongin C) |
| 1169_at | up | 0.017478787 | protocadherin gamma subfamily B, 7 |
| 41285_at | up | 0.017525434 | inositol polyphosphate-5-phosphatase, 40 kDa |
| 37228_at | up | 0.017538384 | polo-like kinase (Drosophila) |
| 35876_s_at | up | 0.017574915 | sphingosine-1-phosphate lyase 1 |
| 32903_at | up | 0.017581104 | transforming growth factor, beta receptor I (activin A receptor type |
| | | | II-like kinase, 53 kDa) |
| 35180_at | down | 0.017588189 | c-Mpl binding protein |
| 1051_g_at | up | 0.017597054 | melan-A |
| 33126_at | down | 0.017609759 | glycosyltransferase AD-017 |
| 33436_at | up | 0.017614337 | SRY (sex determining region Y)-box 9 (campomelic dysplasia, |
| | | | autosomal sex-reversal) |
| 33806_at | up | 0.01762397 | hypothetical protein FLJ22195 |
| 39965_at | up | 0.01764993 | ras-related C3 botulinum toxin substrate 3 (rho family, small GTF |
| | | | binding protein Rac3) |
| 385_at | up | 0.017687295 | |
| 40959_at | up | 0.017704581 | KIAA0599 protein |
| 36356_at | up | 0.017780577 | growth differentiation factor 5 (cartilage-derived morphogenetic |
| | | | protein-1) |
| 39855_at | up | 0.017795155 | Fzr1 protein |
| 40931_at | down | 0.017817731 | CGI-100 protein |
| 492_g_at | up | 0.017826554 | protein tyrosine phosphatase, receptor type, G |
| 1420_s_at | down | 0.017834415 | eukaryotic translation initiation factor 4A, isoform 2 |
| 36407_at | up | 0.017841019 | kallikrein 13 |
| 37185_at | up | 0.017841095 | serine (or cysteine) proteinase inhibitor, clade B (ovalbumin), |
| | | | member 2 |
| 40872_at | down | 0.017872198 | cytochrome c oxidase subunit VIb |
| 37334_at | down | 0.017882062 | heterogeneous nuclear ribonucleoprotein A0 |
| 39185_at | down | 0.017885168 | hypothetical protein 628 |
| 37766_s_at | down | 0.017887937 | proteasome (prosome, macropain) 26S subunit, ATPase, 5 |
| 32005_at | up | 0.017891687 | pro-melanin-concentrating hormone |
| 31688_at | up | 0.017957105 | skin-specific protein |
| 2055_s_at | up | 0.01796478 | integrin, beta 1 (fibronectin receptor, beta polypeptide, antigen |
| | | | CD29 includes MDF2, MSK12) |
| 35214_at | up | 0.018093625 | UDP-glucose dehydrogenase |
| 34161_at | up | 0.018130162 | lactoperoxidase |
| 36392_at | up | 0.018158369 | zinc finger protein 135 (clone pHZ-17) |
| 33310_at | down | 0.018160642 | comparative gene identification 58 |
| 38298_at | up | 0.018174474 | potassium large conductance calcium-activated channel, |
| | | | subfamily M, beta member 1 |
| 39200_s_at | up | 0.018250192 | growth differentiation factor 11 |
| 625_at | up | 0.018283211 | vesicle amine transport protein 1 homolog (T californica) |
| 36834_at | up | 0.018291464 | DKFZP564G202 protein |
| 40950_at | up | 0.018330724 | dynein, cytoplasmic, light intermediate polypeptide 2 |
| 35790_at | down | 0.018330827 | vacuolar protein sorting 26 (yeast) |
| 38915_at | up | 0.018336984 | KIAA0563 gene product |
| 41394_at | up | 0.018360549 | phospholipase D2 |
| 33961_at | up | 0.018361619 | |
| 37602_at | up | 0.018387236 | guanidinoacetate N-methyltransferase |
| 507_s_at | down | 0.018388413 | E74-like factor 2 (ets domain transcription factor) |
| 31637_s_at | up | 0.018396658 | nuclear receptor subfamily 1, group D, member 1 |
| 36797_at | up | 0.018409859 | sialophorin (gpL115, leukosialin, CD43) |
| 39345_at | down | 0.018412516 | Niemann-Pick disease, type C2 |
| 40188_f_at | up | 0.018416817 | |
| 38374_at | down | 0.018474391 | TGFB inducible early growth response |
| 38104_at | down | 0.018535035 | 2,4-dienoyl CoA reductase 1, mitochondrial |
| 35354_at | up | 0.018538623 | synaptogyrin 1 |
| 34336_at | down | 0.018562306 | lysyl-tRNA synthetase |
| 38526_at | up | 0.01860093 | phosphodiesterase 4D, cAMP-specific (phosphodiesterase E3 |
| | | | dunce homolog, Drosophila) |
| 36032_at | down | 0.018630726 | HSPCO34 protein |
| 35485_at | up | 0.01866013 | glutamate receptor, metabotropic 4 |
| 39547_at | up | 0.018672308 | RAN binding protein 9 |
| 37875_at | up | 0.018679056 | glycoprotein A33 (transmembrane) |
| 730_r_at | up | 0.018699817 | |
| 33985_s_at | up | 0.018705931 | heat shock 90 kDa protein 1, beta |
| 39319_at | down | 0.018716388 | lymphocyte cytosolic protein 2 (SH2 domain containing leukocyte |
| | | | protein of 76 kDa) |
| 40094_r_at | up | 0.018725325 | Lutheran blood group (Auberger b antigen included) |
| 36924_r_at | up | 0.018756701 | secretogranin II (chromogranin C) |
| 36306_at | up | 0.018756705 | potassium voltage-gated channel, KQT-like subfamily, member 3 |
| 1704_at | up | 0.018767596 | vav 2 oncogene |
| 41399_at | down | 0.018773794 | KIAA1111 protein |
| 40804_at | up | 0.01880313 | nucleoporin 88 kDa |
| 31837_at | up | 0.018865712 | hypothetical protein BC002942 |
| 35725_at | down | 0.018871291 | karyopherin alpha 3 (importin alpha 4) |
| 38154_at | up | 0.018880449 | |
| 39140_at | down | 0.018895051 | nucleic acid helicase DDXx |
| 34055_at | up | 0.018918709 | activin A receptor, type IB |
| 41804_at | up | 0.018951167 | hypothetical protein FLJ22531 |
| 31701_r_at | up | 0.018965341 | B1 for mucin |
| 32909_at | up | 0.019004154 | aquaporin 5 |
| 32399_at | up | 0.019021041 | ecotropic viral integration site 1 |
| 40496_at | up | 0.01903891 | complement component 1, s subcomponent |
| 36330_at | up | 0.019050873 | cysteine conjugate-beta lyase; cytoplasmic (glutamine |
| | | | transaminase K, kyneurenine aminotransferase) |
| 888_s_at | up | 0.019070811 | LAG1 longevity assurance homolog 1 (S. cerevisiae) |
| 37748_at | down | 0.019075149 | KIAA0232 gene product |
| 41418_at | up | 0.019075413 | latrophilin 1 |
| 40488_at | up | 0.019080462 | dystrophin (muscular dystrophy, Duchenne and Becker types) |
| 40390_at | up | 0.01908294 | serine dehydratase |
| 41078_at | up | 0.019088607 | KIAA0150 protein |
| 38626_at | down | 0.019091364 | KIAA0399 protein |
| 650_s_at | down | 0.019102817 | calcium/calmodulin-dependent protein kinase (CaM kinase) II |
| | | | gamma |
| 39245_at | up | 0.019111468 | |
| 36080_at | up | 0.0191278 | clock homolog (mouse) |
| 39408_at | up | 0.019130961 | hypothetical protein MGC5139 |
| 39872_at | up | 0.019146391 | G-2 and S-phase expressed 1 |
| 36119_at | up | 0.019160158 | caveolin 1, caveolae protein, 22 kDa |
| 36570_at | up | 0.019170443 | calbindin 1, 28 kDa |
| 40063_at | down | 0.01917505 | nuclear domain 10 protein |
| 39113_at | up | 0.019182305 | protein disulfide isomerase related protein (calcium-binding |
| | | | protein, intestinal-related) |
| 36663_at | up | 0.019184809 | natriuretic peptide precursor A |
| 34573_at | up | 0.019216743 | ephrin-A3 |
| 37615_at | up | 0.019228832 | growth factor receptor-bound protein 10 |
| 34306_at | down | 0.019238039 | muscleblind-like (Drosophila) |
| 40344_at | up | 0.019239373 | neuroligin 1 |
| 36375_at | up | 0.019280928 | outer dense fiber of sperm tails 1 |
| 32975_g_at | up | 0.01928292 | homolog of Yeast RRP4 (ribosomal RNA processing 4), 3′-5′- |
| | | | exoribonuclease |
| 692_s_at | up | 0.01928419 | superoxide dismutase 3, extracellular |
| 38088_r_at | up | 0.019334463 | S100 calcium binding protein A4 (calcium protein, calvasculin, |
| | | | metastasin, murine placental homolog) |
| 36139_at | up | 0.01937145 | chromosome 6 open reading frame 4 |
| 33329_at | up | 0.019378158 | nuclear factor I/C (CCAAT-binding transcription factor) |
| 35598_at | up | 0.019416892 | histone 1, H3e |
| 35821_at | down | 0.019431843 | histone deacetylase 3 |
| 41241_at | down | 0.019435076 | asparaginyl-tRNA synthetase |
| 667_at | up | 0.019461528 | arginine vasopressin receptor 2 (nephrogenic diabetes insipidus) |
| 39809_at | down | 0.019462445 | HMG-box containing protein 1 |
| 34851_at | up | 0.019463854 | serine/threonine kinase 6 |
| 1007_s_at | up | 0.0194894 | discoidin domain receptor family, member 1 |
| 567_s_at | up | 0.01953774 | promyelocytic leukemia |
| 40355_at | up | 0.019595751 | AND-1 protein |
| 37562_at | up | 0.019609089 | protocadherin 1 (cadherin-like 1) |
| 39451_i_at | up | 0.019623483 | iduronate 2-sulfatase (Hunter syndrome) |
| 32785_at | down | 0.019631759 | eukaryotic translation initiation factor 3, subunit 10 theta, |
| | | | 150/170 kDa |
| 694_at | up | 0.019702348 | |
| 36276_at | up | 0.019710749 | contactin 2 (axonal) |
| 668_s_at | up | 0.019731276 | matrix metalloproteinase 7 (matrilysin, uterine) |
| 31468_f_at | up | 0.019744635 | glutamate receptor, metabotropic 1 |
| 40418_at | down | 0.019762763 | retinoblastoma binding protein 4 |
| 36051_s_at | up | 0.01982094 | adducin 2 (beta) |
| 34667_at | up | 0.019830749 | nuclear transcription factor, X-box binding 1 |
| 37565_at | down | 0.019838268 | monocyte to macrophage differentiation-associated |
| 36954_at | down | 0.019858282 | KIAA0218 gene product |
| 31746_at | up | 0.019892901 | zinc finger protein 204 |
| 1481_at | up | 0.019894049 | matrix metalloproteinase 12 (macrophage elastase) |
| 666_at | up | 0.019910172 | phosphodiesterase 4A, cAMP-specific (phosphodiesterase E2 |
| | | | dunce homolog, Drosophila) |
| 1141_at | up | 0.019912154 | melanocortin 5 receptor |
| 39037_at | down | 0.019935896 | myeloid/lymphoid or mixed-lineage leukemia (trithorax homolog, |
| | | | Drosophila); translocated to, 2 |
| 33673_r_at | up | 0.019947098 | UDP glycosyltransferase 2 family, polypeptide B17 |
| 33652_at | up | 0.019972991 | a disintegrin and metalloproteinase domain 20 |
| 35534_at | up | 0.020002103 | KIAA0514 gene product |
| 34824_at | down | 0.020009104 | ubiquilin 2 |
| 39108_at | up | 0.020020999 | lanosterol synthase (2,3-oxidosqualene-lanosterol cyclase) |
| 31810_g_at | up | 0.020025692 | contactin 1 |
| 32595_at | down | 0.02002983 | G-rich RNA sequence binding factor 1 |
| 33345_at | up | 0.020031007 | kinesin family member 3C |
| 39294_at | up | 0.020106081 | nuclear receptor subfamily 2, group F, member 1 |
| 1834_at | up | 0.02010754 | putative G protein coupled receptor |
| 33622_at | up | 0.020119043 | calcium channel, voltage-dependent, L type, alpha 1C subunit |
| 40598_at | up | 0.020139375 | START domain containing 5 |
| 34846_at | up | 0.020142879 | calcium/calmodulin-dependent protein kinase (CaM kinase) II beta |
| 32928_at | up | 0.020150947 | POU domain, class 2, transcription factor 3 |
| 37073_at | up | 0.020168835 | eyes absent homolog 1 (Drosophila) |
| 41784_at | down | 0.020217509 | SR rich protein |
| 34184_at | up | 0.020259848 | adenomatous polyposis coli like |
| 38477_at | down | 0.020271492 | diptheria toxin resistance protein required for diphthamide |
| | | | biosynthesis-like 1 (S. cerevisiae) |
| 40260_g_at | up | 0.020317242 | RNA binding motif protein 9 |
| 40740_at | up | 0.020333636 | paired box gene 6 (aniridia, keratitis) |
| 36007_at | up | 0.020396168 | DKFZP586L151 protein |
| 36380_at | up | 0.020398685 | DKFZP434F122 protein |
| 41574_at | down | 0.020400712 | pinin, desmosome associated protein |
| 39879_s_at | up | 0.020473075 | hypothetical protein FLJ10120 |
| 33787_at | up | 0.020483026 | KIAA0537 gene product |
| 33008_at | up | 0.020521776 | olfactory receptor, family 7, subfamily E, member 24 pseudogene |
| 33294_at | down | 0.020522678 | KIAA0116 protein |
| 33241_at | down | 0.020533956 | KIAA0626 gene product |
| 35584_s_at | up | 0.020544608 | calcium channel, voltage-dependent, alpha 1F subunit |
| 36355_at | up | 0.02055766 | involucrin |
| 33681_at | up | 0.020563179 | serine (or cysteine) proteinase inhibitor, clade H (heat shock |
| | | | protein 47), member 1, (collagen binding protein 1) |
| 33558_at | up | 0.020601885 | T-box 5 |
| 34778_at | up | 0.020605113 | |
| 33319_at | down | 0.02062051 | axin 1 |
| 33150_at | down | 0.020623787 | disrupter of silencing 10 |
| 1549_s_at | up | 0.02062695 | serine (or cysteine) proteinase inhibitor, clade B (ovalbumin), |
| | | | member 4 |
| 34274_at | down | 0.020627926 | RNA binding motif protein 16 |
| 32637_r_at | up | 0.020660502 | PI-3-kinase-related kinase SMG-1 |
| 37201_at | up | 0.020782803 | inter-alpha (globulin) inhibitor H4 (plasma Kallikrein-sensitive |
| | | | glycoprotein) |
| 40003_at | up | 0.020809717 | glycoprotein 2 (zymogen granule membrane) |
| 38605_at | down | 0.020824508 | NADH dehydrogenase (ubiquinone) 1 beta subcomplex, 1, 7 kDa |
| 36775_f_at | up | 0.020845451 | proline-rich protein BstNI subfamily 2 |
| 1557_at | down | 0.020870386 | p21/Cdc42/Rac1-activated kinase 1 (STE20 homolog, yeast) |
| 1371_s_at | up | 0.02094109 | cytochrome P450, family 2, subfamily B, polypeptide 6 |
| 504_at | down | 0.020955749 | ubiquitin-conjugating enzyme E2D 3 (UBC4/5 homolog, yeast) |
| 36074_at | up | 0.02103264 | imprinted in Prader-Willi syndrome |
| 41335_at | down | 0.021053426 | DKFZP566O1646 protein |
| 37337_at | down | 0.021053683 | small nuclear ribonucleoprotein polypeptide G |
| 34381_at | down | 0.02108904 | cytochrome c oxidase subunit VIIc |
| 36640_at | up | 0.021092759 | myosin, light polypeptide 2, regulatory, cardiac, slow |
| 36580_at | down | 0.02112571 | hypothetical protein FLJ13910 |
| 34172_s_at | up | 0.021127469 | DNA segment on chromosome X and Y (unique) 155 expressed |
| | | | sequence |
| 37490_at | up | 0.021147824 | solute carrier family 4, anion exchanger, member 3 |
| 40454_at | up | 0.021159268 | FAT tumor suppressor homolog 1 (Drosophila) |
| 37541_at | up | 0.021166157 | selectin P ligand |
| 40261_at | up | 0.021175781 | RNA binding motif protein 9 |
| 32463_at | up | 0.021175784 | Rho GTPase activating protein 6 |
| 38146_at | up | 0.021176618 | suppression of tumorigenicity 18 (breast carcinoma) (zinc finger |
| | | | protein) |
| 34495_r_at | up | 0.021186298 | synaptogyrin 4 |
| 32850_at | down | 0.021186976 | nucleoporin 153 kDa |
| 38963_i_at | up | 0.021187774 | Wiskott-Aldrich syndrome (eczema-thrombocytopenia) |
| 365_at | up | 0.021193084 | cylicin, basic protein of sperm head cytoskeleton 1 |
| 35276_at | up | 0.02120371 | claudin 4 |
| 33875_at | down | 0.021227384 | ATPase, H+ transporting, lysosomal 9 kDa, V0 subunit e |
| 36729_g_at | up | 0.02125517 | adrenergic, alpha-1D-, receptor |
| 34922_at | up | 0.02126488 | cadherin 19, type 2 |
| 41425_at | down | 0.021323848 | Friend leukemia virus integration 1 |
| 41256_at | down | 0.021325224 | eukaryotic translation elongation factor 1 delta (guanine nucleotid |
| | | | exchange protein) |
| 39341_at | up | 0.021340838 | thyroid hormone receptor interactor 6 |
| 40558_at | up | 0.021360635 | guanylate cyclase activator 1B (retina) |
| 41086_at | up | 0.021365747 | regulator of G-protein signalling 20 |
| 38592_s_at | up | 0.021377698 | KIAA0284 protein |
| 41347_at | up | 0.021382759 | iroquois homeobox protein 5 |
| 40411_at | down | 0.021393313 | nuclear receptor coactivator 6 |
| 1344_at | up | 0.02140639 | paired box gene 3 (Waardenburg syndrome 1) |
| 38117_at | up | 0.021409201 | SEC24 related gene family, member C (S. cerevisiae) |
| 34147_g_at | up | 0.021417061 | 8-oxoguanine DNA glycosylase |
| 33363_at | up | 0.021424419 | JTV1 gene |
| 39966_at | up | 0.021450725 | chondroitin sulfate proteoglycan 5 (neuroglycan C) |
| 38499_s_at | up | 0.021471019 | myelin-associated oligodendrocyte basic protein |
| 39914_r_at | up | 0.02147462 | transient receptor potential cation channel, subfamily M, member 2 |
| 36271_at | up | 0.021478981 | KIAA1024 protein |
| 40017_at | up | 0.021500608 | DKFZP586H2123 protein |
| 40141_at | down | 0.021547278 | cullin 4B |
| 39857_at | down | 0.021570023 | syntaxin 11 |
| 34708_at | up | 0.021612712 | ficolin (collagen/fibrinogen domain containing) 3 (Hakata antigen) |
| 40111_g_at | up | 0.02161511 | isocitrate dehydrogenase 3 (NAD+) beta |
| 38779_r_at | up | 0.021635798 | hepatoma-derived growth factor (high-mobility group protein 1- |
| | | | like) |
| 41705_at | up | 0.021662157 | radical fringe homolog (Drosophila) |
| 35533_f_at | up | 0.021728712 | killer cell lectin-like receptor subfamily C, member 4 |
| 34682_at | up | 0.021746147 | hypothetical protein DKFZp566H0824 |
| 40484_g_at | up | 0.021753668 | transcriptional activator of the c-fos promoter |
| 37600_at | up | 0.021776035 | extracellular matrix protein 1 |
| 41830_at | down | 0.021821477 | KIAA0494 gene product |
| 32293_at | up | 0.021838004 | luteinizing hormone/choriogonadotropin receptor |
| 38512_r_at | up | 0.02189719 | ELAV (embryonic lethal, abnormal vision, Drosophila)-like 3 (Hu |
| | | | antigen C) |
| 31585_at | up | 0.021930368 | glutamate receptor, metabotropic 7 |
| 38096_f_at | down | 0.021970254 | major histocompatibility complex, class II, DP beta 1 |
| 39792_at | down | 0.021996389 | heterogeneous nuclear ribonucleoprotein R |
| 33863_at | up | 0.022049912 | hypoxia up-regulated 1 |
| 40596_at | up | 0.022092324 | Treacher Collins-Franceschetti syndrome 1 |
| 32969_r_at | up | 0.022103816 | VGF nerve growth factor inducible |
| 39803_s_at | up | 0.022127147 | chromosome 21 open reading frame 2 |
| 38229_at | up | 0.02213322 | cytochrome P450, family 3, subfamily A, polypeptide 5 |
| | | | pseudogene 2 |
| 32316_s_at | down | 0.022134897 | heat shock 90 kDa protein 1, alpha |
| 32414_at | up | 0.022161556 | |
| 35037_at | up | 0.022179733 | solute carrier family 28 (sodium-coupled nucleoside transporter), |
| | | | member 1 |
| 34502_g_at | up | 0.022183005 | runt-related transcription factor 2 |
| 1744_at | up | 0.022223723 | |
| 36722_s_at | up | 0.022234623 | hepatocyte nuclear factor 4, alpha |
| 35403_at | down | 0.022253864 | KIAA1094 protein |
| 41553_at | up | 0.022276095 | chromosome 8 open reading frame 1 |
| 31904_at | up | 0.022323192 | phosphodiesterase 2A, cGMP-stimulated |
| 1453_at | down | 0.022339505 | MAD, mothers against decapentaplegic homolog 2 (Drosophila) |
| 271_s_at | up | 0.022347943 | cathepsin E |
| 35193_at | down | 0.022417379 | chromosome condensation 1-like |
| 1766_g_at | up | 0.022422468 | caspase 10, apoptosis-related cysteine protease |
| 1890_at | up | 0.022429583 | prostate differentiation factor |
| 346_s_at | up | 0.022432014 | angiotensin II receptor, type 1 |
| 36209_at | down | 0.022446797 | bromodomain containing 2 |
| 1271_g_at | up | 0.022453783 | v-rel reticuloendotheliosis viral oncogene homolog A, nuclear |
| | | | factor of kappa light polypeptide gene enhancer in B-cells 3, p65 |
| | | | (avian) |
| 1608_at | up | 0.022472991 | |
| 36168_at | up | 0.022480506 | fibroblast growth factor receptor 1 (fms-related tyrosine kinase 2, |
| | | | Pfeiffer syndrome) |
| 32014_at | up | 0.022498779 | a disintegrin and metalloproteinase domain 18 |
| 161_at | up | 0.022524961 | RAB9, member RAS oncogene family, pseudogene 1 |
| 313_at | up | 0.022571235 | |
| 37466_at | down | 0.022589707 | RAB7, member RAS oncogene family-like 1 |
| 31521_f_at | down | 0.02267027 | histone 1, H4j |
| 40378_at | up | 0.02267704 | SH3-domain GRB2-like 2 |
| 160037_at | up | 0.022764551 | matrix metalloproteinase 15 (membrane-inserted) |
| 31879_at | down | 0.022766829 | far upstream element (FUSE) binding protein 3 |
| 780_at | up | 0.022771077 | runt-related transcription factor 1 (acute myeloid leukemia 1; aml1 |
| | | | oncogene) |
| 39234_at | up | 0.02279868 | DKFZP586I111 protein |
| 1675_at | down | 0.022841517 | RAS p21 protein activator (GTPase activating protein) 1 |
| 37328_at | down | 0.022861457 | pleckstrin |
| 32952_at | up | 0.0228892 | Retina-derived POU-domain factor-1 |
| 34582_at | up | 0.022906704 | solute carrier family 1 (glial high affinity glutamate transporter), |
| | | | member 2 |
| 38224_at | up | 0.022931589 | small nuclear RNA activating complex, polypeptide 3, 50 kDa |
| 32811_at | up | 0.022951758 | myosin IC |
| 33463_at | up | 0.022978048 | xanthine dehydrogenase |
| 40830_at | up | 0.023005272 | DnaJ (Hsp40) homolog, subfamily C, member 4 |
| 39913_at | up | 0.023012464 | heparan sulfate 6-O-sulfotransferase 1 |
| 1577_at | up | 0.023034692 | androgen receptor (dihydrotestosterone receptor; testicular |
| | | | feminization; spinal and bulbar muscular atrophy; Kennedy |
| | | | disease) |
| 38452_at | up | 0.023045399 | hypothetical protein MGC5466 |
| 37482_at | up | 0.023062615 | aldo-keto reductase family 1, member B10 (aldose reductase) |
| 33471_g_at | up | 0.023074443 | KIAA1719 protein |
| 40322_at | up | 0.023076512 | interleukin 1 receptor-like 1 |
| 41871_at | up | 0.023125317 | lung type-I cell membrane-associated glycoprotein |
| 1598_g_at | up | 0.023128654 | growth arrest-specific 6 |
| 38160_at | down | 0.023139429 | lymphocyte antigen 75 |
| 120_at | up | 0.02321075 | integrin, alpha 1 |
| 32947_at | up | 0.023220361 | sodium channel, voltage-gated, type IX, alpha |
| 38199_at | up | 0.023220366 | similar to RIKEN cDNA 2610307I21 |
| 32047_at | up | 0.023234807 | DNA fragmentation factor, 45 kDa, alpha polypeptide |
| 32080_at | up | 0.023277114 | tetracycline transporter-like protein |
| 39483_s_at | down | 0.023286561 | integrin, beta 1 (fibronectin receptor, beta polypeptide, antigen |
| | | | CD29 includes MDF2, MSK12) |
| 40083_at | down | 0.023290559 | KIAA0625 protein |
| 39832_at | up | 0.02332293 | arsenate resistance protein ARS2 |
| 1627_at | up | 0.02332432 | |
| 2032_s_at | up | 0.023358429 | integrin, alpha V (vitronectin receptor, alpha polypeptide, antigen |
| | | | CD51) |
| 32100_r_at | up | 0.023383121 | galactosamine (N-acetyl)-6-sulfate sulfatase (Morquio syndrome, |
| | | | mucopolysaccharidosis type IVA) |
| 35644_at | up | 0.023398424 | hephaestin |
| 38901_at | up | 0.023439148 | ubiquitin specific protease 19 |
| 40183_at | up | 0.023444505 | coactivator-associated arginine methyltransferase-1 |
| 222_at | up | 0.023456142 | exostoses (multiple) 1 |
| 36327_at | up | 0.023463563 | potassium inwardly-rectifying channel, subfamily J, member 1 |
| 32329_at | up | 0.023467672 | keratin, hair, basic, 6 (monilethrix) |
| 34166_at | up | 0.023483105 | solute carrier family 6 (neurotransmitter transporter, L-proline), |
| | | | member 7 |
| 37690_at | up | 0.023498915 | ilvB (bacterial acetolactate synthase)-like |
| 40856_at | up | 0.023527316 | serine (or cysteine) proteinase inhibitor, clade F (alpha-2 |
| | | | antiplasmin, pigment epithelium derived factor), member 1 |
| 31740_s_at | up | 0.023574982 | paired box gene 4 |
| 40315_at | up | 0.023606902 | serine protease inhibitor, Kazal type, 5 |
| 40085_s_at | down | 0.023614131 | transcription factor CP2 |
| 32620_at | up | 0.023615667 | fetuin B |
| 36972_at | down | 0.023647274 | coated vesicle membrane protein |
| 37784_at | up | 0.023651963 | |
| 37172_at | up | 0.023681682 | carboxypeptidase B2 (plasma, carboxypeptidase U) |
| 39412_at | up | 0.023685437 | tripartite motif-containing 26 |
| 33197_at | up | 0.023691092 | myosin VIIA (Usher syndrome 1B (autosomal recessive, severe)) |
| 39876_at | up | 0.023730024 | ectonucleoside triphosphate diphosphohydrolase 6 (putative |
| | | | function) |
| 1242_at | down | 0.023776473 | Ets2 repressor factor |
| 40457_at | down | 0.023802156 | splicing factor, arginine/serine-rich 3 |
| 33590_at | up | 0.023828936 | |
| 40555_at | down | 0.023877609 | ras homolog gene family, member Q |
| 39706_at | down | 0.023892621 | copine III |
| 31315_at | up | 0.023921194 | immunoglobulin lambda locus |
| 39941_at | down | 0.023929111 | RAD50 homolog (S. cerevisiae) |
| 38649_at | down | 0.023932788 | KIAA0970 protein |
| 33190_g_at | up | 0.023944372 | chromosome 10 open reading frame 6 |
| 37714_at | up | 0.023986452 | growth associated protein 43 |
| 32243_g_at | up | 0.024008044 | crystallin, alpha B |
| 41248_at | down | 0.024018398 | likely ortholog of mouse variant polyadenylation protein CSTF-64 |
| 37664_at | up | 0.024027194 | developmentally regulated GTP binding protein 2 |
| 41867_at | up | 0.024045128 | old astrocyte specifically induced substance |
| 41193_at | down | 0.024051364 | dual specificity phosphatase 6 |
| 35205_at | up | 0.02406628 | cofactor of BRCA1 |
| 40981_at | up | 0.024097695 | helicase with SNF2 domain 1 |
| 38892_at | down | 0.024103087 | KIAA0240 protein |
| 35363_at | down | 0.024128323 | DEAD/H (Asp-Glu-Ala-Asp/His) box polypeptide 17, 72 kDa |
| 36540_at | up | 0.024133889 | Rho-related BTB domain containing 2 |
| 1724_at | up | 0.02414433 | E2F transcription factor 4, p107/p130-binding |
| 40009_at | up | 0.024159688 | fragile X mental retardation 2 |
| 534_s_at | up | 0.02416274 | folate receptor 1 (adult) |
| 37723_at | down | 0.024176482 | cyclin G2 |
| 35489_at | up | 0.024251424 | meprin A, alpha (PABA peptide hydrolase) |
| 227_g_at | down | 0.024262631 | protein kinase, cAMP-dependent, regulatory, type I, alpha (tissue |
| | | | specific extinguisher 1) |
| 1316_at | up | 0.024262768 | thyroid hormone receptor, alpha (erythroblastic leukemia viral (v- |
| | | | erb-a) oncogene homolog, avian) |
| 36018_at | up | 0.024269622 | SRY (sex determining region Y)-box 10 |
| 32728_at | up | 0.024280106 | amphiphysin (Stiff-Man syndrome with breast cancer 128 kDa |
| | | | autoantigen) |
| 1825_at | down | 0.024316799 | IQ motif containing GTPase activating protein 1 |
| 38594_i_at | up | 0.024320041 | KIAA0284 protein |
| 36014_at | up | 0.024334234 | G protein-coupled receptor 126 |
| 1898_at | up | 0.024335806 | tripartite motif-containing 29 |
| 605_at | up | 0.024351877 | vesicle amine transport protein 1 homolog (T californica) |
| 37122_at | up | 0.024415515 | perilipin |
| 34933_at | up | 0.024433571 | paired box gene 9 |
| 39897_at | down | 0.024487745 | splicing factor YT521-B |
| 38558_at | up | 0.024488796 | myelin associated glycoprotein |
| 1072_g_at | up | 0.024497847 | GATA binding protein 2 |
| 37285_at | up | 0.024530431 | aminolevulinate, delta-, synthase 2 (sideroblastic/hypochromic |
| | | | anemia) |
| 33069_f_at | up | 0.02458139 | UDP glycosyltransferase 2 family, polypeptide B15 |
| 36031_at | down | 0.024608101 | inhibitor of growth family, member 1 |
| 37511_at | up | 0.02461872 | B9 protein |
| 39158_at | up | 0.024708448 | activating transcription factor 5 |
| 39744_at | down | 0.024753389 | DEAD/H (Asp-Glu-Ala-Asp/His) box polypeptide 3 |
| 1658_g_at | up | 0.024762036 | protein tyrosine phosphatase, receptor type, R |
| 33346_r_at | up | 0.024763958 | tubulin, gamma 1 |
| 33245_at | down | 0.024776732 | mitogen-activated protein kinase 13 |
| 32066_g_at | up | 0.024824897 | cAMP responsive element modulator |
| 36143_at | down | 0.024857059 | caspase 3, apoptosis-related cysteine protease |
| 36269_at | up | 0.024858179 | a disintegrin-like and metalloprotease (reprolysin type) with |
| | | | thrombospondin type 1 motif, 3 |
| 33071_at | up | 0.024871677 | histone 1, H2bo |
| 32214_at | down | 0.024880537 | thioredoxin-like, 32 kDa |
| 39797_at | down | 0.024903869 | ubiquitin ligase E3 alpha-II |
| 33359_at | up | 0.024907251 | latrophilin 3 |
| 34981_at | up | 0.024912595 | potassium voltage-gated channel, shaker-related subfamily, |
| | | | member 5 |
| 41283_at | down | 0.02498879 | heterogeneous nuclear ribonucleoprotein H3 (2H9) |
| 37134_f_at | up | 0.025015777 | glutamate receptor, ionotropic, N-methyl D-aspartate 1 |
| 35298_at | down | 0.02502439 | eukaryotic translation initiation factor 3, subunit 7 zeta, 66/67 kDa |
| 39625_at | up | 0.025069218 | |
| 35387_r_at | up | 0.025073822 | acetylcholinesterase (YT blood group) |
| 31893_at | up | 0.025085609 | ADP-ribosylation factor-like 2 |
| 34305_at | down | 0.025125133 | poly(rC) binding protein 1 |
| 39195_s_at | up | 0.025149176 | leucine-rich repeats and immunoglobulin-like domains 1 |
| 37745_s_at | up | 0.025167309 | suppression of tumorigenicity 5 |
| 39705_at | up | 0.025174264 | SIN3 homolog B, transcriptional regulator (yeast) |
| 38772_at | up | 0.025198255 | cysteine-rich, angiogenic inducer, 61 |
| 40746_at | up | 0.025242905 | glutamate receptor, ionotropic, AMPA 2 |
| 1482_g_at | up | 0.025285618 | matrix metalloproteinase 12 (macrophage elastase) |
| 34850_at | down | 0.025301859 | ubiquitin-conjugating enzyme E2E 3 (UBC4/5 homolog, yeast) |
| 39123_s_at | up | 0.025338973 | transient receptor potential cation channel, subfamily C, member |
| 186_at | up | 0.025376205 | protein kinase, AMP-activated, alpha 2 catalytic subunit |
| 40108_at | down | 0.025392776 | basic leucine zipper and W2 domains 1 |
| 39517_at | down | 0.025404739 | HTGN29 protein |
| 35997_g_at | up | 0.025409702 | ZW10 interactor anti-sense |
| 37254_at | up | 0.025432778 | zinc finger protein 133 (clone pHZ-13) |
| 32872_at | up | 0.025443671 | |
| 40988_at | down | 0.025483862 | YME1-like 1 (S. cerevisiae) |
| 35813_at | up | 0.025524962 | transportin-SR |
| 31778_at | up | 0.025549358 | gap junction protein, alpha 8, 50 kDa (connexin 50) |
| 41652_at | up | 0.025591657 | collagen, type XI, alpha 2 |
| 34805_at | up | 0.025620228 | hypothetical protein MGC2574 |
| 37720_at | down | 0.025630855 | heat shock 60 kDa protein 1 (chaperonin) |
| 35447_s_at | up | 0.025651802 | acetylserotonin O-methyltransferase |
| 39172_at | down | 0.025654012 | hypothetical protein FLJ14547 |
| 31995_g_at | up | 0.025687138 | ADP-ribosylation factor guanine nucleotide-exchange factor 2 |
| | | | (brefeldin A-inhibited) |
| 36875_at | down | 0.02574627 | inhibitor of Bruton's tyrsoine kinase |
| 32301_at | up | 0.025789564 | guanylate cyclase 1, soluble, alpha 2 |
| 35010_at | up | 0.025810869 | HLA complex group 8 |
| 36367_at | up | 0.025830383 | protocadherin 11 X-linked |
| 37488_at | up | 0.02585489 | farnesyltransferase, CAAX box, beta |
| 726_f_at | up | 0.025905675 | |
| 31935_s_at | up | 0.025909825 | DEAD/H (Asp-Glu-Ala-Asp/His) box polypeptide 11 (CHL1-like |
| | | | helicase homolog, S. cerevisiae) |
| 34819_at | down | 0.02591587 | CD164 antigen, sialomucin |
| 1449_at | down | 0.02591764 | proteasome (prosome, macropain) subunit, alpha type, 4 |
| 35903_at | up | 0.026012105 | oligodendrocyte myelin glycoprotein |
| 36890_at | up | 0.026034077 | periplakin |
| 37794_at | up | 0.026039344 | |
| 34903_at | up | 0.026052906 | KIAA1218 protein |
| 38215_at | up | 0.026063497 | chromosome 22 open reading frame 1 |
| 160022_at | down | 0.026067385 | colony stimulating factor 1 receptor, formerly McDonough feline |
| | | | sarcoma viral (v-fms) oncogene homolog |
| 31727_at | up | 0.026069485 | ectonucleoside triphosphate diphosphohydrolase 2 |
| 33732_at | up | 0.026076534 | adaptor-related protein complex 4, mu 1 subunit |
| 35845_at | down | 0.026142932 | SEC24 related gene family, member B (S. cerevisiae) |
| 1954_at | up | 0.026144402 | kinase insert domain receptor (a type III receptor tyrosine kinase) |
| 39820_at | up | 0.026155871 | RNA polymerase I transcription factor RRN3 |
| 1772_s_at | down | 0.026179299 | farnesyltransferase, CAAX box, alpha |
| 34224_at | up | 0.026179949 | fatty acid desaturase 3 |
| 36445_at | up | 0.026189391 | chemokine (C—C motif) ligand 23 |
| 35614_at | down | 0.026217146 | transcription factor-like 5 (basic helix-loop-helix) |
| 37558_at | up | 0.026217683 | IGF-II mRNA-binding protein 3 |
| 33172_at | up | 0.026290468 | hypothetical protein FLJ10849 |
| 39877_at | up | 0.026319532 | potassium voltage-gated channel, Shaw-related subfamily, |
| | | | member 4 |
| 31922_i_at | up | 0.026326432 | Ac-like transposable element |
| 40534_at | up | 0.026341303 | protein tyrosine phosphatase, receptor type, D |
| 1632_at | up | 0.026362721 | |
| 1411_at | up | 0.026495528 | cytochrome P450, family 11, subfamily B, polypeptide 1 |
| 34576_at | up | 0.026513529 | melanoma antigen, family A, 8 |
| 36037_g_at | up | 0.026518478 | spectrin, beta, erythrocytic (includes spherocytosis, clinical type I) |
| 37736_at | down | 0.02652131 | protein-L-isoaspartate (D-aspartate) O-methyltransferase |
| 33898_at | up | 0.026563809 | microspherule protein 1 |
| 1243_at | up | 0.026567603 | damage-specific DNA binding protein 2, 48 kDa |
| 37969_at | up | 0.02662293 | prostaglandin-endoperoxide synthase 1 (prostaglandin G/H |
| | | | synthase and cyclooxygenase) |
| 38967_at | down | 0.026666699 | chromosome 14 open reading frame 2 |
| 34563_at | up | 0.026707179 | kinesin family member 14 |
| 41637_at | up | 0.026727557 | MYLE protein |
| 33887_at | up | 0.026739087 | hepatocyte growth factor-regulated tyrosine kinase substrate |
| 36568_at | up | 0.026745623 | solute carrier family 17 (sodium-dependent inorganic phosphate |
| | | | cotransporter), member 7 |
| 37470_at | up | 0.026754858 | leukocyte-associated Ig-like receptor 1 |
| 38076_at | up | 0.026783134 | ATP synthase, H+ transporting, mitochondrial F0 complex, subuni |
| | | | c (subunit 9), isoform 1 |
| 598_at | up | 0.026832268 | collagen, type II, alpha 1 (primary osteoarthritis, |
| | | | spondyloepiphyseal dysplasia, congenital) |
| 32540_at | up | 0.026889206 | protein phosphatase 3 (formerly 2B), catalytic subunit, gamma |
| | | | isoform (calcineurin A gamma) |
| 34398_at | up | 0.026895399 | heat shock 105 kDa/110 kDa protein 1 |
| 34880_at | down | 0.02690197 | hypothetical protein MGC10433 |
| 41333_at | down | 0.02691227 | centaurin, beta 2 |
| 687_at | up | 0.026947578 | |
| 35150_at | up | 0.026964489 | tumor necrosis factor receptor superfamily, member 5 |
| 36736_f_at | down | 0.026980201 | phosphoserine phosphatase |
| 31716_at | up | 0.026993707 | protocadherin 1 (cadherin-like 1) |
| 34294_at | up | 0.02707396 | kinesin family member C3 |
| 32898_at | up | 0.027097609 | actin like protein |
| 40858_at | up | 0.02711133 | pregnancy specific beta-1-glycoprotein 1 |
| 39740_g_at | down | 0.02714561 | nascent-polypeptide-associated complex alpha polypeptide |
| 32841_at | down | 0.027165304 | zinc finger protein 9 (a cellular retroviral nucleic acid binding |
| | | | protein) |
| 33253_at | down | 0.027189829 | tripartite motif-containing 14 |
| 38680_at | up | 0.027225635 | small nuclear ribonucleoprotein polypeptide E |
| 40655_at | up | 0.027239968 | huntingtin-associated protein interacting protein (duo) |
| 1671_s_at | up | 0.027242438 | mitogen-activated protein kinase 14 |
| 335_r_at | up | 0.027250575 | |
| 31846_at | up | 0.02727266 | ras homolog gene family, member D |
| 38564_at | up | 0.027279452 | origin recognition complex, subunit 1-like (yeast) |
| 34926_at | up | 0.027289913 | CD1A antigen, a polypeptide |
| 39746_at | down | 0.027291894 | polymerase (RNA) II (DNA directed) polypeptide B, 140 kDa |
| 1041_at | up | 0.027299453 | ephrin-A5 |
| 34159_at | up | 0.027348803 | RAB7, member RAS oncogene family |
| 39972_at | up | 0.027368356 | G protein-coupled receptor 17 |
| 31831_at | up | 0.027395463 | smoothelin |
| 31800_at | up | 0.027462703 | |
| 31594_at | up | 0.027473933 | keratin, hair, acidic, 3A |
| 34154_at | up | 0.02749165 | cholinergic receptor, nicotinic, beta polypeptide 2 (neuronal) |
| 38048_at | up | 0.027500957 | RNA binding protein with multiple splicing |
| 1011_s_at | down | 0.027503898 | tyrosine 3-monooxygenase/tryptophan 5-monooxygenase |
| | | | activation protein, epsilon polypeptide |
| 35075_at | up | 0.027506345 | C18B11 homolog (44.9 kD) |
| 37374_at | down | 0.027538074 | annexin A4 |
| 33719_at | up | 0.027545401 | synaptopodin |
| 31446_s_at | up | 0.027567801 | proline rich 5 (salivary) |
| 34537_at | up | 0.027593937 | potassium inwardly-rectifying channel, subfamily J, member 12 |
| 36274_at | up | 0.027615478 | solute carrier family 7 (cationic amino acid transporter, y+ system) |
| | | | member 1 |
| 36399_at | up | 0.027680522 | pre-mRNA splicing SR protein rA4 |
| 34823_at | down | 0.02770756 | dipeptidylpeptidase 4 (CD26, adenosine deaminase complexing |
| | | | protein 2) |
| 1558_g_at | down | 0.027712082 | p21/Cdc42/Rac1-activated kinase 1 (STE20 homolog, yeast) |
| 1661_i_at | up | 0.027721213 | |
| 36715_at | up | 0.02776357 | adrenergic, alpha-1A-, receptor |
| 39572_at | up | 0.027785371 | glutamate receptor, ionotropic, kainate 2 |
| 38016_at | down | 0.027802172 | heterogeneous nuclear ribonucleoprotein D (AU-rich element RNA |
| | | | binding protein 1, 37 kDa) |
| 38077_at | up | 0.027810469 | collagen, type VI, alpha 3 |
| 40557_at | up | 0.02789479 | guanylate cyclase activator 1B (retina) |
| 34486_at | up | 0.027907141 | |
| 39573_at | up | 0.027923396 | glutamate receptor, ionotropic, kainate 2 |
| 37780_at | up | 0.027968479 | piccolo (presynaptic cytomatrix protein) |
| 32019_at | up | 0.02799539 | DKFZP434C153 protein |
| 31522_f_at | up | 0.028090065 | histone 1, H2bf |
| 685_f_at | down | 0.028154459 | tubulin, alpha 1 (testis specific) |
| 37840_at | up | 0.02820166 | cyclic nucleotide gated channel alpha 1 |
| 40146_at | down | 0.028209265 | RAP1B, member of RAS oncogene family |
| 41002_at | up | 0.028243866 | solute carrier family 16 (monocarboxylic acid transporters), |
| | | | member 5 |
| 32012_at | up | 0.028268131 | pecanex homolog (Drosophila) |
| 31984_at | up | 0.028270556 | |
| 41294_at | up | 0.02828124 | keratin 7 |
| 41633_at | up | 0.028282157 | sentrin/SUMO-specific protease 3 |
| 32184_at | down | 0.028317805 | LIM domain only 2 (rhombotin-like 1) |
| 40913_at | down | 0.028333047 | ATPase, Ca++ transporting, plasma membrane 4 |
| 33996_at | up | 0.028357358 | neuromedin B receptor |
| 34640_at | up | 0.028368648 | transcription factor 1, hepatic; LF-B1, hepatic nuclear factor |
| | | | (HNF1), albumin proximal factor |
| 38962_at | up | 0.028378275 | KIAA0298 gene product |
| 37157_at | up | 0.028380947 | calbindin 2, 29 kDa (calretinin) |
| 2028_s_at | up | 0.028383411 | E2F transcription factor 1 |
| 38198_at | up | 0.028391962 | similar to RIKEN cDNA 2610307I21 |
| 1659_s_at | down | 0.028544189 | Ras homolog enriched in brain 2 |
| 38729_at | up | 0.028553044 | FK506 binding protein 4, 59 kDa |
| 33950_g_at | up | 0.028656928 | corticotropin releasing hormone receptor 2 |
| 41555_at | up | 0.028666961 | heparan sulfate (glucosamine) 3-O-sulfotransferase 1 |
| 32946_r_at | up | 0.028673572 | mannose-binding lectin (protein C) 2, soluble (opsonic defect) |
| 36981_at | down | 0.028721449 | signal recognition particle 9 kDa |
| 31500_at | up | 0.028739978 | N-myc downstream regulated gene 1 |
| 105_at | up | 0.028747273 | nuclear receptor subfamily 1, group I, member 3 |
| 37369_s_at | up | 0.028795072 | nuclear factor of activated T-cells, cytoplasmic, calcineurin- |
| | | | dependent 4 |
| 1900_at | up | 0.028796398 | retinoblastoma 1 (including osteosarcoma) |
| 37358_at | down | 0.028871178 | ubiquitin-conjugating enzyme E2E 1 (UBC4/5 homolog, yeast) |
| 1062_g_at | down | 0.028894304 | interleukin 10 receptor, alpha |
| 247_s_at | up | 0.028908676 | cytochrome P450, family 21, subfamily A, polypeptide 2 |
| 33184_at | up | 0.028926665 | guanylate cyclase activator 1A (retina) |
| 40144_at | down | 0.028926721 | protein tyrosine phosphatase, non-receptor type substrate 1 |
| 1902_at | up | 0.028929735 | excision repair cross-complementing rodent repair deficiency, |
| | | | complementation group 1 (includes overlapping antisense |
| | | | sequence) |
| 237_s_at | down | 0.02901457 | protein phosphatase 2 (formerly 2A), catalytic subunit, alpha |
| | | | isoform |
| 32871_at | up | 0.029039566 | |
| 40129_at | down | 0.029043392 | protein kinase, DNA-activated, catalytic polypeptide |
| 33852_at | down | 0.029046277 | TIA1 cytotoxic granule-associated RNA binding protein |
| 41190_at | up | 0.029048795 | tumor necrosis factor receptor superfamily, member 25 |
| 34505_at | up | 0.029085088 | likely ortholog of mouse myocytic induction/differentiation |
| | | | originator |
| 39911_at | up | 0.029122116 | hypothetical protein LOC51257 |
| 34334_at | up | 0.029129367 | ephrin-B2 |
| 32819_at | down | 0.029198273 | histone 1, H2bk |
| 40012_at | up | 0.02922851 | low density lipoprotein receptor-related protein 8, apolipoprotein e |
| | | | receptor |
| 1505_at | up | 0.029239491 | thymidylate synthetase |
| 35814_at | down | 0.029305093 | dendritic cell protein |
| 39955_at | up | 0.029314355 | deleted in lymphocytic leukemia, 2 |
| 37989_at | up | 0.029335797 | parathyroid hormone-like hormone |
| 33416_at | up | 0.029369792 | KIAA1049 protein |
| 41678_at | up | 0.029375379 | EphB2 |
| 41757_at | down | 0.029390472 | VAMP (vesicle-associated membrane protein)-associated protein |
| | | | B and C |
| 35981_at | up | 0.029401129 | regenerating islet-derived 1 beta (pancreatic stone protein, |
| | | | pancreatic thread protein) |
| 32534_f_at | up | 0.029407674 | vesicle-associated membrane protein 5 (myobrevin) |
| 716_at | up | 0.029510223 | gamma-glutamyltransferase-like activity 1 |
| 41336_at | up | 0.029552307 | DKFZP566O1646 protein |
| 34774_at | down | 0.029555309 | palmitoyl-protein thioesterase 1 (ceroid-lipofuscinosis, neuronal 1, |
| | | | infantile) |
| 1784_s_at | down | 0.029583432 | retinoblastoma binding protein 1 |
| 1517_at | up | 0.029610936 | cytochrome P450, family 2, subfamily F, polypeptide 1 |
| 40700_at | up | 0.029668614 | SP140 nuclear body protein |
| 40323_at | up | 0.029749549 | CD38 antigen (p45) |
| 37238_s_at | up | 0.029796885 | membrane-associated tyrosine- and threonine-specific cdc2- |
| | | | Inhibitory kinase |
| 2086_s_at | up | 0.029837041 | TYRO3 protein tyrosine kinase |
| 34351_at | up | 0.029891028 | phospholipase C, gamma 1 (formerly subtype 148) |
| 467_at | down | 0.029903899 | osteoclast stimulating factor 1 |
| 35571_at | up | 0.029924365 | coagulation factor II (thrombin) receptor-like 3 |
| 33965_at | up | 0.029960532 | chemokine (C—C motif) ligand 1 |
| 41696_at | up | 0.029980756 | hypothetical protein MGC3077 |
| 36285_at | up | 0.030018837 | potassium inwardly-rectifying channel, subfamily J, member 4 |
| 322_at | up | 0.030077773 | phosphoinositide-3-kinase, regulatory subunit, polypeptide 3 (p55 |
| | | | gamma) |
| 38498_at | up | 0.030096154 | crystallin, beta B2 |
| 40836_s_at | up | 0.030118354 | heterogeneous nuclear ribonucleoprotein H3 (2H9) |
| 232_at | up | 0.030119073 | laminin, gamma 1 (formerly LAMB2) |
| 39174_at | down | 0.030122401 | nuclear receptor coactivator 4 |
| 37755_at | up | 0.0301373 | BTB (POZ) domain containing 3 |
| 39967_at | up | 0.030138107 | leucine zipper, down-regulated in cancer 1 |
| 38897_at | up | 0.030160347 | solute carrier family 7 (cationic amino acid transporter, y+ system |
| | | | member 4 |
| 2050_s_at | down | 0.030191058 | ras-related C3 botulinum toxin substrate 1 (rho family, small GTP |
| | | | binding protein Rac1) |
| 35020_at | up | 0.030204148 | paired-like homeobox 2b |
| 32505_at | up | 0.030207295 | NS1-associated protein 1 |
| 33302_at | up | 0.030208659 | sarcospan (Kras oncogene-associated gene) |
| 33799_at | down | 0.030209223 | seven in absentia homolog 2 (Drosophila) |
| 40840_at | up | 0.03025459 | peptidylprolyl isomerase F (cyclophilin F) |
| 191_at | up | 0.030325967 | mucin 8, tracheobronchial |
| 35090_g_at | up | 0.030333092 | neuregulin 2 |
| 40068_at | down | 0.030347738 | syntaxin 5A |
| 39049_at | down | 0.030357581 | chromosome 6 open reading frame 9 |
| 34067_at | up | 0.030369064 | |
| 35930_at | up | 0.030372209 | testis specific protein, Y-linked |
| 33459_at | up | 0.03040696 | |
| 37418_at | up | 0.030409086 | POU domain, class 2, transcription factor 2 |
| 34149_at | up | 0.030409172 | pleiotropic regulator 1 (PRL1homolog, Arabidopsis) |
| 41085_at | up | 0.030435923 | polymerase (DNA directed), epsilon 2 (p59 subunit) |
| 40783_s_at | up | 0.030449434 | phosphatidylinositol 4-kinase, catalytic, alpha polypeptide |
| 892_at | up | 0.030460619 | transmembrane 4 superfamily member 1 |
| 40203_at | down | 0.030488383 | putative translation initiation factor |
| 32002_at | up | 0.030532859 | GDNF family receptor alpha 3 |
| 37463_r_at | up | 0.030546476 | splicing factor 3a, subunit 2, 66 kDa |
| 38714_at | up | 0.03055242 | glycophorin A (includes MN blood group) |
| 34726_at | up | 0.030570264 | calcium channel, voltage-dependent, beta 3 subunit |
| 41374_at | up | 0.030608379 | ribosomal protein S6 kinase, 70 kDa, polypeptide 2 |
| 36023_at | down | 0.030629492 | proline-rich protein HaeIII subfamily 1 |
| 36742_at | up | 0.030653119 | tripartite motif-containing 15 |
| 36771_at | up | 0.030722552 | cannabinoid receptor 2 (macrophage) |
| 159_at | up | 0.030730643 | vascular endothelial growth factor C |
| AFFX-CreX-5_at | up | 0.030730678 | |
| 39429_at | up | 0.030749245 | UV radiation resistance associated gene |
| 34463_at | up | 0.030750243 | deoxyribonuclease I |
| 824_at | down | 0.030756422 | glutathione-S-transferase like; glutathione transferase omega |
| 35593_at | up | 0.030776042 | amine oxidase, copper containing 2 (retina-specific) |
| 1090_f_at | up | 0.030790531 | vacuolar protein sorting 4B (yeast) |
| 33576_at | up | 0.03087025 | KIAA0918 protein |
| 923_at | up | 0.030878031 | ubiquitin-like 4 |
| 39607_at | up | 0.030920096 | myotubularin related protein 9 |
| 34554_at | up | 0.030941865 | glycine receptor, alpha 2 |
| 1858_at | up | 0.030996193 | tumor necrosis factor (ligand) superfamily, member 6 |
| 36433_at | up | 0.031004583 | glycine receptor, alpha 3 |
| 1427_g_at | down | 0.031021674 | Src-like-adaptor |
| 634_at | up | 0.031040949 | protease, serine, 8 (prostasin) |
| 32700_at | down | 0.031048619 | guanylate binding protein 2, interferon-inducible |
| 38492_at | up | 0.031061258 | kynureninase (L-kynurenine hydrolase) |
| 34127_at | up | 0.031061577 | organic cationic transporter-like 3 |
| 1153_f_at | up | 0.031069865 | chorionic gonadotropin, beta polypeptide |
| 35102_at | up | 0.031116602 | zinc finger protein |
| 35325_at | down | 0.031161778 | RAB14, member RAS oncogene family |
| 35638_at | up | 0.031168273 | core-binding factor, runt domain, alpha subunit 2; translocated to, |
| | | | 1; cyclin D-related |
| 37389_at | down | 0.031316111 | small acidic protein |
| 31961_r_at | up | 0.031323988 | |
| 36439_at | up | 0.031332099 | |
| 32254_at | up | 0.031358273 | vesicle-associated membrane protein 2 (synaptobrevin 2) |
| 36701_at | up | 0.03135917 | |
| 1628_at | up | 0.031360464 | |
| 37397_at | down | 0.031394119 | platelet/endothelial cell adhesion molecule (CD31 antigen) |
| 39420_at | down | 0.031398275 | DNA-damage-inducible transcript 3 |
| 32228_at | up | 0.031413344 | adaptor-related protein complex 2, alpha 2 subunit |
| 32053_at | down | 0.031474283 | cyclin T2 |
| 36509_at | down | 0.031476516 | ribosomal protein L35a |
| 36793_at | up | 0.03149691 | hypothetical protein AY099107 |
| 31941_s_at | up | 0.031534979 | ret finger protein-like 3 |
| 33999_f_at | up | 0.031557766 | |
| 34924_at | up | 0.031605825 | kinesin family member 1B |
| 37850_at | up | 0.031616361 | hypothetical protein dJ462O23.2 |
| 1853_at | up | 0.031673028 | wingless-type MMTV integration site family, member 1 |
| 32761_at | up | 0.031711267 | serine/arginine repetitive matrix 2 |
| 35848_at | down | 0.031713305 | retinoic acid induced 17 |
| 33058_at | up | 0.03184771 | cytokeratin type II |
| 635_s_at | up | 0.031851567 | protein phosphatase 2, regulatory subunit B (B56), beta isoform |
| 35411_at | up | 0.031857375 | chromosome 16 open reading frame 7 |
| 32520_at | up | 0.031885468 | nuclear receptor subfamily 1, group D, member 1 |
| 31915_at | up | 0.031889645 | dystrophin related protein 2 |
| 36714_at | up | 0.031890786 | nuclear receptor subfamily 2, group C, member 2 |
| 34847_s_at | up | 0.031912545 | calcium/calmodulin-dependent protein kinase (CaM kinase) II beta |
| 424_s_at | up | 0.031967116 | fibroblast growth factor receptor 1 (fms-related tyrosine kinase 2, |
| | | | Pfeiffer syndrome) |
| 31414_at | up | 0.031975481 | testis-specific transcript, Y-linked 2 |
| 32896_at | up | 0.032009199 | |
| 35417_at | up | 0.03208939 | cubilin (intrinsic factor-cobalamin receptor) |
| 35263_at | down | 0.032117131 | eukaryotic translation initiation factor 4E binding protein 2 |
| 1573_at | up | 0.032134508 | platelet-derived growth factor beta polypeptide (simian sarcoma |
| | | | viral (v-sis) oncogene homolog) |
| 792_s_at | up | 0.032185586 | transcription factor AP-2 alpha (activating enhancer binding |
| | | | protein 2 alpha) |
| 33232_at | down | 0.032194657 | cysteine-rich protein 1 (intestinal) |
| 37545_at | up | 0.032225183 | secretory carrier membrane protein 5 |
| 41630_at | up | 0.032231183 | CGI-62 protein |
| 35285_at | up | 0.032272611 | solute carrier family 4, sodium bicarbonate cotransporter, member 4 |
| 41618_at | up | 0.032282842 | collagen, type XVII, alpha 1 |
| 39497_at | up | 0.032294796 | hypothetical protein FLJ10803 |
| 41324_g_at | up | 0.032328237 | forkhead box M1 |
| 36716_at | up | 0.032332777 | adrenergic, alpha-1A-, receptor |
| 38191_at | up | 0.032337124 | KIAA0645 gene product |
| 36315_i_at | up | 0.032376572 | Sec15B protein |
| 34128_at | up | 0.032402682 | UDP-Gal:betaGlcNAc beta 1,3-galactosyltransferase, polypeptide 5 |
| 33356_at | up | 0.032468294 | trinucleotide repeat containing 3 |
| 31566_at | up | 0.032480202 | |
| 37811_at | up | 0.032501096 | calcium channel, voltage-dependent, alpha 2/delta subunit 2 |
| 37035_at | down | 0.03252466 | stress-associated endoplasmic reticulum protein 1 |
| 37165_f_at | up | 0.032565127 | Rhesus blood group, CcEe antigens |
| 32499_at | up | 0.032569682 | Rho GDP dissociation inhibitor (GDI) gamma |
| 36200_at | up | 0.032605501 | HLA-B associated transcript 8 |
| 31443_at | up | 0.032611589 | |
| 38664_at | down | 0.032630779 | craniofacial development protein 1 |
| 423_at | down | 0.0326721 | Ewing sarcoma breakpoint region 1 |
| 39206_s_at | up | 0.032698078 | aggrecan 1 (chondroitin sulfate proteoglycan 1, large aggregating |
| | | | proteoglycan, antigen identified by monoclonal antibody A0122) |
| 41467_at | up | 0.032750937 | mutS homolog 5 (E. coli) |
| 38755_at | up | 0.032752728 | Fas (TNFRSF6)-associated via death domain |
| 34575_f_at | up | 0.032809836 | melanoma antigen, family A, 5 |
| 35553_at | up | 0.032825078 | TSPY-like |
| 38221_at | up | 0.032832296 | connector enhancer of KSR-like (Drosophila kinase suppressor o |
| | | | ras) |
| 35362_at | up | 0.03290105 | myosin X |
| 35400_at | up | 0.032907138 | |
| 221_s_at | up | 0.032936681 | phosphatidylinositol glycan, class A (paroxysmal nocturnal |
| | | | hemoglobinuria) |
| 36093_at | up | 0.032947133 | KIAA0614 protein |
| 36462_at | up | 0.032953356 | SMYD family member 5 |
| 1892_s_at | up | 0.032967779 | |
| 38766_at | up | 0.033003801 | Snf2-related CBP activator protein |
| 39276_g_at | up | 0.033018192 | calcium channel, voltage-dependent, L type, alpha 1D subunit |
| 37922_at | up | 0.033026389 | transcobalamin II; macrocytic anemia |
| 38390_at | up | 0.033060083 | component of oligomeric golgi complex 2 |
| 40916_at | down | 0.033066997 | hypothetical protein FLJ10097 |
| 36094_at | up | 0.033067781 | troponin T3, skeletal, fast |
| 35184_at | down | 0.033082679 | KIAA0546 protein |
| 35537_at | up | 0.033137937 | tumor necrosis factor receptor superfamily, member 10d, decoy |
| | | | with truncated death domain |
| 40759_at | up | 0.03313989 | matrix metalloproteinase 16 (membrane-inserted) |
| 38084_at | down | 0.033140959 | chromobox homolog 3 (HP1 gamma homolog, Drosophila) |
| 38934_at | up | 0.033155647 | |
| 35995_at | up | 0.033159596 | ZW10 interactor |
| 39000_at | up | 0.033222247 | N-myristoyltransferase 1 |
| 31791_at | up | 0.033224619 | tumor protein p73-like |
| 31392_r_at | up | 0.033262168 | chromosome 1 open reading frame 1 |
| 36248_at | up | 0.033265588 | NAG-5 protein |
| 35564_at | up | 0.033280281 | |
| 223_at | down | 0.033309012 | ubiquitin-conjugating enzyme E2L 3 |
| 37781_at | up | 0.033368336 | neurexin 2 |
| 40295_at | up | 0.033391699 | copine VI (neuronal) |
| 35191_at | up | 0.033424161 | KIAA0375 gene product |
| 171_at | down | 0.033442103 | von Hippel-Lindau binding protein 1 |
| 39046_at | down | 0.033467681 | histone H2A.F/Z variant |
| 36369_at | up | 0.033478368 | polymerase I and transcript release factor |
| 1789_at | down | 0.033529093 | COP9 constitutive photomorphogenic homolog subunit 5 |
| | | | (Arabidopsis) |
| 33121_g_at | down | 0.033549364 | regulator of G-protein signalling 10 |
| 32659_at | down | 0.033576868 | eukaryotic translation initiation factor 2B, subunit 4 delta, 67 kDa |
| 37234_at | up | 0.033609251 | kininogen |
| 457_s_at | down | 0.033618854 | ubiquitin-like 1 (sentrin) |
| 34173_s_at | up | 0.033652359 | contactin 5 |
| 34604_at | up | 0.0336552 | solute carrier family 6 (neurotransmitter transporter, serotonin), |
| | | | member 4 |
| 1155_at | up | 0.033705413 | v-myc myelocytomatosis viral oncogene homolog 2 (avian) |
| 35316_at | down | 0.033716548 | Ras-related GTP-binding protein |
| 34355_at | down | 0.033779716 | methyl CpG binding protein 2 (Rett syndrome) |
| 1156_at | up | 0.033793231 | Sp1 transcription factor |
| 36516_at | up | 0.033803067 | zinc finger protein ZFP100 |
| 31321_at | up | 0.033837372 | pancreatic beta cell growth factor |
| 40097_at | down | 0.033852776 | eukaryotic translation initiation factor 1A, Y chromosome |
| 33839_at | up | 0.033863607 | inositol 1,4,5-triphosphate receptor, type 2 |
| 34626_at | up | 0.033878835 | hypermethylated in cancer 1 |
| 326_i_at | down | 0.03389412 | |
| 40239_g_at | up | 0.033906971 | G protein-coupled receptor, family C, group 5, member B |
| 34722_at | up | 0.033910912 | tissue inhibitor of metalloproteinase 2 |
| 35454_at | up | 0.033944357 | KIAA0450 gene product |
| 1613_s_at | up | 0.033952839 | ubiquitin specific protease 6 (Tre-2 oncogene) |
| 35693_at | up | 0.033996591 | hippocalcin-like 1 |
| 1095_s_at | up | 0.034001673 | hepatocyte growth factor (hepapoietin A; scatter factor) |
| 41573_at | down | 0.034024859 | Sp3 transcription factor |
| 33677_at | down | 0.034077303 | ribosomal protein L24 |
| 41388_at | up | 0.03408507 | Meis1, myeloid ecotropic viral integration site 1 homolog 2 |
| | | | (mouse) |
| 32409_at | up | 0.034121256 | phosphatidylinositol glycan class O |
| 37788_at | up | 0.03412756 | |
| 38980_at | down | 0.034131969 | mitogen-activated protein kinase kinase kinase 7 interacting |
| | | | protein 2 |
| 34897_at | up | 0.034163537 | protein phosphatase 4, regulatory subunit 2 |
| 32221_at | down | 0.034177991 | mitochondrial ribosomal protein S18B |
| 38323_at | down | 0.034293103 | carboxypeptidase, vitellogenic-like |
| 33570_at | up | 0.034304188 | NK2 transcription factor related, locus 5 (Drosophila) |
| 36318_at | up | 0.034313433 | homolog of rat orphan transporter v7-3 |
| 35967_at | up | 0.034376104 | aryl hydrocarbon receptor nuclear translocator |
| 31659_at | up | 0.034390954 | DKFZP434K091 protein |
| 31661_at | up | 0.034425691 | |
| 41756_at | down | 0.034438173 | XPA binding protein 1 |
| 33046_f_at | up | 0.034440875 | empty spiracles homolog 1 (Drosophila) |
| 37666_at | up | 0.034522353 | proteasome (prosome, macropain) subunit, beta type, 5 |
| 32425_at | up | 0.034526343 | cholinergic receptor, nicotinic, alpha polypeptide 2 (neuronal) |
| 40571_at | down | 0.03453232 | myosin VA (heavy polypeptide 12, myoxin) |
| 33219_at | down | 0.034561079 | pVHL-interacting deubiquitinating enzyme 1 |
| 32267_at | up | 0.034563899 | zinc finger protein 345 |
| 31510_s_at | down | 0.034573927 | H3 histone, family 3B (H3.3B) |
| 39652_at | up | 0.034588394 | chemokine (C motif) ligand 1 |
| 1782_s_at | up | 0.034594134 | stathmin 1/oncoprotein 18 |
| 38819_at | up | 0.034640515 | PTK7 protein tyrosine kinase 7 |
| 39221_at | down | 0.034659495 | leukocyte immunoglobulin-like receptor, subfamily B (with TM and |
| | | | ITIM domains), member 2 |
| 37813_at | up | 0.034669603 | |
| 38056_at | up | 0.034690705 | KIAA0195 gene product |
| 527_at | up | 0.034691647 | centromere protein A, 17 kDa |
| 33137_at | up | 0.034815653 | latent transforming growth factor beta binding protein 4 |
| 34743_at | up | 0.03491869 | scribble |
| 40954_at | up | 0.034930625 | FXYD domain containing ion transport regulator 2 |
| 32739_at | up | 0.034938417 | phosphoglucomutase 3 |
| 39747_at | down | 0.034979203 | polymerase (RNA) II (DNA directed) polypeptide G |
| 40672_at | up | 0.035009921 | kynureninase (L-kynurenine hydrolase) |
| 34607_at | up | 0.0350146 | inducible T-cell co-stimulator |
| 32991_f_at | up | 0.035068269 | amelogenin (Y chromosome) |
| 31314_at | up | 0.035072962 | bone morphogenetic protein 3 (osteogenic) |
| 33178_at | up | 0.035088856 | jagged 1 (Alagille syndrome) |
| 37832_at | up | 0.035109738 | DKFZP564I122 protein |
| 802_at | down | 0.035128062 | TAF12 RNA polymerase II, TATA box binding protein (TBP)- |
| | | | associated factor, 20 kDa |
| 32025_at | down | 0.035193294 | transcription factor 7-like 2 (T-cell specific, HMG-box) |
| 39086_g_at | down | 0.035197215 | single-stranded DNA binding protein |
| 39777_at | down | 0.035201873 | protein associated with Myc |
| 35260_at | up | 0.03524273 | Mix interactor |
| 33247_at | down | 0.03528389 | proteasome (prosome, macropain) 26S subunit, non-ATPase, 14 |
| 39179_at | up | 0.035361402 | proteoglycan 2, bone marrow (natural killer cell activator, |
| | | | eosinophil granule major basic protein) |
| 35038_at | up | 0.035365152 | myosin binding protein C, cardiac |
| 35742_at | up | 0.0353725 | hypothetical gene BC008967 |
| 38308_g_at | up | 0.035420841 | neurochondrin |
| 37177_at | down | 0.035452242 | CD58 antigen, (lymphocyte function-associated antigen 3) |
| 38066_at | up | 0.035462531 | NAD(P)H dehydrogenase, quinone 1 |
| 38528_at | up | 0.035497003 | acetyl-Coenzyme A carboxylase alpha |
| 32248_at | down | 0.035497896 | hypothetical protein PRO2730 |
| 32548_at | down | 0.035500275 | unactive progesterone receptor, 23 kD |
| 32263_at | up | 0.035509211 | cyclin B2 |
| 32622_at | up | 0.03554138 | dynamin 2 |
| 35327_at | down | 0.03561689 | eukaryotic translation initiation factor 3, subunit 3 gamma, 40 kDa |
| 37001_at | down | 0.035620522 | calpain 2, (m/II) large subunit |
| 35207_at | up | 0.035647227 | sodium channel, nonvoltage-gated 1 alpha |
| 37697_s_at | down | 0.035661664 | voltage-dependent anion channel 2 |
| 34637_f_at | up | 0.035677011 | alcohol dehydrogenase 1A (class I), alpha polypeptide |
| 40238_at | up | 0.035681038 | G protein-coupled receptor, family C, group 5, member B |
| 41657_at | up | 0.035748093 | serine/threonine kinase 11 (Peutz-Jeghers syndrome) |
| 38072_at | down | 0.035800174 | hypothetical protein dJ465N24.2.1 |
| 35949_at | up | 0.035817369 | KIAA0774 protein |
| 39133_at | down | 0.035822484 | GCN5 general control of amino-acid synthesis 5-like 1 (yeast) |
| 440_at | up | 0.035839885 | nuclear factor I/C (CCAAT-binding transcription factor) |
| 39032_at | down | 0.035842888 | transforming growth factor beta-stimulated protein TSC-22 |
| 36859_at | up | 0.03584934 | non-metastatic cells 5, protein expressed in (nucleoside- |
| | | | diphosphate kinase) |
| 32075_at | up | 0.035852074 | zinc finger protein 161 homolog (mouse) |
| 41007_at | up | 0.03590906 | myozenin 3 |
| 37908_at | down | 0.035935871 | guanine nucleotide binding protein (G protein), gamma 11 |
| 39111_s_at | down | 0.035951483 | peptidylprolyl isomerase (cyclophilin)-like 2 |
| 39390_at | down | 0.035991974 | nucleoporin 133 kDa |
| 1536_at | up | 0.036029396 | CDC6 cell division cycle 6 homolog (S. cerevisiae) |
| 40810_at | down | 0.036034327 | SWI/SNF related, matrix associated, actin dependent regulator of |
| | | | chromatin, subfamily c, member 1 |
| 39015_f_at | up | 0.036068618 | keratin 6A |
| 31439_f_at | up | 0.036084452 | Rhesus blood group, CcEe antigens |
| 38291_at | up | 0.036104104 | proenkephalin |
| 39217_at | up | 0.036118403 | |
| 728_at | up | 0.03612594 | |
| 40267_s_at | up | 0.036136261 | KIAA1036 protein |
| 35442_at | up | 0.036186437 | KIAA0792 gene product |
| 39034_at | down | 0.036243812 | DKFZP564O123 protein |
| 33227_at | down | 0.036245055 | interleukin 10 receptor, beta |
| 39927_at | up | 0.036258257 | Rho GTPase activating protein 5 |
| 31365_f_at | up | 0.036274538 | nuclear factor of activated T-cells 5, tonicity-responsive |
| 883_s_at | up | 0.036301176 | pim-1 oncogene |
| 1512_at | down | 0.036307971 | dual-specificity tyrosine-(Y)-phosphorylation regulated kinase 1A |
| 35011_at | down | 0.036313265 | HECT type E3 ubiquitin ligase |
| 38204_at | up | 0.036318873 | KIAA0406 gene product |
| 37381_g_at | down | 0.036360287 | general transcription factor IIB |
| 162_at | up | 0.036372555 | ubiquitin specific protease 11 |
| 34568_at | up | 0.036382821 | keratin, hair, acidic, 3B |
| 34160_at | down | 0.036395515 | actin, gamma 1 |
| 41872_at | up | 0.036421973 | deafness, autosomal dominant 5 |
| 40636_at | up | 0.036435919 | flotillin 1 |
| 1382_at | down | 0.036530269 | replication protein A1, 70 kDa |
| 36360_at | up | 0.036567094 | KIAA0507 protein |
| 39542_at | up | 0.036640558 | ectodermal-neural cortex (with BTB-like domain) |
| 1765_at | up | 0.036649057 | caspase 10, apoptosis-related cysteine protease |
| 837_s_at | down | 0.03665956 | malic enzyme 1, NADP(+)-dependent, cytosolic |
| 1949_at | up | 0.036688112 | angiopoietin 1 |
| 37135_f_at | up | 0.036721619 | glutamate receptor, ionotropic, N-methyl D-aspartate 1 |
| 40754_at | up | 0.036753843 | general transcription factor IIH, polypeptide 3, 34 kDa |
| 39288_at | up | 0.036831137 | nectin-like protein 1 |
| 33580_r_at | up | 0.036856676 | galanin receptor 3 |
| 990_at | up | 0.036936172 | fms-related tyrosine kinase 1 (vascular endothelial growth |
| | | | factor/vascular permeability factor receptor) |
| 41780_at | down | 0.036941323 | protein tyrosine phosphatase, receptor type, f polypeptide |
| | | | (PTPRF), interacting protein (liprin), alpha 1 |
| 31412_at | up | 0.036983521 | PTPN13-like, Y-linked |
| 38544_at | up | 0.036991342 | inhibin, alpha |
| 39760_at | down | 0.036998817 | quaking homolog, KH domain RNA binding (mouse) |
| 160026_at | up | 0.037003493 | protein kinase, X-linked |
| 37844_at | down | 0.037057085 | class I cytokine receptor |
| 38627_at | up | 0.037109589 | hepatic leukemia factor |
| AFFX-M27830_3_at | up | 0.037136535 | |
| 39601_at | up | 0.037182176 | Ras association (RalGDS/AF-6) domain family 1 |
| 39212_at | up | 0.037250199 | hypothetical protein FLJ11191 |
| 35451_s_at | up | 0.037307942 | SCAN domain containing 2 |
| 34064_s_at | up | 0.037309471 | natural cytotoxicity triggering receptor 2 |
| 36956_at | up | 0.037350527 | solute carrier family 20 (phosphate transporter), member 2 |
| 41355_at | down | 0.037373862 | B-cell CLL/lymphoma 11A (zinc finger protein) |
| 39208_i_at | down | 0.037381904 | pro-platelet basic protein (chemokine (C—X—C motif) ligand 7) |
| 38415_at | down | 0.037413264 | protein tyrosine phosphatase type IVA, member 2 |
| 38445_at | up | 0.037433428 | actin related protein 2/3 complex, subunit 2, 34 kDa |
| 1322_at | up | 0.037477325 | |
| 38466_at | up | 0.037501653 | cathepsin K (pycnodysostosis) |
| 36779_at | up | 0.037509117 | fatty acid binding protein 6, ileal (gastrotropin) |
| 1295_at | down | 0.037566519 | v-rel reticuloendotheliosis viral oncogene homolog A, nuclear |
| | | | factor of kappa light polypeptide gene enhancer in B-cells 3, p65 |
| | | | (avian) |
| 37423_at | up | 0.037568568 | solute carrier family 12 (sodium/potassium/chloride transporters), |
| | | | member 2 |
| 41406_at | down | 0.037620194 | hypothetical protein FLJ21919 |
| 34112_r_at | up | 0.037686408 | |
| 34038_at | up | 0.037720153 | solute carrier family 6 (neurotransmitter transporter, GABA), |
| | | | member 13 |
| 38781_at | up | 0.037740959 | glutathione S-transferase A2 |
| 33045_r_at | up | 0.037754364 | empty spiracles homolog 1 (Drosophila) |
| 33420_g_at | down | 0.037813208 | apoptosis inhibitor 5 |
| 34877_at | down | 0.037874468 | Janus kinase 1 (a protein tyrosine kinase) |
| 39920_r_at | up | 0.037908818 | C1q-related factor |
| 40617_at | down | 0.037913324 | hypothetical protein FLJ20274 |
| 31602_at | up | 0.037939657 | T-box 6 |
| 39190_s_at | up | 0.03794319 | |
| 38158_at | up | 0.037955678 | extra spindle poles like 1 (S. cerevisiae) |
| 39563_at | up | 0.037962522 | KIAA0268 protein |
| 38393_at | down | 0.037967247 | KIAA0247 gene product |
| 37986_at | down | 0.038002259 | erythropoietin receptor |
| 33293_at | up | 0.038041132 | Fas apoptotic inhibitory molecule 2 |
| 35146_at | up | 0.0380469 | transforming growth factor beta 1 induced transcript 1 |
| 34566_at | up | 0.038085141 | calcitonin-related polypeptide, beta |
| 41246_at | up | 0.038116844 | serine (or cysteine) proteinase inhibitor, clade E (nexin, |
| | | | plasminogen activator inhibitor type 1), member 2 |
| 39426_at | down | 0.03813583 | transcription elongation regulator 1 (CA150) |
| 39608_at | up | 0.038173191 | single-minded homolog 2 (Drosophila) |
| 41858_at | up | 0.038174864 | FGF receptor activating protein 1 |
| 37668_at | down | 0.038273197 | complement component 1, q subcomponent binding protein |
| 35947_at | up | 0.038343221 | transglutaminase 1 (K polypeptide epidermal type I, protein- |
| | | | glutamine-gamma-glutamyltransferase) |
| 41129_at | down | 0.038356011 | KIAA0033 protein |
| 40268_at | up | 0.038387842 | FOS-like antigen 2 |
| 36617_at | up | 0.038390439 | inhibitor of DNA binding 1, dominant negative helix-loop-helix |
| | | | protein |
| 39946_at | up | 0.038392089 | pancreatitis-associated protein |
| 35297_at | down | 0.038407506 | NADH dehydrogenase (ubiquinone) 1, alpha/beta subcomplex, 1, |
| | | | 8 kDa |
| 41172_at | down | 0.038407527 | retinol dehydrogenase 11 (all-trans and 9-cis) |
| 32980_f_at | down | 0.038440496 | histone 1, H2bc |
| 39364_s_at | up | 0.038449579 | protein phosphatase 1, regulatory (inhibitor) subunit 3C |
| 33060_g_at | up | 0.038481349 | |
| 39109_at | up | 0.038509158 | chromosome 20 open reading frame 1 |
| 2041_i_at | up | 0.038513823 | v-abl Abelson murine leukemia viral oncogene homolog 1 |
| 37984_s_at | down | 0.038552881 | ADP-ribosylation factor 6 |
| 36746_s_at | up | 0.038564507 | calcitonin receptor |
| 39616_at | up | 0.038593624 | |
| 1270_at | up | 0.038616745 | RAP1, GTPase activating protein 1 |
| 35464_at | up | 0.038622074 | interleukin 11 |
| 41187_at | down | 0.038647669 | myosin regulatory light chain MRLC2 |
| 39143_at | down | 0.038679527 | nuclear factor of activated T-cells, cytoplasmic, calcineurin- |
| | | | dependent 1 |
| 34260_at | up | 0.038707405 | KIAA0683 gene product |
| 37567_at | up | 0.038711751 | sal-like 2 (Drosophila) |
| 35612_at | up | 0.038714873 | DKFZP564P1916 protein |
| 37858_at | up | 0.038730644 | collagen-like tail subunit (single strand of homotrimer) of |
| | | | asymmetric acetylcholinesterase |
| 34984_at | up | 0.038737233 | transient receptor potential cation channel, subfamily C, member |
| 33949_at | up | 0.03874733 | corticotropin releasing hormone receptor 2 |
| 342_at | up | 0.038767213 | ectonucleotide pyrophosphatase/phosphodiesterase 1 |
| 33052_at | up | 0.038797008 | phospholipase A2, group X |
| 41423_at | up | 0.038802753 | calsyntenin 3 |
| 37112_at | down | 0.038914634 | chromosome 6 open reading frame 32 |
| 1754_at | up | 0.038967239 | death-associated protein 6 |
| 33025_at | up | 0.038968578 | chromosome 20 open reading frame 10 |
| 627_g_at | up | 0.039082 | arginine vasopressin receptor 1B |
| 34731_at | down | 0.039093403 | programmed cell death 11 |
| 32568_at | up | 0.039115444 | BTG family, member 3 |
| 36881_at | down | 0.039125362 | electron-transfer-flavoprotein, beta polypeptide |
| 41686_s_at | up | 0.039157522 | NY-REN-7 antigen |
| 38487_at | up | 0.039168582 | stabilin 1 |
| 40417_at | down | 0.039177693 | chaperonin containing TCP1, subunit 5 (epsilon) |
| 35849_at | up | 0.039215248 | phosphatidylserine receptor |
| 33243_at | down | 0.039241844 | TNF-induced protein |
| 32346_at | up | 0.039243412 | |
| 40461_at | up | 0.039253806 | triple homeobox 1 |
| 40028_at | up | 0.039264393 | LOC92346 |
| 34932_at | up | 0.039274406 | melanoma antigen, family C, 1 |
| 37648_at | up | 0.039293338 | KIAA0153 protein |
| 32542_at | down | 0.039325252 | four and a half LIM domains 1 |
| 39209_r_at | down | 0.039349881 | pro-platelet basic protein (chemokine (C—X—C motif) ligand 7) |
| 41819_at | down | 0.039389573 | FYN binding protein (FYB-120/130) |
| 31492_at | down | 0.039426982 | muscle specific gene |
| 38481_at | down | 0.039431725 | replication protein A1, 70 kDa |
| 36004_at | up | 0.039498058 | inhibitor of kappa light polypeptide gene enhancer in B-cells, |
| | | | kinase gamma |
| 31551_at | up | 0.039538216 | gamma-aminobutyric acid (GABA) receptor, rho 2 |
| 40613_at | down | 0.039628778 | chromosome 6 open reading frame 62 |
| 37970_at | up | 0.039662257 | mitogen-activated protein kinase 8 interacting protein 3 |
| 1666_at | up | 0.039665359 | interferon, alpha 1 |
| 115_at | down | 0.039688446 | thrombospondin 1 |
| 2037_s_at | up | 0.039733454 | ribosomal protein S6 kinase, 70 kDa, polypeptide 1 |
| 38152_at | up | 0.039735408 | loss of heterozygosity, 11, chromosomal region 2, gene A |
| 36169_at | down | 0.039738748 | NADH dehydrogenase (ubiquinone) 1 alpha subcomplex, 1, |
| | | | 7.5 kDa |
| AFFX-BioB-3_at | up | 0.039745177 | |
| 40353_at | up | 0.039779913 | |
| 31409_at | up | 0.039789728 | serine (or cysteine) proteinase inhibitor, clade B (ovalbumin), |
| | | | member 10 |
| 37049_g_at | up | 0.039808882 | translocase of outer mitochondrial membrane 34 |
| 36473_at | up | 0.039827121 | ubiquitin specific protease 20 |
| 35743_at | up | 0.039844257 | cleavage and polyadenylation specific factor 4, 30 kDa |
| 38444_at | up | 0.039897913 | cysteine and glycine-rich protein 3 (cardiac LIM protein) |
| 32557_at | up | 0.039917401 | U2 small nuclear ribonucleoprotein auxiliary factor (65 kD) |
| 34539_at | up | 0.039968141 | olfactory receptor, family 7, subfamily A, member 126 pseudogene |
| 36851_g_at | up | 0.040105766 | Putative prostate cancer tumor suppressor |
| 32648_at | up | 0.040119322 | delta-like 1 homolog (Drosophila) |
| 39030_at | up | 0.040121358 | Rab acceptor 1 (prenylated) |
| 39391_at | down | 0.04013934 | associated molecule with the SH3 domain of STAM |
| 41822_at | up | 0.040160905 | zinc finger protein |
| 32818_at | up | 0.040201801 | tenascin C (hexabrachion) |
| 39313_at | up | 0.040229309 | protein kinase, lysine deficient 1 |
| 37926_at | up | 0.040231459 | Kruppel-like factor 5 (intestinal) |
| 875_g_at | up | 0.040259866 | chemokine (C—C motif) ligand 2 |
| 31487_at | up | 0.040270313 | fasciculation and elongation protein zeta 2 (zygin II) |
| 38613_at | up | 0.040286427 | putative cyclin G1 interacting protein |
| 2007_g_at | up | 0.040353971 | Janus kinase 3 (a protein tyrosine kinase, leukocyte) |
| 39136_at | down | 0.040366437 | oxidative-stress responsive 1 |
| 41782_g_at | up | 0.0403846 | protein tyrosine phosphatase, receptor type, f polypeptide |
| | | | (PTPRF), interacting protein (liprin), alpha 1 |
| 35749_at | up | 0.040386806 | transcriptional adaptor 3 (NGG1 homolog, yeast)-like |
| 39331_at | up | 0.040419286 | tubulin, beta polypeptide |
| 36476_at | down | 0.040425881 | bromodomain containing 8 |
| 40247_at | up | 0.040437893 | solute carrier family 9 (sodium/hydrogen exchanger), isoform 7 |
| 40167_s_at | down | 0.040463083 | likely ortholog of mouse WD-40-repeat-containing protein with a |
| | | | SOCS box 2 |
| 32034_at | down | 0.040506981 | zinc finger protein 217 |
| 40567_at | down | 0.040518867 | tubulin, alpha 3 |
| 40691_at | down | 0.040548176 | zinc finger protein 274 |
| 37005_at | up | 0.040561003 | neuroblastoma, suppression of tumorigenicity 1 |
| 40371_at | up | 0.040591204 | dopamine receptor D2 |
| 31388_at | up | 0.040627727 | early lymphoid activation protein |
| AFFX-CreX-3_at | up | 0.040647806 | |
| 41499_at | up | 0.040698611 | v-ski sarcoma viral oncogene homolog (avian) |
| 38131_at | up | 0.040768509 | prostaglandin E synthase |
| 40266_at | up | 0.04078716 | KIAA1036 protein |
| 39324_at | up | 0.040788061 | |
| 1154_at | up | 0.040819001 | eukaryotic translation initiation factor 2, subunit 1 alpha, 35 kDa |
| 413_at | up | 0.040842837 | homeo box D9 |
| 34299_at | down | 0.040881701 | zinc finger protein 278 |
| 39050_at | up | 0.040889932 | poly(A) binding protein, nuclear 1 |
| 35408_i_at | down | 0.040944508 | zinc finger protein 44 (KOX 7) |
| 41720_r_at | up | 0.040965049 | fatty acid desaturase 1 |
| 1392_at | up | 0.041028012 | G protein-coupled receptor kinase 6 |
| 37011_at | down | 0.041037942 | allograft inflammatory factor 1 |
| 40430_at | up | 0.041117487 | hypothetical protein FLJ35779 |
| 40439_at | down | 0.041157278 | arsA arsenite transporter, ATP-binding, homolog 1 (bacterial) |
| 36745_at | up | 0.041195119 | |
| 39105_at | down | 0.041201015 | vasodilator-stimulated phosphoprotein |
| 37941_at | up | 0.041220219 | myosin binding protein C, fast type |
| 1806_at | up | 0.041223406 | MCF.2 cell line derived transforming sequence |
| 34049_at | down | 0.041282808 | |
| 41718_g_at | up | 0.041288593 | fatty acid desaturase 1 |
| 34738_at | up | 0.041289183 | serine hydroxymethyltransferase 1 (soluble) |
| 40379_at | up | 0.041411241 | cytochrome P450, family 2, subfamily E, polypeptide 1 |
| 32646_at | up | 0.041464958 | KIAA0449 protein |
| 720_at | up | 0.041465792 | heat shock transcription factor 4 |
| 33151_s_at | up | 0.041485117 | disrupter of silencing 10 |
| 39970_at | up | 0.041555279 | nuclear receptor subfamily 0, group B, member 1 |
| 375_at | up | 0.041589703 | glutathione S-transferase theta 1 |
| 33697_at | up | 0.041601882 | purinergic receptor P2X, ligand-gated ion channel, 7 |
| 420_at | up | 0.041622259 | melanocortin 2 receptor (adrenocorticotropic hormone) |
| 40733_f_at | up | 0.041645239 | msh homeo box homolog 2 (Drosophila) |
| 763_at | down | 0.041702481 | glia maturation factor, beta |
| 2005_s_at | up | 0.0417241 | Janus kinase 3 (a protein tyrosine kinase, leukocyte) |
| 41651_at | down | 0.041743121 | KIAA1033 protein |
| 35991_at | down | 0.041767326 | LSM6 homolog, U6 small nuclear RNA associated (S. cerevisiae) |
| 38814_at | down | 0.041813234 | ATPase, H+ transporting, lysosomal 13 kDa, V1 subunit G isoform 1 |
| 38548_at | up | 0.041829912 | cytochrome P450, family 2, subfamily C, polypeptide 8 |
| 38312_at | up | 0.041984212 | |
| 37955_at | down | 0.042020127 | transmembrane protein 4 |
| 31357_at | up | 0.042037339 | |
| 34192_at | down | 0.042048499 | KIAA0532 protein |
| 33336_at | up | 0.042053513 | solute carrier family 4, anion exchanger, member 1 (erythrocyte |
| | | | membrane protein band 3, Diego blood group) |
| 632_at | up | 0.042123503 | glycogen synthase kinase 3 alpha |
| 1168_at | up | 0.042146694 | protocadherin beta 17 pseudogene |
| 38794_at | down | 0.042176671 | upstream binding transcription factor, RNA polymerase I |
| 36562_at | up | 0.042240759 | KIAA0427 gene product |
| 36952_at | up | 0.042241044 | hydroxyacyl-Coenzyme A dehydrogenase/3-ketoacyl-Coenzyme A |
| | | | thiolase/enoyl-Coenzyme A hydratase (trifunctional protein), alpha |
| | | | subunit |
| 37994_at | down | 0.042302692 | fragile X mental retardation 1 |
| 1750_at | up | 0.042309315 | phenylalanine-tRNA synthetase-like |
| 1914_at | up | 0.042374368 | cyclin A1 |
| 37330_at | up | 0.0424156 | aldehyde dehydrogenase 4 family, member A1 |
| 36939_at | up | 0.042424587 | glycoprotein M6A |
| 32573_at | down | 0.042442348 | splicing factor, arginine/serine-rich 9 |
| 33997_at | up | 0.042551104 | |
| 1875_f_at | up | 0.042561561 | postmeiotic segregation increased 2-like 3 |
| 35953_at | up | 0.042571185 | carboxypeptidase N, polypeptide 1, 50 kD |
| 32258_r_at | down | 0.042616045 | telomeric repeat binding factor (NIMA-interacting) 1 |
| 32493_at | up | 0.04264112 | thyrotrophic embryonic factor |
| 41557_at | down | 0.042735256 | KIAA0052 protein |
| 35673_at | up | 0.042751453 | Rho guanine nucleotide exchange factor (GEF) 5 |
| 1255_g_at | up | 0.042771637 | guanylate cyclase activator 1A (retina) |
| 648_at | up | 0.042778333 | arginine vasopressin receptor 1B |
| 1574_s_at | up | 0.042812441 | interleukin 4 |
| 1380_at | up | 0.042829999 | fibroblast growth factor 7 (keratinocyte growth factor) |
| 40701_at | up | 0.042832741 | ubiquitin specific protease 13 (isopeptidase T-3) |
| 33945_at | down | 0.042849117 | tumor necrosis factor (ligand) superfamily, member 5 (hyper-IgM |
| | | | syndrome) |
| 40731_at | up | 0.04287054 | chromobox homolog 5 (HP1 alpha homolog, Drosophila) |
| 33637_g_at | up | 0.042880647 | cancer/testis antigen 1 |
| 1115_at | down | 0.042942517 | platelet factor 4 (chemokine (C—X—C motif) ligand 4) |
| 37977_at | up | 0.042975756 | deltex homolog 2 (Drosophila) |
| 34809_at | down | 0.042989623 | KIAA0999 protein |
| 38105_at | down | 0.04300635 | hypothetical protein FLJ11021 similar to splicing factor, |
| | | | arginine/serine-rich 4 |
| 33522_at | up | 0.043053962 | agouti signaling protein, nonagouti homolag (mouse) |
| 32717_at | up | 0.043086938 | neuralized-like (Drosophila) |
| 1377_at | down | 0.043126879 | nuclear factor of kappa light polypeptide gene enhancer in B-cells |
| | | | 1 (p105) |
| 36596_r_at | up | 0.043217487 | glycine amidinotransferase (L-arginine:glycine amidinotransferase |
| AFFX-BioDn-3_at | up | 0.043247969 | |
| 1477_s_at | up | 0.04330999 | cytochrome P450, family 2, subfamily C, polypeptide 18 |
| 39842_at | up | 0.043318734 | cytokine receptor-like factor 1 |
| 1903_at | down | 0.043361718 | |
| 40777_at | down | 0.04336893 | catenin (cadherin-associated protein), beta 1, 88 kDa |
| 34636_at | up | 0.043370973 | arachidonate 15-lipoxygenase |
| 39645_r_at | up | 0.043372248 | arrestin 3, retinal (X-arrestin) |
| 41764_at | down | 0.043388296 | apolipoprotein C-I |
| 36488_at | down | 0.043398698 | EGF-like-domain, multiple 5 |
| 491_at | up | 0.04341321 | protein tyrosine phosphatase, receptor type, G |
| 37218_at | up | 0.043488344 | BTG family, member 3 |
| 33498_at | up | 0.043497555 | regenerating islet-derived-like, pancreatic stone protein-like, |
| | | | pancreatic thread protein-like (rat) |
| 640_at | up | 0.043523098 | angiotensin II receptor-like 2 |
| 36845_at | down | 0.043540169 | nuclear matrix protein NXP2 |
| 35783_at | down | 0.043700506 | vesicle-associated membrane protein 3 (cellubrevin) |
| 38529_at | up | 0.043708018 | acetyl-Coenzyme A carboxylase beta |
| 31947_r_at | up | 0.043725153 | forkhead box G1A |
| 40689_at | down | 0.043739673 | sel-1 suppressor of lin-12-like (C. elegans) |
| 34088_at | up | 0.043767248 | neurexophilin 4 |
| 34884_at | up | 0.043790003 | carbamoyl-phosphate synthetase 1, mitochondrial |
| 35056_at | up | 0.043793206 | arylsulfatase F |
| 37348_s_at | down | 0.043822957 | high mobility group nucleosomal binding domain 3 |
| 40132_g_at | down | 0.04383039 | follistatin-like 1 |
| 34422_r_at | up | 0.043832201 | uncoupling protein 3 (mitochondrial, proton carrier) |
| 36659_at | up | 0.043859511 | collagen, type IV, alpha 2 |
| 35722_at | down | 0.04386591 | UPF2 regulator of nonsense transcripts homolog (yeast) |
| 34356_at | down | 0.043974258 | SRB7 suppressor of RNA polymerase B homolog (yeast) |
| 33540_at | up | 0.044014718 | |
| 296_at | down | 0.04402855 | |
| 41147_at | down | 0.044084481 | hypothetical protein MGC4276 similar to CG8198 |
| 40610_at | down | 0.044101702 | zinc finger RNA binding protein |
| 41208_at | down | 0.044106895 | S164 protein |
| 31986_at | up | 0.044122485 | |
| 39462_s_at | up | 0.044182359 | cyclin M2 |
| 40958_at | up | 0.044246117 | KIAA0599 protein |
| 39063_at | up | 0.044305104 | actin, alpha, cardiac muscle |
| 36754_at | up | 0.044401821 | adenylate cyclase activating polypeptide 1 (pituitary) |
| 975_at | up | 0.04454837 | serine/threonine kinase 18 |
| 41543_at | up | 0.044555245 | lymphoid nuclear protein related to AF4 |
| 39821_s_at | up | 0.04462269 | growth arrest and DNA-damage-inducible, beta |
| 38942_r_at | up | 0.044661343 | AD024 protein |
| 39834_at | up | 0.044677883 | cholinergic receptor, nicotinic, epsilon polypeptide |
| 41158_at | up | 0.044679844 | proteolipid protein 1 (Pelizaeus-Merzbacher disease, spastic |
| | | | paraplegia 2, uncomplicated) |
| 35954_at | up | 0.044713 | prodynorphin |
| 38952_s_at | up | 0.04475812 | collagen, type XIII, alpha 1 |
| 1731_at | up | 0.044857816 | platelet-derived growth factor receptor, alpha polypeptide |
| 40067_at | down | 0.044859698 | E74-like factor 1 (ets domain transcription factor) |
| 38174_at | up | 0.044860824 | pleckstrin and Sec7 domain protein |
| 1473_s_at | up | 0.044866751 | v-myb myeloblastosis viral oncogene homolog (avian) |
| 34475_at | up | 0.044903408 | |
| 39095_at | up | 0.044919175 | myosin, heavy polypeptide 7, cardiac muscle, beta |
| 34771_at | up | 0.044938431 | phosphatidic acid phosphatase type 2C |
| 39824_at | up | 0.04494232 | protein tyrosine phosphatase type IVA, member 3 |
| 33583_r_at | up | 0.044980903 | RNA binding motif, single stranded interacting protein |
| 38567_at | down | 0.045086218 | CD1D antigen, d polypeptide |
| 33213_g_at | up | 0.04509451 | ribosome binding protein 1 homolog 180 kDa (dog) |
| 34799_at | up | 0.045130417 | intraflagellar transport protein IFT20 |
| 31708_at | down | 0.045135218 | ribosomal protein L30 |
| 39187_at | up | 0.045161232 | runt-related transcription factor 2 |
| 39175_at | down | 0.045229902 | phosphofructokina |
| 38340_at | up | 0.045251115 | huntingtin interacting protein-1-related |
| 38919_at | up | 0.045257342 | chromosome 6 open reading frame 84 |
| 32836_at | up | 0.045281983 | 1-acylglycerol-3-phosphate O-acyltransferase 1 (lysophosphatidic |
| | | | acid acyltransferase, alpha) |
| AFFX-BioB-M_st | up | 0.045314371 | |
| 110_at | up | 0.045320404 | chondroitin sulfate proteoglycan 4 (melanoma-associated) |
| 36457_at | down | 0.045332883 | guanine monphosphate synthetase |
| 1038_s_at | down | 0.045424969 | interferon gamma receptor 1 |
| 32707_at | up | 0.045428446 | katanin p60 (ATPase-containing) subunit A 1 |
| 40772_at | up | 0.04548016 | hypothetical protein FLJ22269 |
| 36995_at | up | 0.045488385 | alpha-1-microglobulin/bikunin precursor |
| 37439_at | up | 0.045504275 | solute carrier family 30 (zinc transporter), member 4 |
| 32023_at | up | 0.045513124 | |
| 40975_s_at | up | 0.04553262 | tuftelin interacting protein 11 |
| 39684_at | up | 0.045568029 | membrane protein, palmitoylated 3 (MAGUK p55 subfamily |
| | | | member 3) |
| 33534_at | up | 0.045610323 | endothelial cell-specific molecule 1 |
| 39738_at | down | 0.045611646 | myosin, heavy polypeptide 9, non-muscle |
| 36171_at | down | 0.045654778 | activated RNA polymerase II transcription cofactor 4 |
| 34885_at | up | 0.045703542 | synaptogyrin 2 |
| 32525_r_at | up | 0.045882979 | junctional adhesion molecule 3 |
| 41206_r_at | down | 0.045915305 | cytochrome c oxidase subunit VIa polypeptide 1 |
| 31858_at | down | 0.045925116 | nuclear transport factor 2 |
| 32480_at | up | 0.045946721 | homeo box C4 |
| 37801_at | up | 0.045990579 | ATPase, H+ transporting, lysosomal V0 subunit a isoform 2 |
| 41211_at | up | 0.045997988 | RNA binding motif protein 12 |
| 40326_at | up | 0.046028247 | cerebellin 1 precursor |
| 35425_at | up | 0.046028539 | BarH-like homeobox 2 |
| 38447_at | up | 0.046105867 | adrenergic, beta, receptor kinase 1 |
| 36687_at | down | 0.046108202 | cytochrome c oxidase subunit VIIb |
| 1648_at | up | 0.046115812 | oncostatin M receptor |
| 41386_i_at | up | 0.046126282 | KIAA0346 protein |
| 34182_at | up | 0.046136684 | N-deacetylase/N-sulfotransferase (heparan glucosaminyl) 1 |
| 37084_at | up | 0.04615034 | lysozyme homolog |
| 37102_at | up | 0.046158495 | breast cancer metastasis-suppressor 1 |
| 38888_at | up | 0.046186078 | leucine-rich, glioma inactivated 1 |
| 36333_at | down | 0.046189903 | ribosomal protein L7 |
| 33404_at | up | 0.046205759 | adenylyl cyclase-associated protein 2 |
| 41321_s_at | up | 0.046241974 | nucleolar protein family A, member 2 (H/ACA small nucleolar |
| | | | RNPs) |
| 1406_at | up | 0.046355671 | nuclear receptor subfamily 2, group C, member 1 |
| 33897_at | up | 0.046389018 | phosphatidylinositol 4-kinase type II |
| 34280_at | up | 0.046453241 | gamma-aminobutyric acid (GABA) A receptor, epsilon |
| 1037_at | up | 0.046542336 | B melanoma antigen |
| 40901_at | down | 0.046554909 | striatin, calmodulin binding protein 3 |
| 38311_at | down | 0.046571724 | TGFB-induced factor 2 (TALE family homeobox) |
| 37449_i_at | down | 0.046577216 | GNAS complex locus |
| 506_s_at | up | 0.046586309 | signal transducer and activator of transcription 5A |
| 38820_at | down | 0.046590628 | 15 kDa selenoprotein |
| 40138_at | up | 0.046591745 | COP9 subunit 6 (MOV34 homolog, 34 kD) |
| 38075_at | down | 0.046592172 | synaptophysin-like protein |
| 32705_at | up | 0.04659497 | cytochrome P450, family 3, subfamily A, polypeptide 7 |
| 38328_at | down | 0.046624433 | solute carrier family 25, member 13 (citrin) |
| 33931_at | down | 0.046631191 | glutathione peroxidase 4 (phospholipid hydroperoxidase) |
| 1388_g_at | down | 0.046638995 | vitamin D (1,25-dihydroxyvitamin D3) receptor |
| 36396_at | up | 0.046663781 | |
| 31878_at | down | 0.046669447 | ATP-binding cassette, sub-family F (GCN20), member 2 |
| 35727_at | down | 0.046749257 | uridine kinase-like 1 |
| 31824_at | up | 0.046757144 | malic enzyme 1, NADP(+)-dependent, cytosolic |
| 34741_at | up | 0.046861972 | transcription factor Dp-2 (E2F dimerization partner 2) |
| 39189_at | up | 0.046938514 | potassium voltage-gated channel, Shaw-related subfamily, |
| | | | member 4 |
| 37195_at | up | 0.046947354 | cytochrome P450, family 11, subfamily A, polypeptide 1 |
| 41778_at | up | 0.046964409 | solute carrier family 1 (neutral amino acid transporter), member 5 |
| 40561_at | up | 0.046977023 | T-cell leukemia, homeobox 2 |
| 31318_at | up | 0.047010382 | |
| 31985_at | up | 0.047097209 | pleckstrin homology domain interacting protein |
| 31571_at | up | 0.047105137 | polymerase (RNA) III (DNA directed) (32 kD) |
| 34179_at | up | 0.047151817 | zinc finger protein 297 |
| 39005_s_at | down | 0.047173316 | zinc finger protein 294 |
| 39634_at | up | 0.047190272 | slit homolog 2 (Drosophila) |
| 38359_at | up | 0.047233322 | RAS guanyl releasing protein 2 (calcium and DAG-regulated) |
| 39767_at | down | 0.047273537 | chaperonin containing TCP1, subunit 8 (theta) |
| 35368_at | down | 0.047276393 | zinc finger protein 207 |
| 40727_at | down | 0.047283047 | anaphase-promoting complex subunit 10 |
| 38593_r_at | up | 0.047357278 | KIAA0284 protein |
| 2010_at | down | 0.047362414 | S-phase kinase-associated protein 1A (p19A) |
| 35575_f_at | up | 0.047365962 | zinc finger protein 253 |
| 1910_s_at | up | 0.04741841 | B-cell CLL/lymphoma 2 |
| 184_at | up | 0.047445638 | angiotensin II receptor-like 1 |
| 38461_at | up | 0.04746726 | nebulin |
| 38997_at | up | 0.047512317 | solute carrier family 25 (mitochondrial carrier; citrate transporter), |
| | | | member 1 |
| 38449_at | up | 0.047540057 | WD repeat domain 23 |
| 36511_at | down | 0.047649025 | SAC1 suppressor of actin mutations 1-like (yeast) |
| 39660_at | up | 0.047681544 | defensin, beta 1 |
| 1134_at | up | 0.04771627 | activated p21cdc42Hs kinase |
| 350_at | down | 0.047748166 | zinc finger protein 161 |
| 38108_at | up | 0.047777085 | palmitoyl-protein thioesterase 2 |
| 41572_r_at | down | 0.047800461 | v-rel reticuloendotheliosis viral oncogene homolog (avian) |
| 36430_at | up | 0.047801528 | adrenomedullin receptor |
| 183_at | up | 0.047811102 | microtubule-associated protein 2 |
| 33424_at | down | 0.047825363 | ribophorin I |
| 36289_f_at | up | 0.047833702 | fucosyltransferase 6 (alpha (1,3) fucosyltransferase) |
| 352_at | up | 0.047842757 | phosphotidylinositol transfer protein |
| 37481_at | down | 0.047846427 | cell division cycle 40 homolog (yeast) |
| 38671_at | up | 0.04786849 | plexin D1 |
| 36421_at | up | 0.04790502 | |
| 40687_at | up | 0.047916691 | gap junction protein, alpha 4, 37 kDa (connexin 37) |
| 33903_at | up | 0.047941918 | death-associated protein kinase 3 |
| 40002_r_at | up | 0.048072194 | chorea acanthocytosis |
| 33309_at | down | 0.048101663 | comparative gene identification 58 |
| 40831_at | down | 0.048137628 | DKFZP586B0923 protein |
| 35907_at | up | 0.048159735 | cyclin F |
| 494_at | up | 0.048164571 | interleukin 13 |
| 32112_s_at | down | 0.048170984 | absent in melanoma 1 |
| 34253_at | down | 0.048212712 | nucleoporin 160 kDa |
| 36998_s_at | down | 0.048223756 | spinocerebellar ataxia 2 (olivopontocerebellar ataxia 2, autosomal |
| | | | dominant, ataxin 2) |
| 34217_at | up | 0.048263151 | Kruppel-like factor 7 (ubiquitous) |
| 671_at | down | 0.048295042 | secreted protein, acidic, cysteine-rich (osteonectin) |
| 36934_at | down | 0.048313588 | chromosome 20 open reading frame 111 |
| 33589_at | up | 0.048381236 | |
| 34811_at | down | 0.048384646 | ATP synthase, H+ transporting, mitochondrial F0 complex, subun |
| | | | c (subunit 9) isoform 3 |
| 41420_at | down | 0.048387986 | insulin-like growth factor binding protein 5 |
| 1961_f_at | up | 0.048390228 | nitric oxide synthase 3 (endothelial cell) |
| 32778_at | down | 0.048408 | inositol 1,4,5-triphosphate receptor, type 1 |
| 1376_at | up | 0.048423676 | ligase I, DNA, ATP-dependent |
| 31648_at | up | 0.048427152 | chromosome 6 open reading frame 54 |
| 36533_at | up | 0.048431505 | prostaglandin I2 (prostacyclin) synthase |
| 691_g_at | up | 0.048436464 | procollagen-proline, 2-oxoglutarate 4-dioxygenase (proline 4- |
| | | | hydroxylase), beta polypeptide (protein disulfide isomerase; |
| | | | thyroid hormone binding protein p55) |
| 823_at | up | 0.048447199 | chemokine (C—X3—C motif) ligand 1 |
| 39254_at | up | 0.048494182 | retinoic acid induced 14 |
| 31826_at | up | 0.04850013 | KIAA0674 protein |
| 35247_at | down | 0.048510979 | small nuclear RNA activating complex, polypeptide 5, 19 kDa |
| 777_at | down | 0.048547239 | GDP dissociation inhibitor 2 |
| 36561_at | down | 0.048563389 | propionyl Coenzyme A carboxylase, beta polypeptide |
| 32597_at | down | 0.048567647 | retinoblastoma-like 2 (p130) |
| 39315_at | up | 0.048579702 | angiopoietin 1 |
| 33099_at | up | 0.048604195 | fucosyltransferase 5 (alpha (1,3) fucosyltransferase) |
| 37383_f_at | up | 0.048648959 | major histocompatibility complex, class I, C |
| 36479_at | up | 0.048655756 | growth arrest-specific 8 |
| 35804_at | down | 0.04866351 | ash2 (absent, small, or homeotic)-like (Drosophila) |
| 39258_at | down | 0.048682346 | ring finger protein 126 |
| 31560_at | up | 0.04868959 | interleukin 1 receptor-like 2 |
| 34694_at | up | 0.048706288 | SWI/SNF related, matrix associated, actin dependent regulator of |
| | | | chromatin, subfamily d, member 2 |
| 37398_at | down | 0.04874917 | platelet/endothelial cell adhesion molecule (CD31 antigen) |
| 33701_at | up | 0.048783164 | phenylalanine hydroxylase |
| 40338_at | up | 0.048799432 | |
| 38047_at | up | 0.048809968 | RNA binding protein with multiple splicing |
| 1552_i_at | up | 0.048820655 | cytochrome P450, family 2, subfamily A, polypeptide 13 |
| 32997_at | up | 0.04888841 | G antigen, family B, 1 (prostate associated) |
| 33506_at | up | 0.048920322 | inositol polyphosphate-4-phosphatase, type I, 107 kDa |
| 36257_at | up | 0.049004519 | |
| 896_at | up | 0.049060786 | mucin 2, intestinal/tracheal |
| 37101_at | up | 0.049085465 | breast cancer metastasis-suppressor 1 |
| 37612_at | up | 0.049087566 | parvalbumin |
| 33636_at | up | 0.0490977 | cancer/testis antigen 1 |
| 31349_at | up | 0.049121601 | DNA-binding protein amplifying expression of surfactant protein B |
| 39624_at | up | 0.049180262 | leukotriene B4 receptor |
| 33327_at | up | 0.04919815 | chromosome 11 open reading frame 9 |
| 33952_at | up | 0.049220745 | zinc finger protein 306 |
| 34631_at | up | 0.04924553 | eyes absent homolog 4 (Drosophila) |
| 33890_at | up | 0.049248261 | regulator of G-protein signalling 5 |
| 33646_g_at | up | 0.049269594 | GM2 ganglioside activator protein |
| 39065_s_at | down | 0.049283287 | tetratricopeptide repeat domain 3 |
| 32783_at | up | 0.049303682 | fibulin 2 |
| 35843_at | down | 0.049309055 | NIMA (never in mitosis gene a)-related kinase 9 |
| 41613_at | up | 0.04933168 | KIAA0329 gene product |
| 33912_at | down | 0.049387377 | zinc metalloproteinase (STE24 homolog, yeast) |
| 34093_at | up | 0.049407315 | |
| 32772_s_at | up | 0.049413791 | UDP-Gal:betaGlcNAc beta 1,4-galactosyltransferase, polypeptide 1 |
| 34402_at | down | 0.049425825 | unr-interacting protein |
| 39372_at | down | 0.049477707 | fatty acid desaturase 1 |
| 1254_at | up | 0.04948114 | guanylate cyclase activator 1A (retina) |
| 36121_at | up | 0.049509564 | epsin 2 |
| 33011_at | up | 0.049521701 | neurotensin receptor 2 |
| 37732_at | down | 0.049539909 | RING1 and YY1 binding protein |
| 36863_at | up | 0.049676408 | hyaluronan-mediated motility receptor (RHAMM) |
| 36010_at | up | 0.049689666 | mesenchyme homeo box 1 |
| 38355_at | down | 0.049749088 | DEAD/H (Asp-Glu-Ala-Asp/His) box polypeptide, Y chromosome |
| 41594_at | down | 0.049757578 | Janus kinase 1 (a protein tyrosine kinase) |
| 37538_at | up | 0.049804213 | |
| 41864_at | up | 0.04983549 | |
| 447_g_at | down | 0.0498497 | casein kinase 1, gamma 2 |
| 1647_at | down | 0.049914966 | IQ motif containing GTPase activating protein 2 |
| 38666_at | down | 0.050017281 | pleckstrin homology, Sec7 and coiled-coil domains 1 (cytohesin 1) |
| 37074_at | up | 0.05006693 | syntrophin, beta 1 (dystrophin-associated protein A1, 59 kDa, basi |
| | | | component 1) |
| 38753_at | down | 0.050071335 | exportin, tRNA (nuclear export receptor for tRNAs) |
| 37460_at | up | 0.050130808 | T-cell lymphoma invasion and metastasis 1 |
| 32924_at | up | 0.050139399 | matrix metalloproteinase 24 (membrane-inserted) |
| 33939_at | up | 0.05014943 | potassium voltage-gated channel, shaker-related subfamily, |
| | | | member 1 (episodic ataxia with myokymia) |
| 35942_at | up | 0.050154516 | |
| 41775_at | up | 0.050197141 | isoprenylcysteine carboxyl methyltransferase |
| 35200_at | up | 0.050298469 | high mobility group AT-hook 2 |
| 34889_at | down | 0.050400452 | ATPase, H+ transporting, lysosomal 70 kDa, V1 subunit A |
| 40927_at | up | 0.050411621 | solute carrier family 6 (neurotransmitter transporter, creatine), |
| | | | member 8 |
| 1170_at | up | 0.050472551 | |
| 34647_at | down | 0.050512762 | DEAD/H (Asp-Glu-Ala-Asp/His) box polypeptide 5 (RNA helicase, |
| | | | 68 kDa) |
| 595_at | down | 0.050514625 | tumor necrosis factor, alpha-induced protein 3 |
| 513_at | up | 0.050548181 | mitogen-activated protein kinase kinase 5 |
| 36567_at | up | 0.05061091 | solute carrier family 17 (sodium-dependent inorganic phosphate |
| | | | cotransporter), member 7 |
| 35386_at | up | 0.050611701 | acetylcholinesterase (YT blood group) |
| 34103_at | up | 0.050662538 | |
| 39176_f_at | up | 0.050705598 | carboxyl ester lipase (bile salt-stimulated lipase) |
| 34078_s_at | up | 0.050714993 | cytochrome P450, family 2, subfamily C, polypeptide 19 |
| 362_at | up | 0.050721464 | protein kinase C, zeta |
| 31341_at | up | 0.05078886 | potassium voltage-gated channel, Shaw-related subfamily, |
| | | | member 3 |
| 140_s_at | down | 0.050824342 | splicing factor, arginine/serine-rich 10 (transformer 2 homolog, |
| | | | Drosophila) |
| 38486_at | up | 0.050860569 | troponin I, skeletal, slow |
| 40607_at | down | 0.050870068 | dihydropyrimidinase-like 2 |
| 41563_at | up | 0.050883454 | transient receptor potential cation channel, subfamily M, member 1 |
| 38771_at | down | 0.050919217 | histone deacetylase 1 |
| 36514_at | down | 0.050919704 | cell growth regulatory with ring finger domain |
| 1689_at | up | 0.050938547 | protocadherin 16 dachsous-like (Drosophila) |
| 33287_at | up | 0.051003471 | hypothetical protein HSPC109 |
| 334_s_at | up | 0.051007767 | |
| 35956_s_at | up | 0.05102897 | pregnancy specific beta-1-glycoprotein 7 |
| 39704_s_at | down | 0.051031896 | high mobility group AT-hook 1 |
| 885_g_at | up | 0.051041395 | integrin, alpha 3 (antigen CD49C, alpha 3 subunit of VLA-3 |
| | | | receptor) |
| 35530_f_at | up | 0.051160162 | immunoglobulin lambda locus |
| 33458_r_at | down | 0.051179962 | histone 1, H2bc |
| 1330_at | up | 0.051206939 | mitogen-activated protein kinase kinase kinase 3 |
| 35178_at | up | 0.051239142 | WNT inhibitory factor 1 |
| 36556_at | up | 0.051298489 | KIAA0672 gene product |
| 34124_at | up | 0.051343537 | mitochondrial translational release factor 1-like |
| 32869_at | up | 0.051360804 | MRE11 meiotic recombination 11 homolog A (S. cerevisiae) |
| 38527_at | down | 0.051367943 | non-POU domain containing, octamer-binding |
| 38568_at | down | 0.051403125 | tumor protein p53-binding protein |
| 39023_at | down | 0.051454387 | isocitrate dehydrogenase 1 (NADP+), soluble |
| 41650_at | up | 0.051471647 | WD40 protein Ciao1 |
| 32582_at | up | 0.051507501 | myosin, heavy polypeptide 11, smooth muscle |
| 33350_s_at | down | 0.051519969 | JM5 protein |
| 36098_at | down | 0.051540433 | splicing factor, arginine/serine-rich 1 (splicing factor 2, alternate |
| | | | splicing factor) |
| 35499_at | up | 0.051549928 | hypothetical protein FLJ11336 |
| 31584_at | down | 0.051651619 | tumor protein, translationally-controlled 1 |
| 33660_at | down | 0.051688017 | ribosomal protein L5 |
| 36895_at | down | 0.051724914 | origin recognition complex, subunit 3-like (yeast) |
| 33228_g_at | down | 0.051742066 | interleukin 10 receptor, beta |
| 31752_at | up | 0.051948954 | hypothetical protein FLJ23142 |
| 418_at | up | 0.051966256 | antigen identified by monoclonal antibody Ki-67 |
| 39401_at | up | 0.052038202 | ribosomal protein S13 |
| 1343_s_at | up | 0.052108915 | serine (or cysteine) proteinase inhibitor, clade B (ovalbumin), |
| | | | member 3 |
| 855_at | down | 0.052145184 | programmed cell death 2 |
| 1174_at | up | 0.052217952 | |
| 37152_at | down | 0.052254523 | peroxisome proliferative activated receptor, delta |
| 37931_at | up | 0.052279349 | centromere protein B, 80 kDa |
| 41530_at | down | 0.052346679 | acetyl-Coenzyme A acyltransferase 2 (mitochondrial 3-oxoacyl- |
| | | | Coenzyme A thiolase) |
| 1859_s_at | up | 0.05236121 | Mdm2, transformed 3T3 cell double minute 2, p53 binding protein |
| | | | (mouse) |
| 35975_at | down | 0.052380431 | myeloid/lymphoid or mixed-lineage leukemia (trithorax homolog, |
| | | | Drosophila); translocated to, 3 |
| 33101_g_at | up | 0.052389477 | fetuin B |
| 1974_s_at | up | 0.052421103 | tumor protein p53 (Li-Fraumeni syndrome) |
| 1280_i_at | up | 0.052503614 | |
| 38600_r_at | up | 0.052520183 | |
| 35426_at | up | 0.052533016 | SPPL2b |
| 31978_at | up | 0.052541422 | kinesin family member 25 |
| 38746_at | up | 0.052620875 | integrin, beta 4 |
| 41153_f_at | down | 0.052679177 | catenin (cadherin-associated protein), alpha 1, 102 kDa |
| 35812_at | up | 0.052688828 | transportin-SR |
| 40788_at | up | 0.052750269 | adenylate kinase 2 |
| 40324_r_at | up | 0.052791746 | topoisomerase (DNA) III beta |
| 35271_at | down | 0.052803954 | ARP3 actin-related protein 3 homolog (yeast) |
| 38910_at | up | 0.052829015 | ATP synthase mitochondrial F1 complex assembly factor 2 |
| 38095_i_at | down | 0.052863898 | major histocompatibility complex, class II, DP beta 1 |
| 35267_g_at | down | 0.052865754 | bladder cancer associated protein |
| 38945_at | up | 0.052875372 | metal-regulatory transcription factor 1 |
| 38522_s_at | down | 0.052897744 | CD22 antigen |
| 31567_at | up | 0.052906998 | gamma-aminobutyric acid (GABA) A receptor, gamma 3 |
| 37099_at | down | 0.052981643 | arachidonate 5-lipoxygenase-activating protein |
| 562_g_at | up | 0.053109055 | follicle stimulating hormone receptor |
| 33906_at | up | 0.053130467 | Sjogren's syndrome/scleroderma autoantigen 1 |
| 40846_g_at | down | 0.053163872 | interleukin enhancer binding factor 3, 90 kDa |
| 34312_at | down | 0.053202442 | nuclear receptor coactivator 2 |
| 38644_at | up | 0.053219276 | paxillin |
| 33834_at | up | 0.053224091 | chemokine (C—X—C motif) ligand 12 (stromal cell-derived factor 1) |
| 155_s_at | down | 0.053379913 | ubiquitin-like 1 (sentrin) |
| 31363_at | up | 0.053396497 | CCR4-NOT transcription complex, subunit 2 |
| 32966_at | up | 0.053413878 | apolipoprotein F |
| 35227_at | up | 0.053429325 | retinoblastoma binding protein 8 |
| 1537_at | up | 0.053442744 | epidermal growth factor receptor (erythroblastic leukemia viral (v- |
| | | | erb-b) oncogene homolog, avian) |
| 32122_at | up | 0.053448125 | sulfite oxidase |
| 35515_at | up | 0.053507235 | tektin 2 (testicular) |
| 40387_at | down | 0.053544896 | endothelial differentiation, lysophosphatidic acid G-protein-couple |
| | | | receptor, 2 |
| 1718_at | down | 0.053617875 | actin related protein 2/3 complex, subunit 2, 34 kDa |
| 33341_at | down | 0.053655896 | guanine nucleotide binding protein (G protein), beta polypeptide 1 |
| 40269_at | down | 0.053675027 | PRP18 pre-mRNA processing factor 18 homolog (yeast) |
| 37000_at | down | 0.053751225 | DKFZP564B167 protein |
| 1667_s_at | up | 0.053766642 | cytochrome P450, family 4, subfamily B, polypeptide 1 |
| 37993_at | up | 0.053772854 | ATP synthase, H+ transporting, mitochondrial F1 complex, delta |
| | | | subunit |
| 40883_at | up | 0.053801821 | syntaxin 16 |
| 31420_at | up | 0.053822167 | immunoglobulin lambda variable (IV)/OR22-1 |
| 37980_at | down | 0.053851354 | CBF1 interacting corepressor |
| 1451_s_at | up | 0.05389717 | osteopath specific factor 2 (fasciclin I-like) |
| 39118_at | down | 0.05391293 | DnaJ (Hsp40) homolog, subfamily A, member 1 |
| 39265_at | up | 0.053944254 | type 1 tumor necrosis factor receptor shedding aminopeptidase |
| | | | regulator |
| 34569_at | up | 0.053973736 | SRY (sex determining region Y)-box 11 |
| 160_g_at | up | 0.054005608 | |
| 35168_f_at | down | 0.054037696 | collagen, type XVI, alpha 1 |
| 40015_at | up | 0.054067609 | KIAA0303 protein |
| 36219_at | down | 0.054111813 | similar to Caenorhabditis elegans protein C42C1.9 |
| 33068_f_at | up | 0.054171933 | UDP glycosyltransferase 2 family, polypeptide B15 |
| 538_at | up | 0.054185653 | CD34 antigen |
| 39503_s_at | up | 0.054254903 | dihydropyrimidinase-like 4 |
| 38427_at | up | 0.054256236 | collagen, type XV, alpha 1 |
| 33448_at | up | 0.05426235 | serine protease inhibitor, Kunitz type 1 |
| 36870_at | down | 0.054274318 | KIAA0804 protein |
| 41680_at | up | 0.054307826 | chromosome 1 open reading frame 34 |
| 38463_s_at | down | 0.054344711 | adenosine monophosphate deaminase (isoform E) |
| 36362_at | up | 0.054379239 | solute carrier family 12 (sodium/chloride transporters), member 3 |
| 32704_at | down | 0.054445472 | dedicator of cyto-kinesis 2 |
| 39168_at | up | 0.054468601 | Ac-like transposable element |
| 40587_s_at | down | 0.054472775 | eukaryotic translation elongation factor 1 epsilon 1 |
| 37081_at | up | 0.054493153 | dynein, axonemal, heavy polypeptide 7 |
| 33447_at | down | 0.05449949 | myosin regulatory light chain MRCL3 |
| 33205_at | up | 0.054582892 | suppressor of Ty 3 homolog (S. cerevisiae) |
| 41462_at | down | 0.054609547 | sorting nexin 2 |
| 41625_at | down | 0.054610798 | thyroid hormone receptor-associated protein, 240 kDa subunit |
| 32175_at | down | 0.054611527 | CDC10 cell division cycle 10 homolog (S. cerevisiae) |
| 36099_at | down | 0.054636307 | splicing factor, arginine/serine-rich 1 (splicing factor 2, alternate |
| | | | splicing factor) |
| 39349_at | up | 0.054649873 | HMT1 hnRNP methyltransferase-like 1 (S. cerevisiae) |
| 38367_s_at | up | 0.054689416 | complement component 4 binding protein, beta |
| 1474_s_at | up | 0.054758106 | v-myb myeloblastosis viral oncogene homolog (avian) |
| 40429_r_at | up | 0.054807695 | |
| 40502_r_at | up | 0.054812914 | myosin binding protein C, slow type |
| 34232_at | up | 0.054826808 | natural killer-tumor recognition sequence |
| 36148_at | up | 0.054862015 | amyloid beta (A4) precursor-like protein 1 |
| 1040_s_at | up | 0.054877884 | abl-interactor 2 |
| 41692_at | down | 0.054891324 | synaptojanin 1 |
| 32569_at | down | 0.054893855 | platelet-activating factor acetylhydrolase, isoform lb, alpha subunit |
| | | | 45 kDa |
| 160030_at | up | 0.054898491 | growth hormone receptor |
| 32452_at | up | 0.054953745 | cyclin-dependent kinase 3 |
| 36639_at | up | 0.054982119 | adenylosuccinate lyase |
| 40357_at | up | 0.054984105 | inhibin, beta A (activin A, activin AB alpha polypeptide) |
| 31437_r_at | up | 0.05500989 | estrogen receptor 2 (ER beta) |
| 38802_at | down | 0.055010478 | progesterone receptor membrane component 1 |
| 41837_at | up | 0.055099827 | chromosome 14 open reading frame 132 |
| 261_s_at | up | 0.055127541 | apolipoprotein B (including Ag(x) antigen) |
| 38083_at | down | 0.0551325 | Notch homolog 2 (Drosophila) |
| 35611_at | up | 0.055135445 | zinc finger protein 37 homolog (mouse) |
| 41812_s_at | down | 0.055143341 | nucleoporin 210 |
| 37535_at | down | 0.055180046 | cAMP responsive element binding protein 1 |
| 38800_at | up | 0.05524435 | stathmin-like 2 |
| 41663_at | up | 0.0552624 | |
| 36575_at | up | 0.055284709 | regulator of G-protein signalling 1 |
| 39739_at | down | 0.055289363 | nascent-polypeptide-associated complex alpha polypeptide |
| 38562_g_at | up | 0.055320447 | down-regulated in metastasis |
| 41052_s_at | up | 0.055330253 | calcium channel, voltage-dependent, P/Q type, alpha 1A subunit |
| 39548_at | up | 0.055386674 | neuronal PAS domain protein 2 |
| 31929_at | down | 0.055389172 | regulatory factor X, 3 (influences HLA class II expression) |
| 1946_at | up | 0.055451877 | Wilms tumor associated protein |
| 32324_at | down | 0.055453311 | tyrosine 3-monooxygenase/tryptophan 5-monooxygenase |
| | | | activation protein, beta polypeptide |
| 38660_at | up | 0.055562135 | cytochrome c oxidase subunit VIa polypeptide 2 |
| 38370_at | down | 0.055580846 | |
| 32702_at | up | 0.055632752 | trophinin associated protein (tastin) |
| 41184_s_at | down | 0.055648432 | proteasome (prosome, macropain) subunit, beta type, 8 (large |
| | | | multifunctional protease 7) |
| 33542_at | up | 0.05569065 | |
| 34444_at | up | 0.055727944 | chromosome X open reading frame 1 |
| 33043_at | up | 0.055762089 | eukaryotic translation elongation factor 1 gamma |
| 34461_at | up | 0.055819194 | synaptonemal complex protein 1 |
| 40243_at | up | 0.055858811 | metallo phosphoesterase |
| 36599_at | down | 0.055883675 | malic enzyme 2, NAD(+)-dependent, mitochondrial |
| 32026_s_at | down | 0.055895289 | PDZ domain containing guanine nucleotide exchange factor (GEF 1 |
| 33272_at | up | 0.055916736 | serum amyloid A1 |
| 35412_at | up | 0.055920677 | cytochrome P450, family 4, subfamily A, polypeptide 11 |
| 31753_at | up | 0.056077168 | |
| 32537_at | up | 0.05609059 | lipidosin |
| 36411_s_at | up | 0.056166675 | ELAV (embryonic lethal, abnormal vision, Drosophila)-like 2 (Hu |
| | | | antigen B) |
| 31844_at | down | 0.056320576 | homogentisate 1,2-dioxygenase (homogentisate oxidase) |
| 530_at | up | 0.056385304 | nuclear factor of kappa light polypeptide gene enhancer in B-cells |
| | | | inhibitor-like 2 |
| 903_at | down | 0.056390908 | protein phosphatase 2, regulatory subunit B (B56), alpha isoform |
| 39283_at | up | 0.056439846 | apical protein-like (Xenopus laevis) |
| 38406_f_at | up | 0.05649877 | prostaglandin D2 synthase 21 kDa (brain) |
| 39239_at | up | 0.056512111 | CD8 antigen, beta polypeptide 1 (p37) |
| 32481_at | up | 0.056673095 | |
| 37808_at | up | 0.056785668 | sorting nexin 7 |
| 37775_at | up | 0.056808833 | septin 6 |
| 938_at | up | 0.056826365 | |
| 34921_at | up | 0.056875557 | KIAA0420 gene product |
| 36472_at | down | 0.056888332 | N-myc (and STAT) interactor |
| 33154_at | down | 0.05689138 | proteasome (prosome, macropain) subunit, beta type, 4 |
| 41173_at | up | 0.05691759 | |
| 37651_at | down | 0.056919669 | REST corepressor |
| 39344_at | down | 0.056934629 | transformer-2 alpha (htra-2 alpha) |
| 1548_s_at | up | 0.056952857 | interleukin 10 |
| 34574_at | up | 0.056969408 | melanoma antigen, family A, 11 |
| 36493_at | down | 0.056992416 | lymphocyte-specific protein 1 |
| 41243_at | down | 0.057169482 | solute carrier family 35, member E2 |
| 34446_at | down | 0.057273151 | KIAA0471 gene product |
| 37595_at | up | 0.057288566 | |
| 40317_at | up | 0.057307104 | amiloride-sensitive cation channel 1, neuronal (degenerin) |
| 1948_f_at | up | 0.057328317 | nitric oxide synthase 2A (inducible, hepatocytes) |
| 39973_at | up | 0.057344468 | leprecan-like 2 protein |
| 40785_g_at | down | 0.057345337 | protein phosphatase 2, regulatory subunit B (B56), gamma |
| | | | isoform |
| 32076_at | up | 0.057364647 | Down syndrome critical region gene 1-like 1 |
| 35514_at | up | 0.057373421 | Rho family guanine-nucleotide exchange factor |
| 41152_f_at | down | 0.057442439 | ribosomal protein L36a |
| 38395_at | down | 0.057443145 | NADH dehydrogenase (ubiquinone) Fe—S protein 1, 75 kDa |
| | | | (NADH-coenzyme Q reductase) |
| 160042_s_at | up | 0.057472079 | homeo box B6 |
| 388_at | up | 0.057530222 | phosphoinositide-3-kinase, regulatory subunit, polypeptide 2 (p85 |
| | | | beta) |
| 41214_at | down | 0.057534645 | ribosomal protein S4, Y-linked |
| 35969_at | up | 0.057696203 | M-phase phosphoprotein 9 |
| 38023_at | up | 0.057706011 | phosphotidylinositol transfer protein |
| 39306_at | up | 0.057708653 | protease, serine, 16 (thymus) |
| 33246_at | up | 0.057726548 | mitogen-activated protein kinase 13 |
| 32809_at | down | 0.057746743 | |
| 37862_at | up | 0.057768456 | dihydrolipoamide branched chain transacylase (E2 component of |
| | | | branched chain keto acid dehydrogenase complex; maple syrup |
| | | | urine disease) |
| 1886_at | up | 0.057823252 | wingless-type MMTV integration site family, member 7A |
| 36519_at | down | 0.057885294 | excision repair cross-complementing rodent repair deficiency, |
| | | | complementation group 1 (includes overlapping antisense |
| | | | sequence) |
| 36465_at | up | 0.058006837 | interferon regulatory factor 5 |
| 1236_s_at | up | 0.058010665 | neurofibromin 1 (neurofibromatosis, von Recklinghausen disease, |
| | | | Watson disease) |
| 38299_at | up | 0.058034422 | interleukin 6 (interferon, beta 2) |
| 35928_at | up | 0.058057431 | thyroid peroxidase |
| 32029_at | up | 0.058077367 | 3-phosphoinositide dependent protein kinase-1 |
| 36104_at | down | 0.058084978 | ubiquinol-cytochrome c reductase hinge protein |
| 37893_at | down | 0.05809969 | protein tyrosine phosphatase, non-receptor type 2 |
| 713_at | up | 0.05811789 | |
| 32918_at | up | 0.058151217 | |
| 37917_at | down | 0.05818336 | hypothetical protein FLJ20323 |
| 1788_s_at | up | 0.058232158 | dual specificity phosphatase 4 |
| 31769_at | up | 0.058233033 | wingless-type MMTV integration site family, member 8B |
| 39219_at | down | 0.058252593 | CCAAT/enhancer binding protein (C/EBP), gamma |
| 416_s_at | up | 0.058290651 | homeo box C5 |
| 33457_at | down | 0.058377788 | retinoblastoma-associated protein 140 |
| 37718_at | down | 0.058387995 | SNF-1 related kinase |
| 34053_at | up | 0.058414295 | zona pellucida binding protein |
| 38245_i_at | up | 0.058439157 | mitogen-activated protein kinase kinase kinase kinase 5 |
| 37943_at | down | 0.05843963 | zinc finger, FYVE domain containing 26 |
| 40401_at | up | 0.058454018 | docking protein 5 |
| 39048_at | up | 0.0585224 | Notch homolog 4 (Drosophila) |
| 41023_at | up | 0.058574201 | complement component 8, alpha polypeptide |
| 37824_at | up | 0.058584019 | KIAA1074 protein |
| 39819_at | up | 0.058650541 | RNA polymerase I transcription factor RRN3 |
| 41231_f_at | down | 0.058672904 | high-mobility group nucleosomal binding domain 2 |
| 682_at | up | 0.058711408 | interferon, alpha 8 |
| 38931_at | down | 0.058759428 | zinc finger protein, X-linked |
| 40509_at | down | 0.058761269 | electron-transfer-flavoprotein, alpha polypeptide (glutaric aciduria |
| | | | II) |
| 32306_g_at | up | 0.058783564 | collagen, type I, alpha 2 |
| 38223_at | down | 0.058796236 | TBC1 domain family, member 8 (with GRAM domain) |
| 37920_at | up | 0.058825806 | paired-like homeodomain transcription factor 1 |
| 41821_at | down | 0.05885646 | cell division cycle 2-like 5 (cholinesterase-related cell division |
| | | | controller) |
| 40575_at | up | 0.058887769 | discs, large (Drosophila) homolog 5 |
| 33714_at | up | 0.058937876 | high-mobility group box 3 |
| 36778_at | down | 0.059000095 | ocular albinism 1 (Nettleship-Falls) |
| 369_s_at | up | 0.059042852 | ubiquitin-conjugating enzyme E2H (UBC8 homolog, yeast) |
| 34612_at | up | 0.05904774 | calbindin 3, (vitamin D-dependent calcium binding protein) |
| 38364_at | down | 0.059052258 | B lymphocyte gene 1 |
| 40952_at | up | 0.059083867 | BTG3 associated nuclear protein |
| 1008_f_at | up | 0.059087627 | protein kinase, interferon-inducible double stranded RNA |
| | | | dependent |
| 38930_at | up | 0.059243009 | |
| 37568_at | up | 0.059295057 | |
| 40011_s_at | up | 0.059317671 | fragile X mental retardation 2 |
| 34482_at | down | 0.059336699 | hypothetical protein MGC4701 |
| 38620_at | up | 0.059347346 | golgi SNAP receptor complex member 2 |
| 32641_at | down | 0.059370035 | androgen-induced proliferation inhibitor |
| 41848_f_at | up | 0.059393969 | interleukin 24 |
| 1446_at | down | 0.059425354 | proteasome (prosome, macropain) subunit, alpha type, 2 |
| 1611_s_at | up | 0.059473457 | interferon, gamma |
| 34519_at | up | 0.05950626 | natriuretic peptide receptor C/guanylate cyclase C (atrionatriuretic |
| | | | peptide receptor C) |
| 34792_at | down | 0.059523377 | S-adenosylhomocysteine hydrolase-like 1 |
| 1787_at | down | 0.05953562 | cyclin-dependent kinase inhibitor 1C (p57, Kip2) |
| 36975_at | down | 0.059558803 | hypothetical protein MGC8721 |
| 34337_s_at | down | 0.05957962 | likely ortholog of mouse metal response element binding |
| | | | transcription factor 2 |
| 39491_s_at | up | 0.059588745 | |
| 37623_at | up | 0.059601491 | nuclear receptor subfamily 4, group A, member 2 |
| 37483_at | down | 0.059686507 | histone deacetylase 9 |
| 937_at | down | 0.059741003 | |
| 32168_s_at | down | 0.059758424 | Down syndrome critical region gene 1 |
| 37524_at | down | 0.059785317 | serine/threonine kinase 17b (apoptosis-inducing) |
| 31776_at | up | 0.059797735 | |
| 37590_g_at | up | 0.059839604 | |
| 1113_at | up | 0.060099594 | bone morphogenetic protein 2 |
| 34595_at | up | 0.060161002 | myosin IA |
| 40608_at | down | 0.060169789 | ribosomal protein S13 |
| 36935_at | down | 0.060188988 | RAS p21 protein activator (GTPase activating protein) 1 |
| 32246_g_at | down | 0.060205029 | chromosome 14 open reading frame 92 |
| 34891_at | down | 0.060240172 | dynein, cytoplasmic, light polypeptide 1 |
| 40337_at | up | 0.060277334 | fucosyltransferase 1 (galactoside 2-alpha-L-fucosyltransferase, |
| | | | Bombay phenotype included) |
| 35326_at | up | 0.060313969 | Yip1 interacting factor homolog (S. cerevisiae) |
| 38983_at | down | 0.060318989 | NADH dehydrogenase (ubiquinone) 1 alpha subcomplex, 6, |
| | | | 14 kDa |
| 38783_at | up | 0.060335185 | mucin 1, transmembrane |
| 36597_at | down | 0.060379177 | nucleolar and coiled-body phosphoprotein 1 |
| 1445_at | up | 0.060466022 | chemokine (C—C motif) receptor-like 2 |
| 40212_at | up | 0.06048741 | kinase suppressor of ras |
| 1057_at | up | 0.060533082 | cellular retinoic acid binding protein 2 |
| 35992_at | up | 0.060585199 | musculin (activated B-cell factor-1) |
| 40084_at | down | 0.060604175 | transcription factor CP2 |
| 38685_at | down | 0.060642908 | syntaxin 12 |
| 35089_at | up | 0.060731492 | neuregulin 2 |
| 38672_at | up | 0.060809774 | protein phosphatase 1, regulatory subunit 10 |
| 38454_g_at | down | 0.060868973 | intercellular adhesion molecule 2 |
| 34208_at | up | 0.060876759 | solute carrier family 12, (potassium-chloride transporter) member 5 |
| 39278_at | up | 0.060889133 | transglutaminase 4 (prostate) |
| 37581_at | down | 0.060941394 | protein phosphatase 6, catalytic subunit |
| 38442_at | up | 0.060971291 | microfibrillar-associated protein 2 |
| 37634_at | up | 0.060996525 | progestagen-associated endometrial protein (placental protein 14 |
| | | | pregnancy-associated endometrial alpha-2-globulin, alpha uterine |
| | | | protein) |
| 729_i_at | up | 0.061028761 | |
| 33402_at | up | 0.061073887 | KDEL (Lys-Asp-Glu-Leu) endoplasmic reticulum protein retention |
| | | | receptor 3 |
| 41824_at | down | 0.061082771 | CGI-48 protein |
| 35377_at | up | 0.061238393 | DKFZP434M154 protein |
| 39205_at | up | 0.061272223 | hypothetical protein PP1665 |
| 584_s_at | down | 0.061289672 | X-ray repair complementing defective repair in Chinese hamster |
| | | | cells 5 (double-strand-break rejoining; Ku autoantigen, 80 kDa) |
| 38261_at | down | 0.061331903 | ATP-binding cassette, sub-family C (CFTR/MRP), member 3 |
| 40937_at | up | 0.061382943 | zinc finger protein 291 |
| 33376_at | up | 0.061386605 | N-deacetylase/N-sulfotransferase (heparan glucosaminyl) 1 |
| 39571_at | up | 0.0614091 | hypothetical protein DKFZp434G2311 |
| 36670_at | up | 0.061452772 | autoantigen |
| 31675_s_at | down | 0.06149695 | phosphatase and tensin homolog (mutated in multiple advanced |
| | | | cancers 1), pseudogene 1 |
| 34730_g_at | up | 0.061538175 | trophinin |
| 41556_s_at | up | 0.061577406 | heparan sulfate (glucosamine) 3-O-sulfotransferase 1 |
| 35737_at | up | 0.061613514 | high mobility group nucleosomal binding domain 4 |
| 36064_at | up | 0.061678211 | potassium voltage-gated channel, KQT-like subfamily, member 2 |
| 34829_at | up | 0.061682903 | dyskeratosis congenita 1, dyskerin |
| 1592_at | up | 0.061762496 | topoisomerase (DNA) II alpha 170 kDa |
| 34657_at | down | 0.06176476 | A kinase (PRKA) anchor protein 11 |
| 33254_at | down | 0.061846647 | ecotropic viral integration site 5 |
| 1367_f_at | down | 0.061855363 | ubiquitin C |
| 38060_at | down | 0.061883861 | NADH dehydrogenase (ubiquinone) Fe—S protein 5, 15 kDa |
| | | | (NADH-coenzyme Q reductase) |
| 33797_at | up | 0.06191586 | M-phase phosphoprotein 10 (U3 small nucleolar |
| | | | ribonucleoprotein) |
| 35294_at | down | 0.061934664 | Sjogren syndrome antigen A2 (60 kDa, ribonucleoprotein |
| | | | autoantigen SS-A/Ro) |
| 39437_at | up | 0.061948929 | zinc finger protein 289, ID1 regulated |
| 34723_at | down | 0.061958734 | COX11 homolog, cytochrome c oxidase assembly protein (yeast) |
| 40800_at | up | 0.061977237 | HN1 like |
| 40300_g_at | up | 0.062012415 | G-protein coupled receptor |
| 2013_at | up | 0.062019318 | transcription factor Dp-2 (E2F dimerization partner 2) |
| 398_at | up | 0.062027909 | DEAD/H (Asp-Glu-Ala-Asp/His) box polypeptide 18 (Myc- |
| | | | regulated) |
| 35700_at | up | 0.062054825 | chromodomain helicase DNA binding protein 2 |
| 37176_at | up | 0.0620627 | hyaluronoglucosaminidase 1 |
| 35906_at | up | 0.062070421 | solute carrier family 5 (sodium/glucose cotransporter), member 1 |
| 38306_at | down | 0.062100311 | brefeldin A-inhibited guanine nucleotide-exchange protein 1 |
| 33943_at | down | 0.062127606 | ferritin, heavy polypeptide 1 |
| 31652_at | up | 0.062177255 | KIAA1000 protein |
| 39115_at | down | 0.06219621 | cysteine-rich with EGF-like domains 1 |
| 1897_at | up | 0.062201284 | transforming growth factor, beta receptor III (betaglycan, 300 kDa) |
| 288_s_at | down | 0.062457541 | lamin B receptor |
| 904_s_at | up | 0.06249421 | topoisomerase (DNA) II alpha 170 kDa |
| 34544_at | down | 0.06257331 | zinc finger protein 267 |
| 38622_at | up | 0.062579122 | hypothetical protein BC004409 |
| 32099_at | down | 0.062599116 | scaffold attachment factor B2 |
| 33485_at | down | 0.062675228 | ribosomal protein L4 |
| 38778_at | down | 0.062677106 | KIAA1046 protein |
| 966_at | up | 0.0627393 | RAD54-like (S. cerevisiae) |
| 34111_s_at | up | 0.062792399 | |
| 35272_at | down | 0.062795602 | guanine nucleotide binding protein (G protein), gamma 5 |
| 32807_at | down | 0.062914078 | DKFZP566C134 protein |
| 37913_at | up | 0.062920328 | dihydrofolate reductase |
| 36226_r_at | up | 0.062926066 | splicing factor proline/glutamine rich (polypyirmidine tract binding |
| | | | protein associated) |
| 39885_at | down | 0.062939041 | putative dimethyladenosine transferase |
| 34571_at | up | 0.063007099 | guanine nucleotide binding protein (G protein), alpha transducing |
| | | | activity polypeptide 2 |
| 39602_at | up | 0.063009221 | myosin VIIA and Rab interacting protein |
| 34594_at | down | 0.063034238 | related to the N terminus of tre |
| 40809_at | up | 0.063035272 | syntrophin, beta 2 (dystrophin-associated protein A1, 59 kDa, basi |
| | | | component 2) |
| 40707_at | up | 0.06310375 | M-phase phosphoprotein 9 |
| 33684_at | up | 0.0631076 | wingless-type MMTV integration site family, member 2B |
| 31973_at | up | 0.063114368 | calcium channel, voltage-dependent, alpha 1G subunit |
| 32382_at | up | 0.063143488 | uroplakin 1B |
| 102_at | up | 0.0632572 | homeodomain interacting protein kinase 3 |
| 38818_at | down | 0.063335303 | serine palmitoyltransferase, long chain base subunit 1 |
| 32549_at | up | 0.063359153 | pregnancy-associated plasma protein A |
| 32780_at | up | 0.063441097 | bullous pemphigoid antigen 1, 230/240 kDa |
| 37586_at | up | 0.063463781 | zinc finger protein 142 (clone pHZ-49) |
| 31728_at | up | 0.063480732 | major histocompatibility complex, class II, DO alpha |
| 33324_s_at | up | 0.063481517 | cell division cycle 2, G1 to S and G2 to M |
| 33722_at | down | 0.063552526 | attractin |
| 31618_at | up | 0.063629282 | |
| 37231_at | up | 0.063647129 | discs, large homolog 7 (Drosophila) |
| 33295_at | up | 0.063653906 | Duffy blood group |
| 38344_at | down | 0.0636827 | Alstrom syndrome 1 |
| 630_at | up | 0.063683304 | dCMP deaminase |
| 39828_at | up | 0.063688282 | ADP-ribosylation factor-like 7 |
| 36932_at | down | 0.063720811 | general transcription factor IIIC, polypeptide 2, beta 110 kDa |
| 1213_at | down | 0.063729631 | SFRS protein kinase 2 |
| 36757_at | down | 0.063735666 | histone 1, H3h |
| 41856_at | up | 0.063822614 | |
| 40717_at | up | 0.063953051 | cathepsin L2 |
| 37142_at | up | 0.064046062 | GDNF family receptor alpha 1 |
| 34772_at | up | 0.064054956 | coronin, actin binding protein, 2B |
| 36060_at | down | 0.064069461 | signal recognition particle 54 kDa |
| 1291_s_at | up | 0.064069798 | fibroblast growth factor receptor 4 |
| 40029_at | up | 0.064078515 | EGF-like-domain, multiple 3 |
| 37072_at | up | 0.064097818 | cyclic nucleotide gated channel beta 1 |
| 38860_at | up | 0.064166427 | phosphodiesterase 4C, cAMP-specific (phosphodiesterase E1 |
| | | | dunce homolog, Drosophila) |
| 1490_at | up | 0.064176408 | v-myc myelocytomatosis viral oncogene homolog 1, lung |
| | | | carcinoma derived (avian) |
| 38679_g_at | down | 0.064193026 | small nuclear ribonucleoprotein polypeptide E |
| 31946_s_at | up | 0.064273083 | forkhead box G1A |
| 38266_at | down | 0.064299188 | retinoblastoma binding protein 6 |
| 33334_at | down | 0.064353716 | acylphosphatase 1, erythrocyte (common) type |
| 1737_s_at | up | 0.064389565 | insulin-like growth factor binding protein 4 |
| 1781_at | up | 0.064434279 | ELK1, member of ETS oncogene family |
| 39396_at | down | 0.064438229 | lysophospholipase I |
| 34063_at | up | 0.064446601 | RecQ protein-like 5 |
| 37230_at | down | 0.064472303 | KIAA0469 gene product |
| 32265_at | up | 0.064485263 | nuclear receptor subfamily 4, group A, member 1 |
| 40832_s_at | down | 0.064506377 | lamina-associated polypeptide 1B |
| 35356_at | down | 0.064514009 | hypothetical protein MGC9651 |
| 40539_at | up | 0.064543391 | myosin IXB |
| 40984_at | up | 0.064566113 | gamma tubulin ring complex protein (76p gene) |
| 41376_i_at | up | 0.064571434 | UDP glycosyltransferase 2 family, polypeptide B7 |
| 41576_at | up | 0.064620473 | |
| 273_g_at | up | 0.064629736 | gastrin-releasing peptide |
| 37022_at | up | 0.064629783 | proline arginine-rich end leucine-rich repeat protein |
| 722_at | up | 0.06464731 | RCD1 required for cell differentiation1 homolog (S. pombe) |
| 34077_at | up | 0.064651773 | chemokine (C—X—C motif) receptor 3 |
| 37341_at | up | 0.064654132 | glutamate dehydrogenase 1 |
| 39481_at | up | 0.06467974 | long-chain fatty-acyl elongase |
| 36436_at | up | 0.064760378 | leukocyte cell-derived chemotaxin 2 |
| 35716_at | up | 0.064783583 | sulfotransferase family, cytosolic, 1C, member 1 |
| 133_at | down | 0.064821382 | cathepsin C |
| 36312_at | down | 0.064882797 | serine (or cysteine) proteinase inhibitor, clade B (ovalbumin), |
| | | | member 8 |
| 41000_at | down | 0.064912113 | checkpoint suppressor 1 |
| 31677_at | up | 0.064958769 | |
| 1528_at | up | 0.06500764 | hypothetical gene CG030 |
| 36864_at | up | 0.065008406 | peroxisomal biogenesis factor 3 |
| 36325_at | up | 0.065037727 | crystallin, beta A1 |
| 33413_at | down | 0.065039089 | protein tyrosine phosphatase type IVA, member 1 |
| 40336_at | up | 0.0651239 | ferredoxin reductase |
| 41597_s_at | down | 0.065192685 | SEC22 vesicle trafficking protein-like 1 (S. cerevisiae) |
| 39536_at | up | 0.065201347 | homeo box (H6 family) 1 |
| 1073_at | down | 0.065224227 | transcription elongation factor A (SII), 1 |
| 39594_f_at | up | 0.065236518 | metallothionein 1H |
| 32974_at | up | 0.065273556 | homolog of Yeast RRP4 (ribosomal RNA processing 4), 3′-5′- |
| | | | exoribonuclease |
| 37126_at | up | 0.065330653 | Sjogren syndrome antigen A1 (52 kDa, ribonucleoprotein |
| | | | autoantigen SS-A/Ro) |
| 34693_at | down | 0.065343616 | sialyltransferase |
| 37925_r_at | up | 0.065370621 | apolipoprotein M |
| 1190_at | up | 0.065390496 | protein tyrosine phosphatase, receptor type, O |
| 39243_s_at | down | 0.065560845 | PC4 and SFRS1 interacting protein 2 |
| 40333_at | up | 0.065610099 | bone morphogenetic protein 4 |
| 40428_i_at | up | 0.065620187 | |
| 35703_at | down | 0.065621274 | platelet-derived growth factor alpha polypeptide |
| 33754_at | up | 0.065631647 | thyroid transcription factor 1 |
| 36620_at | down | 0.065767238 | superoxide dismutase 1, soluble (amyotrophic lateral sclerosis 1 |
| | | | (adult)) |
| 40710_at | up | 0.065846675 | calmegin |
| 38629_at | up | 0.065898386 | microtubule-associated protein tau |
| 41395_at | up | 0.06595199 | carbohydrate (keratan sulfate Gal-6) sulfotransferase 1 |
| 32489_at | up | 0.065955036 | glutamate receptor, ionotropic, N-methyl D-aspartate 2B |
| 35022_at | up | 0.065980914 | SRY (sex determining region Y)-box 5 |
| 33861_at | down | 0.066186823 | CCR4-NOT transcription complex, subunit 2 |
| 39394_at | up | 0.066198374 | |
| 32986_s_at | up | 0.066223752 | MAD, mothers against decapentaplegic homolog 9 (Drosophila) |
| 1707_g_at | up | 0.066240761 | v-raf murine sarcoma 3611 viral oncogene homolog 1 |
| 35078_at | up | 0.066266338 | intercellular adhesion molecule 4, Landsteiner-Wiener blood group |
| 32846_s_at | down | 0.066279462 | kinectin 1 (kinesin receptor) |
| 39615_at | up | 0.066293934 | KIAA1026 protein |
| 39552_at | down | 0.06636328 | phosphatase and tensin homolog (mutated in multiple advanced |
| | | | cancers 1) |
| 41297_at | up | 0.066367138 | mannosidase, alpha, class 1A, member 2 |
| 36290_s_at | up | 0.0663784 | fucosyltransferase 6 (alpha (1,3) fucosyltransferase) |
| 33367_s_at | down | 0.066406849 | ornithine decarboxylase antizyme inhibitor |
| 40536_f_at | up | 0.066482666 | translation initiation factor IF2 |
| 32148_at | up | 0.066539346 | FERM, RhoGEF (ARHGEF) and pleckstrin domain protein 1 |
| | | | (chondrocyte-derived) |
| 1542_at | up | 0.066618173 | epidermal growth factor (beta-urogastrone) |
| 31419_r_at | up | 0.066633328 | |
| 39096_at | down | 0.066635074 | SON DNA binding protein |
| 34762_at | up | 0.066729339 | ring finger protein (C3HC4 type) 8 |
| 36672_at | down | 0.066753245 | prolylcarboxypeptidase (angiotensinase C) |
| 39475_at | up | 0.066768992 | chromosome 4 open reading frame 9 |
| 947_at | up | 0.066788523 | MCM7 minichromosome maintenance deficient 7 (S. cerevisiae) |
| 894_g_at | up | 0.06685321 | ubiquitin carrier protein |
| 34654_at | down | 0.066869319 | myotubularin related protein 1 |
| 36449_s_at | up | 0.066888403 | peptide YY |
| 40125_at | down | 0.066905911 | calnexin |
| 39629_at | up | 0.066929858 | phospholipase A2, group V |
| 40444_s_at | up | 0.066945781 | catenin (cadherin-associated protein), delta 1 |
| 581_at | up | 0.066955198 | laminin, beta 1 |
| 38715_at | up | 0.066966094 | glycophorin B (includes Ss blood group) |
| 32835_at | down | 0.066974462 | sudD suppressor of bimD6 homolog (A. nidulans) |
| 39510_r_at | down | 0.067021841 | programmed cell death 4 (neoplastic transformation inhibitor) |
| 41535_at | down | 0.06707185 | CDK2-associated protein 1 |
| 32667_at | up | 0.067095946 | collagen, type IV, alpha 5 (Alport syndrome) |
| 31971_at | up | 0.067178117 | putative GR6 protein |
| 789_at | up | 0.067241382 | early growth response 1 |
| 39636_at | up | 0.067253181 | |
| 31765_at | up | 0.067273333 | KIAA0694 gene product |
| 35776_at | up | 0.067316705 | intersectin 1 (SH3 domain protein) |
| 39125_at | up | 0.067337952 | transient receptor potential cation channel, subfamily C, member 1 |
| 34384_at | down | 0.067348878 | ATP-binding cassette, sub-family C (CFTR/MRP), member 1 |
| 1540_f_at | up | 0.067365851 | interferon, alpha 5 |
| 961_at | up | 0.067375409 | neurofibromin 1 (neurofibromatosis, von Recklinghausen disease, |
| | | | Watson disease) |
| 35069_at | up | 0.067384697 | hypothetical protein similar to preferentially expressed antigen of |
| | | | melanoma |
| 32754_at | up | 0.067393559 | tropomyosin 3 |
| 33872_at | up | 0.067430249 | latrophilin 2 |
| 701_s_at | up | 0.067454963 | |
| 32380_at | up | 0.067457336 | plakophilin 1 (ectodermal dysplasia/skin fragility syndrome) |
| 34749_at | down | 0.06748559 | solute carrier family 31 (copper transporters), member 2 |
| 40980_at | up | 0.067495072 | helicase with SNF2 domain 1 |
| 39327_at | up | 0.067577451 | Melanoma associated gene |
| 31663_at | up | 0.06777859 | myeloid/lymphoid or mixed-lineage leukemia (trithorax homolog, |
| | | | Drosophila); translocated to, 4 |
| 39148_s_at | up | 0.067828539 | alpha thalassemia/mental retardation syndrome X-linked (RAD54 |
| | | | homolog, S. cerevisiae) |
| 36616_at | down | 0.06785433 | DAZ associated protein 2 |
| 37530_s_at | up | 0.067868284 | reelin |
| 41091_at | down | 0.067977726 | fetal Alzheimer antigen |
| 35064_at | up | 0.067980534 | tripartite motif-containing 31 |
| 40153_at | down | 0.067983399 | transporter 1, ATP-binding cassette, sub-family B (MDR/TAP) |
| 34623_at | up | 0.06798768 | defensin, alpha 5, Paneth cell-specific |
| 39036_g_at | down | 0.068092911 | progestin induced protein |
| 33886_at | down | 0.068114085 | spectrin SH3 domain binding protein 1 |
| 40815_g_at | down | 0.068233922 | iduronate 2-sulfatase (Hunter syndrome) |
| 1000_at | up | 0.068270711 | mitogen-activated protein kinase 3 |
| 41296_s_at | down | 0.068276144 | START domain containing 7 |
| 38648_at | down | 0.06827716 | zinc finger protein 384 |
| 1994_at | up | 0.068310712 | activating transcription factor 2 |
| 35781_g_at | up | 0.068345587 | KIAA0657 protein |
| 37235_g_at | up | 0.068372273 | kininogen |
| 32639_at | up | 0.068383912 | nucleoporin-like protein 1 |
| 41470_at | up | 0.068392895 | prominin 1 |
| 723_s_at | down | 0.068462859 | |
| 39180_at | down | 0.068510684 | fusion, derived from t(12; 16) malignant liposarcoma |
| 33905_at | down | 0.068659685 | methyl-CpG binding domain protein 2 |
| 38459_g_at | up | 0.068816314 | cytochrome b-5 |
| 36673_at | down | 0.068888528 | mannose phosphate isomerase |
| 160036_at | up | 0.068911333 | estrogen-related receptor beta |
| 31660_at | up | 0.068977387 | DKFZP434A062 protein |
| 38200_at | up | 0.069062386 | faciogenital dysplasia (Aarskog-Scott syndrome) |
| 33651_at | up | 0.069111018 | aquaporin 8 |
| 842_at | up | 0.069200967 | protein kinase C binding protein 1 |
| 208_at | up | 0.069215423 | catenin (cadherin-associated protein), alpha 2 |
| 37140_s_at | up | 0.069281532 | ectodermal dysplasia 1, anhidrotic |
| 32418_at | up | 0.069291623 | phosphodiesterase 1C, calmodulin-dependent 70 kDa |
| 36214_at | down | 0.069400426 | Kruppel-like factor 4 (gut) |
| 36685_at | down | 0.069578272 | adenosylmethionine decarboxylase 1 |
| 36311_at | up | 0.0696169 | phosphodiesterase 1A, calmodulin-dependent |
| 35675_at | up | 0.06965234 | vinexin beta (SH3-containing adaptor molecule-1) |
| 40162_s_at | up | 0.069667733 | cartilage oligomeric matrix protein (pseudoachondroplasia, |
| | | | epiphyseal dysplasia 1, multiple) |
| 38102_at | down | 0.069691466 | hypothetical protein FLJ34588 |
| 193_at | down | 0.069842253 | TAF9 RNA polymerase II, TATA box binding protein (TBP)- |
| | | | associated factor, 32 kDa |
| 32039_at | down | 0.069879541 | adaptor-related protein complex 3, beta 1 subunit |
| 35604_at | up | 0.069916727 | endonuclease G-like 1 |
| 39346_at | down | 0.069917202 | KH domain containing, RNA binding, signal transduction |
| | | | associated 1 |
| 35844_at | up | 0.070023077 | syndecan 4 (amphiglycan, ryudocan) |
| 711_at | up | 0.070029379 | |
| 39378_at | down | 0.070031282 | beclin 1 (coiled-coil, myosin-like BCL2 interacting protein) |
| 2017_s_at | up | 0.070139758 | cyclin D1 (PRAD1: parathyroid adenomatosis 1) |
| 41361_at | up | 0.070337448 | CCR4-NOT transcription complex, subunit 8 |
| 36387_at | up | 0.070387113 | |
| 40569_at | up | 0.070393476 | zinc finger protein 42 (myeloid-specific retinoic acid-responsive) |
| 40489_at | up | 0.070419101 | dentatorubral-pallidoluysian atrophy (atrophin-1) |
| 37650_at | down | 0.070599809 | makorin, ring finger protein, 1 |
| 1798_at | down | 0.07062824 | LIV-1 protein, estrogen regulated |
| 35881_at | up | 0.070726932 | |
| 40545_at | down | 0.070878906 | proline synthetase co-transcribed homolog (bacterial) |
| 41839_at | up | 0.0708905 | growth arrest-specific 1 |
| 32282_at | up | 0.070924055 | |
| 39210_at | up | 0.070930588 | fucosyltransferase 4 (alpha (1,3) fucosyltransferase, myeloid- |
| | | | specific) |
| 31558_at | up | 0.070984093 | Hr44 antigen |
| 34383_at | down | 0.070998576 | ubiquitin specific protease 1 |
| 33179_at | up | 0.071011949 | protein phosphatase 1, regulatory (inhibitor) subunit 2 |
| 36598_s_at | up | 0.071022431 | inositol polyphosphate phosphatase-like 1 |
| 390_at | up | 0.071049442 | chemokine (C—C motif) receptor 4 |
| 33526_at | up | 0.071061356 | neuropeptide Y receptor Y2 |
| 1748_s_at | up | 0.071122402 | Kruppel-like factor 1 (erythroid) |
| 40835_at | up | 0.071138085 | metastasis-associated 1-like 1 |
| 36669_at | up | 0.071169146 | FBJ murine osteosarcoma viral oncogene homolog B |
| 33162_at | down | 0.071262469 | insulin receptor |
| 35039_at | up | 0.071324095 | KIAA0276 protein |
| 33390_at | up | 0.071340473 | serine/threonine kinase 17b (apoptosis-inducing) |
| 33044_f_at | up | 0.071406367 | empty spiracles homolog 1 (Drosophila) |
| 33248_at | up | 0.071526167 | |
| 37409_at | down | 0.07160345 | SFRS protein kinase 2 |
| 31783_at | down | 0.071657185 | renin binding protein |
| 41080_at | up | 0.071729743 | H2A histone family, member B |
| 519_g_at | up | 0.071740889 | nuclear receptor subfamily 1, group H, member 2 |
| 38079_at | up | 0.07176621 | guanine nucleotide binding protein (G protein), gamma 12 |
| 41723_s_at | down | 0.071880267 | major histocompatibility complex, class II, DR beta 1 |
| 37560_at | up | 0.0719212 | FLJ00133 protein |
| 32867_at | up | 0.071922807 | choroideremia (Rab escort protein 1) |
| 41803_g_at | up | 0.072091533 | hypothetical protein FLJ22531 |
| 419_at | up | 0.072110323 | antigen identified by monoclonal antibody Ki-67 |
| 39146_at | up | 0.07212862 | alpha thalassemia/mental retardation syndrome X-linked (RAD54 |
| | | | homolog, S. cerevisiae) |
| 41175_at | down | 0.072189347 | core-binding factor, beta subunit |
| 34491_at | up | 0.072260618 | 2′-5′-oligoadenylate synthetase-like |
| 36683_at | up | 0.072283268 | matrix Gla protein |
| 37598_at | down | 0.072298575 | Ras association (RaIGDS/AF-6) domain family 2 |
| 37403_at | down | 0.072370544 | annexin A1 |
| 35862_at | up | 0.072384347 | solute carrier family 15 (H+/peptide transporter), member 2 |
| 34242_at | up | 0.072461287 | chromosome 20 open reading frame 194 |
| 331_at | up | 0.072463612 | |
| 41638_at | down | 0.072496969 | KIAA0073 protein |
| 215_g_at | up | 0.072524414 | msh homeo box homolog 1 (Drosophila) |
| 31642_at | up | 0.072608877 | |
| 36322_at | up | 0.072612703 | fucosyltransferase 7 (alpha (1, 3) fucosyltransferase) |
| 812_at | down | 0.07262422 | protein phosphatase 1, regulatory (inhibitor) subunit 2 |
| 41345_at | up | 0.072637402 | purine-rich element binding protein A |
| 34940_at | down | 0.072766213 | |
| 35909_at | up | 0.072779379 | pleckstrin homology-like domain, family A, member 1 |
| 38856_at | up | 0.072867435 | KIAA1233 protein |
| 37911_at | up | 0.072887305 | syntaxin 4A (placental) |
| 36373_at | up | 0.072953876 | zinc finger, X-linked, duplicated A |
| 37751_at | down | 0.07295976 | KIAA0255 gene product |
| 39733_at | down | 0.073064777 | homocysteine-inducible, endoplasmic reticulum stress-inducible, |
| | | | ubiquitin-like domain member 1 |
| 33337_at | down | 0.073072611 | degenerative spermatocyte homolog, lipid desaturase (Drosophila) |
| 35718_at | down | 0.07317749 | SP110 nuclear body protein |
| 32253_at | down | 0.073249284 | arginine-glutamic acid dipeptide (RE) repeats |
| 37229_at | down | 0.07336482 | ataxia telangiectasia and Rad3 related |
| 34912_at | up | 0.073371211 | death-associated protein kinase 2 |
| 41008_at | up | 0.073394717 | KIAA0888 protein |
| 41286_at | up | 0.073511778 | tumor-associated calcium signal transducer 2 |
| 1476_s_at | up | 0.073525062 | v-myb myeloblastosis viral oncogene homolog (avian) |
| 657_at | up | 0.073549188 | protocadherin gamma subfamily C, 3 |
| 40698_at | down | 0.073559018 | C-type (calcium dependent, carbohydrate-recognition domain) |
| | | | lectin, superfamily member 2 (activation-Induced) |
| 633_s_at | up | 0.073596989 | transcription factor Dp-2 (E2F dimerization partner 2) |
| 36006_at | up | 0.073614666 | SRY (sex determining region Y)-box 12 |
| 1930_at | down | 0.073728457 | ATP-binding cassette, sub-family C (CFTR/MRP), member 3 |
| 1123_at | up | 0.073740648 | growth hormone releasing hormone receptor |
| 33146_at | down | 0.073857687 | myeloid cell leukemia sequence 1 (BCL2-related) |
| 38575_at | down | 0.073876745 | mucosa associated lymphoid tissue lymphoma translocation gene 1 |
| 36506_at | down | 0.073893875 | A kinase (PRKA) anchor protein (yotiao) 9 |
| 32337_at | down | 0.073926169 | ribosomal protein L21 |
| 37385_at | down | 0.073942562 | peptidyl-prolyl isomerase G (cyclophilin G) |
| 1585_at | up | 0.073949466 | v-erb-b2 erythroblastic leukemia viral oncogene homolog 3 (avian |
| 33811_at | down | 0.073960943 | cell cycle progression 8 protein |
| 37306_at | down | 0.073995502 | cytoplasmic FMR1 interacting protein 1 |
| 35761_at | down | 0.07401577 | aminoadipate-semialdehyde dehydrogenase-phosphopantetheiny |
| | | | transferase |
| 39776_at | up | 0.074189511 | unc-51-like kinase 2 (C. elegans) |
| 41060_at | up | 0.07422555 | cyclin E1 |
| 37676_at | down | 0.07427798 | phosphodiesterase 8A |
| 31839_at | down | 0.074281268 | splicing factor 4 |
| 34998_at | down | 0.074281619 | protein arginine N-methyltransferase 3(hnRNP methyltransferase |
| | | | S. cerevisiae)-like 3 |
| 2018_at | up | 0.074304439 | gap junction protein alpha 1, 43 kDa (connexin 43) |
| 41004_at | down | 0.074346366 | topoisomerase (DNA) III alpha |
| 36571_at | down | 0.074361104 | topoisomerase (DNA) II beta 180 kDa |
| 33249_at | up | 0.074378692 | nuclear receptor subfamily 3, group C, member 2 |
| 34953_i_at | up | 0.074406032 | phosphodiesterase 5A, cGMP-specific |
| 1708_at | up | 0.074445148 | mitogen-activated protein kinase 10 |
| 32674_at | down | 0.074454592 | NP220 nuclear protein |
| 1204_at | up | 0.07450712 | diacylglycerol kinase, gamma 90 kDa |
| AFFX-BioC-3_st | up | 0.074577771 | |
| 39353_at | down | 0.074613199 | heat shock 10 kDa protein 1 (chaperonin 10) |
| 31863_at | down | 0.074748576 | KIAA0179 protein |
| 38838_at | up | 0.074755593 | polymyositis/scleroderma autoantigen 1, 75 kDa |
| 36035_at | up | 0.074835381 | GPAA1P anchor attachment protein 1 homolog (yeast) |
| 40422_at | down | 0.074893541 | insulin-like growth factor binding protein 2, 36 kDa |
| 34987_s_at | down | 0.07495392 | heterogeneous nuclear ribonucleoprotein A1 |
| 36124_at | up | 0.074965842 | mercaptopyruvate sulfurtransferase |
| 35657_at | up | 0.074965943 | TAR (HIV) RNA binding protein 2 |
| 36849_at | up | 0.074984491 | PTPL1-associated RhoGAP 1 |
| 32182_at | down | 0.074998717 | serine/threonine kinase 38 like |
| 32758_g_at | down | 0.075062841 | RAE1 RNA export 1 homolog (S. pombe) |
| 461_at | down | 0.075106762 | N-acylsphingosine amidohydrolase (acid ceramidase) 1 |
| 37175_at | up | 0.075116156 | serine (or cysteine) proteinase inhibitor, clade C (antithrombin), |
| | | | member 1 |
| 34268_at | down | 0.075146357 | regulator of G-protein signalling 19 |
| 528_at | up | 0.075153984 | heat shock 27 kDa protein 3 |
| 36224_g_at | down | 0.075175637 | splicing factor proline/glutamine rich (polypyrimidine tract binding |
| | | | protein associated) |
| 33194_at | up | 0.07528018 | RCD1 required for cell differentiation1 homolog (S. pombe) |
| 38990_at | down | 0.075291777 | F-box only protein 9 |
| 901_g_at | up | 0.075464454 | phospholipase C, beta 4 |
| 37962_r_at | down | 0.07548297 | syntaxin binding protein 3 |
| 34445_at | down | 0.075645748 | KIAA0471 gene product |
| 40051_at | up | 0.075647797 | translocation associated membrane protein 2 |
| 34391_at | down | 0.07567032 | immunoglobulin (CD79A) binding protein 1 |
| 40151_s_at | down | 0.075685419 | peroxisome receptor 1 |
| 32183_at | down | 0.075709577 | splicing factor, arginine/serine-rich 11 |
| 40024_at | up | 0.075728682 | src homology three (SH3) and cysteine rich domain |
| 34611_at | up | 0.075745185 | zinc finger protein 192 |
| 35590_s_at | up | 0.075756397 | gastric inhibitory polypeptide receptor |
| 38637_at | up | 0.075790154 | lysyl oxidase |
| 36114_r_at | up | 0.07582013 | troponin T1, skeletal, slow |
| 39017_at | down | 0.075913788 | LSM1 homolog, U6 small nuclear RNA associated (S. cerevisiae) |
| 40947_at | up | 0.075914155 | hypothetical protein FLJ12671 |
| 39782_at | down | 0.075915309 | nuclear DNA-binding protein |
| 1845_at | down | 0.075997345 | mitogen-activated protein kinase kinase 4 |
| 36145_at | up | 0.076021749 | fuse-binding protein-interacting repressor |
| 32958_at | down | 0.076024781 | G protein-coupled receptor, family C, group 5, member B |
| 41131_f_at | down | 0.076033961 | heterogeneous nuclear ribonucleoprotein H2 (H′) |
| 40516_at | down | 0.076092957 | aryl hydrocarbon receptor |
| 35161_at | up | 0.07609887 | likely ortholog of mouse ubiquitin conjugating enzyme 7 interactin |
| | | | protein 5 |
| 38656_s_at | down | 0.076198725 | hypothetical protein MGC5576 |
| 34869_at | up | 0.076217032 | LIM domain binding 3 |
| 40982_at | down | 0.076234385 | hypothetical protein FLJ10534 |
| 36364_at | up | 0.07623929 | 4-aminobutyrate aminotransferase |
| 35751_at | down | 0.076289997 | succinate dehydrogenase complex, subunit B, iron sulfur (Ip) |
| 34907_at | up | 0.076316825 | apoptosis-associated tyrosine kinase |
| 33111_at | up | 0.076325246 | natural killer cell receptor, immunoglobulin superfamily member |
| 1500_at | up | 0.076347224 | Wilms tumor 1 |
| 41155_at | down | 0.076354576 | catenin (cadherin-associated protein), alpha 1, 102 kDa |
| 39107_at | up | 0.076418762 | lanosterol synthase (2,3-oxidosqualene-lanosterol cyclase) |
| 41815_at | down | 0.076423878 | spectrin repeat containing, nuclear envelope 2 |
| 33382_at | down | 0.076464267 | N-acylsphingosine amidohydrolase (acid ceramidase)-like |
| 690_s_at | up | 0.076507955 | kinase insert domain receptor (a type III receptor tyrosine kinase) |
| 38699_at | up | 0.076517467 | tubulin, beta, 5 |
| 32315_at | down | 0.076580486 | ribosomal protein S24 |
| 32394_s_at | down | 0.076586658 | ribosomal protein L23 |
| 1323_at | down | 0.076619803 | ubiquitin B |
| 520_at | up | 0.076637949 | mitogen-activated protein kinase kinase kinase 12 |
| 31977_at | up | 0.076645346 | guanylate cyclase 2D, membrane (retina-specific) |
| 40874_at | down | 0.076645607 | endothelial differentiation-related factor 1 |
| 41474_at | down | 0.076668881 | kinesin heavy chain member 2 |
| 1508_at | up | 0.076750896 | integrin, alpha 9 |
| 41252_s_at | down | 0.07680594 | chorionic somatomammotropin hormone 2 |
| 35449_at | down | 0.076867169 | killer cell lectin-like receptor subfamily B, member 1 |
| 38478_at | up | 0.07688698 | splicing factor, arginine/serine-rich 8 (suppressor-of-white-apricot |
| | | | homolog, Drosophila) |
| 33188_at | up | 0.076919217 | peptidylprolyl isomerase (cyclophilin)-like 2 |
| 41753_at | up | 0.076936025 | actinin, alpha 4 |
| 35309_at | up | 0.076946588 | suppression of tumorigenicity 14 (colon carcinoma, matriptase, |
| | | | epithin) |
| 41586_at | up | 0.076953724 | fibroblast growth factor 18 |
| 33729_at | down | 0.076996832 | solute carrier family 25 (mitochondrial carrier, brain), member 14 |
| 37448_s_at | down | 0.07699865 | GNAS complex locus |
| 2084_s_at | up | 0.077059452 | ets variant gene 4 (E1A enhancer binding protein, E1AF) |
| 35118_at | up | 0.077061841 | lecithin-cholesterol acyltransferase |
| 40327_at | up | 0.077082465 | homeo box B13 |
| 38244_at | up | 0.077091148 | hypothetical protein FLJ10178 |
| 32853_at | down | 0.077112143 | translocase of outer mitochondrial membrane 70 homolog A |
| | | | (yeast) |
| 38726_at | up | 0.077113456 | dolichyl-phosphate mannosyltransferase polypeptide 2, regulatory |
| | | | subunit |
| 32219_at | down | 0.077119778 | tousled-like kinase 1 |
| 39555_at | down | 0.077128151 | inhibitor of growth family, member 1-like |
| 35518_at | up | 0.07719372 | protein phosphatase 1, regulatory (inhibitor) subunit 3A (glycogen |
| | | | and sarcoplasmic reticulum binding subunit, skeletal muscle) |
| 39494_at | up | 0.07720618 | |
| 37023_at | down | 0.077401057 | lymphocyte cytosolic protein 1 (L-plastin) |
| 41859_at | up | 0.077419258 | uronyl-2-sulfotransferase |
| 40543_at | up | 0.077431976 | achaete-scute complex-like 1 (Drosophila) |
| 1232_s_at | up | 0.077484241 | insulin-like growth factor binding protein 1 |
| 41199_s_at | down | 0.07749603 | splicing factor proline/glutamine rich (polypyrimidine tract binding |
| | | | protein associated) |
| 32300_s_at | up | 0.077509791 | tyrosine hydroxylase |
| 41546_at | up | 0.077594704 | cyclin-dependent kinase 6 |
| 40044_at | up | 0.077618336 | ELL gene (11-19 lysine-rich leukemia gene) |
| 34372_at | down | 0.077632667 | upstream regulatory element binding protein 1 |
| 32379_f_at | up | 0.07769327 | pyruvate kinase, muscle |
| 31475_at | up | 0.07772506 | tankyrase, TRF1-interacting ankyrin-related ADP-ribose |
| | | | polymerase |
| 35322_at | down | 0.077745028 | Kelch-like ECH-associated protein 1 |
| 33741_at | down | 0.077773426 | ATPase, H+ transporting, lysosomal 50/57 kDa, V1 subunit H |
| 36207_at | down | 0.077808208 | SEC14-like 1 (S. cerevisiae) |
| 39504_at | up | 0.077809572 | gap junction protein, alpha 12, 47 kDa |
| 35946_at | up | 0.077814129 | NEL-like 1 (chicken) |
| 39303_at | up | 0.077857901 | beta-transducin repeat containing |
| 34946_at | down | 0.077936804 | immunoglobulin superfamily, member 6 |
| 34628_at | up | 0.07797503 | TAF4b RNA polymerase II, TATA box binding protein (TBP)- |
| | | | associated factor, 105 kDa |
| 38045_at | up | 0.077978903 | catenin (cadherin-associated protein), delta 2 (neural plakophilin- |
| | | | related arm-repeat protein) |
| 38392_at | down | 0.077981436 | actin related protein 2/3 complex, subunit 5, 16 kDa |
| 41825_at | up | 0.077994663 | PTEN induced putative kinase 1 |
| 31333_at | up | 0.078003393 | tolloid-like 1 |
| 34686_at | up | 0.078059255 | adenylate cyclase 2 (brain) |
| 34269_at | down | 0.078146471 | erythroid differentiation-related factor 1 |
| 37725_at | down | 0.078156806 | protein phosphatase 1, catalytic subunit, gamma isoform |
| 1266_s_at | up | 0.078163951 | lymphocyte-specific protein tyrosine kinase |
| 1616_at | up | 0.078185303 | fibroblast growth factor 9 (glia-activating factor) |
| 37563_at | up | 0.078201931 | SLIT-ROBO Rho GTPase activating protein 2 |
| 39336_at | up | 0.078228671 | ADP-ribosylation factor 3 |
| 33698_at | up | 0.07823379 | KIAA1052 protein |
| 31408_at | up | 0.07830084 | retinal pigment epithelium-derived rhodopsin homolog |
| 38900_at | up | 0.078345513 | paired box gene 3 (Waardenburg syndrome 1) |
| 32983_at | up | 0.078360832 | adrenergic, alpha-1B-, receptor |
| 32252_at | up | 0.078369125 | transthyretin (prealbumin, amyloidosis type I) |
| 32675_at | down | 0.078370483 | bone marrow stromal cell antigen 1 |
| 1058_at | down | 0.07837205 | WAS protein family, member 3 |
| 34915_at | up | 0.078441436 | solute carrier family 8 (sodium/calcium exchanger), member 1 |
| 41506_at | down | 0.078473991 | mitogen-activated protein kinase-activated protein kinase 5 |
| 160028_s_at | up | 0.078607486 | ret proto-oncogene (multiple endocrine neoplasia and medullary |
| | | | thyroid carcinoma 1, Hirschsprung disease) |
| 1085_s_at | down | 0.07863644 | phospholipase C, gamma 2 (phosphatidylinositol-specific) |
| 36471_f_at | up | 0.078683795 | dystrobrevin, alpha |
| 34873_at | up | 0.078715546 | nebulette |
| 37045_at | down | 0.078754743 | sorting nexin 19 |
| 1682_s_at | up | 0.078774726 | ATP-binding cassette, sub-family B (MDR/TAP), member 1 |
| 557_s_at | up | 0.078820073 | somatostatin receptor 3 |
| 41853_at | down | 0.078881429 | phosphoribosyl pyrophosphate synthetase-associated protein 2 |
| 1207_at | up | 0.078910905 | cyclin-dependent kinase 6 |
| 36228_at | up | 0.078961242 | glycine-N-acyltransferase |
| 1776_at | up | 0.079007645 | Ras-related associated with diabetes |
| 39653_at | up | 0.079043266 | protection of telomeres 1 |
| 1652_at | up | 0.079084497 | pim-2 oncogene |
| 969_s_at | down | 0.079111129 | ubiquitin specific protease 9, X chromosome (fat facets-like |
| | | | Drosophila) |
| 33847_s_at | down | 0.079127006 | cyclin-dependent kinase inhibitor 1B (p27, Kip1) |
| 32073_at | down | 0.07922512 | KIAA0677 gene product |
| 31403_at | up | 0.079247982 | solute carrier family 18 (vesicular monoamine), member 1 |
| 32776_at | down | 0.079253268 | v-ral simian leukemia viral oncogene homolog B (ras related; GTP |
| | | | binding protein) |
| 40585_at | down | 0.079269329 | adenylate cyclase 7 |
| 36696_at | up | 0.079410568 | phosphatidylinositol glycan, class C, pseudogene 1 |
| 1921_at | up | 0.079426983 | |
| 40377_at | up | 0.079435641 | KIAA0682 gene product |
| 32916_at | down | 0.079476859 | protein tyrosine phosphatase, receptor type, E |
| 39444_at | down | 0.079505682 | splicing factor 3b, subunit 1, 155 kDa |
| 32160_at | down | 0.079628498 | seven in absentia homolog 1 (Drosophila) |
| 33929_at | up | 0.079641537 | glypican 1 |
| 379_at | down | 0.079657938 | ATP binding protein associated with cell differentiation |
| 31862_at | up | 0.07967263 | wingless-type MMTV integration site family, member 5A |
| 33027_at | up | 0.079685137 | |
| 36641_at | down | 0.079685686 | capping protein (actin filament) muscle Z-line, alpha 2 |
| 36703_at | up | 0.079773179 | chemokine (C—C motif) ligand 25 |
| 32447_at | up | 0.079824503 | nuclear receptor subfamily 5, group A, member 1 |
| 39449_at | up | 0.079834205 | S-phase kinase-associated protein 2 (p45) |
| 39057_at | up | 0.079838689 | kinesin 2 60/70 kDa |
| 32792_at | down | 0.07988668 | GCIP-interacting protein p29 |
| 31461_at | up | 0.079924048 | proteasome (prosome, macropain) 26S subunit, non-ATPase, 4, |
| | | | pseudogene |
| 38801_at | down | 0.079926213 | VAMP (vesicle-associated membrane protein)-associated protein |
| | | | A, 33 kDa |
| 1018_at | up | 0.079992699 | wingless-type MMTV integration site family, member 10B |
| 37646_at | down | 0.079999644 | polymerase (DNA directed), delta 3 |
| 40093_at | up | 0.080005061 | Lutheran blood group (Auberger b antigen included) |
| 41855_at | down | 0.080076262 | histone acetyltransferase 1 |
| 40116_at | up | 0.080078498 | phosphofructokinase, liver |
| 40483_at | up | 0.080102456 | transcriptional activator of the c-fos promoter |
| 37214_g_at | down | 0.080139379 | deoxyribonuclease I-like 1 |
| 1975_s_at | up | 0.080172198 | insulin-like growth factor 1 (somatomedin C) |
| 32653_at | up | 0.080182256 | bromodomain containing 8 |
| 33375_at | up | 0.080187989 | myosin VI |
| 37632_s_at | down | 0.080207214 | zuotin related factor 1 |
| 37353_g_at | down | 0.080208739 | nuclear antigen Sp100 |
| AFFX-BioB-5_at | up | 0.080316663 | |
| 34529_at | up | 0.080325324 | |
| 31459_i_at | up | 0.080332933 | immunoglobulin lambda locus |
| 35524_at | up | 0.080365829 | complement component 8, gamma polypeptide |
| 37144_at | up | 0.080399764 | protein inhibitor of activated STAT protein PIASy |
| 33312_at | down | 0.080410486 | crystallin, alpha A |
| 37278_at | up | 0.080412116 | tafazzin (cardiomyopathy, dilated 3A (X-linked); endocardial |
| | | | fibroelastosis 2; Barth syndrome) |
| 35973_at | down | 0.080418152 | huntingtin interacting protein 14 |
| 38469_at | up | 0.080430213 | transmembrane 4 superfamily member 3 |
| 35218_at | down | 0.080433054 | programmed cell death 10 |
| 38750_at | up | 0.080450803 | Notch homolog 3 (Drosophila) |
| 38396_at | up | 0.080454362 | |
| 37493_at | down | 0.080471928 | colony stimulating factor 2 receptor, beta, low-affinity (granulocyte |
| | | | macrophage) |
| 38024_at | up | 0.080480542 | rap2 interacting protein x |
| 34300_at | down | 0.080530264 | zinc finger protein 278 |
| 32367_at | down | 0.080576466 | signal-regulatory protein beta 1 |
| 32504_at | down | 0.080592308 | sorting nexin 27 |
| 1452_at | down | 0.080634481 | LIM domain only 4 |
| 33859_at | down | 0.080676216 | sin3-associated polypeptide, 18 kDa |
| 34462_at | up | 0.080685447 | sodium channel, nonvoltage-gated 1, delta |
| 34653_at | up | 0.080686415 | receptor-associated protein of the synapse, 43 kD |
| 33466_at | up | 0.080709789 | hypothetical gene supported by AF038182; BC009203 |
| 33880_at | up | 0.080756283 | fatty-acid-Coenzyme A ligase, long-chain 3 |
| 1504_s_at | up | 0.080866712 | fibroblast growth factor 12 |
| 41245_at | up | 0.080874178 | growth differentiation factor 10 |
| 1220_g_at | up | 0.080887 | interferon regulatory factor 2 |
| 38411_at | down | 0.080931281 | |
| 32017_at | up | 0.08094505 | par-6 partitioning defective 6 homolog beta (C. elegans) |
| 38581_at | down | 0.080999597 | guanine nucleotide binding protein (G protein), q polypeptide |
| 32615_at | down | 0.081013831 | aspartyl-tRNA synthetase |
| 35206_at | up | 0.081072588 | centrosomal protein 2 |
| 2083_at | up | 0.081109896 | cholecystokinin B receptor |
| 35183_at | up | 0.081139697 | ATP-binding cassette, sub-family A (ABC1), member 3 |
| 39337_at | down | 0.081179863 | H2A histone family, member Z |
| 32805_at | up | 0.081185775 | aido-keto reductase family 1, member C2 (dihydrodiol |
| | | | dehydrogenase 2; bile acid binding protein; 3-alpha hydroxysteroid |
| | | | dehydrogenase, type III) |
| 39287_at | up | 0.081237036 | |
| 35213_at | up | 0.081275089 | WW domain binding protein 4 (formin binding protein 21) |
| 32090_at | up | 0.081301613 | nicotinamide nucleotide adenylyltransferase 2 |
| 39661_s_at | up | 0.081331131 | solute carrier family 29 (nucleoside transporters), member 2 |
| 41163_at | down | 0.081429809 | integral type I protein |
| 37382_at | up | 0.081475975 | ribosomal protein S26 |
| 31932_f_at | down | 0.0814765 | basic transcription factor 3 |
| 31354_r_at | up | 0.081482365 | forkhead box E2 |
| 33771_at | up | 0.081500647 | T-cell activation leucine repeat-rich protein |
| 40994_at | up | 0.08156926 | G protein-coupled receptor kinase 5 |
| 41219_at | down | 0.081589471 | KIAA0570 gene product |
| 38349_at | down | 0.081612444 | itchy homolog E3 ubiquitin protein ligase (mouse) |
| 35767_at | down | 0.081661911 | GABA(A) receptor-associated protein-like 2 |
| 1894_f_at | up | 0.08166614 | |
| 41846_at | up | 0.081743099 | cone-rod homeobox |
| 38082_at | down | 0.081774309 | KIAA0650 protein |
| 39581_at | down | 0.081796001 | cystatin A (stefin A) |
| 38566_at | up | 0.081805999 | collagen, type X, alpha 1(Schmid metaphyseal chondrodysplasia) |
| 35378_at | up | 0.081840606 | luteinizing hormone beta polypeptide |
| 39788_at | down | 0.081846683 | plakophilin 4 |
| 40358_at | up | 0.081926303 | GLI-Kruppel family member GLI3 (Greig cephalopolysyndactyly |
| | | | syndrome) |
| 35980_at | up | 0.081980276 | phospholipase C, beta 1 (phosphoinositide-specific) |
| 1790_s_at | up | 0.081983581 | |
| 35916_s_at | down | 0.082014903 | heterogeneous nuclear ribonucleoprotein A3 |
| 39611_at | up | 0.082030651 | |
| 32876_s_at | up | 0.082062451 | rhodopsin (opsin 2, rod pigment) (retinitis pigmentosa 4, |
| | | | autosomal dominant) |
| 35754_at | down | 0.082097137 | transmembrane trafficking protein |
| 32574_at | up | 0.082104017 | sphingomyelin phosphodiesterase 1, acid lysosomal (acid |
| | | | sphingomyelinase) |
| 41565_at | up | 0.08210775 | ataxin 2 related protein |
| 31340_at | up | 0.082177875 | matrix metalloproteinase 20 (enamelysin) |
| 219_i_at | up | 0.08227682 | microtubule-associated protein 2 |
| 32968_s_at | up | 0.082355877 | seizure related 6 homolog (mouse)-like |
| 41212_r_at | down | 0.082413942 | Williams-Beuren syndrome chromosome region 1 |
| 38824_at | down | 0.082607893 | HIV-1 Tat interactive protein 2, 30 kDa |
| 38134_at | down | 0.082648743 | pleiomorphic adenoma gene 1 |
| 1128_s_at | up | 0.082677345 | chemokine (C—C motif) receptor 1 |
| 31393_r_at | up | 0.082784321 | undifferentiated embryonic cell transcription factor 1 |
| 1521_at | down | 0.08282553 | non-metastatic cells 1, protein (NM23A) expressed in |
| 37523_at | up | 0.082918602 | acyl-Coenzyme A dehydrogenase, long chain |
| 32244_at | down | 0.082923664 | chromosome 14 open reading frame 92 |
| 38845_at | up | 0.082976532 | pyruvate dehydrogenase kinase, isoenzyme 2 |
| 39759_at | down | 0.082990311 | quaking homolog, KH domain RNA binding (mouse) |
| 1742_at | up | 0.082991136 | v-erb-b2 erythroblastic leukemia viral oncogene homolog 3 (avian) |
| 39817_s_at | up | 0.083028753 | putative c-Myc-responsive |
| 36510_at | down | 0.083073712 | general transcription factor IIF, polypeptide 2, 30 kDa |
| 38087_s_at | down | 0.083134517 | S100 calcium binding protein A4 (calcium protein, calvasculin, |
| | | | metastasin, murine placental homolog) |
| 188_at | up | 0.083158625 | ephrin-B1 |
| 32400_at | up | 0.083178012 | myeloid/lymphoid or mixed-lineage leukemia (trithorax homolog, |
| | | | Drosophila); translocated to, 1 |
| 36753_at | down | 0.083213097 | leukocyte immunoglobulin-like receptor, subfamily B (with TM and |
| | | | ITIM domains), member 4 |
| 37635_at | up | 0.083222309 | trichohyalin |
| 39268_at | down | 0.083225995 | potassium voltage-gated channel, subfamily F, member 1 |
| 40586_at | up | 0.08323434 | eukaryotic translation elongation factor 1 epsilon 1 |
| 39452_s_at | up | 0.083253328 | spectrin, beta, non-erythrocytic 1 |
| 31563_at | up | 0.083296186 | |
| 37681_i_at | up | 0.083326759 | matrin 3 |
| 37426_at | up | 0.083400663 | trinucleotide repeat containing 9 |
| 40286_r_at | up | 0.08342016 | |
| 39524_at | up | 0.08346012 | rhodopsin (opsin 2, rod pigment) (retinitis pigmentosa 4, |
| | | | autosomal dominant) |
| 1668_s_at | down | 0.08347108 | von Hippel-Lindau syndrome |
| 835_at | up | 0.083523429 | PDGFA associated protein 1 |
| 37661_at | down | 0.083561216 | ATPase, Ca++ transporting, plasma membrane 1 |
| 35702_at | up | 0.083576593 | hydroxysteroid (11-beta) dehydrogenase 1 |
| 32460_at | up | 0.083625268 | gamma-aminobutyric acid (GABA) A receptor, beta 2 |
| 40732_at | down | 0.083646527 | nuclear protein, ataxia-telangiectasia locus |
| 32276_at | down | 0.083660424 | ribosomal protein L32 |
| 35652_g_at | down | 0.083671345 | mitogen-activated protein kinase kinase kinase 4 |
| 41429_at | up | 0.083680874 | protein phosphatase 2 (formerly 2A), regulatory subunit A (PR 65) |
| | | | beta isoform |
| 33994_g_at | up | 0.083688844 | myosin, light polypeptide 6, alkali, smooth muscle and non-muscle |
| 38284_at | up | 0.083709948 | myeloid/lymphoid or mixed-lineage leukemia 4 |
| 40592_at | up | 0.08373631 | iduronate 2-sulfatase (Hunter syndrome) |
| 39519_at | down | 0.083740037 | KIAA0692 protein |
| 37871_at | up | 0.083797679 | islet amyloid polypeptide |
| 38635_at | down | 0.083816871 | signal sequence receptor, delta (translocon-associated protein |
| | | | delta) |
| 40246_at | down | 0.083844581 | discs, large (Drosophila) homolog 1 |
| 33948_at | up | 0.083864694 | corticotropin releasing hormone receptor 2 |
| 36087_at | up | 0.083865581 | KIAA0409 protein |
| 515_s_at | up | 0.083916267 | Cas-Br-M (murine) ecotropic retroviral transforming sequence b |
| 38948_at | up | 0.083927389 | protein phosphatase 1, regulatory subunit 3D |
| 32500_at | up | 0.083952172 | |
| 1450_g_at | down | 0.083974175 | proteasome (prosome, macropain) subunit, alpha type, 4 |
| 34469_at | up | 0.084001125 | ABO blood group (transferase A, alpha 1-3-N- |
| | | | acetylgalactosaminyltransferase; transferase B, alpha 1-3- |
| | | | galactosyltransferase) |
| 39010_at | down | 0.084111642 | endosulfine alpha |
| 38409_at | down | 0.084142918 | sperm specific antigen 2 |
| 33237_at | down | 0.084175575 | RNA helicase |
| 31502_at | up | 0.084292511 | annexin A2 |
| 559_s_at | up | 0.084353486 | T-cell leukemia, homeobox 1 |
| 34760_at | down | 0.08437959 | C-type lectin BIMLEC precursor |
| 1464_at | up | 0.084410452 | |
| 38295_at | down | 0.084421315 | pre-B-cell leukemia transcription factor 2 |
| 40318_at | up | 0.084546986 | dynein, cytoplasmic, intermediate polypeptide 1 |
| 38350_f_at | down | 0.084601034 | tubulin, alpha 2 |
| 41870_at | up | 0.084650654 | lung type-I cell membrane-associated glycoprotein |
| 32058_at | up | 0.084659847 | carbohydrate sulfotransferase 10 |
| 36994_at | up | 0.084686096 | ATPase, H+ transporting, lysosomal 16 kDa, V0 subunit c |
| 38681_at | down | 0.084759916 | eukaryotic translation initiation factor 3, subunit 6 48 kDa |
| 1069_at | down | 0.08476962 | prostaglandin-endoperoxide synthase 2 (prostaglandin G/H |
| | | | synthase and cyclooxygenase) |
| 40407_at | down | 0.084769798 | karyopherin alpha 2 (RAG cohort 1, importin alpha 1) |
| 36986_at | up | 0.084799441 | lysophospholipase II |
| 36102_at | down | 0.084832731 | voltage-dependent anion channel 3 |
| 38972_at | down | 0.08489681 | hypothetical protein BC013764 |
| 1854_at | up | 0.084942696 | v-myb myeloblastosis viral oncogene homolog (avian)-like 2 |
| 32152_at | up | 0.084966808 | ankyrin 1, erythrocytic |
| 31968_at | up | 0.084975489 | |
| 39673_i_at | up | 0.084986743 | extracellular matrix protein 2, female organ and adipocyte specific |
| 1441_s_at | down | 0.085018907 | tumor necrosis factor receptor superfamily, member 6 |
| 38619_at | up | 0.085083592 | golgl SNAP receptor complex member 2 |
| 37138_at | up | 0.085132288 | KIAA0809 protein |
| 39793_at | down | 0.085149958 | glioblastoma amplified sequence |
| 1060_g_at | up | 0.085200706 | neurotrophic tyrosine kinase, receptor, type 3 |
| 36647_at | down | 0.085228019 | hypothetical protein FLJ10326 |
| 40155_at | down | 0.085237267 | actin binding LIM protein 1 |
| 36538_at | up | 0.085374834 | protein phosphatase 1, regulatory (inhibitor) subunit 13B |
| 41405_at | up | 0.08539692 | secreted frizzled-related protein 4 |
| 33842_at | up | 0.085425991 | hypothetical protein FLJ11560 |
| 644_at | up | 0.085440532 | ras homolog gene family, member N |
| 1615_at | up | 0.085579584 | BCL2-like 1 |
| 40102_at | down | 0.08561703 | oxysterol binding protein-like 2 |
| 40824_at | down | 0.085638027 | exportin 7 |
| 37820_at | up | 0.085679373 | proline dehydrogenase (oxidase) 2 |
| 36310_at | up | 0.085731979 | keratin, hair, acidic, 1 |
| 1935_at | up | 0.085805182 | Mdm4, transformed 3T3 cell double minute 4, p53 binding protein |
| | | | (mouse) |
| 40218_at | up | 0.085816577 | CDP-diacylglycerol synthase (phosphatidate cytidylyltransferase) 1 |
| 33201_at | up | 0.085854381 | lethal giant larvae homolog 1 (Drosophila) |
| 34439_at | up | 0.085866067 | absent in melanoma 2 |
| 40210_at | down | 0.086009164 | RAB13, member RAS oncogene family |
| 41768_at | down | 0.086063925 | protein kinase, cAMP-dependent, regulatory, type I, alpha (tissue |
| | | | specific extinguisher 1) |
| 33089_s_at | down | 0.08610984 | ephrin-A2 |
| 33914_r_at | up | 0.086112896 | ferrochelatase (protoporphyria) |
| 35512_at | up | 0.086129344 | |
| 37951_at | up | 0.086142772 | deleted in liver cancer 1 |
| 40033_at | up | 0.086265844 | mitogen-activated protein kinase 11 |
| 40801_at | down | 0.08636791 | DKFZP434C212 protein |
| 41662_at | down | 0.086379918 | DKFZP566B183 protein |
| 40437_at | down | 0.086444658 | DKFZP564G2022 protein |
| 34326_at | down | 0.08654165 | coatomer protein complex, subunit beta |
| 38924_s_at | down | 0.086590576 | spectrin SH3 domain binding protein 1 |
| 39721_s_at | up | 0.086598108 | ephrin-B1 |
| 36283_at | up | 0.086609599 | DEAD/H (Asp-Glu-Ala-Asp/His) box polypeptide 6 (RNA helicase |
| | | | 54 kDa) |
| 41125_r_at | up | 0.086618972 | ectonucleotide pyrophosphatase/phosphodiesterase 2 (autotaxin) |
| 38985_at | down | 0.086620716 | leptin receptor overlapping transcript-like 1 |
| 33705_at | down | 0.086752173 | phosphodiesterase 4B, cAMP-specific (phosphodiesterase E4 |
| | | | dunce homolog, Drosophila) |
| 33378_at | down | 0.086759624 | IDN3 protein |
| 37706_at | up | 0.086771534 | golgi apparatus protein 1 |
| 40126_at | up | 0.086915064 | paired mesoderm homeo box 1 |
| 38040_at | down | 0.086916527 | splicing factor 30, survival of motor neuron-related |
| 1711_at | up | 0.086923062 | tumor protein p53 binding protein, 1 |
| 1317_at | up | 0.086948161 | macrophage stimulating 1 receptor (c-met-related tyrosine kinase) |
| 38403_at | down | 0.086971119 | lysosomal-associated membrane protein 2 |
| 33657_at | down | 0.086989849 | ribosomal protein L34 |
| 36537_at | down | 0.087068338 | Rho-specific guanine nucleotide exchange factor p114 |
| 37027_at | down | 0.087167604 | AHNAK nucleoprotein (desmoyokin) |
| 1149_at | down | 0.087172748 | |
| 34725_at | up | 0.087190996 | glucocorticoid receptor DNA binding factor 1 |
| 38035_at | down | 0.087230646 | myotubularin related protein 6 |
| 1489_s_at | up | 0.087275505 | v-myc myelocytomatosis viral oncogene homolog 1, lung |
| | | | carcinoma derived (avian) |
| 340_at | up | 0.087320254 | matrilin 3 |
| 521_at | up | 0.087361038 | metallothionein IV |
| 39717_g_at | down | 0.087391993 | mitochondrial ribosomal protein L33 |
| 40633_at | up | 0.087439293 | mitotic control protein dis3 homolog |
| 36165_at | down | 0.087457854 | cytochrome c oxidase subunit VIc |
| 31735_at | up | 0.087477585 | type 1 protein phosphatase inhibitor |
| 31417_at | up | 0.087483514 | hypocretin (orexin) neuropeptide precursor |
| 38062_at | up | 0.087507618 | guanine nucleotide exchange factor for Rap1 |
| 38106_at | down | 0.087527635 | TGF beta-inducible nuclear protein 1 |
| 34272_at | up | 0.087592461 | regulator of G-protein signalling 4 |
| 34225_at | up | 0.087596552 | Wolf-Hirschhorn syndrome candidate 2 |
| 41712_at | down | 0.087689972 | likely ortholog of mouse aquarius |
| 378_s_at | up | 0.087796914 | GPI anchored molecule like protein |
| 31981_at | up | 0.087831344 | crystallin, beta B3 |
| 899_at | up | 0.087862483 | Indian hedgehog homolog (Drosophila) |
| 40933_f_at | up | 0.087878586 | zinc finger, DHHC domain containing 18 |
| 32590_at | down | 0.087886008 | nucleolin |
| 1397_at | down | 0.088027982 | mitogen-activated protein kinase kinase kinase 11 |
| 38821_at | down | 0.088036116 | progesterone receptor membrane component 2 |
| 41185_f_at | down | 0.088044825 | SMT3 suppressor of mif two 3 homolog 2 (yeast) |
| 40384_at | up | 0.088045667 | nephronophthisis 1 (juvenile) |
| 39019_at | down | 0.088056769 | lysosomal-associated protein transmembrane 4 alpha |
| 676_g_at | up | 0.088222263 | interferon induced transmembrane protein 1 (9-27) |
| 41389_s_at | up | 0.088364655 | renal tumor antigen |
| 239_at | down | 0.08844228 | cathepsin D (lysosomal aspartyl protease) |
| 1842_at | down | 0.088450446 | |
| 34134_at | up | 0.088462035 | |
| 36885_at | down | 0.08846417 | spleen tyrosine kinase |
| 39489_g_at | up | 0.088500732 | protocadherin 9 |
| 35857_at | up | 0.088519565 | glutamate receptor, ionotropic, kainate 1 |
| 37639_at | up | 0.088520319 | hepsin (transmembrane protease, serine 1) |
| AFFX- | up | 0.088539866 | transferrin receptor (p90, CD71) |
| HUMTFRR/M11507_5_at | | | |
| 35367_at | down | 0.088573711 | lectin, galactoside-binding, soluble, 3 (galectin 3) |
| 35957_at | down | 0.088641618 | stannin |
| 31458_at | up | 0.088655659 | cytokeratin 2 |
| 33192_g_at | down | 0.08877997 | chromosome 10 open reading frame 6 |
| 36523_at | down | 0.0887804 | ATPase, Cu++ transporting, alpha polypeptide (Menkes |
| | | | syndrome) |
| 32658_at | up | 0.08879375 | SAC2 suppressor of actin mutations 2-like (yeast) |
| 36547_r_at | up | 0.088847765 | KIAA0542 gene product |
| 32866_at | up | 0.08900188 | KIAA0605 gene product |
| 36336_s_at | up | 0.089108577 | KIAA0963 protein |
| 38031_at | down | 0.089210079 | KIAA0111 gene product |
| 32771_at | up | 0.089236711 | group-specific component (vitamin D binding protein) |
| 33368_at | down | 0.089256532 | protease, serine, 15 |
| 34441_at | up | 0.089294655 | |
| 38258_at | up | 0.089315852 | |
| 34143_at | up | 0.089329234 | cholinergic receptor, nicotinic, alpha polypeptide 1 (muscle) |
| 39199_at | up | 0.08937154 | |
| 37034_at | down | 0.089374662 | acidic (leucine-rich) nuclear phosphoprotein 32 family, member A |
| 578_at | up | 0.089382491 | recombination activating gene 2 |
| 35866_at | down | 0.089442342 | zinc finger protein 268 |
| 551_at | down | 0.089610262 | E1A binding protein p300 |
| 32003_at | up | 0.089725235 | methionine adenosyltransferase I, alpha |
| 1541_f_at | up | 0.089862891 | interferon, alpha 6 |
| 39466_s_at | up | 0.089866338 | |
| 324_f_at | down | 0.089873012 | |
| 41310_f_at | up | 0.089892054 | nuclear receptor subfamily 2, group F, member 6 |
| 41808_at | down | 0.090154156 | cyclin D binding myb-like transcription factor 1 |
| 33037_at | up | 0.090155577 | carbohydrate (N-acetylglucosamine 6-O) sulfotransferase 7 |
| 31466_at | up | 0.090205249 | major histocompatibility complex, class II, DR beta 6 |
| | | | (pseudogene) |
| 37323_r_at | up | 0.090262072 | hydroxyprostaglandin dehydrogenase 15-(NAD) |
| 2091_at | up | 0.090282876 | wingless-type MMTV integration site family, member 4 |
| 32477_at | up | 0.090315491 | calpain 9 (nCL-4) |
| 34832_s_at | up | 0.090482967 | KIAA0763 gene product |
| AFFX-PheX-5_at | up | 0.090493351 | |
| 39141_at | up | 0.090696236 | ATP-binding cassette, sub-family F (GCN20), member 1 |
| 38402_at | down | 0.090789867 | lysosomal-associated membrane protein 2 |
| 34109_at | up | 0.090803494 | proline dehydrogenase (oxidase) 1 |
| 40440_at | up | 0.090928291 | PAI-1 mRNA-binding protein |
| 34387_at | down | 0.091050579 | KIAA0205 gene product |
| 40639_at | up | 0.09105652 | SCO cytochrome oxidase deficient homolog 2 (yeast) |
| 33289_f_at | down | 0.091058 | zinc finger protein 263 |
| 41476_at | up | 0.091067637 | |
| 35661_g_at | up | 0.091173127 | S-antigen; retina and pineal gland (arrestin) |
| 39959_at | up | 0.091197187 | ubiquitin D |
| 35129_at | up | 0.091246944 | sperm adhesion molecule 1 (PH-20 hyaluronidase, zona pellucida |
| | | | binding) |
| 1879_at | down | 0.091260491 | related RAS viral (r-ras) oncogene homolog |
| 37734_at | down | 0.091272658 | disco-interacting protein 2 (Drosophila) homolog |
| 39680_at | up | 0.09128097 | statherin |
| 1300_at | up | 0.091302081 | X-ray repair complementing defective repair in Chinese hamster |
| | | | cells 2 |
| 35494_at | up | 0.091514972 | |
| AFFX-DapX-3_at | up | 0.091526914 | |
| 35159_at | up | 0.091541071 | tubulin-specific chaperone e |
| 1686_g_at | down | 0.091586384 | S-phase response (cyclin-related) |
| 38243_at | up | 0.091608235 | neural cell adhesion molecule 1 |
| 41772_at | up | 0.091636193 | monoamine oxidase A |
| 36468_at | up | 0.091669779 | dystrobrevin, alpha |
| 31912_at | up | 0.091687981 | forkhead box H1 |
| 1966_i_at | up | 0.091745212 | nitric oxide synthase 2C |
| 32392_s_at | up | 0.091772778 | UDP glycosyltransferase 1 family, polypeptide A4 |
| 40854_at | down | 0.091958912 | ubiquinol-cytochrome c reductase core protein II |
| 37326_at | down | 0.091968595 | proteolipid protein 2 (colonic epithelium-enriched) |
| 33835_at | up | 0.092020494 | KIAA0721 protein |
| 32666_at | up | 0.092098111 | chemokine (C—X—C motif) ligand 12 (stromal cell-derived factor 1) |
| 34101_at | up | 0.092129007 | |
| 33792_at | up | 0.092178872 | prostate stem cell antigen |
| 38008_at | up | 0.092208168 | DNA segment on chromosome 4 (unique) 234 expressed |
| | | | sequence |
| 33103_s_at | down | 0.092213362 | adducin 3 (gamma) |
| 1448_at | down | 0.092287738 | proteasome (prosome, macropain) subunit, alpha type, 3 |
| 36213_at | up | 0.092306281 | malignant fibrous histiocytoma amplified sequence 1 |
| 37677_at | down | 0.092335261 | phosphoglycerate kinase 1 |
| 40374_at | up | 0.092357025 | cardiac ankyrin repeat protein |
| 34264_at | up | 0.092400894 | RUN and SH3 domain containing 1 |
| 36925_at | up | 0.092424153 | heat shock 70 kDa protein 2 |
| 39003_at | down | 0.092431788 | pituitary tumor-transforming 1 interacting protein |
| 41795_at | down | 0.09245412 | NCK adaptor protein 1 |
| 31906_at | down | 0.092499322 | heat shock factor binding protein 1 |
| 34188_at | down | 0.092551592 | |
| 38019_at | up | 0.092684992 | casein kinase 1, epsilon |
| 38673_s_at | down | 0.092735744 | cyclin-dependent kinase inhibitor 1C (p57, Kip2) |
| 34814_at | down | 0.092753688 | SUMO-1 activating enzyme subunit 2 |
| 38212_at | up | 0.092799885 | UDP-N-acetyl-alpha-D-galactosamine: (N-acetylneuraminyl)- |
| | | | galactosylglucosylceramide N-acetylgalactosaminyltransferase |
| | | | (GalNAc-T) |
| 31379_at | up | 0.092814218 | HFSE-1 protein |
| 34578_at | up | 0.092823397 | sarcoglycan, gamma (35 kDa dystrophin-associated glycoprotein) |
| 41545_at | up | 0.092876791 | cyclin-dependent kinase 6 |
| 38037_at | up | 0.092905054 | diphtheria toxin receptor (heparin-binding epidermal growth factor- |
| | | | like growth factor) |
| 41788_i_at | down | 0.092989382 | KIAA0669 gene product |
| 32624_at | down | 0.093011872 | likely ortholog of mouse tuberin-like protein 1 |
| 34122_at | up | 0.093042118 | casein beta |
| 36553_at | down | 0.093052406 | acetylserotonin O-methyltransferase-like |
| 32679_at | up | 0.093083461 | likely ortholog of chicken chondrocyte protein with a poly-proline |
| | | | region |
| 37117_at | up | 0.093191269 | Rho GTPase activating protein 8 |
| 36077_at | down | 0.093212186 | RAB, member of RAS oncogene family-like 4 |
| 38854_at | up | 0.093242092 | KIAA0635 gene product |
| 34118_at | up | 0.093250389 | ATPase, Na+/K+ transporting, beta 2 polypeptide |
| 36414_s_at | down | 0.093282245 | calcium/calmodulin-dependent serine protein kinase (MAGUK |
| | | | family) |
| 32094_at | up | 0.093290429 | carbohydrate (chondroitin 6) sulfotransferase 3 |
| 41137_at | up | 0.093310928 | protein phosphatase 1, regulatory (inhibitor) subunit 12B |
| 41149_at | up | 0.093394734 | exonuclease NEF-sp |
| 35268_at | down | 0.093406671 | axotrophin |
| 33352_at | down | 0.093411658 | histone 2, H2be |
| 31463_s_at | down | 0.093412524 | |
| 38190_r_at | up | 0.093548584 | KIAA0645 gene product |
| 40864_at | down | 0.093610502 | ras-related C3 botulinum toxin substrate 1 (rho family, small GTP |
| | | | binding protein Rac1) |
| 40903_at | down | 0.093640616 | ATPase, H+ transporting, lysosomal interacting protein 2 |
| 31874_at | down | 0.093647357 | growth arrest-specific 2 like 1 |
| 33820_g_at | down | 0.093671525 | lactate dehydrogenase B |
| 35311_at | down | 0.093773374 | cellular repressor of E1A-stimulated genes |
| 36350_at | up | 0.093885805 | |
| 39663_at | down | 0.093917507 | mannosidase, alpha, class 2A, member 1 |
| 35149_at | up | 0.093949795 | tumor necrosis factor receptor superfamily, member 5 |
| 38451_at | down | 0.093966652 | ubiquinol-cytochrome c reductase (6.4 kD) subunit |
| 31330_at | down | 0.093994243 | ribosomal protein S19 |
| 34329_at | down | 0.094055721 | p21 (CDKN1A)-activated kinase 2 |
| 40919_at | up | 0.094079635 | somatostatin receptor 2 |
| 41215_s_at | down | 0.094096263 | inhibitor of DNA binding 2, dominant negative helix-loop-helix |
| | | | protein |
| 31901_at | up | 0.094118872 | potassium voltage-gated channel, shaker-related subfamily, beta |
| | | | member 2 |
| 1081_at | down | 0.094220232 | ornithine decarboxylase 1 |
| 41194_at | down | 0.094258686 | signal recognition particle 14 kDa (homologous Alu RNA binding |
| | | | protein) |
| 37098_at | up | 0.09434038 | protoporphyrinogen oxidase |
| 39056_at | down | 0.094461372 | phosphoribosylaminoimidazole carboxylase, |
| | | | phosphoribosylaminoimidazole succinocarboxamide synthetase |
| 37527_at | up | 0.094565974 | ELK3, ETS-domain protein (SRF accessory protein 2) |
| 35998_at | up | 0.094588743 | |
| 35328_at | up | 0.094596866 | |
| 31815_r_at | up | 0.094601451 | low density lipoprotein receptor-related protein 3 |
| 34282_at | down | 0.094675793 | nuclear factor (erythroid-derived 2)-like 3 |
| 36463_at | down | 0.094675835 | BCL2-associated athanogene 5 |
| 37679_at | down | 0.094863772 | interferon-related developmental regulator 1 |
| 34062_at | up | 0.094905599 | ets variant gene 2 |
| 40080_at | up | 0.094934258 | KIAA0090 protein |
| 41414_at | up | 0.094942383 | chromosome 22 open reading frame 3 |
| 40977_f_at | up | 0.095014531 | cerebellar degeneration-related protein 1, 34 kDa |
| 35897_r_at | up | 0.095044456 | brain-specific angiogenesis inhibitor 1 |
| 1428_at | down | 0.095078801 | |
| 37773_at | up | 0.095087394 | KIAA1005 protein |
| 40431_at | down | 0.095109067 | KIAA0431 protein |
| 36203_at | down | 0.095163871 | ornithine decarboxylase 1 |
| 444_g_at | up | 0.095292483 | homeo box D4 |
| 1244_at | up | 0.095328326 | signal transducer and activator of transcription 2, 113 kDa |
| 39995_s_at | up | 0.095391047 | WW domain containing oxidoreductase |
| 39178_at | down | 0.095462475 | reticulon 1 |
| 31706_at | up | 0.09547677 | |
| 40023_at | up | 0.095557348 | brain-derived neurotrophic factor |
| 34472_at | up | 0.095594647 | frizzled homolog 6 (Drosophila) |
| 38950_r_at | up | 0.095606306 | matrix metalloproteinase 23B |
| 1852_at | up | 0.095612496 | tumor necrosis factor (TNF superfamily, member 2) |
| 41174_at | up | 0.095643232 | RAN binding protein 2-like 1 |
| 34761_r_at | down | 0.095664289 | a disintegrin and metalloproteinase domain 9 (meltrin gamma) |
| 40449_at | up | 0.095715432 | replication factor C (activator 1) 1, 145 kDa |
| 35826_at | up | 0.095727306 | suppressor of Ty 5 homolog (S. cerevisiae) |
| 700_s_at | up | 0.09583438 | |
| 582_g_at | down | 0.095875255 | nuclear receptor subfamily 2, group C, member 1 |
| 39244_at | down | 0.095915812 | RAB4A, member RAS oncogene family |
| 32899_s_at | up | 0.095919044 | RAR-related orphan receptor A |
| 32296_at | up | 0.095927535 | carbonic anhydrase VA, mitochondrial |
| 36679_at | down | 0.095972007 | signal recognition particle receptor (‘docking protein’) |
| 31885_at | up | 0.095999682 | protein tyrosine phosphatase, non-receptor type 3 |
| 368_at | up | 0.096090928 | trophoblast glycoprotein |
| 36313_at | down | 0.096111046 | ecotropic viral integration site 2A |
| 41367_at | up | 0.09615194 | hepatocyte nuclear factor 4, gamma |
| 34610_at | down | 0.096189258 | guanine nucleotide binding protein (G protein), beta polypeptide 2 |
| | | | like 1 |
| 41093_at | up | 0.096218086 | opioid binding protein/cell adhesion molecule-like |
| 38093_at | down | 0.096246266 | chromosome 14 open reading frame 32 |
| 35345_at | up | 0.096248258 | 3-hydroxy-3-methylglutaryl-Coenzyme A synthase 2 |
| | | | (mitochondrial) |
| 40976_at | up | 0.09628133 | katanin p80 (WD repeat containing) subunit B 1 |
| 32295_at | up | 0.096300607 | HLA complex group 9 |
| 1636_g_at | up | 0.096306453 | v-abl Abelson murine leukemia viral oncogene homolog 1 |
| 35595_at | up | 0.096330447 | calcitonin gene-related peptide-receptor component protein |
| 40533_at | up | 0.096342646 | baculoviral IAP repeat-containing 5 (survivin) |
| 1261_i_at | up | 0.096407666 | glutathione S-transferase A2 |
| 32567_at | up | 0.096421885 | choline kinase |
| 36100_at | up | 0.096473819 | vascular endothelial growth factor |
| 40256_at | up | 0.09651525 | |
| 37747_at | down | 0.096606126 | annexin A5 |
| 37764_at | up | 0.096642303 | holocarboxylase synthetase (biotin-[proprionyl-Coenzyme A- |
| | | | carboxylase (ATP-hydrolysing)] ligase) |
| 34613_at | up | 0.096690958 | KIAA1086 protein |
| 615_s_at | up | 0.09674634 | parathyroid hormone-like hormone |
| 31827_s_at | up | 0.096759998 | trans-golgi network protein 2 |
| 32011_g_at | up | 0.096816474 | hypothetical protein EAN57 |
| 33086_at | up | 0.096895282 | prophet of Pit1, paired-like homeodomain transcription factor |
| 34942_at | up | 0.097018589 | hypothetical protein FLJ38993 |
| 39578_at | up | 0.097121586 | hairless |
| 41460_at | down | 0.097154944 | RNA binding motif protein 14 |
| 31713_s_at | up | 0.097197158 | discs, large (Drosophila) homolog-associated protein 2 |
| 32277_at | up | 0.097229418 | potassium voltage-gated channel, shaker-related subfamily, |
| | | | member 3 |
| 462_at | up | 0.097234978 | nuclear factor I/B |
| 35187_at | down | 0.097264305 | |
| 38033_at | up | 0.097281795 | DKFZP564M1416 protein |
| 464_s_at | down | 0.097305326 | interferon-induced protein 35 |
| 41120_at | up | 0.097408034 | aminomethyltransferase (glycine cleavage system protein T) |
| 40381_at | up | 0.097503532 | KIAA0972 protein |
| 41587_g_at | up | 0.097533335 | fibroblast growth factor 18 |
| 39825_at | up | 0.097585237 | solute carrier family 25 (mitochondrial carrier; citrate transporter), |
| | | | member 1 |
| 1822_at | up | 0.097654682 | |
| 32178_r_at | down | 0.097713503 | synaptosomal-associated protein, 23 kDa |
| 1447_at | down | 0.097772909 | proteasome (prosome, macropain) subunit, beta type, 1 |
| 33930_at | down | 0.097850141 | chromosome 14 open reading frame 163 |
| 36353_at | up | 0.097867455 | hairy and enhancer of split (Drosophila) homolog 2 |
| 35093_at | up | 0.097907561 | purinergic receptor P2X-like 1, orphan receptor |
| 1357_at | down | 0.09799221 | ubiquitin specific protease 4 (proto-oncogene) |
| 37280_at | up | 0.098054947 | MAD, mothers against decapentaplegic homolog 1 (Drosophila) |
| 1907_at | up | 0.098069395 | retinoblastoma-like 1 (p107) |
| 33141_at | up | 0.098156986 | hydroxysteroid (17-beta) dehydrogenase 1 |
| 39686_g_at | down | 0.098160911 | like mouse brain protein E46 |
| 37673_at | down | 0.09818696 | neutral sphingomyelinase (N-SMase) activation associated factor |
| 38110_at | down | 0.098232139 | syndecan binding protein (syntenin) |
| 34879_at | down | 0.098241379 | dolichyl-phosphate mannosyltransferase polypeptide 1, catalytic |
| | | | subunit |
| 38609_at | up | 0.098269966 | sarcoglycan, alpha (50 kDa dystrophin-associated glycoprotein) |
| 37492_at | up | 0.098274979 | KIAA0500 protein |
| 34100_at | up | 0.098277118 | |
| 37996_s_at | up | 0.098280565 | dystrophia myotonica-protein kinase |
| 35824_at | down | 0.098305072 | zinc finger protein 238 |
| 40086_at | down | 0.098345702 | friend of EBNA2 |
| 32083_at | down | 0.098363125 | transmembrane 7 superfamily member 1 (upregulated in kidney) |
| 34977_at | down | 0.09838151 | sialic acid binding lg-like lectin 7 |
| 33161_at | down | 0.098418566 | hypothetical protein MGC15523 |
| 37861_at | up | 0.098434036 | CD1E antigen, e polypeptide |
| 38784_g_at | up | 0.098470912 | mucin 1, transmembrane |
| 38020_at | down | 0.098546526 | KIAA0652 gene product |
| 808_at | up | 0.098588848 | RAB27B, member RAS oncogene family |
| 1695_at | down | 0.098764335 | neural precursor cell expressed, developmentally down-regulated 8 |
| 36130_f_at | up | 0.098794417 | metallothionein 1E (functional) |
| 31612_at | up | 0.098800032 | secretoglobin, family 1D, member 1 |
| 37900_at | down | 0.099027962 | peroxisomal biogenesis factor 11B |
| 33809_at | up | 0.099045406 | guanine nucleotide binding protein (G protein), alpha inhibiting |
| | | | activity polypeptide 1 |
| 38722_at | up | 0.099099373 | collagen, type VI, alpha 1 |
| 36522_at | up | 0.099155422 | mucoepidermoid carcinoma translocated 1 |
| 2051_at | up | 0.099167839 | O-6-methylguanine-DNA methyltransferase |
| 38907_at | up | 0.099379332 | |
| 40860_s_at | up | 0.099381562 | nuclear protein UKp68 |
| 40880_r_at | up | 0.099383953 | chloride channel 3 |
| 41370_at | down | 0.099417017 | U5 snRNP-specific 40 kDa protein (hPrp8-binding) |
| 36968_s_at | down | 0.099572253 | Opa-interacting protein 2 |
| 32325_at | up | 0.099581291 | |
| 33524_at | up | 0.099592485 | v-crk sarcoma virus CT10 oncogene homolog (avian)-like |
| 33388_at | up | 0.099596344 | testis expressed gene 261 |
| 35321_at | down | 0.099608404 | tousled-like kinase 2 |
| 36946_at | down | 0.099667092 | dual-specificity tyrosine-(Y)-phosphorylation regulated kinase 1A |
| 1348_s_at | down | 0.099835885 | proplonyl Coenzyme A carboxylase, alpha polypeptide |
| 41737_at | down | 0.099885912 | serine/arginine repetitive matrix 1 |
| 39558_s_at | up | 0.099902424 | |
| 39474_s_at | up | 0.099944447 | cysteine knot superfamily 1, BMP antagonist 1 |
| 38825_at | up | 0.099970407 | fibrinogen, A alpha polypeptide |
| 38164_at | down | 0.099971672 | retinitis pigmentosa GTPase regulator |

### Example 3

### Prostate Tumor Diagnosis Through Bloodcell Multigene Signatures

### Introduction

Prostate cancer is the second largest cancer killer of men in the Unites States and Europe. It has been estimated that in 2000, in the U.S., 180,400 men were diagnosed with prostate cancer and approximately 32,000 died in that year alone (Greenlee et al., CA Cancer J Clin. 2000; 50(1):7-33). Current techniques for the screening and risk assessment of prostate cancer, as a prerequisite to surgical biopsy procedures, are based upon the measurement of either individual serum biomarkers, or expression of individual genes in circulating malignant cells (Oesterling et al., JAMA 1993; 270(7):860-4; Seaman et al., Urol Clin North Am.; 1993; 20(4):653-63; and Catalona et al., Urology 2000; 56(2):255). These techniques, which include RT-PCR, possess a number of limitations, including lack of specificity and accuracy in the diagnosis and, also a lack of prognostic information. This ultimately yields high numbers of false positive diagnoses, and consequently unnecessarily large numbers of surgical biopsies.

Since its clinical approval by the FDA in 1986, prostate specific antigen (PSA) has been recognized as the most useful biomarker for diagnosis and surveillance of prostate cancer. In current clinical practice, a serum PSA assay (which quantifies levels of PSA protein as ng/ml in sera prepared from peripheral blood), in conjunction with digital rectal examination (DRE), is used to indicate which patients should undergo prostate biopsy. Prostate cancer statistics have clearly shown that pre-screening prior to biopsy has led to both an increase in the number of men diagnosed with cancer and a decline in age of diagnosis due to earlier detection (Roberts et al., Urology. 2000; 56(5):817-22). It was estimated that during 1997, over 750,000 prostate biopsies were performed following PSA and DRE screening (ref). However, although for example PSA is an effective indicator of prostate cancer when serum levels are high, diagnostic assays based on this marker become more ambiguous when levels are only moderately elevated, i.e. between 2-10 ng/ml. Abnormal findings from DRE have also been attributed to various benign conditions, thus contributing to this low accuracy of cancer detection rates prior to biopsy (Roberts et al., Urology. 2000; 56(5):817-22). A false-positive pre-biopsy diagnosis of cancer has been reported in 40-6% of men with both abnormal DRE and PSA levels greater than 4 ng/ml, resulting in a high percent of unnecessary prostate biopsies (Bangma et al., J Urol. 1997:157(6):2191-6; Smith et al., Cancer. 1997; 80(9):1852-6; Roberts et al., Urology. 2000; 56(5):817-22).

Although methods have been investigated to increase specificity of PSA, such as the use of free/total PSA measurements (Catalona et al., Urology 2000; 56(2):255), a recent review of screening programs has suggested that even though the use of free/total PSA serum measurements may reduce the need for biopsy by up to 40%, approximately 12% of all tumors would still be missed with this assay (Neal et al., Eur J Cancer 2000; 36(10):1316-21). In addition, serum quantitation of biomarkers, such as PSA, does not reliably correlate with important cancer variables such as biological aggressiveness, does not allow outcome predictions in patients with hormone refractory disease (Murphy et al et al., Cancer. 1998; 83(11):2259-69), and does not identify the presence of soft tissue metastasis (Beckett et al., Clin Cancer Res. 1999; 5(12):4034-40). For example, in over half of patients with metastatic disease, diagnosis was made only after radical surgery for the localized tumor (Olsson C A. Urol Clin North Am. 1997; 24(2):367-78). Current research has implied that important diagnostic and prognostic information will be derived only after surgical procedures, where biopsy or radical prostatectomy specimens are surgically removed, and tumor pathology is classified by histological grade and Gleason score. Unfortunately, these invasive methods also have some limitations, such as the need for a highly trained pathologist to interpret the degree of tumor pathology and the histological grade of clinical specimens and the requirement for repeat biopsies in some patients because of missed tumors (Lee et al., Prostate 1999; 39(3):213-8; Noguchi et al. Int J Urol 1999; 6:7-12), and one study reported that up to 26% of men with an initial non-cancer diagnosis were reported positive for prostate cancer on a repeated biopsy performed within one year (O'Dowd et al., Urology. 2000; 55(4):553-9). Patient discomfort and stress is also very high in diagnostic tests based on surgical tissue removal.

There is growing evidence that individuals with prostate cancer and other forms of malignant disease exhibit immune responses that can be detected at the level of altered gene expression in leukocytes circulating in peripheral blood. Furthermore, the use of microarray technology allows the simultaneous measurement of the expression levels of up to 14,000 genes transcribed in circulating leukocytes derived from the blood of prostate cancer patients and control individuals. This technology described in this invention demonstrates that individuals suffering from prostate cancer exhibit a conserved pattern, or signature, of gene expression levels in their peripheral blood leukocytes, which is distinct from the corresponding pattern of expression in leukocytes from control subjects. In addition, cpatients with prostate tumors at different histological grades may yield distinct expression signatures that reflect the biological stage and aggressiveness of the tumor, and that information can thus be employed to differentiate among tumors at different pathological stages.

This Example demonstrates a novel technique that does not require invasive surgery, yet provides an accurate diagnosis of prostate cancer, and may also provides detailed prognostic information on the stage and biological aggressiveness of the tumor. Investigators have begun to employ microarray technology, based upon sample cDNA probe hybridization to DNA microarrays, to identify and isolate genes differentially expressed in prostate tumor tissues and prostate cancer cell lines. Recent studies have identified genes that may be involved in hormone refractory prostate cancer (Amler et al., Cancer Res. 2000; 60(21):6134-41), and genes that are potential targets for prostate cancer therapy. Many others have applied microarray technology to investigate the LNCaP tumor model cell line series, which re-capitulates some of the major biological stages of prostate tumor progression. These studies have identified genes thought to play a role in the progression of prostate cancer from androgen and bone cell-growth dependence to autonomous metastatic ability (Clelland et al., Am. J. Hum. Genet. 2000; 67:(4) 8).

An alternative approach to these experiments has been the development of prostate tissue microarrays, where sectioned tumor tissue is arrayed and immobilized onto the microarray surfaces. Tissue arrays allow more detailed analysis of gene expression within individual prostate tumor cells and has been used to determine and compare profiles of gene expression between tissues of men from ethnic populations that have both low and high risk for developing cancer. Cole et al. (Nat. Genet. 1999: 21(1 Suppl):3841) have proposed the use of tissue microarrays to determine a combined, detailed histological and gene expression 3D reconstruction of the anatomy of normal and prostate malignant tissues, which may ultimately provide vital information in the cellular progression of the disease.

Two new studies have been published that are likely to be of great clinical significance for the management of prostate cancer. In one investigation, published in Nature, Dhanasekaran et al., employed normal and neoplastic prostate specimens and cDNA microarrays to analyze and identify gene expression patterns of normal and tumor tissue (Dhanasekaran et al., infra). This study was the first to report specific expression signatures that could distinguish prostate tissue, including normal prostate (adjacent to the tumor site), BPH, localized prostate cancer and metastatic, hormone refractory disease. More recently, a group has employed Affymetrix GeneChip microarrays to analyze prostate tumor specimens and compare gene expression levels among samples of known stages of prostate cancer (Luo et al., Mol Carcinog 2002; 33(1):25-35). Cluster analysis of the measured expression levels identified gene-specific expression patterns from highly aggressive prostate tumors that were distinct from patterns of gene expression in organ confined disease tissue (Luo et al., supra).

However, although these investigations of solid tumors provide detailed information on the pathology and malignant process of the tumor, invasive surgery is always necessary to obtain the tumor tissue studied. In contrast, this Example investigates the feasibility of a microarray-based diagnostic test that measures the levels of RNA transcribed from peripheral blood leukocytes of each individual at risk for prostate cancer, and thus does not require surgery to obtain each diagnostic sample.

Studies have shown that cancer patients exhibit immune responses that differ from those of control individuals. These studies have also demonstrated that this response can be detected at the level of altered expression of individual genes, e.g., cytokine genes, in leukocytes within peripheral blood. This example employs microarray technology to quantify the gene expression levels of thousands of genes in each prostate cancer patient and control subject's blood sample, permitting the determination of leukocyte gene expression patterns, or signatures, for each prostate cancer patient and control subject analyzed. Pattern analysis algorithms compare these expression signatures, and define patterns that can distinguish both between normal individuals and those with cancer, and also between patients with prostate tumors at different stages of biological progression. Identification of a leukocyte multigene expression signature specific to prostate cancer, and also characteristic for pathologically defined stages of prostate cancer, provides both diagnostic and prognostic information on individual tumors, and thus play a vital role in prostate cancer pre-biopsy population screens.

The results from this experiment form the basis of a pre-biopsy diagnostic screen. A clinical assay initially involves the hybridization of a labeled probe synthesized from RNA extracted from a blood sample drawn from the individual at risk for prostate cancer to a microarray containing a number of genes that are differentially expressed between cancer patients and control individuals. The resultant expression pattern is then compared to a set of known multigene signatures, specific for individual stages of tumor progression for a non-invasive prostate cancer diagnostic assay that can yield both diagnostic and staging information for each individual at risk.

Furthermore, since this assay will measure gene expression within leukocytes, instead of circulating malignant cells, and does not rely on the measurement of biomolecules secreted from malignant cells, the resultant assay is sensitive and accurate, and capable of detecting tumors that are still at an early stage of malignancy. Such an assay serves as an important pre-screen that can, with a minimum of patient discomfort, identify men with prostate cancer.

Data from investigations of ratios of T cell subsets provides some evidence to correlate serum cytokine levels with mRNA levels (detected by RT-PCR), within peripheral blood from cancer patients. It is therefore likely that the differential prognostic serum levels measured from prostate cancer patient blood will also be detected at the level of mRNA expression. The experimental procedure follows the expression levels of each of the genes mentioned above, and thus determine whether previously reported increases or decreases in serum protein levels in cancer patients correlate with the corresponding mRNA levels detected by microarray analysis.

In the work described by Veltri et al., Strom et al., supra and in the other investigations described above, mRNA levels have been quantified individually by RT-PCR techniques. However, this would be extremely time-consuming if many genes were to be analyzed in one experiment. In contrast, this Example employs microarray technology to quantify mRNA transcripts, which allows the simultaneous analysis of thousands of genes expressed in peripheral blood leukocytes. The complex differential gene expression measured using this approach identifies patterns or signatures of gene expression that differ between prostate cancer patients and control subjects, and thus forms the basis of a diagnostic technique.

It seems clear that the use of multiple gene products for the determination of expression signatures provides considerably more detailed information on tumor stage and prognosis than can be provided by the quantitation of individual serum protein levels.

It should also be noted that although leukocyte gene expression levels will be measured, if, e.g., malignant prostate cells were also present in the blood of patients, then gene expression of these cells will also be quantified. However, it seems likely that the detection of gene expression of malignant cells within blood would actually increase the specificity of this analysis, as mRNA levels arising from circulating metastatic cells would differ from mRNA levels in patients with no metastatic cells in their blood stream.

### Microarray Technology

Oligonucleotide Microarrays. There are two major types of microarray technology; spotted cDNA arrays and manufactured oligonucleotide arrays. The present invention employs high density oligonucleotide Affymetrix® GeneChip arrays (reviewed in Schena at el., Trends Biotechnol. 1999; 16(7):301-6). The Affymetrix system was chosen due to: 1) the large numbers of gene sequences represented within the array, 2) the highly developed Affymetrix protocols for probe preparation and microarray hybridization, and 3) the built-in multiple internal standards. In addition, custom designed normalization software for accurate comparison of results between each individual hybridization accommodates the experimental plan, which involves a direct comparison between individual microarray experiments.

A recent investigation of the reproducibility of DNA microarrays has highlighted some problems of reproducibility and comparison among array hybridizations, and the need for replicate experiments. Use of the Affymetrix system eliminates some of the problems that are associated with other microarray technologies, because it provides a significantly lower variation between experiments.

Pattern Analysis Algorithms and Computer Analysis. After scanning of each microarray to detect the hybridization signals, Affymetrix® Microarray Suite software is employed for image acquisition and normalization of the fluorescent signals using internal standards. Analysis of the resultant signal intensities over each oligonucleotide, or data point, within each experiment then falls into two main categories: Supervised Learning Algorithms (Golub et al., infra; Slonim et al., 1999; Yeang et al., Bioinformatics. 2001; 17 Suppl 1:S316-22; Ramaswamy et al., infra); and Hierarchical Clustering (Eisen et al., infra; Alizadeh et al., infra; Perou et al., Nature 2000; 406(6797):747-52). All algorithms employed have the capacity to analyze the very large data-sets, and allow comparisons of multiple experiments and multiple points within a single experiment.

Affymetrix oligonucleotide microarray technology is employed to simultaneously measure the expression levels of up to about 14,000 genes transcribed in circulating leukocytes derived from the peripheral blood of 40 prostate cancer patients and 20 control individuals. Briefly, leukocytes are extracted from whole blood obtained from prostate cancer patients and healthy controls, and the RNA isolated from these cells is employed to synthesize cDNA, which is then itself employed as a template to synthesize labeled cRNA for hybridization to Affymetrix microarrays. The expression patterns generated for each individual subject sample are compared using data analysis algorithms that have the ability to identify and record multigene expression levels as patterns or multigene signatures.

In a specific experiment, leukocytes are collected and subject to sample processing and microarray hybridization. Expression data is derived from microarray hybridization plus data-analysis algorithms to generate multigene expression patterns. The evidence shows that circulating blood leukocytes in individuals suffering from prostate cancer exhibit a characteristic signature of gene expression levels that is different from the signature exhibited by circulating leukocytes from control subjects. Multigene expression signatures in individuals with prostate cancer are specific to the aggressiveness of the tumor from the individual examined, and thus reflect the stage the malignancy has reached in the patient.

### Materials and Methods

Prostate Cancer Subjects. Prostate cancer patients are derived from those undergoing radical prostatectomy (n=50 per year), and those undergoing radiation seed implant therapy (n=150 per year). The total population of prostate cancer patients will be screened for possible recruitment into this study. Informed consent is obtained, according to Institutional Board Regulations. Blood drawing takes place prior to radical surgery or seed implantation.

Each patient recruited to participate in this study is provided with a questionnaire designed to obtain both demographic information and information on current general heath. The questionnaire is approved by the Institutional Review Board. Clinical information and pathology reports is also collected for this study. This documentation includes patient history of serum PSA tests, all results of prostatic needle biopsy (Gleason's stage) and/or clinical and pathological analysis of tumor tissue following surgery (TNM scale, pT stage). CBCs are performed on all recruited patients following blood drawing. Each patient record also has dates of any previous needle biopsy, or other surgical procedures (on average 3-6 months prior to the biopsy).

Exclusion Criteria. Patients are excluded if: 1) they have had surgery or other physical trauma less than six weeks prior to blood collection, 2) if they have abnormal CBCs, 3) if they have a current infection, 4) if they have autoimmune disease, 5) if they have had chronic use of immunosuppressants or anti-inflammatory medication. These exclusion criteria have been designed to reduce the likelihood of including prostate cancer patients that exhibit leukocyte gene expression that is different from healthy control subjects, but that arises from factors other than growth and development of a prostate tumor, such as an immune response to surgery or the presence of an infectious agent.

Expression signature assays include the screening, recruitment, blood drawing and leukocyte sample preparation of prostate cancer patients. Following removal of red blood cells, the leukocyte cell samples are stable at −70° C. for long periods of time. For each subject, blood will be drawn, processed to isolate leukocyte cells, and then stored at −70° C. Subjects are chosen for complete processing (which involves the extraction of RNA, synthesis of cDNA and cRNA, and microarray hybridization) based on the criteria described below.

Microarray analysis measures gene expression levels from 40 of the leukocyte samples collected. The expression data are subjected to supervised learning and clustering algorithms to identify and determine leukocyte gene expression patterns that distinguish between prostate cancer patients and healthy controls.

The expression data generated are then used to distinguish among leukocyte gene expression patterns of prostate cancer patients at different diagnosed stages of tumor progression. All patients undergoing treatment, and who are recruited into this study, will have documented reports following needle biopsy (a Gleason score can be documented for each subject). For those patients undergoing radiation seed implantation, further pathological information are not available. Tumors of prostate cancer patients with clinically localized disease can be staged after prostatectomy by the TNM scale (T1, T2 and T3), and also given a more accurate pT stage. The expression data only of men with pathological staging, and thus only of those who will undergo radical surgery are evaluated. Assuming a conservative 20% recruitment of all radical prostatectomy patients (which is below current recruitment levels of prostate cancer subjects), greater than 20 subjects are recruited over the two year period of this proposal.

This experiment involves recruitment of subjects, extraction of leukocytes and completed sample processing for every prostate cancer patient who satisfies the following criteria: undergoes radical prostatectomy or radiation seed implantation, consents to take part in this proposal, does not fall within the exclusion criteria, and has detailed tumor stage information available.

Control Subjects. Twenty control male subjects, approximately age-matched to prostate cancer patients, are recruited from the staff and staff relatives. Informed consent is obtained, according to Institutional Board Regulations. Each control subject recruited to participate in this study is provided with a questionnaire to obtain both demographic information and information on current general heath. The questionnaire is approved by the Institutional Review Board. Information collected through the completion of this questionnaire is employed as described above, as well as to determine that a control subject is unlikely to have an undiagnosed prostate tumor, or other solid tumor, that may effect leukocyte gene expression. Blood samples are drawn by a trained phlebotomist from the antecubital vein using a needle and evacuated tube. For each control subject chosen to take part in this study, serum PSA levels are measured, and CBC counts performed.

Exclusion criteria for controls. Control subjects are excluded from this study if: 1) they have serum PSA levels >4 ng/ml, 2) if they have abnormal CBCs, 3) if they have experienced discomfort while urinating, 4) if they have a first-degree relative diagnosed with prostate cancer or any other solid tumor, 5) if they have documented a current infection, 6) if they have autoimmune disease, 7) if they have had surgery or other physical trauma less than six weeks prior to blood collection, 8) if they have had chronic use of immunosuppressants or anti-inflammatory medication.

Potential Problems Arising from Factors Other than Prostate Cancer. During recruitment of both prostate cancer patients and control subjects, it is clear that attention must be paid to the possibility that the mRNA levels of some of the genes expressed in leukocytes, in both patients and control subjects, may change because of underlying inflammatory disease states or other illness. As described above, both prostate cancer patients and control subjects are otherwise normal healthy individuals with no history of autoimmune disease or current infection. It is unlikely that any control subject has an undiagnosed prostate or other solid tumor.

However, it is well known that individuals possess different immune complements, and these may well be detected within individual experiments. Flagging is a method employed to normalize between patient samples and thus will be employed to reduce some of the inter-subject variability that may be detected following microarray hybridization. Any gene found to be significantly differentially expressed (>3 fold change) between two or more of the normal control individuals, will be “flagged”, which subsequently removes this gene from any further analysis. This method was successfully used to remove inter-subject variation from both multiple patient samples such as total lymph nodes, and also from multiple cell lines of different lineages that were employed to identify profiles of gene expression in B cell lymphomas (Alizadeh et al., supra).

It should be noted that the algorithms described in detail below have been successfully employed to identify gene expression profiles that distinguish complex tumor tissue from normal non-disease tissue (that has not undergone micro-dissection procedures), and thus are not hindered by complex patterns of total gene expression.

Use of the Affymetrix Oligonucleotide Microarray Technology. The Affymetrix system appears to be better suited to the present project than a cDNA microarray-based system. Therefore, Affymetrix Human Genome U133A oligonucleotide microarrays are employed to analyze gene expression signatures in peripheral blood leukocytes taken from the prostate cancer patients described above, and in corresponding cells from control subjects recruited during this study. This array is an upgraded version of the HU95A arrays employed in the preliminary studies, and will soon replace this array. The arrays are comparable with each other.

Affymetrix Human U133A oligonucleotide microarrays contain about 14,000 individual human sequence verified oligonucleotides, representing Unigene, GenBank and TIGR database clusters that have been previously characterized by function and disease association. The specific gene products described above are all represented on this microarray and thus are included in all analytical procedures. Furthermore, many other genes known to be involved in immune responses are also included on this microarray, such as multiple cytokines and growth factors, e.g., osteopontin, which has been found to be up-regulated in prostate tumor models (Thalmann et al., Cancer Res. 1999; 54(10):2577-81), and shown functionally to play a role in cell mediated immunity.

Sample Processing, Probe Preparation and Microarray Hybridization. All blood samples are processed immediately following collection; leukocytes are extracted from blood using lysis buffers and centrifugation, according to standard procedures. The leukocytes are stable at −70° C. for long periods of time (>6 months, Qiagen). The storage of leukocytes at that temperature allows the retrospective determination of which samples are to be hybridized to GeneChips, after a detailed analysis of all available patient history and a confirmed histological analysis of biopsy samples and prostate tissue in the case of patients undergoing surgery. All patient and control samples chosen for RNA extraction are processed in duplicate, by splitting the white blood cell sample extracted from whole blood and processing the duplicate samples identically thereafter. The need for replicate microarray experiments has been previously highlighted, so each sample is processed in duplicate. This experimental design was based on the reproducibility of Affymetrix arrays, hybridization protocols and scanning (mean R2=0.98 and 0.967 for repeat hybridization, and duplicate sample processing respectively), and previously reported use of duplicate hybridizations in microarray experiments (Chen et al., J Cell Biol 2000; 151:1321-36).

Data Analysis. Following image acquisition and normalization using Affymetrix software and protocols, the data analysis employs two major algorithm types; Hierarchical Clustering (Eisen et al., infra; Alizadeh et al., infra; Perou et al., infra) and Supervised Learning Algorithms; Group Classification (Golub et al., supra; Slonim et al., infra), and Support Vector Machine (Yeang et al., supra; Ramaswamy et al., infra). Use of each of these techniques is described in detail below.

Hierarchical Clustering. Leukocyte expression signatures discriminate between cancer patients and control, matched subjects, and also to attempt to distinguish among individual stages of the prostate tumors analyzed. Data analysis initially employs a hierarchical clustering algorithm that has been successfully applied to classify gene expression data in several studies of human tumors, and is briefly described as follows. The Cluster program (M. Eisen), employs a fast two-way clustering that is based upon a similarity metric between genes and experimental samples. A standard Pearson correlation coefficient is employed to perform multiple iterations of similarity measurements between each data point (microarray probeset intensity value) within the vertical axis, thus expression levels between every gene in the data-set. Relationships among genes are represented by a tree, whose branch distance lengths reflect the degree of similarity between genes. This distance can be calculated depending on the amount of constraint needed; as a single-linkage cluster (where Cluster calculates the minimal distance between two genes), an average-linkage (calculates the average distance), or complete-linkage cluster which is the most conservative measurement of gene expression similarity that calculations the maximum distance.

The clustering procedures yield a binary tree where genes are near each other on the tree if they are strongly correlated, and branches of similarly expressed genes group into discrete nodes. The same algorithm is then applied to cluster the experimental samples according to their overall patterns of gene expression.

A graphic display of the intensities of the genes by individual subjects is then created in the program TreeView (M. Eisen). Intensity of each gene is normalized by median centering and represented by a color scheme varying from red for high intensities to green for low. The genes are ordered along the vertical axis using the binary tree from the first cluster analysis. The subjects are arranged across the horizontal axis according to the second binary tree. This visual representation of the data shows clusters of genes that exhibit similar expression intensity among each individual subject.

Hierarchical clustering is performed on all 40 prostate cancer patients and 20 control subjects recruited during this study. The gene expression data will correctly classify patients from controls. It should be noted that the hierarchical Clustering algorithm will cluster only those genes that exhibit a similar pattern of leukocyte expression among subjects. Thus, differential gene expression that arose, e.g., from an irregular immune response in only one individual will not be included in the cluster of similarly expressed genes among all subjects. Although this may result in some genes being removed from analysis due to variable levels in some subjects, this algorithm will act to reduce the influence of the many non-PCa related gene expression changes that may be detected when analyzing so many data points.

Expression profiles can distinguish prostate tumor samples according to the stage of tumor aggressiveness. The results derived from the clustering algorithms should correlate with tumor stage, e.g., all patients with a defined stage of T3 should cluster together in a sub-node, away from sub-nodes of different staged tumors. To analyze Cluster results all TreeView readout data are compared with the detailed surgical report pathology provided for each patient employed in this analysis to identify clusters of patient samples that fall within similar clinical and pathological tumor stages. Such an approach has been successfully applied to distinguish among populations of both B-cell lymphomas (Alizadeh et al., supra), multiple breast tumors (Perou et al., 2000) and prostate tumor tissue (Dhanasekaran et al., supra).

It may prove useful to perform a supervised clustering experiment, as this Example employs a surrogate tissue in which differences in the patterns of gene expression of leukocytes from tumor patients may be more subtle than the differences obtained from analysis of the tumor tissue itself. Supervised clustering can be performed using adjustments within the Cluster program. For example, for the initial data analysis each sample was given equal weighting i.e., each sample was assigned equal importance (and thus defined as unsupervised). If the weighting of the samples is altered and the data is then analyzed in Cluster using GORDER, which provides a constraint on the algorithm to keep the samples in particular groups (e.g., groups of prostate cancer patients at disease T2 versus groups of patient at T3), the horizontal axis of gene similarities will be defined by this order. In this instance, branch length within and between nodes can be employed to identify genes with similar expression patterns between the selected groups.

Finally, genes that are significantly differentially expressed between subgroups of patients and/or subgroups of controls (that have not been removed by flagging procedures) may have strong weighting on the final clustering results. This may alter the final nodes of the clusters and even skew the overall cluster data. Therefore statistical tests, such as the student T-or Wilcoxin test (ensuring that in each instance there are sufficient sample numbers for analysis), are performed to identify, and then remove from analysis, genes significantly differentially expressed between, all control subjects. This procedure should help to greatly reduce the inter-subject variation.

Supervised Learning Algorithms. Supervised learning algorithms are based on an initial definition of the subject groups to be distinguished by the algorithm. A sub-set of each group is employed to determine characteristics that can separate the two groups, in this case gene expression levels. The characters, or genes, that play a role in the separation, are then used on a test set of data (the remaining subjects), to call each test sample. Two algorithms employed for this analysis are briefly described below.

Group Classification. Group Classification (Golub et al., 1999; Slonim et al., 1999), has been recently used to investigate genetic differences between leukemia's, elucidating gene expression distinctions between two forms of this disease. This algorithm will be used to evaluate and compare the results generated through the hierarchical clustering method. Following procedures employed by Golub et al., (supra) subjects are divided into two sets: the “training set” includes 20 prostate cancer patients and 10 normal control subjects; the “test set” includes an additional 20 tumor patients and 10 control subjects. A multigene expression signature is constructed using the 30 subjects from the “training set”, as follows. First, all genes are sorted by the degree of correlation between the expression level and subject diagnosis, in this case being positive or negative for prostate cancer. A correlation metric which measures relative class separation is used [correlation metric P(g,c)=(μ1−μ2)/(σ1+σ2) where g=the expression vector of a gene over n samples and c=the diagnostic class vector]. The significance levels of these correlations is then determined using a permutation test called “neighborhood analysis” Taking the significantly correlated genes, different subsets of genes are then tested to find the best model for classifying diagnosis using cross validation procedures within the “training set”.

The final model is then used with the “test set” of additional patients and controls, to see if subjects can be correctly classified with a positive or negative tumor diagnosis. Each gene “votes” for cancer or normal diagnosis, based on whether its expression level is closer to the mean expression level of prostate cancer patients or of normal controls. This vote is weighted by the degree of correlation between the gene and diagnostic group. Votes across all genes are summed to make a final classification of diagnosis, provided there is sufficient prediction strength as measured by the margin of “victory” [prediction strength=(V_{winner}−Vₗₒₛₑᵣ)/(V_{winner}+Vₗₒₛₑᵣ) where V is then number of “Votes” received for each diagnosis]. Classification of subjects is evaluated in terms of error rate (% incorrect classifications) and “no-call” rate (% of samples considered “uncertain”).

Support Vector Machine. A support vector machine (SVM) supervised learning algorithm has recently been employed to perform multiclass cancer diagnosis of 14 different tumor classes and control tissues, and is employed for analysis of the prostate and control sample leukocyte gene expression data. Data input for this algorithm is similar to group classification, whereby subjects are divided into training and test sets. The training set is then characterized by labeling or classing each subject as positive +1 (e.g., prostate cancer samples) or negative −1 (e.g., control samples). SVM finds a hyperplane, w, which separates positive and negative training samples and maximizes the margin, or distance, between the samples and the hyperplane, where f(x)=w·x+b. The geometric property can be imposed by means of the following optimization problem: minimize½∥w²∥ subject to yᵢ(w·xᵢ+b)≧1, for all i (where x is the input data, e.g. expression level; y is the class label +1 or −1). The discriminate function can be written as f(x)=Σwᵢyᵢ(x·xᵢ)+b, where wᵢ's and b can be obtained from solving the quadratic function. The hyperplane is then employed for classification of the test set, where an unknown test samples position relevant to the hyperplane determines its class, and the confidence of each SVM prediction is based on (and is proportional to), the distance of the test sample from the hyperplane.

The SVM described above results in a binary classification, which is employed to distinguish between the two groups of 40 prostate cancer patients and 20 control subjects. Evaluation of the ability of the algorithm to correctly group patients and controls will determine which genes are major effectors in the classification, and the statistical power of each for each sample.

In the papers referenced above, a one-versus-all (OVA) approach has also been employed to perform multiclass prediction. The OVA builds k (the number of classes) binary classifiers which distinguish one class from all the other lumped together (Yeang et al., 2001; Ramaswamy et al., 2001). For a test sample x, the binary classifier outputs form a k-vector f(x)=(fᵢ(x), . . . ,fₖ(x)). If f (x) is a real number (i.e., a predicted class with confidence value), then the predictor finds the maximum of fᵢ(x) and assigns the sample to the corresponding class label. Using this approach, Ramaswamy et al., created a multiclass cancer gene expression database from 144 human cancers and normal tissues from a total of 14 classes, and demonstrated a 78% accurate classification/diagnosis of the correct cancer or control tissue over the set of test samples (n=54) (Ramaswamy et al., supra). An OVA approach, where k=each stage of prostate tumor (i.e., T1-3(4)) can be employed here.

### Analysis of Leukocyte Gene Expression and The Multigene Expression Signatures Determined Following Data Analysis

Quantitative RT-PCR. Although recent reports have documented the reliability and reproducibility of microarray analysis, this powerful technology is still in its infancy and it may be necessary to perform additional confirmation of the expression results obtained. Therefore, gene specific primers are designed for a number of genes seen to be differentially regulated among leukocytes obtained from cancer patients and controls, and employed for assay via real-time RT-PCR of leukocyte transcript levels. The actual number of genes employed for validation of results depends on the number of genes found in this assay to be differentially expressed. Microarray experiments performed by other researchers, and cited above, are available as guidelines for this analysis. Genes chosen for this analysis include those identified in previous studies that are differentially regulated between leukocytes from patients with a solid tumor relative to leukocytes from control subjects (and are thus positive controls), and also genes included in the multigene signatures deduced through the data analysis. For each gene analyzed, RT-PCR analysis is used confirm and validate the outcome of the microarray analysis.

### Example 4

### Breast Cancer Diagnosis Bloodcell Multigene Signatures

### Introduction

Breast cancer is the second leading cause of cancer deaths in North American women. It has been estimated that in 2002, 203,500 new cases of breast cancer were diagnosed in the US, with approximately 39,600 deaths in that year alone (Jemal et al., 2002) Current techniques for the screening of breast cancer, as a prerequisite to biopsy for diagnostic evaluation of the detected mass, include physical breast examination and mammography. These techniques possess a number of limitations, including lack of specificity and accuracy in the diagnosis and, also a lack of cancer stage and prognostic information. This ultimately yields high numbers of false positive diagnoses, and consequently unnecessarily large numbers of surgical biopsies. The rationale behind this proposal is based on two sources of data: 1) Current scientific literature, in which there is growing evidence that individuals with breast cancer and other forms of malignant disease such as prostate cancer, exhibit immune responses that can be detected at the level of altered gene expression in leukocytes circulating in peripheral blood. Quantitation of the mRNA transcripts in leukocytes of a number of individual genes has demonstrated associations between gene expression levels and the presence of a tumor in patients with breast and prostate cancer. 2) Preliminary results from a microarray study investigating gene expression changes in men with prostate cancer. Initial results from this study have been striking; supervised cluster analysis of peripheral leukocyte gene expression data, using transcript level measurements of thousands of genes from eleven prostate cancer patients and seven matched control subjects, resulted in a classification of all the subjects into their correct group.

The use of microarray technology allows the simultaneous measurement of the expression levels of up to 14,000 genes transcribed in circulating leukocytes derived from the blood of breast cancer patients and control individuals. This technology, demonstrates that women suffering from breast cancer exhibit a conserved pattern, or signature, of gene expression levels in their peripheral blood leukocytes, which is distinct from the corresponding pattern of expression in leukocytes from control subjects. Patients with breast cancers at different histological grades, yield distinct expression signatures that reflect the biological stage and aggressiveness of the cancer, and that information can thus be employed to differentiate among breast cancers at different pathological stages.

This Example demonstrates a novel technique that does not require invasive techniques to obtain tumor tissue, yet provides an accurate diagnosis of breast cancer, and also provides detailed prognostic information on the stage and biological aggressiveness of the tumor. The success of this project would yield a much needed, non-invasive tool for stage-specific diagnosis of the disease, and thus serve as an important screening tool to identify women with breast cancer.

Although mortality rates have decreased over the past decade, through pre-symptomatic screening programs and major improvement in breast cancer treatment, breast cancer survival rates decrease dramatically in women with a more advanced stage at diagnosis and it has been estimated that only half of all breast cancers are localized at the time of diagnosis. Thus, effective management of breast cancer relies heavily on an early diagnosis, coupled with a need to obtain accurate information on the classification and stage of the cancer itself, and thus limitations of traditional diagnostic and prognostic techniques may currently hinder the management of breast cancer.

Although frequently advocated, evidence to support the use of breast self examinations (BSE) in screening programs is weak. To date, no study has evaluated the effectiveness of clinical breast examinations (CBE) as a stand-alone screening technique. Widespread adoption of screening mammography, which utilizes ionizing radiation to image breast tissue, has been accredited with the dramatic increase in incidence rates of breast cancer between 1980-1987, illustrating the benefit of screening programs in identifying the presence of breast cancer. However, there has been much recent controversy over the benefits versus the risks of regular mammography (reviewed in Humphrey et al., supra). Thus, in large mammography screening programs, for every one hundred dollars spent on screening thirty three will have been spent on the evaluation of false positive results (Elmore et al., N Engl J Med. 1998; 338(16):1089-96).

Other techniques include the use of ultrasound in the evaluation of palpable or mammographically identified masses, and the use of serum tumor markers for the detection of breast cancer, such as CA15.3, lack sensitivity and specificity (Chan D. W., 2001) and research has focused on the use of PCR-based approaches for tumor micrometastasis detection. Quantitative RT-PCR analysis of gene products, in malignant cells that have survived detachment from the original tumor site and circulation within peripheral blood, has been employed to identify metastatic disease, and detection of circulating levels of the mRNAs transcribed by the genes CK-19, MUC1, CEA and mammoglobin, has been reported to provide both diagnostic and prognostic information on breast cancer (Berois et al., Eur J Cancer 2000; 36(6):717-23). However, problems with these methods have been documented such as the detection of CK-19 pseudogenes that can contaminate results, and the lack of replication and lack of tissue specific expression detected following analysis of mammoglobin.

Research has implied that some important diagnostic and prognostic information will be derived only after surgical procedures, and in current clinical practice an actual diagnosis of breast cancer is made following pathologic review of a tissue specimen. Breast tissue can be obtained by the methods of excisional or incisional biopsy, where the entire palpable mass or a section of the mass (respectively) is surgically removed. Although accurate, these techniques are very painful for the patients and lead to extensive scarring (which can mimic a malignancy on physical or mammographic examination).

Differential Expression Of Individual Genes In Leukocytes From Patients With Breast Cancer. The tumor derived antigen 90K (Mc-2 BP) is a widely expressed, secreted glycoprotein found in the serum of healthy individuals. Levels of the 90k protein are significantly increased in the serum of patients with breast cancer, and Fusco et al., showed that 90K serum protein levels were also elevated in 20% of patients with no clinical evidence of the disease (et al., Int J Cancer. 1998; 79(1):236). Fusco et al., additionally showed that transcript levels of the 90K gene were also higher in patients versus controls, and they suggest that peripheral blood cell monocytes (isolated from whole blood) may be activated in response to breast cancer growth and progression.

Martin et al., performed an targeted microarray based investigation using RNA template derived from blood of breast cancer patients (DCIS to Stage IV), (et al., Proc Natl Acad Sci USA.; 98(5):2646-51). Genes were chosen to be placed on the array if they were found to be differentially expressed between breast cancer tissue and control breast tissue by differential display (n=170). Cluster analysis identified a group of 12 genes that were elevated in 77% of the subjects with more aggressive cancer, including the genes maspin, CD44 and HER2. However, although the authors hypothesize that they are detecting disseminated breast cancer cells (Martin et al., supra), they suggest that their results may also arise from the detection of mRNA transcripts within the leukocytes themselves. Evidence to support this alternate hypothesis also comes from preliminary studies measured leukocyte expression levels were measured in prostate cancer patients and control subjects. The genes maspin, CD44 and HER2 were all found to be significantly expressed above background levels in all subjects, and HER2 was shown to be up-regulated in prostate cancer patients. As described above, experiments that show the accurate classification of prostate cancer subject and healthy control subjects into their respective groups, based on the expression levels of over 1500 gene, provide evidence to support breast cancer diagnosis though leukocyte expression signatures. The genes employed above for classification of prostate cancer will not necessarily be the exact genes employed for classification of breast cancer. However, extensive literature has been published documenting the common similarities between breast and prostate cancer, including incidence and mortality rates, risk factors, initiation of transformation, and roles of androgens and estrogens (reviewed in Lopez-Otin & Diamandis Endocr Rev.; 19(4):365-96). These data, along with results presented infra, provide evidence that growth and development of a breast cancer will exert an effect on the immune system that can be detected at the level of altered gene expression in peripheral blood leukocytes.

This Example employs microarray technology to quantify mRNA transcripts, which allows the simultaneous analysis of thousands of genes expressed in peripheral blood leukocytes. The complex differential gene expression measured using this approach identifies patterns or signatures of gene expression that differ between breast cancer patients and control subjects, and thus forms the basis of a diagnostic technique.

It seems clear that the use of multiple gene products for the determination of expression signatures provides considerably more detailed information on tumor stage and prognosis than can be provided by the quantitation of individual serum protein levels. It should also be noted that although leukocyte gene expression levels will be measured, if, e.g., malignant breast cells were also present in the blood of patients, then gene expression of these cells will also be quantified. It seems likely that the detection of gene expression in malignant cells within blood would actually increase the specificity of this analysis, as mRNA levels arising from circulating metastatic cells would differ from mRNA levels in patients with no metastatic cells in their blood stream.

Affymetrix oligonucleotide microarray technology is employed to simultaneously measure the expression levels of up to about 14,000 genes transcribed in circulating leukocytes derived from the peripheral blood of 55 breast cancer patients and 25 control individuals as described above. In a specific experiment, leukocytes are collected and subjected to sample processing and microarray hybridization. Expression data derived from microarray hybridization plus data-analysis algorithms to generate multigene expression patterns is used for analysis. These data show that circulating blood leukocytes in individuals suffering from breast cancer exhibit a characteristic signature of gene expression levels that is different from the signature exhibited by circulating leukocytes from control subjects. Multigene expression signatures in individuals with breast cancer are specific to the aggressiveness of the tumor from the individual examined, and thus reflect the stage the malignancy has reached in the patient.

### Materials And Methods

Breast cancer subjects. The experimental approach measures leukocyte gene expression levels in 55 breast cancer patients, and 25 matched control subjects, with duplicate sample processing of each subject. Duplicate processing was performed to permit the robustness of a cancer-specific gene expression signature to be determined. The microarray technology and pattern analysis algorithms and analysis are the same as for the prostate study in Example 3.

All breast cancer patients are potential candidates for enrollment into this study, and this total population of breast cancer patients is screened for possible recruitment into this study. Informed consent is obtained, according to Institutional Board Regulations. Blood drawing takes place following initial diagnosis or confirmation of breast cancer diagnosis, and prior to the onset of treatment for the disease. Treatment options for breast cancer are generally directed by the stage that the tumor has reached in that individual. For example, treatment for Stages I and II most often involves a combination of surgery and radiation therapy and/or adjunct systemic therapy. Treatment for stage III, which is characterized by lymph node involvement, may alternatively start with chemotherapy, followed by surgery and radiation therapy. Patients from stages I, and II, and stage III will be included only if recruitment and blood drawing was performed prior to the initiation of therapy. Additionally, patients with advanced metastatic disease may also be recruited if they are screened for participation prior to the onset of treatment for localized and metastatic disease.

Annual estimates of patients available at stages I, II, and III are about 80-100. It is not contemplated to specifically screen and exclude patients based on actual tumor stage, or the presence of metastatic disease. This broad inclusion should allow recruitment of at least 20 patients from stages I-III.

Each patient recruited to participate in this study is provided with a questionnaire designed to obtain both demographic information and information on current general heath. The questionnaire is approved by the Institutional Review Board. Clinical information, biopsy reports (including dates of biopsy), and any further pathology reports are also collected for this study. This documentation includes all patient history, all results of any mammography, ultrasound, and core needle biopsy. CBCs is performed on all recruited patients following blood drawing.

Exclusion Criteria for Patients. Patients will be excluded from this study if: 1) they have had surgery or other physical trauma less than six weeks prior to blood collection, 2) if they have abnormal CBCs, 3) if they have a current infection, 4) if they have autoimmune disease, 5) if they have had chronic use of immunosuppressants or anti-inflammatory medication. These exclusion criteria have been designed to reduce the likelihood of including breast cancer patients that exhibit leukocyte gene expression that is different from healthy control subjects, but that arises from factors other than growth and development of a breast cancer, such as an immune response to surgery or the presence of an infectious agent.

Control subjects. Twenty-five control female subjects, approximately age-matched to breast cancer patients, are recruited from the staff and staff relatives. Informed consent is obtained, according to IRB regulations. Each control subject recruited to participate in this study is provided with a questionnaire to obtain both demographic information and information on current general heath. The questionnaire is approved by the Institutional Review Board. Information collected through the completion of this questionnaire is employed as described, as well as to determine that a control subject is unlikely to have an undiagnosed breast, or other solid tumor, that may effect leukocyte gene expression. Blood samples are drawn by a trained phlebotomist from the antecubital vein using a needle and evacuated tube. For each control subject chosen to take part in this study, CBC counts are performed. Clinical Breast Examinations for control subjects are also performed. Control subjects are informed, in writing, of the results of their CBE.

Exclusion Criteria for Controls. Control subjects are excluded from this study if: 1) they have abnormal CBCs, 2) they have a high risk factor for developing breast cancer, such as two first-degree relatives with the disease, 3) if they have a first-degree relative diagnosed any other solid tumor, 4) if they have documented a current infection, 5) if they have autoimmune disease, 6) if they have had surgery or other physical trauma less than six weeks prior to blood collection, 7) if they have had chronic use of immunosuppressants or anti-inflammatory medication. Control subjects are excluded if a palpable mass is detected by CBE.

Potential Problems Arising from Factors Other Titan Breast Cancer. During recruitment of both breast cancer patients and control subjects, it is clear that attention must be paid to the possibility that the mRNA levels of some of the genes expressed in leukocytes, in both patients and control subjects, may change because of underlying inflammatory disease states or other illness. As described above, both breast cancer patients and control subjects are otherwise normal healthy individuals with no history of autoimmune disease or current infection. It is unlikely that any control subject has an undiagnosed breast carcinoma or other solid tumor.

However, it is well known that individuals possess different immune complements, and these may well be detected within these experiments. Flagging is a method employed to normalize between patient samples and this will be employed to reduce some of the inter-subject variability that may be detected following microarray hybridization. Any gene found to be significantly differentially expressed (>3 fold change) between two or more of the normal control individuals, will be “flagged”, which subsequently removes this gene from any further analysis. This method was successfully used to remove inter-subject variation from both multiple patient samples such as total lymph nodes, and also from multiple cell lines of different lineages that were employed to identify profiles of gene expression in B cell lymphomas (Alizadeh et al., Nature 2000; 403(6769):503-11). It should be noted that however that this approach to remove gene expression variability, or “noise”, was not employed in the preliminary studies, as supervised hierarchical clustering analysis was performed, where expression noise can be removed from the data set prior to input into the data analysis algorithms. Furthermore, flagging genes may eliminate too many genes from analysis. With this in mind, expression analysis is performed on the data sets pre- and post-flagging.

The algorithms described in detail below have been successfully employed to identify gene expression profiles that distinguish complex tumor tissue from normal non-disease tissue (that has not undergone micro-dissection procedures), and thus are not hindered by complex patterns of total gene expression.

Use of the Affymetrix Oligonucleotide Microarray Technology. The Affymetrix system appears to be better suited to the present project than a cDNA microarray-based system. Therefore, Affymetrix Human Genome U133A oligonucleotide microarrays are employed to analyze gene expression signatures in peripheral blood leukocytes taken from the breast cancer patients described above, and in corresponding cells from control subjects recruited during this study. This array is an upgraded version of the HU95A arrays employed in the preliminary studies, and will soon replace this array. The arrays are comparable with each other.

Affymetrix Human U133A oligonucleotide microarrays contain about 14,000 individual human sequence verified oligonucleotides, representing Unigene, GenBank and TIGR database clusters that have been previously characterized by function and disease association. The specific gene products described above are all represented on this microarray and thus are included in all analytical procedures. Furthermore, many other genes known to be involved in immune responses are also included on this microarray, such as multiple cytokines and growth factors, and e.g. maspin, which has been found to be down-regulated in breast cancer mouse models.

Sample Processing, Probe Preparation and Microarray Hybridization. All blood samples are processed immediately following collection; leukocytes are extracted from blood using lysis buffers and centrifugation, according to standard procedures. The storage of leukocytes at that temperature allows the retrospective determination of which samples are to be hybridized to GeneChips, after a detailed analysis of all available patient history and a confirmed histological analysis of biopsy samples (and tissue, in the case of patients undergoing surgery after their participation in this study). All patient and control samples chosen for RNA extraction are then processed in duplicate, by splitting the white blood cell sample extracted from whole blood and processing the duplicate samples identically thereafter.

Replicate Sample Processing Versus Non-Replicates. The need for replicate microarray experiments has been previously highlighted (Lee et al., 2000). There is much discussion in the scientific community on the need for replication, and biostatisticians have suggested that a lack of replication will restrict the use of formal statistical tests. Sources of variation and necessary levels of replication vary considerably among the array platforms to be employed, however Dudoit et al., have suggested that many considerations on replication are applicable to both cDNA and oligonucleotide platforms (Statist. Sincia. 2000; 12, 111-139).

The term biological replication can have two meanings; “actual biological replication” is the replication of array processing and hybridization involving mRNA from different extractions from the same sample or individual, and “biological replication”, where target mRNA comes from, e.g., different version of a cell line, or different individuals. These forms of replication are very different in nature, with the latter involving a much greater degree of variation in measurements (Yang et al., Nat Rev Genet. 2002; 3(8):579-88). For the efficient design of this study the choice of biological replication is very important. For example, it may be that variation between individuals will be larger than other sources of variation (i.e. experimental), and thus it may be inefficient to perform replicate arrays from a small number of samples. However, Simon et al., Genet Epidemiol. 2002; 23(1):21-36, have suggested several motivations for performing actual biological replication, as this replication provides an estimation of the reproducibility of the experimental procedures, it permits the identification and discarding of “bad” arrays, and actual biological replication can improve precision of the estimate of the expression profile for a given RNA sample though the averaging of multiple arrays. Furthermore, replicate samples are extremely useful when attempting to establish that a classification between diseases is robust, which is particularly true for class discovery algorithms, where the large number of genes make it relatively easy to discover interesting patterns of gene expression, even in random datasets.

The Affymetrix system provides a significantly lower variation between experiments, suggesting that the need for 3 or more replicates can be reduced. Additionally, each sample is processed in duplicate, thus performing actual biological replications. The above considerations, in particular that the robustness of the classification is deemed essential, coupled with the frequently reported use of duplicate hybridizations in Affymetrix oligonucleotide array experiments, and the use of actual biological replicates in two landmark papers on identification of breast cancer expression profiles (Perou et al., Nature 2000; 406(6797):747-52; Van t'Veer et al., Nature 2002; 415(6871):530-6) justifies the use of duplicate sample processing.

Data Analysis. All data analysis is performed as described for prostate cancer expression profiling in Example 3.

### Analysis of Leukocyte Gene Expression and the Multigene Expression Signatures Determined Following Data Analysis

Quantitative RT-PCR to Confirm the Results of the Microarray Experiments. For validation of microarray results, primers are designed to amplify a number of genes seen to be differentially regulated among leukocytes obtained from breast cancer patients and controls, and employed for assay via real-time RT-PCR of leukocyte transcript levels. The actual number of genes employed for validation of results depends on the number of genes found to be differentially expressed. Microarray experiments performed by other researchers, and cited above, are available as guidelines in determining the number of gene that need to be analyzed to validate the microarray results. Genes chosen for this analysis include those identified in previous studies that are differentially regulated between leukocytes from patients with a solid tumor relative to leukocytes from control subjects (and are thus positive controls), and also genes included in the multigene signatures deduced through the data analysis. For each gene analyzed, RT-PCR analysis is used to confirm and validate the outcome of the microarray analysis.

### Example 5

### Psychiatric Illness with Multigene Expression Classification

### Introduction

Previous studies have shown associations between white blood cell (leukocyte) gene expression levels and the psychiatric disorders bipolar disorder (BPD) and schizophrenia (SZ). As shown in the Example above, patients with SZ have a characteristic leukocyte multigene expression pattern or signature that differs from healthy control subjects. The positive expression data results collected for schizophrenics (n=8) and healthy controls (n=5), in addition to the contrasting gene expression differences reported between healthy controls, and patients with BPD or SZ [Spleiss et al., Mol Psychiatry 1998; 3, 512-20; Ilani et al., Proc Natl Acad Sci USA 2001; 98(2), 625-628)], establishes that specific leukocyte multigene expression profiles can differentially classify psychiatric illness.

This Example generates gene expression data from patients with BPD and SZ. The data create classifying multigene expression profiles for each of the disorders, using hierarchical clustering and supervised learning algorithms, that can be used to correctly distinguish leukocyte samples taken from patients with either BPD or SZ. This in turn leads to improved treatment targeting for patients with BPD and SZ, following classification with multigene expression profiles. This work also establishes the ability to define those at risk for the development of BPD and SZ based on the multigene expression signatures.

### Rationale

The psychiatric disorders to be investigated during this proposed study, BPD and SZ, have incidences in the general population of approximately 1%. Susceptibility to these disorders includes a large but variable genetic component, and there are efforts currently underway to find genes that play roles in the development of the diseases, through linkage analysis and association studies. Several chromosome regions and genes have been suggested as candidates for disease loci (Tsai et al., J Affect Disord 2001; 64, 185-93; Cloninger et al., Am. J. Med. Genet. 1998; 81, 275-281). Physical, biological and environmental factors such as birth trauma low birth weight, poor fetal nutrition, viral infection, autoimmune processes and winter/spring birth are also thought to contribute to the risk of developing BPD and SZ, as they may impact the developing brain either in utero or during postnatal development Kinney et al., J Affect Disord 1998; 50, 117-24; Gunduz et al., Schizophr Res. 1999; 40, 237-433). There are currently no genetic or biochemical markers or tests which can specifically predict the onset of these psychiatric illnesses or differentiate between the disorders.

A biological assay providing information that could help classify BPD and SZ, and define susceptibility at an early stage, especially in high risk families, may allow targeted treatment strategies to commence before the onset of many symptoms.

There is a growing literature illustrating the usefulness of global gene expression measurements in the characterization and classification of diseases and their subtypes, such as prediction of patient survival time, and response and sensitivity to treatment (see e.g., Sorlie et al., Proc Natl Acad Sci USA 2001; 98, 10869-74 In parallel to the above Examples, and of interest to this Example, Hoffman et al. recently described the first disease classification, by microarray analysis of brain tissue, between Rett syndrome patients and controls (Colantuoni et al., Neurobiol Dis. 2001; 8, 847-65).

To date, published microarray analyses of samples from patients with SZ and BPD have focused on analysis of post mortem brain tissue to investigate their etiologies. Upon microarray analysis of prefontal cortex, one group has suggested SZ is a disease of the synapse, and that expression analysis of genes involved in the regulation of presynaptic function may elucidate different sub-types or etiologies of SZ (Mirnics et al., Trends Neurosci, 2001; 24, 479-86). The results of a study employing Affymetrix GeneChips showed altered expression of genes involved in different functions, such as myelination, again providing detailed data on biological processes in the brain of SZ patients.

Although microarray analysis of brain samples has, and will provide important information on the etiology and pathogenesis of BPD and SZ, it is obvious that use of brain samples from living patients for molecular diagnostic classification is not feasible. Thus, the development of any multigene expression-based classification of BPD and SZ should focus on a tissue that is accessible. Peripheral blood samples are easily obtained and most significantly, it has previously been reported that SZ and BPD patients have altered levels of multiple gene products that are expressed in blood leukocytes. In a recent report, Ilani et al. measured the mRNA levels of the Dopamine D3 receptor gene in leukocytes from SZ patients and matched control subjects. They demonstrated that in SZ patients, transcripts of the D3 receptor were significantly elevated, and that this 2-3 fold increase in expression was not affected by antipsychotic drug treatments (typical or atypical). Moreover, non-medicated SZ patients were found to exhibit the same patterns of gene expression, suggesting that drug treatment itself does not effect gene expression of the D3 receptor in peripheral blood leukocytes (PBLs). A similar study performed on a larger patient population both confirmed the above observation and suggested that measurement of D3 receptor mRNA may also be useful in the classification of symptom severity subgroups Kwak et al., BMC Med Genet. 2001; 2(1):3).

Although an early report using post mortem brains described a decrease in D₃ receptor levels in SZ brains when compared to brains from control subjects, more recent studies have suggested that in some areas of the brain the D₃ receptor levels are increased in non-medicated SZ patients, and that the elevation is reduced by neuroleptic medication (Joyce et al., Ann N Y Acad. Sci. 1999; 877, 595-613).

Decreases in levels of D₃ receptor mRNA in PBLs have been observed in Parkinson's disease compared to controls with similar down-regulation of D₃ receptor in Parkinson's brains (Gurlnen et al., Nature 2001; 411, 86-9, while in Alzheimer's disease (AD), a reduction of PBL dopamine D₂-like receptors was reported (Barbanti et al., Mech Ageing Dev 2000; 120, 65-75, consistent with the levels of D₂-like receptors in brains of AD patients, compared to control subjects. The studies lend support to use of surrogate peripheral markers in classification of psychiatric/neurological disorders, although the question remains, whether peripheral markers simply reflect brain expression levels, or alternatively may have functions in disease processes. Interestingly Levite et al. concludes from a recent study, that dopamine receptor levels on human T-cells actually reflect increased/decreased lymphocyte functionality, and report their observations that upon stimulation by a dopamine receptor agonist that mimics the effect of dopamine, the 13 receptor expression on T-cells is stimulated and results in the further activation of T-cell function (Eur J Immunol. 2001; 31, 3504-12).

A study of leukocyte inositol monophosphatase (IMPase) mRNA from BPD patients and control subjects showed decreased expression in BPD, with the greatest decrease observed in non-drug treated patients (Nemanov et al., Int J Neuropsychopharmcol. 1999; 2, 25-29). Additionally, a measurement and comparison of leukocyte G protein alpha subunit mRNAs in BPD patients compared with mRNA levels in unipolar patients and control subjects, showed a significant increase of transcript levels in the BPD group compared to both other groups (Spleiss, supra).

Results from previous studies of BPD and SZ show alterations in the concentrations of immune response mediators in blood. There also appear to be differences between the disorders in the profile and magnitude of IRS mediator changes compared to control subjects, with the literature including instances of increased serum soluble Interleukin-2 (IL-2) receptor in BPD. In SZ there is further evidence to suggest the presence of altered leukocyte gene expression (see e.g., Lin et al., Schizophr Res. 1998; 32(1), 9-15), and although there are contradictory findings and some reports suggesting that neuroleptic medication may confound these studies by causing alterations in IRS markers, the majority of the studies using neuroleptic naive or non-medicated SZ patients show IRS activation in SZ. IRS gene products reported to be up-regulated in blood from SZ and BPD patients, and that are represented on the microarrays that will be utilized in the proposed study include; IL-6, IL-1 receptor antagonist (Akiyama et al., Schizophr Res. 1999; 37(1), 97-106, IL-2 and IL-2 receptor (Tsai et al, supra). CD4, CD8, CD4/CD8 ratio, CD3 (as measured by levels of CD+ cells in blood samples) [66; 67] and TNF-α. VLA-4 receptor expression on CD4+ and CD-8+T cells was also found to be increased in SZ, and differential regulation of the IRS-associated HSP-60 and HSP-70 have been observed in patients with SZ.

Most recently Tang et al have used a rat model to show global gene expression changes in leukocytes, that result from experimentally induced ischemic strokes, hemorrhagic strokes, sham surgeries, kainate-induced seizures, hypoxia, and insulin-induced hypoglycemia (Ann Neurol. 2001; 50, 699-707). The specific and characteristic patterns of multigene expression observed for each experimental state lends supports to the paradigm of “surrogate markers”, where a pathological insult or process may be confined to a particular organ or process, but can induce a characteristic alteration in the overall expression profile of circulating leukocytes, thereby demonstrating that medical and neurological diseases can cause disease-specific changes to gene expression in leukocytes.

Example 2, supra, reports that men with SZ exhibit a characteristic pattern of leukocyte gene expression that differs from the gene expression pattern of healthy control subjects, and is diagnostic for the disease. This preliminary study generated very encouraging positive data demonstrating that eight SZ patients exhibit a leukocyte gene expression pattern that differentiates them from five healthy controls subjects. Two BPD patients were also analyzed and were shown to cluster into a subnode of the tree diagram discreetly from the SZ subjects. However, the preliminary study involved microarray gene expression analysis of only a small number of SZ (n=8), BPD (n=2), and control subjects (n=5). This Example analyzes 25 male BPD and 25 male SZ subjects.

Although it has been suggested that the expression of single genes could be employed for the diagnosis of psychiatric disorders such as SZ, it seems clear that the measurement of multiple gene products as markers of disease provides considerably more detailed information for diagnosis and thus a more robust classifier than single marker analysis. Significantly, Hakak et al., showed a marked improvement in the separation of SZ subjects from control subjects when many brain markers were employed for analysis (n=35), compared to analysis of few markers (n-6), following linear discriminant analysis (Hakak et al., Proc. Natl. Acad Sci U.S.A. 2001; 98: 4746-51).

### Research Design and Methods

Overview. Microarray analysis measures the expression of leukocyte samples from 25 BPD and 25 SZ male patients between the ages of 25-60. Subjects are recruited from the residents of a psychiatric center and four community residential facilities. Gene expression data from the proposed study is analyzed employing hierarchical clustering, and supervised learning algorithms, and expression classifying signatures are identified (Ramaswamy et al., Proc Natl Acad Sci USA 2001; 98(26): 15149-54; Golub et al., Science; 286(5439):531-7).

Subject groups. Male White and African American SZ and BPD subjects are recruited into this study.

Medication Profiles of BPD/SZ Subjects. The BPD/SZ subjects recruited for this study primarily suffer from severe illness. In the primary study facility: (a) the SZ patient population comprised approximately, 35% paranoid, 35% residual and 20% disorganized SZ; (b) The BPD patients comprised approximately: 20% DSM 296.40 (most recent episode hypomanic), 15% DSM 296.44 (most recent episode manic, severe with psychotic features), 30% DSM 296.60 (most recent episode mixed, unspecified), 20% DSM 296.64 (most recent episode mixed, severe with psychotic features) and 10% DSM 296.80 (BPD NOS). Close to all of the patients were treated with neuroleptics during their admissions.

Subject Recruitment and Diagnosis. Patients are chart screened for eligibility. For patients interested in participating, informed consent is obtained in accordance with IRB regulations. Diagnostic interviews using the SCID will be conducted. The Brief Psychiatric Rating Scale (BPRS) (Overall et al., Psychol Rep. 1962; 10, 799-812) Clinical Global Impression (CGI) Mini-Mental State Exam (MMSE) (Folstein. J Psychiat Res. 1975, 12, 189-198), Scale for the Assessment of Negative Symptoms (SANS) [20-22], and Scale for the Assessment of Positive Symptoms (SAPS) will be conducted by the Research Nurse and the Psychiatrist C.I. at the patient's ward or residence.

Medical and Psychiatric Assessments and Exclusions. Chart records of patient subject's medical examinations including the admission examination is assessed. Medical and psychiatric history information is requested from facilities for all previously recorded admissions, for the purpose of defining a lifetime psychiatric diagnosis and to determine medical eligibility for the study. A lifetime medication history for each patient is also generated from hospital charts and records requested from other facilities.

A list of medical exclusions at the chart level has been generated and includes current or recent-infectious diseases, autoimmune diseases, proliferative disorders, and recent physical trauma or surgery, and chronic immunosuppressant or anti-inflammatory medication use.

Blood work. As part of the study procedure, CBC counts with differentials. CBC white cell counts outside of normal reference ranges, and clinically significant abnormal SMAC values or thyroid function test values will be used as exclusions.

Drugs screening. Results from urine screening for drugs of abuse including marijuana, cocaine, stimulants, barbiturates and heroin, performed at the time of admission are examined. Patients who test positive and those who refuse to be tested are excluded from the study. AU subjects are also questioned about cigarette smoking; number smoked/day and years of smoking are recorded. Alcohol intake and drug abuse history are also recorded.

Sample Collection. Fifteen ml blood samples are drawn from the antecubital vein by a study team research nurse at the patient's ward or residence. Bloods are processed immediately to isolate and purify leukocytes.

cRNA Synthesis and GeneChip Hybridization. High density Affymetrix GeneChip arrays were used in preliminary studies due to: 1) the large numbers of gene sequences represented within the array, 2) the highly developed protocols for probe preparation and microarray hybridization, and 3) the built-in multiple internal standards, plus custom designed normalization software for accurate comparison of results between each individual hybridizations. This latter point is of great importance, since the experimental plan involves a direct comparison between individual microarray experiments. Affymetrix Human U133A microarrays, which contain sequence-verified oligos representing nearly 20,000 individual genes, are employed to analyze gene expression signatures in blood leukocytes from the SZ and BPD subjects recruited during this study. This array is an upgraded version of the HU95A arrays employed in the preliminary studies. Both arrays contain all genes described above, and the arrays are comparable with each other. All blood samples are processed immediately following collection. All subjects samples chosen for RNA extraction are processed in duplicate, by splitting the leukocyte sample extracted from whole blood and processing them identically thereafter.

Replicate Sample processing. The need for replicate microarray experiments is axiomatic. Sources of variation and necessary levels of replication vary considerably among the different array platforms, however Churchill has suggested that many considerations are applicable to both cDNA and oligo platforms. The term biological replication can have two meanings; “actual biological replication” is replication of array processing and hybridization, involving mRNA from different extractions from the same sample or individual, and “biological replication” where target mRNA comes from different versions of a cell line, or different individuals. These forms of replication are very different in nature, with the latter involving a much greater degree of variation in measurements. Several reasons for performing actual biological replication have been suggested; this replication provides an estimate of the experimental reproducibility, it permits the identification of “bad” arrays, and actual biological replication can improve precision of the expression profile for a given RNA sample though the averaging of multiple arrays. Replicate samples are also extremely useful when attempting to establish that a disease classification is robust, which is particularly true for class discovery algorithms where the large number of genes make it relatively easy to discover interesting patterns of expression, even in random datasets. Each subject was processed in duplicate, and perform actual biological replications. The above considerations, in particular that the robustness of the classification is deemed essential, coupled with the reproducibility of Affymetrix arrays, hybridization protocols and scanning (mean r2=0.967 for repeat experiments justifies the use of duplicate sample processing.

Data Analysis. Affymetrix Software Suite is employed for image acquisition and normalization of the fluorescent signals. Analysis of signal intensities over each probeset within each experiment will fall into two main categories; Hierarchical Clustering (see e.g., Alizadeh et al., Nature 2000; 403(6769):503-11) and Supervised Learning Algorithms (Ramaswamy et al., supra). In addition, group difference testing is performed using SAS GLM procedures, including multivariate analysis of variance (MANOVA), used to test factors such as smoking status and medications as confounds in the group analyses. Finally, in the preliminary analysis a permutation analysis was employed to assess the subject cluster reliability. A Bootstrapping Cluster analysis will be implemented for reliability investigations.

Hierarchical Clustering. A hierarchical clustering algorithm Eisen et al., Proc Natl Acad. Sci. 1998; 95(25):14863-8), has been successfully applied to classify gene expression data (Alizadeh et al., supra), and is described in Example A, supra. Specifically, a Student's two-tailed t-test is performed across the genes expressed in the subjects leukocytes, and then employed Cluster to perform a supervised analysis on the genes found to be differentially expressed (p<0.1), resulting in firstly a classification of SZ and control subjects into their respective groups, and then a classification of BPD from SZ subjects. For this Example, these and other analysis of variance procedures are used for supervised cluster analysis of SZ and BD. The resultant clusters will represent multigene expression signatures specific for the diagnosis and that are useful for testing classification.

Supervised Learning Algorithms. Supervised learning algorithms are described in detail in Example A, supra.

Validation Using Quantitative RT-PCR. Microarray data are validated by real-time RT-PCR on genes randomly chosen from those observed to be differentially regulated among leukocytes obtained from psychiatric patients. Gene-specific primers are designed and employed for the SYBR Green PCR assay. Specifically, reverse transcribed cDNA is processed in duplicate from each patient RNA sample. Real-time PCR assays are then performed in triplicate for each cDNA sample. This experimental replication allows accurate confirmation and validation of the expression data from microarray analysis.

Additional Approach to the Development of Multigene Expression Signatures. The Affymetrix GeneChip human U133 series contains a second U133B array, with an additional 15,000 oligo sequences derived from characterized genes and non-redundant EST sequences. Use of this second array may extend the analysis with the aim of increasing the complexity of leukocyte specific multigene signatures.

This Example results in the creation of leukocyte multigene expression signatures that can classify leukocyte samples by the patient diagnostic groups (BPD and SZ), and that can be used to predict the class of unknown samples. Recruitment of additional patients from the subject groups ultimately allows the power of the expression signatures to be calculated. SZ and BPD-specific expression multigene expression signatures can be generated from multiple racial groups and female subjects, and further studies can determine the ability to assess or predict patient response to treatment based on leukocyte multigene expression signatures measured at admission, and/or by collection of longitudinal expression profile data following patient admission and during treatment, to determine correlates of treatment response. A longitudinal study of families with members at increased risk of developing psychiatric disorders because of illness in other family members can be performed. Gene expression patterns can be detected that classify psychiatric patients by diagnosis, are present in premorbid/prodromal subjects, and establish whether it is possible to predict risk of psychiatric illness from prodromal samples, potentially allowing for targeting of treatment to at-risk individuals such as those with schizotaxia. Disease-specific classification of psychiatric illness has multiple clinical uses, such as a diagnostic support to the psychiatrist on initial presentation of the patient. Also of major importance for psychiatric genetics research, multigene signatures can be employed to assay members of large SZ and BPD pedigrees employed for genetic linkage studies. Affected members, having an accurate biological classification of diagnosis, may help to avoid compounding errors in linkage studies.

### Example 6

### Schizophrenia Diagnosis with Leukocyte Multigene Signatures

This Example generates gene expression data from neuroleptic naive schizophrenic patients, in order to avoid the potential confounder of neuroleptic drug-derived gene expression changes. Additionally, an increased number of chronic neuroleptic-treated schizophrenics and healthy control subject's cases are tested in the gene expression dataset. The data generated in this proposed study, along with previously collected data, permit classifying multigene expression profile, using hierarchical clustering and supervised learning algorithms, that can correctly distinguish leukocyte gene expression levels of schizophrenic patients from control subjects. This in turn provides diagnostic information from leukocyte multigene signatures and defines those at risk for SZ development. This also establishes the ability to develop multigene expression signatures for other psychiatric disease.

### Background

Example 2, supra, generated very encouraging positive data demonstrating that SZ patients exhibit a leukocyte gene expression pattern that differentiates them from controls.

However, this study involved microarray gene expression analysis of only a small number of schizophrenic patients (n=8) and control subjects (n=5). This Example analyzes 32 male schizophrenic patients and 14 control male subjects, from multiple ethnic groups. Furthermore, the eight schizophrenic patients analyzed in the preliminary study had medication profiles that were diverse and included several different classes of atypical and typical neuroleptic medications: Subject 493: Olanzapine, Depakote, Risperidone., Subject 494: Chloral Hydrate, Zyprexa., Subject 495: Loxapine, Benztropine, Seroquel, Vistaril., Subject 535: Clozapine, Artane., Subject 588: Haloperidol, Haloperidol Decanoate, Cogentin, Depakote., Subject 630: Olanzapine, Risperidone., Subject 631: Haloperidol, Clozapine. There is growing evidence supporting disease specific alterations of leukocyte gene expression in SZ, but it has also been shown that neuroleptic medications can disturb IRS mediator concentrations in blood (Muller et al., Eur Arch Psychiatry Clin Neurosci 1997; 247: 308-13)].

In order to prove the presence of a signature, this Example performs the multigene expression analysis of neuroleptic naive schizophrenics, employing data analysis algorithms that identify common gene expression signatures between naive, and medicated SZ subjects, that can be utilized for classification of SZ subjects from healthy control subjects.

Measurement of Multiple Markers. Although it has been suggested that gene expression could be employed for the diagnosis of SZ using a single marker (Ilani et al., supra)], it seems clear that the measurement of multiple gene products as markers of SZ provides considerably more detailed information for identification of the disease and thus a more robust classifier than single marker analysis. Significantly, Hakak et al., supra, showed a marked improvement in the separation of SZ subjects from control subjects when many brain markers were employed for analysis (n=35), compared to analysis of few markers (n-6), following linear discriminant analysis. The use of multiple markers may in future also, e.g. allow the classification of biological subgroups of schizophrenic patients who respond to different treatments.

mRNA levels quantified by RT-PCR techniques is extremely time-consuming if many genes are analyzed in one experiment. By employing microarray technology, mRNA levels of thousands of genes expressed in peripheral blood leukocytes can be quantified, including genes coding for all of the markers described above. Global differential gene expression measured using the microarray approach identifies patterns or signatures of gene expression that differ between schizophrenic patients and control subjects, and thus form the basis of the diagnostic technique.

### Research Design and Methods

Overview. Microarray analysis measures the expression of leukocyte samples from 20 neuroleptic-naive SZ patients, 12 medicated SZ patients and 14 age-matched control subjects. Neuroleptic naive subjects are recruited from an urban emergency room. The study team clinical staff obtains informed consent, and a 15 ml blood sample is collected from each subject prior to a first neuroleptic dose. Blood samples are processed to isolate and purify the leukocytes and the samples are then stored. Patient notes and admission and discharge diagnoses are reviewed by the study team after twelve weeks, and samples from patients who have a confirmed SZ diagnosis will be further processed for microarray expression analysis. Neuroleptic-treated SZ patients are recruited from the residents of a psychiatric facility or community residential facilities. Control subjects are recruited from the staff. Gene Expression data from the proposed study are collated with the existing preliminary study data, and analyzed employing analysis of variance procedures, hierarchical clustering, and supervised learning algorithms.

Neuroleptic-Naive Schizophrenic Patients. Twenty neuroleptic naive SZ patients between the ages of 21-65 are completed during this study. Patients presenting at an ER are screened for inclusion in the study. It is estimated, that up to about 50% of the neuroleptic naive subjects initially considered to have SZ and recruited into this study, may later be diagnosed as having disorders other than SZ. Potential subjects are thus recruited and blood samples drawn but not processed to completion until retrospective formal diagnosis by the study team.

Subjects are recruited based on their initial psychiatric evaluation performed by a resident psychiatrist and nurse. For patients interested in participating, informed consent is obtained in accordance with regulations. The neuroleptic naive status of candidate patients is ascertained from a combination of sources including patient's report of their own status, and other significant sources such as patient's family member reports, and/or psychiatrist or therapist reporting from private care or if they have been outpatients at other facilities, and other collateral information. Patient's initial medical examination information is used to determine general health. Medical exclusion information for this study are ascertained by questioning of the subject and from family members and/or other collateral information. Medical exclusions include current or recent-infectious diseases, abnormal CBC counts, autoimmune diseases, proliferative disorders, and recent physical trauma or surgery, chronic immunosuppressant or anti-inflammatory medication use.

Retrospective Neuroleptic-Naive Subject Diagnosis. The initial SZ diagnosis given to a proportion of neuroleptic naive subjects who are recruited into this study could be changed during the course of their admission. In order to maximize the number of microarray-analyzed samples from subjects who are correctly categorized as SZ, initial blood processing on all neuroleptic-naive patients who carry either a SZ diagnosis or a “rule-out” SZ diagnosis, following initial assessment and SCID diagnosis, is performed. This initial process includes the isolation and purification of the leukocytes, and storage of samples at −70° C., which ensures RNA stability for >6 months (Qiagen). The study team reviews subject's notes and diagnosis twelve weeks after the subject's admission. This time period will allow for a fuller set of notes to be created, and also for acquisition of patient notes and history from any other sources or institutions. Additionally, if a subject has been discharged, his discharge diagnosis and summary are present/available in the notes. Following this retrospective confirmation of subject's diagnosis, 20 subjects were selected for GeneChip analysis.

Neuroleptic-Treated Schizophrenic subjects. Twelve male neuroleptic-treated SZ subjects between the ages of 21-65 are completed in this study. Subjects will be recruited from a psychiatric center and community facilities. Male residents of the five facilities are screened. Exclusions at the chart level will include a diagnosis other than SZ. Patients are interviewed as to their interest in participating in the study and informed consent is obtained in accordance with IRB regulations. Records from previous hospitalizations are obtained and also used to confirm the schizophrenia diagnosis. Medical exclusions will be identical to those described for neuroleptic naive patient.

Schizophrenia Diagnosis of Subjects. A psychiatric diagnostic and assessment interview is conducted by the study team using the SCID [5] in order to confirm the RPC chart diagnosis (neuroleptic-treated) or initial ER assessment (neuroleptic-naive) diagnosis for each subject. Patient records from previous treatment providers are obtained and also used to confirm the psychiatric diagnosis. Diagnostic interviews for the SCID will be conducted by the SCID trained members of the study team and the research nurse who is also SCID trained and certified. For neuroleptic-naive subjects, initial SCID diagnosis is retrospectively compared to subject's notes after 12 weeks, and only samples from subjects where there is agreement between the sources will be further processed for GeneChip analysis.

The Brief Psychiatric Rating Scale (BPRS), Clinical Global Impression (CGI), Mini-Mental State Exam (MMSE), Scale for the Assessment of Negative Symptoms (SANS) (Andreasen et al., Br J Psychiatry 1989; Suppl (7), 49-58. 89), and Scale for the Assessment of Positive Symptoms (SAPS) (Andreasen et al., Psychopathology 1995; 28: 7-17) are conducted by the study team or research nurse during the diagnostic and assessment interview. These scales are used to assist in the diagnostic process and to descriptively characterize the subjects.

Drugs Abuse Screening. Results from comprehensive urine screening for drugs of abuse including marijuana, cocaine, stimulants, barbiturates and heroin, performed at the time of admission or on the day of the study blood draw will be examined. Patients who refuse to be tested are excluded. Subjects are also questioned about cigarette smoking and number of cigarettes smoked per day.

Control Subjects. Fourteen male control subjects aged 21-65 are recruited from staff. The ages of the control subjects completed are defined by the patient sample and adjusted to maximize the similarity in ages between the groups. Controls complete a form (with the assistance of the study team) documenting that neither they nor their first degree relatives have a history of SZ, other psychotic disorders, mood disorders or of paranoid, schizoid, or schizotypal personality disorder. Current medication use and medical history are recorded. Medical exclusions are identical to those described for neuroleptic naive patients.

Blood Sample Collection, cRNA Synthesis and Hybridization. A fifteen ml blood sample is drawn from the antecubital vein by a phlebotomist or nurse. A CBC is performed on each blood sample. Blood is processed immediately to isolate and purify leukocytes, stored at 70° C. and stored for further processing. Leukocytes are extracted from blood samples immediately following collection. The leukocytes are stable at −70° C. (>6 months, Qiagen), and storage at that temperature allows the retrospective determination of which samples are to be hybridized to GeneChips, after a detailed analysis of all available patient history and a confirmed diagnosis of SZ. Samples chosen for RNA extraction are processed in duplicate, by splitting the extracted leukocyte samples and processing them identically thereafter. High density Affymetrix GeneChips and data analysis are described in Example 3.

Quantitative RT-PCR. Microarray analysis data are validated performing real-time RT-PCR on genes randomly chosen from those observed to be differentially regulated among leukocytes obtained from SZ patients and controls. Gene-specific primers are designed and employed for the SYBR Green PCR assay. Specifically, reverse transcribed cDNA is processed in duplicate from each patient RNA sample. Real-time PCR assays are then performed in triplicate for each cDNA sample. This replication should allow accurate confirmation and validation of the expression data from microarray analysis. This Example provides a leukocyte multigene expression signature that can classify leukocyte samples into SZ patient or control subject groups, which can be used to predict the class of unknown samples. A multigene expression signature that classifies leukocyte samples from both neuroleptic naive and medicated SZs is necessary because drug induced changes to gene expression patterns are a potentially confounding factor and may mask the disease specific signature for SZ. Recruitment of additional patients from all subject groups, and the inclusion of female subjects, ultimately will allow the power of the expression signatures to be calculated. This is facilitated by ongoing interactions with clinicians at all study sites, and should greatly facilitate the ultimate clinical application of the results.

This Example further establishes the ability to develop a database of specific leukocyte multigene expression signatures for other psychiatric disorders including bipolar disorder, schizoaffective disorder and major depression, which will in turn permit biological diagnosis of psychiatric patients. A longitudinal study, recruiting families with members at increased risk of developing SZ because of illness in other family members, is possible.

### Example 7

### Alzheimer's Disease Diagnosis with Leukocyte Multigene Signatures

The NINCDS-ADRDA and DSM-IV criteria are currently widely used for diagnosis of probable Alzheimer's disease (AD). These clinical criteria have a number of limitations, including lack of specificity and sensitivity in the diagnosis, and have an error rate of about 10% even in academic research centers. Furthermore, diagnosis based on cognitive function can only be made post symptomatically, at which time medications that may inhibit AD development or delay its progression will likely be ineffective. The imaging and biological marker diagnostic methods currently under development have additional drawbacks in terms of their need for highly specialized equipment, and specificity and sensitivity respectively, and thus may not be useful for early screening.

The present Example produces pilot data for development of a biological classification of AD patients, based on high-density microarray measurement of transcribed white blood cell (leukocyte) RNA. The rationale behind this proposal is based on two sources of data: 1) Current scientific literature, in which there is growing evidence that individuals with AD exhibit immune and other responses, that can be detected at the level of altered gene expression in circulating peripheral leukocytes. Quantitation of the mRNA transcripts in leukocytes of a number of individual genes has demonstrated associations between gene expression levels and the presence of AD. 2) Preliminary results from a microarray study by the PI, investigating gene expression changes in men with schizophrenia (Example 2, supra). Initial results from this expression study have been striking: supervised cluster analysis of peripheral leukocyte gene expression data, using transcript level measurements of thousands of genes from seven schizophrenic patients and five matched control subjects, resulted in a classification of all the subjects into their correct group. These results provide evidence to suggest that a surrogate tissue can be successfully employed for classification of a neuropsychiatric disease.

Utilizing a similar microarray strategy, this Example shows that individuals suffering from AD exhibit a conserved pattern of gene expression levels in their peripheral blood leukocytes, which is distinct from the pattern of expression in peripheral blood leukocytes from control subjects. This study provides a clinical assay that is minimally invasive, and has the capacity to identify AD sufferers, and can also provide important pre-symptomatic and early stage diagnostic information.

### BACKGROUND

Alzheimer's disease (AD) is the most common cause of degenerative dementia, representing about 65% of cases and affecting about four million Americans. Increased life expectancy, especially in the developed world has been accompanied by large increases in the AD rate, as its prevalence appears to double for every five years of age increase (Katzman et al., (2001) In Iqbal, K., Sisdia, S. S., and Winblad, B. (eds), Alzheimer's Disease: Advances in Etiology, Pathogenesis and Therapeutics. John Wiley and Sons, Ltd. Chichester, England, pp. 11-21). AD is believed to have a long preclinical phase, followed by a mild cognitive impairment (MCI), characterized by mild memory loss. AD dementia then follows with progressive deficits across multiple cognitive domains, including attention, memory, verbal ability, visuospatial skill, problem solving and reasoning, and along with stroke may be the third most common cause of death in the U.S. (Ewbank et al., Am J Public Health 1999; 89: 90-92). The growing economic and social costs of AD have made it a major public health issue, and prompted intensive study of its etiology and pathogenesis in order to facilitate development of preventative and therapeutic treatments.

Susceptibility to AD has a significant genetic component, and the discoveries of presenilin 1 and 2 (PS1, PS2), and amyloid precursor protein (APP) gene mutations that result in the familial forms of AD (early onset), have helped to elucidate the disease etiology Tandon et al., Genome Biol. Reviews 2002; 3: 3014). However, familial AD accounts for only approximately 2% of all AD cases and although genetic risk factors for sporadic AD have been identified, for example the presence of the epsilon 4 allele of Apolipoprotein E (APOE4) (Farrer et al., JAMA 1997; 278: 1349-56), many cases of AD do not carry the APOE4 allele and have no known associated gene mutations. Therefore the remaining genetic effect in AD has yet to be identified, and likely involves several genes of small effect. There are major efforts underway to find genes that play a role in the development of the sporadic AD, through linkage analysis and association studies.

Several chromosome regions and many potential genes, including the TNF-alpha and the estrogen receptor alpha genes have been suggested as possible candidates, although there are some concerns with candidate gene association reproducibility.

Epidemiological studies have begun to show that early detection and treatment of AD may be associated with a more favorable outcome, involving both overall risk and also progression and severity of disease. A biological assay providing information that could help identify and classify AD and define susceptibility at an early stage, especially in high-risk families, could provide a great public health benefit. Such an assay would potentially allow for targeted treatment strategies to commence before the onset of many symptoms. Recent studies have also indicated the need for early and accurate differential diagnosis of AD from other dementias.

Diagnosis of AD is commony performed using the NINCDS-ADRDA and DSM-IV criteria with direct patient assessment and interviews with family members. The criteria can provide a diagnosis of probable AD primarily based on cognitive function. Dementia severity can also be stratified according to the Mini-Mental State Examination (MMSE). Unfortunately, these diagnostic tools are inadequate for early diagnosis of abnormal changes in the brain that likely began long before cognitive impairment. Thus, even though highly skilled and experienced practitioners in a research center setting can achieve about 90% accuracy in patients meeting clinical criteria for dementia, several studies have documented the high levels of unrecognized dementia in the general community (Galasko et al., Arch Neurol 1994; 51: 888-95). In addition, it has also been shown that the clinical criteria are unable to predict neuroimaging findings, suggesting that brain imaging is currently necessary in the diagnostic evaluation of dementia (Chui et al., Neurology 1997; 49: 925-35). In order to improve the specificity of AD diagnosis and to develop pre-symptomatic and early stage diagnosis, neuroimaging (e.g., magnetic resonance imaging (MRI), positron emission tomography (PET), and single photon emission computed tomography (SPECT)) and biological marker detection techniques are under investigation in many studies. A few of these new methods and assays are described and the potential benefits and problems associated with each are discussed below.

Using fMRI, Bookheimer et al reported an increased magnitude of and extent of brain activation in the hippocampus, parietal and prefrontal cortex during a challenging memory test in subjects with the APOE4 allele, compared to those without an APOE4 allele, and concluded that during performance of a memory task, persons at risk of developing AD have preclinical compensatory increases in blood flow to those regions (Bookheimer et al., N Eng J Med 2000; 343: 450-6). However, neuroimaging techniques are sophisticated and relatively expensive, and require a high degree of operator skill and interpretation. Implementation of these methodologies into the general clinical setting may prove difficult, and even in specialist centers evaluation may take several hours of patient and clinician time. Additionally, it is expected that an increase in current specificity and sensitivity of the techniques may require further development for practical use

Methods for the early detection, and diagnosis of AD by measurement of biological markers in CSF are currently under development and include measures of Aβ, tau and phosphorylated tau proteins, as they are intimately involved in the senile plaques and neurofibrillary tangles of AD. CSF levels of Aβ are decreased, and levels of tau and phosphorylated tau are increased in AD. However, their levels are variable and neither has the sensitivity and specificity for routine use or for screening CSF-derived measurements of biomarkers requires that patients to undergo a lumbar puncture. The requirement for a lumbar puncture, which is a fairly invasive procedure that causes some discomfort, would probably mean a CSF-based assay would be unsuitable for population screening and for future pre-symptomatic detection of AD.

In a large study of non-demented-, non-AD demented-, and possible or probable AD-subject groups, utilizing urine AD7C-NTP measurements, Munzar et al showed significant differences between the subject groups. There was however, considerable overlap in urinary AD7C-NTP levels between the groups, showing a lack of specificity (Munzar et al., Neurol Clin Neurophysiol 2002; 2002: 2-8).

Measurements of Aβ42 in non-demented elderly subjects showed that, after 3 years, however, only those with upper quartile levels of Aβ42 were significantly more likely to develop AD than those in the lowest quartile (Mayeux et al. Ann Neurol 1999; 46: 412-16). However, other studies have found inconsistent findings.

Serum Melanotransferrin (P97) was assayed in a group of possible and probable AD subjects, and healthy controls and significantly higher P97 was found in the possible/probable AD group, although there was overlap between the subject groups (Feldman et al., J Alzheimers Dis 2001; 3: 507-16). In a similar study, Kim et al. measured serum P97 in controls, and AD and non-AD dementia subject groups and reported a significant difference between the AD group and the non-AD and normal control groups (also with the AD group elevated compared to the others), but no significant difference between the non-AD dementia group and the control group. α-1 antichymotrypsin (ACT) levels were measured in serum from AD, VD, and healthy control subjects and were found to be significantly higher in the AD group than the other two groups, although ACT levels in the VD and control groups showed no difference. However, a lack of specificity of serum marker was inferred by the overlap between subject groups.

Tan et al. measured the CD45RO and CD45RA isoforms of CD45 on T-cells from AD, MCI, non-AD dementia, and age matched healthy control subject groups. They found significantly lowered CD45RA and increased CD45RO/CD45RA ratio in the AD patient group and in the MCI group, compared to the healthy control subjects. The non-AD dementia group did not differ significantly from the healthy control group, and there was considerable overlap in the CD45 isoform levels between the subject groups.

Currently the CSF assays for Aβ and Tau have problems of specificity and sensitivity due to highly variable levels in CSF samples. Additionally, diagnostic assays requiring CSF samples are relatively invasive, would cause patient discomfort, may need a hospital setting and may require patient sedation. These factors may discourage use of CSF-based assays for population and pre-symptomatic screening, even if the assays themselves are improved. Although minimally invasive, the blood, blood-fraction and urine-based AD biomarker assays under development also have a relative lack of specificity.

Current antemortem AD diagnosis has variable accuracy and only produces a probable diagnosis. There is therefore a need for a sensitive and specific biological assay for AD diagnosis that can be performed using an accessible tissue, at relatively low cost, and without the requirement for sophisticated equipment at the site of sample collection. This would allow for regular screening of pre-symptomatic subjects, and could also be used to assess the effectiveness of medications in the prevention and/or delay of symptoms.

To date, published microarray analyses of AD have focused on analysis of post mortem brain tissue to investigate the etiology of AD. Areas of the brain affected by the progression of the disease have been studied with exciting early results. Using cDNA microarrays, Hata et al. identified genes found to be differentially expressed between AD brain hippocampus and parietal cortex (but not differentially expressed in control subjects brain), and suggested that these genes may be regulated in response to neurofibrillary tangle-related destruction and are thus potential therapeutic targets (Biochem Biophys Res Comm 2001; 284: 310-16). Further dissection of the hippocampus was performed by Colangelo et al., who employed Affymetrix arrays to identify gene expression specific to AD in the hippocampal cornu ammonis 1 (J Neurosci Res 2002; 70: 462), while Loring et al., investigated expression in AD cingulate and amygdala brain sections (DNA Cell Biol. 2001; 20: 683). Strikingly similar results were reported from both studies, including the generalized depression in brain gene transcription, decreases in many known transcription factors, neurotrophic factors, and signaling elements involved in the synaptic pathway and also the up-regulation of genes involved in inflammatory, stress and immune and responses. These experiments have thus employed a global gene expression analysis to validate several theories of AD pathology and have identified pathways for future drug targeting.

Although microarray analysis of brain samples has and will provide important information on the etiology and pathogenesis of AD in brain tissue, it is obvious that use of brain samples from living patients for molecular diagnostic classification is not feasible. Thus, the development of any multigene expression-based classification of AD should focus on a tissue that is accessible. Peripheral blood samples are easily obtained and most significantly, it has previously been reported that patients with AD have altered levels of multiple gene products that are expressed in blood leukocytes.

In a recent study, Schipper et al. measured plasma levels of HO-1 protein in early sporadic AD, normal elderly control (NEC), normal younger control, age-associated cognitive decline (AACD), non-AD dementia, non-dementing neurologic illness and chronic medical disorder groups of subjects (Neurology 2000; 54: 1297-1304). The authors found that compared to the NEC group, the AD group had significantly lower HO-1 protein levels. Lymphocyte HO-1 mRNA levels were also measured for each subject, and were found to be significantly lower in AD relative to NEC, and levels were also found to be decreased compared to the AACD, non-AD dementia, non-dementing neurologic illness, and chronic medical condition groups. In addition, HO-1 mRNA levels were also lower in the AACD group compared to the NEC group suggesting a use for this transcript as a peripheral marker of both AD and age-associated cognitive decline. Transcript levels of the heat shock protein HSP-70 were also reported as a potential marker for AD. mRNA levels of HSP-70 in mononuclear blood cells were measured by Northern blot analysis, and although no correlation was observed between HSP-70 and aging, mRNA levels were found to be significantly lower in AD patients when compared to both VD patients and non-demented control subjects. In addition to the reports described above, further evidence for differential gene expression in AD leukocytes comes from multiple studies describing changes in the immune system in AD patients that result in abnormalities of peripheral blood lymphocytes, such as the multiple increases in circulating and in-vitro produced cytokines including CD4, CD25, and CD28 antigen, and decreases in CD7 and CD8, and the increase in T-lymphocyte IL-6 receptor 62. It therefore seems likely that for multiple genes, differential gene expression will be associated with the alteration in T-cell phenotype and dysfunctional immunity in AD.

Example 2, supra, reports that men with schizophrenia (SZ) exhibit a characteristic pattern of leukocyte gene expression, that differs from the gene expression pattern of healthy control subjects, and would thus be diagnostic for the disease. This study has generated very encouraging positive data by demonstrating that SZ patients exhibit a leukocyte gene expression pattern that differentiates them from controls. In addition, the seven schizophrenic patients analyzed in the study had medication profiles that were diverse and included several different classes of atypical and typical neuroleptic medications, providing some evidence to suggest that SZ subject classification from control subjects is not directed by a specific medication profile. As reported below, these studies now include the analysis of additional subject numbers, including neuroleptic naive SZ subjects, to allow further development of a SZ leukocyte classifier. Data for comparison of multigene expression signatures between different psychiatric disorders are being generated. One major depression patient and two bipolar disorder patients, all of whom were receiving neuroleptics are recruited. Preliminary cluster analysis of leukocyte gene expression data showed a distinct separation of the bipolar and major depression subjects from SZ subjects, with an internal separation of bipolar from major depression. This data does show that the leukocyte expression signatures are disease specific and can be used to classify between different neuropsychiatric disorders.

Although it has been suggested that gene expression could be employed diagnostically for AD, using single markers, it seems clear that the measurement of multiple gene products as markers of AD provides considerably more detailed information for identification of the disease, and thus a more robust classifier than single (or dual) marker analysis. A recent investigation on SZ supports this assumption; Hakak et al., showed a marked improvement in a brain expression classification of SZ subjects from control subjects when many brain markers were employed for analysis (n=35), compared to analysis of few markers (n-6), following linear discriminant analysis.

### Design and Methods

Overview. Microarray analysis measures the expression of leukocyte samples from 20 AD patients and 20 age-matched healthy control subjects. The study team obtains informed consent, and a 15 ml blood sample is collected from each subject prior to initial medication. Blood samples are processed to isolate and purify the leukocytes and the samples are stored prior to RNA purification, cRNA synthesis and GeneChip hybridization and scanning. Gene Expression data is analyzed by ANOVA testing, and by employing hierarchical clustering, and supervised learning algorithms.

Subject groups. Male AD patients and control subjects from all ethnic groups are recruited. There have been multiple reports in the literature of the ability of microarray analysis to accurately classify disease tissues even though micro-dissection was not performed to remove multiple non-disease cell types within the tissue. Additionally, a recent report illustrated a classification of leukemia when whole blood was employed for initial RNA extraction (Armstrong et al., Nat Genetics 2002; 30: 41-47). These papers suggest that the algorithms employed to determine signatures of gene expression are not confounded by either complex tissues (with only a sub-section containing the cells of interest), or inter-subject variation of genes in total peripheral blood. It is unlikely that expression variability due to ethnicity will mask an AD-specific leukocyte multigene expression signature.

Recruitment of AD Patients. AD subjects are recruited based on their initial evaluation and a diagnosis of probable AD. Candidate patients are approached and interviewed as to their interest in participating in the study. For patients interested in participating, informed consent is obtained. If possible, recruitment is limited to patients who have not yet received medication for AD, however medicated patients may be recruited into the study to ensure completion. Evidence from the SZ studies (Example 2, supra) suggest that neuroleptic medication does not primarily direct and/or mask leukocyte classifiers of disease. In addition, and if necessary for the AD research, subjects receiving a diverse range of medication treatments are recruited. This approach will decrease the likelihood that detected gene expression patterns are induced by a specific medication.

Patient's initial medical examination information is used to determine general health. Medical exclusion information for this study is ascertained by questioning of the subject and from family members and/or other collateral information. Medical exclusions include current or recent-infectious diseases, autoimmune diseases, proliferative disorders, and recent physical trauma or surgery, chronic immunosuppressant or anti-inflammatory medication use. Patients with CBC white cell counts outside of normal ranges are also excluded.

These selection and exclusion criteria have been designed to reduce the likelihood of detecting AD leukocyte gene expression patterns that differ from matched control subject gene expression patterns, but that arise not from the disease process but from other factors such as medication or the presence of an infectious agent.

Male Control Subjects. Twenty male control subjects are recruited from the staff and the local community. Subjects are in the age range of 65 and older. Control subject age is defined by the patient sample as the ages of the control subjects are adjusted to meet the mean age of the patients, so as to maximize the similarity in ages between the groups. Thus control subject recruitment is initiated following the completion of AD subject recruitment. Control subjects are asked to complete a form documenting that neither they nor their first-degree relatives have a history of AD. Forms are also completed listing current medication use and medical history. Medical exclusions are identical to those described for AD patients above.

Blood Sample Collection. Fifteen ml Blood samples are drawn from the antecubital vein. A CBC is performed on each blood sample. Bloods are processed immediately to isolate and purify leukocytes, and stored for further processing.

Quantitative RT-PCR. Microarray analysis data are validated as described above by performing real-time RT-PCR on genes randomly chosen from those observed to be differentially regulated among leukocytes obtained from AD patients and controls. This Example results in the creation of a leukocyte multigene expression signature that can classify leukocyte samples into AD patient or control groups and can be used to predict the class of unknown samples (using a supervised learning approach). Recruitment of additional patient and control subjects and the inclusion of female subjects, allows the power of the expression signatures to be calculated. The data generated from this work permits investigation of the specificity of the multigene expression signatures by generating expression signature data for different forms of non-AD dementia. Longitudinal studies can be designed to generate multigene expression pattern data from pre-clinical subjects at risk of AD (through familial mutations or APOE4 alleles), and to investigate the feasibility of early diagnosis of AD utilizing multigene expression signature data.

Gene expression patterns that classify AD patients can be determined to be present in subjects prior to the onset of symptoms. It is thus possible to predict risk of AD from pre symptomatic subject's samples, potentially allowing for targeting of treatment to at-risk individuals.

A diagnosis of AD with improved specificity and sensitivity has multiple clinical uses, such as a diagnostic support to the clinician on initial presentation of the patient. Also of major importance for AD genetics research, multigene signatures could be employed to assay members of AD pedigrees employed for genetic linkage studies. Affected members, having an accurate biological classification of diagnosis may help to avoid compounding errors in linkage studies.

### Example 8

### Detection of Genetic Alterations Through Gene Expression in Surrogate Samples

Surrogate tissue can also be used to identify genetic defects or sequence alterations, such as mutations or polymorphisms, associated with, or resulting in, or contributing to, a physical state or susceptibility to a physical state. Genes/ESTs/sequences are shown to have altered expression in a surrogate tissue between the “disease” and “healthy” samples or subjects, and are potential candidates for having DNA mutations or alterations such as polymorphisms, that are related to the disease or physical state of interest.

The benefit of this objective is that it will necessitate sequencing of a smaller number of genes, to identify candidate “disease” genes, than currently used in other methods for discovering “disease” genes. Also, use of the present method for analysis of gene expression in surrogate tissues (e.g., blood leukocytes) allows freedom of subject choice, and in the case of SZ, does not require access to postmortem brain tissue, or tumor biopsy tissue for the identification of susceptibility genes for cancer development.

This method can be employed for any physical state with a genetic component. Specific applications for SZ and prostate cancer are outlined below in Examples 8A and 8B. A list of candidates for further examination for prostate cancer is provided in Example 8B.

### Example 8A

### Schizophrenia

Schizophrenia (SZ) is a complex disorder with a high heritability and approximately ten-fold increased risk in first-degree relatives. Genome scans are widely used in the search for SZ linkage regions, as prerequisite for identification and mutation screening of candidate SZ susceptibility genes. Studies to date possess a number of limitations, including lack of reproducible, strong linkage findings, and the large breadth of chromosomal areas identified, which can contain potentially hundreds of genes.

It is also believed that multiple genes of small or moderate effect may contribute to SZ susceptibility, and therefore need to be identified within the linkage regions. However, linkage studies have highlighted a number of chromosomal regions that may harbor genes that contribute to SZ. The difficult task is to identify susceptibility alleles among the large numbers of genes within these regions. Sequence analysis and association testing for all the genes within regions of linkage would be an overwhelming task and a more focused approach for candidate gene identification of is required. One embodiment of this method is designed, based on integration of linkage and gene expression data, for discovery and validation of SZ candidate genes.

Feasibility of this embodiment of the method was investigated using preliminary study gene expression measurements (from Example 2 above) of about 12,000 genes and ESTs from eight SZ patients and five control subjects (CS).

These preliminary study findings were very positive: 9774 genes and ESTs were mapped to the genome, and sorted and ranked by significance level of differential expression. In this particular example, genes were considered to be “expressed” if they had a GeneChip intensity of ≧100 intensity units (IU) (intensity values that were calculated through Affymetrix MAS 5.0 from a scaling factor of 100 for the data), and 1042 of the mapped, “expressed” genes were differentially expressed (p<0.05) between the eight SZ subjects and five healthy CS groups (note that use of an additional SZ subject has increased the number of genes found to be significantly differentially expressed from that described in Example 2).

Mapped-gene expression data were then filtered using increasing GeneChip intensity thresholds, and the ten top ranking genes were each scored as mapping either to a region of SZ linkage (1), or to another genome region (O). The ten top ranked gene's scores were summed and recorded. When all mapped genes were included in the analysis (zero intensity filter) 2/10 genes fell within a region of linkage. A filter of increasing expression level stringency was applied in 20 IU increments, excluding genes for which less than two subject's IU values equaled or exceeded the IU threshold for that gene. Thirty complete, independent sets of randomized mapping data were generated and used to determine the frequency of random gene mapping to a linkage region.

For the real GeneChip expression data, when the IU cutoff reached 100, the number of linked genes climbed from 2/10 to 4/10. This was of interest because it was considered that GeneChip IU levels ≧100 indicated real gene expression, rather than background signal. As the IU filter threshold was further increased, the number of linkage regions genes within the top ten rose, reaching a maximum score of 6/10 at cutoff IU levels between 560 and 620. Scores of 6/10 between the 560 and 620 IU cutoffs were considered to be significantly higher than the background linkage region scores for the same IU cutoffs (p=0.028).

Since higher IU levels reflect increasing gene expression, the peak of SZ-linked region genes between the 560 and 620 IU cutoffs indicates the range of expression levels at which the noise of the system from in-specific differential gene expression has been filtered out. The remaining genes show disease-specific differential gene expression. Therefore, the overabundance or enrichment of top ranking genes that map to SZ linkage regions, seen at those cutoff levels, may provide the best candidate genes for DNA sequence analysis to search for gene and/or promoter, enhancer or splicing mutations.

At IU cutoffs over 620, the number of SZ-linkage region genes then fell back as the threshold was increased, dropping to a plateau of 2/10 at an IU cutoff of 720. The decreased representation of SZ-linked region genes in the top ten differentially expressed genes at IU cutoffs greater than 620 may be due to increasing representation of leukocyte-specific gene expression at these higher levels. This representation is likely due to, or reflective of, alterations in leukocyte expression of immune response mediator (IRS) and other genes, previously reported for SZ, and also due to the multigene expression patterns observed in the preliminary data for this study. Using this preliminary data, it was discovered that among the genes most significantly differentially expressed in leukocytes, between SZ and control subjects, there is a significant overrepresentation of genes from areas of reported linkage to SZ.

### Methods

Autosomal genes were sorted by increasing two tailed t-test significance level of differential expression (p value). For the purposes of this example genes/ESTs were designated “expressed” if they had a GeneChip intensity of ≧100 intensity units (IU), and 1042 of the mapped, expressed genes were found to be differentially expressed (p<0.05) between the SZ and healthy CS groups.

Mapped-gene/EST expression data were then filtered using increasing GeneChip intensity thresholds, and the ten top ranking genes were each scored as mapping either to a region of SZ linkage (1), or to another genome region (O). The ten top ranked gene/EST's scores were summed and recorded. When all autosomal mapped genes/ESTs were included in the analysis (zero intensity filter) 2/10 genes/ESTs fell within a region of linkage.

Genome mapping. Genes and ESTs represented as oligonucleotide probe-sets on the Affymetrix HU95A version 2 arrays, were mapped to their chromosomal sequence locations using the Ensemble Human Genome Browser (80%) and NCBI Human Genome Resource databases (20%). A total of 9774 genes and ESTs were mapped using these automated approaches, Genes without mapping data were excluded from the dataset.

A sample of genes and ESTs (n=81) that had not been mapped by the automated approach, were mapped manually. There was no significant difference in the proportion of linkage area genes, when the manual and automated mapping approaches were compared (p=0.689), indicating that the automated gene mapping approach was not biased in the genes that it mapped.

### Results and Discussion

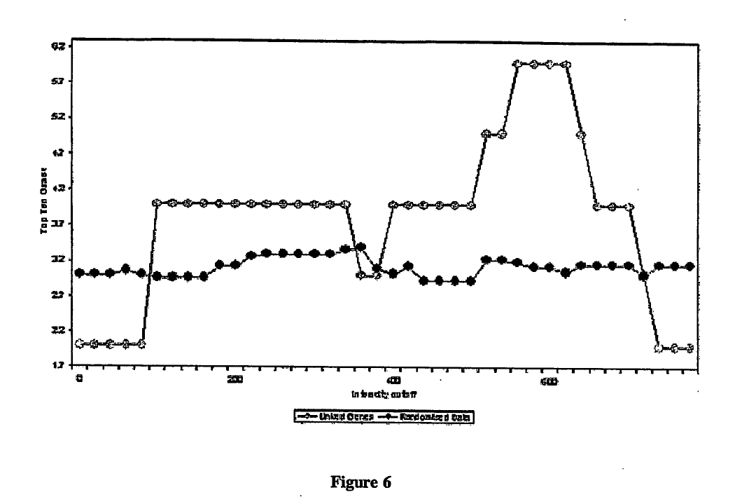

As illustrated in FIG. 6, a filter of increasing expression level stringency was applied in 20 IU increments, excluding genes for which less than two subject's IU values equaled or exceeded the IU threshold for that gene. Genes/ESTs that mapped to regions of linkage were assigned a score of 1. Genes/ESTs mapping to other areas of the genome were scored 0. The dataset was filtered with increasing stringency, using signal intensity cutoffs in 20 unit steps (i.e., ≧0, 20, 40, 60, . . . ). For each intensity cutoff, the number of genes/ESTs within the top 10 of all genes/ESTs, that map to regions of linkage were counted, and the y-axis values for the filled red circles each indicate the sum total of linked genes/ESTs within the top 10 genes/ESTs that were present, using the x-axis intensity cutoff level. The filled black circles indicate sum total of randomly occurring linkage areas within the top ten gene/ESTs. Thirty complete, independent sets of randomized mapping data were generated and used to determine the frequency of random gene mapping to a linkage region.

This reasoning was based on the hypothesis that IU levels reflected increasing gene expression. Therefore, the peak of SZ-linked region genes between the 560 and 620 IU cutoffs indicates the range of expression levels at which the noise of the system from in-specific differential gene expression has been filtered out, leaving genes that show disease-specific differential gene expression. Accordingly, the overabundance or enrichment of top ranking genes that map to SZ linkage regions, seen at those cutoff levels, may provide the best candidate genes for DNA sequence analysis to search for gene and/or promoter and/or enhancer mutations or alterations.

A recent genome scan meta-analysis (GSMA) was used to select linkage regions for this preliminary analysis of gene expression data. (Lewis et al. Am. J. Hum. Genet. 2003; 73:34-48). In this approach a rank-based meta-analysis was applied to autosomal data from 20 genome scans. Marker data was assigned to individual 30-cM bins and the bins were ranked by linkage scores, with weightings for sample sizes. Permutation testing was used to calculate the probabilities of the observed bin ranks, and 19 autosomal regions were identified where p<0.05 for weighted and/or unweighted analyses. Accordingly, in one embodiment, genes/ESTs identified in the present invention that map to the regions identified in the Lewis study are considered as being potentially SZ susceptibility loci.

The results demonstrate that that thirty three percent of the genes and ESTs were mapped to regions where linkage has been reported in a genome scan meta-analysis of 20 genome scans (Lewis et al., Am. J. Hum. Genet. 73:34-48, 2003).

Prevalence of Significantly Differentially Expressed Genes is Enriched in Areas of Linkage to SZ. The total number of genes that map to SZ-linked areas were then compared with the total for genes that map to non SZ-linked areas of the genome. Interestingly, there was a 3.83 fold excess over expected values of significantly differentially expressed genes (p<0.05) mapped within SZ-linkage areas, compared to the total number of genes/ESTs that map to areas of SZ linkage. This enrichment finding further suggests that some of these differentially expressed genes may be good candidates for being “disease or Susceptibility genes” for SZ.

Linkage Data is not a Prerequisite or Requirement for Practice of the Invented Method.

In many complex diseases, disorders and physical states, linkage data is not strong or reliable, or may not be available. One preferred embodiment of the present invention involves utilization of altered expression of surrogate tissue in a subject or subjects, for the identification of candidate sequences for testing by sequence analysis, without further selection based on whether genes/ESTs or nucleotide sequences lie at or near a region reported or considered to be linked to the disease, disorder or physical state being investigated.

### Example 8B

### Prostate Cancer

There is also substantial evidence of a significant hereditary component in susceptibility to—and of familial aggregation of—prostate cancer (PCa), with epidemiological studies having demonstrated a 2-3 fold increased risk of PCa amongst first-degree relatives of PCa patients (Whittemore et al, Am J Epidemiol. 1995, 141, 732-40). Although there are issues of heterogeneity, multiple studies have identified areas of linkage to the disease (Easton et al., The Prostate, 57: 261-269, 2003; Janer et al., The Prostate, 57: 309-319, 2003; Brown et al., Brit J. Cancer, 90: 510-514, 2004; Witte et al., The Prostate, 57: 298-308, 2003; Cunningham et al., The Prostate, 57:335-346, 2003; Verhage et al. Familial Cancer, 2: 57-67, 2003). Several of the linkage regions have been identified and confirmed in independent populations. Regions identified to date include 1p36, 17q11, 19 p13, 20q13 and Xq27-28. These results to date indicate the presence of multiple PCa susceptibility loci, and several individual genes within the regions have been identified as potential candidate PCa susceptibility alleles, these include RNA-SEL and ELAC2 (Carpten et al., Nat Genet, 30: 181-184, 2002; Tavtigian-S V., Nat Genet, 27:172-180, 2001).

The methods of the present invention were used to analyze expression data from men with PCa (n=11) and male control subjects (n=7). About 40% of the genes and expressed sequence tags (ESTs), represented on the HU95A version 2 GeneChip microarrays, used in this example were considered to be expressed (by the selected cutoff) in the leukocyte samples used, indicating that this accessible surrogate tissue is useful for the discovery and/or identification of candidate genes/ESTs by measurement of differential expression of genes/ESTs. About 599 genes were significantly differentially expressed between the PCa patient and control subject groups (p<0.05).

Mapping to the human genome was performed as described above.

### Results

Differentially Expressed Genes Map to Areas of PCa Linkage. When the differentially expressed genes were ranked by significance level and mapped to the human genome as above, 55% of the 20 most significant genes were mapped close to regions of published replication-confirmed linkage to PCa. In order to control for any potential issues of PCa-linked genome regions possibly being over represented on the microarray, and to investigate the number of PCa linkage region genes that would be expected to appear in the top 20 by chance alone, repeated randomizations of the data were performed, and these were found to consistently result in about 20% of the top 20 genes mapping within regions of linkage to prostate cancer.

This strongly suggests to that the present invention will be useful for the rational discovery and/or detection and/or assay of potential candidate genes for mutation screening.

### Example 8C

### Potential Candidate Genes or ESTs

Initial examination of pilot expression data and linkage regions has indicated a number of genes that are differentially expressed in PCa, and that map to regions of PCa linkage. Several are described below.

On candidate gene which was found to be significantly differentially expressed between PCa patients and healthy controls, and that maps to a region of linkage, is the potassium voltage-gated channel, shaker-related subfamily, beta member 2 (HKvbeta2.2) gene, which was mapped to 1p36.3 (within 6cM of the positive LOD score region). This gene also was found to be upregulated in PCa subject group (p=0.000041) (Gibbs et al., Am J Hum Genet, 64: 776-787, 1999). This gene is of additional interest because there is evidence of voltage-gated potassium ion channel protein overexpression in PCa specimens, and potassium channel blocking agents demonstrated growth inhibition in the LNCaP prostate tumor cell line (Abdul and Hoosein, Cancer Letters, 186: 99-105, 2002).

A second potassium channel gene that is significantly differentially expressed between PCa patients and healthy controls, and that maps to a region of linkage, is the Shaw type potassium voltage-gated channel Kv3.3 (KCNC3) gene. This gene was mapped to 19q13.3-q13.4, and was upregulated in PCa subject group (P=0.0017).

These findings indicate the utility of this invention for discovery, identification, detection, and evaluation of genes that are likely candidates for involvement in PCa susceptibility. Use of surrogate tissues and/or cells and/or organs (in PCa, peripheral blood leukocytes) permits of subject choice, and in the case of PCa, does not depend on the ability to acquire normal prostate or prostate tumor tissue, thus broadening the availability of samples by avoiding the requirement for a prostate biopsy.

### Example 8D

### Proposed Study for Identification of Candidate Genes in Schizophrenia

This proposed study is designed to test the feasibility of expression and linkage mapping as a method for discovering candidate genes within linkage regions, and to perform mutation analysis of the candidate genes. The longer term aims for this research are to extend this research to other psychiatric disorders and other diseases, disorders and physical states and all ethnicities.

Study Design. Blood leukocytes from twenty male and female SZ patients of non-Hispanic Caucasian ethnicity and twenty healthy control subjects between the ages of 2165 will be collected over the two year period of this study. Affymetrix GeneChip microarray (e.g., U133A) technology will be employed to measure global gene expression in the leukocyte samples, and significance testing will be conducted to identify genes differentially expressed between the two subject groups.

Genes and ESTs that are significantly differentially (p<0.05) expressed between the patient and control groups will be finely mapped to their genomic locations. The alignment settings will be stringent, only matches that have greater that about 98% identity or less than or more than 98% identity will be considered. In addition, significantly differentially expressed genes and ESTs that map within or near flanking markers of linkage to SZ will be cataloged and sorted by patient/control differential expression significance or level. Genes that map between or near the two markers of regions of linkage that has been will be included. Particular focus may be on areas previously shown or suggested to be linked to SZ, may include eg. 1q21-22, 6p22-24m, 6q21-22, 8p21m 10p1-15, 13q32, 22q11-13, and may also include 1q23.3-q31.1, 2p12-q22.1, 3p25.3-p22.1, 5q23.2-q34, 11q22.3-24.1, 6pter-p22.3, 2q22.1-q23.3, 1p13.3-q23.3, 8p22-p21.1, 6q15-q23.2, 6p22.3-p21.1, 10pter-p14, 14pter-q13.1, 15q21.3-q26.1, 16 p13-q12.2, 17q21.33-q24.3, 18q22.1-qter, 20 p12.3-p11, 22pter-q12.3 (Lewis et al., Am J Hum Genet. 2003; 73(1):3448).

Candidate genes cataloged as described above that have altered expression between the patient and control groups and that may also be included based on other factors eg. known or predicted to be expressed in the brain, will be selected The candidate genes/ESTs or sequences, including 5′ and 3′ untranslated regions, controlling regions and all intron/exon boundaries will be sequenced in all patients and controls to determine mutations or sequence alterations.

Future studies such as evaluation using gene chips or other microarrays or other technologies, with more genes/ESTs or sequences (e.g., U133 plus 2.0 from Affymetrix), may also include the investigation of genes/ESTs or sequences that have altered expression or eg. are differentially regulated between subjects with and without, and between different psychiatric disorders such as bipolar disorder and major depression and other disease, disorders or physical states.

### Example 9

### Refinement of Analysis to Detect Genetic Alterations

The following methods can be used, (either individually, or in combinations of one or more additional methods), but are not a requirement for the practice of the invention. One or more of these refinements can be used in conjunction with the initial invention to facilitate identification genetic defects by evaluating RNA expression in “surrogate” tissue.

Refinement One. Employment of the present method preferentially selects evaluation of genes or ESTs or other sequences of interest that are physically located within, near, or in the region of an area of linkage to the disease, disorder or physical state of interest. Such selection increases the likelihood that sequencing of a candidate loci meeting this criteria will yield a mutation or other genetic defect or alteration that is related to the disease, disorder or physical state of interest.

Refinement Two. The present method employs expression level-based exclusion filtering criteria to remove potentially spurious and/or non-relevant RNA expression data from data sets, following identification of candidates as described above. This technique can be applied by utilizing lower and upper expression level cutoffs. This is relevant to the present invention since it may be difficult to identify candidates among very low level expressors. Therefore, by using a “surrogate” or non-directly related biological sample tissue, any observed differential expression may be a product of non-physiological expression alterations. This rationale also applies in the case of high expressors, again because of the use of “surrogate” or non-directly related biological sample source. In this case, high expressors can be excluded as being of physiological importance in that sample or subject, unless, there is evidence the genes/ESTs/sequences under investigation have physiological relevance to the sample.

Refinement Three. The present method employs statistical testing to determine the significance of the differential expression between experimental groups being tested, i.e. “disease” and “healthy” or different physical state groups. This enables sorting or ranking of the genes/ESTs/sequences under investigation by the significance of their differential expression. Their relative significance can then be a factor in the selection of candidate genes/ESTs/sequences that are further selected for sequencing in search of genetic alterations or defects.

Refinement Four. A fourth refinement is the use of the size and/or degree of expression difference between experimental groups being tested, i.e., between “disease” and “healthy” or physical state groups. The genes/ESTs/sequences under investigation can then be sorted and/or ranked by the size and/or degree of their differential expression, and their relative expression difference size will then be a factor in the selection of candidate genes/ESTs/sequences that are further selected for sequencing in search of genetic alterations or defects.

Refinement Five. The present invention also exploits expression information relating to the disease and/or condition and/or state under investigation. Information from studies or databases or other sources can be utilized as a method for filtering genes/ESTs/sequences to aid in the choice of candidates for further investigation by sequencing or other methods. Utilization of disease specific, tissue specific, or other specific expression information could also be a factor in deciding whether to exclude or include genes/ESTs/sequences from further analysis.

Refinement Six. Another refinement concerns use of expression information relating to organs, tissues, cells that are related to the disease or physical state under investigation. Information from studies or databases or other sources can be utilized as a method for filtering genes/ESTs/sequences to facilitate the selection of candidates for further investigation by sequencing or other methods.

This additional method is best applied by using it as a factor in the selection of candidates for further investigation, i.e., assessing whether genes/ESTs/sequences under consideration are expressed or differentially expressed or have altered expression, in tissues associated with to the disease or physical state under investigation. Thus, for example, for schizophrenia, preference or priority for further investigation may be given to genes/ESTs/sequences that are expressed in the brain or central nervous system. Conversely these type of criteria could also be utilized in exclusion genes/ESTs/sequences from further analysis.

Refinement Seven. This invention exploits information concerning gene, loci, sequence and expression information relating to the disease, disorder or physical state under investigation. Information from studies or databases or other sources can be utilized as a method for selecting genes/ESTs/sequences to measure/assay based on expression levels in order to assess samples for the potential presence of mutated and/or altered genes and/or sequences. For any disease, disorder or physical state under investigation, information from studies or databases or other sources is utilized to generate listings of genes/ESTs/sequences as potential candidates. For example, where a previous study has named a gene as being of interest or shown association with, or has suggested biological or genetic expression or activity or function, in a disease, disorder or physical state, there is a rationale for its consideration as a candidate disease gene.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

1. A method for evaluating a physical state of a subject, which method comprises comparing (i) an expression profile of surrogate cells from the subject with (ii) a normal expression profile of surrogate cells from a normal subject or subjects, wherein a difference between the expression profiles is indicative of the physical state of the subject under investigation.

2. A method for evaluating a disease or disorder of a subject, which method comprises comparing (i) an expression profile of surrogate cells from the subject with (ii) a normal expression profile of surrogate cells from a normal subject or subjects, wherein a difference between the expression profiles is indicative of the disease or disorder of the subject under investigation.

3. (canceled)

4. (canceled)

5. (canceled)

6. The method according to claim 2, wherein the subject is a human.

7. The method according to claim 2, wherein the surrogate cells are peripheral blood leukocytes.

8. The method according to claim 7 wherein the peripheral blood leukocytes are selected from the group consisting of monocytes, macrophages, lymphocytes, granulocytes, neutrophils, basophils, and eosinophils, or other white blood cell types or subtypes.

9. The method according to claim 2, wherein the disease is the presence of a cancer in the subject.

10. The method according to claim 9, wherein the cancer is prostate cancer or breast cancer.

11. (canceled)

12. The method according to claim 2, wherein the disease is the presence of a neurological disorder.

13. The method according to claim 12, wherein the neurological disorder is a neurodegenerative disease or Alzheimer's disease.

14. (canceled)

15. The method according to claim 2, wherein the disorder is a psychiatric disorder or a mood disorder.

16. The method according to claim 15, wherein the disorder is selected from the group consisting of schizophrenia, bipolar disorder and major depression.

17. (canceled)

18. (canceled)

19. (canceled)

20. (canceled)

21. (canceled)

22. (canceled)

23. (canceled)

24. (canceled)

25. The method according to claim 2, wherein evaluating the disease or disorder is selected from the group consisting of classifying the disease or disorder, diagnosing the presence of a disease or disorder, determining the prognosis of the subject, monitoring a therapy, selecting a therapy, and assessment of susceptibility for the disease or disorder.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. The method according to claim 2, which comprises obtaining an expression profile on a nucleic acid microarray.

40. The method according to claim 39, wherein the microarray is an oligonucleotide microarray or a cDNA microarray.

41. (canceled)

42. The method according to claim 2, which comprises obtaining an expression profile with reverse transcriptase-polymerase chain reaction (RT-PCR).

43. A method for evaluating a physical state of a subject, which method comprises comparing an expression profile of surrogate cells from the subject with an expression profile of surrogate cells from a known subject or subjects determined to have the physical state, wherein a similarity in the expression profiles indicates that the subject has the same physical state as the known subject.

44. A method for monitoring a physical state of a subject, which method comprises comparing an expression profile of surrogate cells from the subject with an expression profile of surrogate cells from a known subject or subjects determined to have the physical state and have a known degree of that physical state, wherein a similarity in the expression profiles indicates that the subject has a similar degree of that physical state as the known subject.

45. A method for evaluating a treatment or therapy in a subject, which method comprises comparing an expression profile of surrogate cells from the subject after the treatment or therapy with an expression profile of surrogate cells from the subject prior to treatment or therapy, wherein a difference in the expression profiles indicates an effect of the treatment or therapy on the subject.

46. The method according to claim 45, whereby the treatment is exposure to a candidate therapeutic compound.

47. A method for evaluating a treatment or therapy in a subject, which method comprises comparing the expression profile of the subject after exposing the subject to the treatment or therapy with a normal expression profile of surrogate cells from a normal subject or subjects, wherein a similarity of the expression profiles is indicative of a therapeutic benefit of the treatment or therapy on the subject.

48. The method according to claim 47, whereby the treatment is exposure to a candidate therapeutic compound.

49. A method for evaluating a treatment or therapy in a subject, which method comprises comparing the expression profile of the subject after exposing the subject to the treatment or therapy with an expression profile of surrogate cells from other subjects with the same physical state prior to exposure to different therapies, wherein a similarity of the expression profiles is indicative of low treatment or therapy benefit on the subject.

50. The method according to claim 49, whereby the treatment is exposure to a candidate therapeutic compound.

51. (canceled)

52. (canceled)

53. (canceled)

54. (canceled)

55. A method for predicting a response to treatment or therapy in a subject, which method comprises comparing an expression profile of nucleic acids from surrogate cells from the subject prior to exposing the subject to a treatment or therapy, with an expression profile of nucleic acids from surrogate cells from other subjects with the same physical state prior to exposure to different therapies, wherein a similarity in the expression profiles predicts an effect of the treatment or therapy on the subject based on the effect of that therapy on another subject or subjects having a similar expression profile.

56. A method for choice of treatment or therapy for a subject, which method comprises comparing an expression profile of nucleic acids from surrogate cells from the subject prior to exposing the subject to a treatment or therapy with an expression profile of nucleic acids from surrogate cells from other subjects with the same physical state prior to exposure to different treatment or therapies, wherein a similarity in the expression profiles predicts an effect of the treatment or therapy on the subject based on the effect of that therapy on another subject or subjects having a similar expression profile.

57. (canceled)

58. (canceled)

59. (canceled)

60. (canceled)

61. (canceled)

62. (canceled)

63. (canceled)

64. A method for identifying a nucleic acid containing a sequence alteration that results in and/or contributes to a disease or disorder, and/or results in and/or contributes to susceptibility for a disease or disorder, which method comprises (a) selecting a nucleic acid that has altered expression in a surrogate cell from a subject with the disease or disorder, when compared to a surrogate cell from a normal subject or subjects; and (b) comparing the sequence of the nucleic acid, including the entire transcribed region, plus upstream and downstream controlling elements, from the subject with disease or disorder and the normal subject or subjects, wherein a sequence difference indicates that the nucleic acid sequence results in and/or contributes to a disease or disorder, and/or results in or contributes to susceptibility for a disease or disorder.

65. The method of claim 64, wherein the nucleic acid is adjacent to, near to, or within, a region of genetic linkage to the physical state.

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. The method according to claim 64, wherein the disorder is a psychiatric disorder or a mood disorder.

79. The method according to claim 78, wherein the disorder is selected from the group consisting of schizophrenia, bipolar disorder and major depression.

80. (canceled)

81. (canceled)

82. (canceled)

83. (canceled)

84. (canceled)

85. A method for diagnosing a disease or disorder in a subject by the detection of a nucleic acid alteration in said subject, wherein the nucleic acid alteration is identified using the method of claim 64.

86. A method for determining the prognosis of a subject having a disease or disorder by the detection of a nucleic acid alteration in said subject, wherein the nucleic acid alteration is identified using the method of claim 64.

87. (canceled)

88. A method for determining the susceptibility of a subject for developing a disease or disorder by the detection of a nucleic acid alteration in said subject, wherein the nucleic acid alteration is identified using the method of claim 64.

89. (canceled)

90. A method for developing therapeutic compounds to be administered to a subject with a disease or disorder resulting from and/or contributed to, by a nucleic acid sequence alteration identified by the method of claim 64, whereby the therapeutic compounds are designed to normalize the function or expression of the altered nucleic sequence.

91. (canceled)

92. (canceled)

93. A method for treating a patient suffering from a disease or a disorder resulting from and/or contributed to, by a nucleic acid sequence alteration that had been previously identified using the method of claim 64, comprising administering to a patient in need of such treatment therapeutically effective amounts of a normal counterpart of the nucleic acid sequence.

94. (canceled)

95. (canceled)

96. (canceled)

97. A method for treating a subject with a disease or disorder resulting from and/or contributed to, by an altered expression level of a nucleic acid identified to have altered expression using the method of claim 2, comprising administering to a subject in need of such treatment therapeutically effective amounts of the nucleic acid or therapeutically effective amounts of inhibitory nucleic acid sequence specific for the nucleic acid.

98. (canceled)

99. (canceled)

100. (canceled)

101. (canceled)

102. (canceled)

103. (canceled)

104. A method for developing therapeutic compounds for a disease or disorder resulting from and/or contributed to, by an altered expression level of a nucleic acid identified to have altered expression using the method of claim 2, whereby the therapy is designed to normalize the function or expression of the nucleic sequence.
